SEQUENCE LISTING (27 sequences)

SEQ ID NO: 1 - DNA, 1143 bp, Bacillus lentus
  CDS (1)..(1140)
  sig_peptide (1)..(81)
  mat_peptide (334)..(1140)
atgaagaaaccgttggggaaaattgtcgcaagcaccgcactactcatttctgttgcttttagttcatcgatcgcatcggctgctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattagccgtgtgcaagccccagctgcccataaccgtggattgacaggttctggtgtaaaagttgctgtcctcgatacagggatatccactcatccagatctaaatattcgtggtggcgcaagctttgtaccaggggaaccgtcgactcaagatgggaatgggcatggcacgcatgtggccgggacgatcgctgctttaaacaattcgattggcgttcttggcgtagctcctagcgctgagctatacgctgttaaagtcctaggggcgagcggttcaggttcggtcagctcgattgcccaaggattggaatgggcagggaacaatggcatgcacgttgctaatttgagtttaggaagcccttcgccaagtgccacactcgagcaagctgttaatagcgcgacttctagaggcgttcttgttgtagcggcatctgggaattcaggtgcaggctcaatcagctatccggcgcgctatgcgaacgcaatggcagtcggagctactgatcaaaacaacaaccgcgctagcttttcacagtatggcgcaggccttgacattgtcgcacccggggtaaacgtgcagagcacatacccaggttcaacatatgccagcttaaacggtacatcgatggctactcctcatgttgcaggtgcggccgcccttgttaaacaaaagaacccatcttggtctaatgtacaaattcgaaatcatctaaagaatacggcaactagtttaggaagcacgaacttgtatggaagcggacttgttaacgcagaagcggcaacgcgttaa

SEQ ID NO: 2 - protein, 380 aa, Bacillus lentus
MKKPLGKIVASTALLISVAFSSSIASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 4 - protein, 270 aa, Bacillus lentus
AQSVPWGISRVQAPAAHNRGLTGSGVKVAVLDTGIDSTHPDLNIRGGASFVPGEPSTQDGNGHGTHVAGTIAALDNSIGVLGVAPSAELYAVKVLGASGSGSVSSIAQGLEWAGNNGMDVANLSLGSPSPSATLEQAVNSATSRGVLVVAASGNSGAGSISYPARYANAMAVGATDQNNNRASFSQYGAELDIVAPGVNVQSTYPGSTYASLNGTSMATPHVAGAAALVLQKNPSWSNVQIRNHLKNTATSLGSTNLYGSGLVNAEAATR

SEQ ID NO: 5 - protein, 275 aa, Bacillus amyloliquefaciens [sequence omitted]

SEQ ID NO: 6 - protein, 269 aa, Artificial sequence
  Synthetic construct
AQSVPWGIERVQAPAAHNRGLTGSGVKVAVLDTGISTHPDLNIRGGASFVPGEPSTQDENGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVLSTYPGSTYASLNGTSMATPHVAGAAALVKQKNPSWSNVQIRNHLKNTATSLGSTNEYGSGLVNAEAATR

SEQ ID NO: 7 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGPGLDIVAPGVNVLSTWPGSTYASLNGTSMATPHVAGAAALVKQKNPSWSNVQIRNHLKNTATSLGDTNEYGSGLVNAEAATR

SEQ ID NO: 8 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTAAALDNSIGVLGVAPSAELYAVKVLGASGSGSVSSIAQGLEWAGNNGMHVANLSLGSPSPSATLEQAVNSATSRGVLVVAASGNSGEPSISYPARYANAMAVGATDQNNNRASFSQYGPGLDIVAPGVNVQSTYPGSTYASLNGTSMATPHVAGAAALVKQKNPSWSNVQIRNHLKNTATSLGSTNEYGSGLVNAEAATR

SEQ ID NO: 9 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGPGLDIVAPGVDILSTWPGGTYAVLNGTSMATPHVAGAAALVKQKNPSWSNVQIRNHLKNTATSLGSTNEYGSGLVNAEAATR

SEQ ID NO: 10 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGEPEISYPARYANAMAVGATDQNNNRASFSQYGPGLDIVAPGVDILSTWPGGTYAVLNGTSMATPHVAGAAALVKQKNPSWSNVQIRNHLKNTATSLGSTNEYGSGLVNAEAATR

SEQ ID NO: 11 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGADEISYPARYANAMAVGATDQNNNRASFSQYGPGLDIVAPGVDILSTWPGGTYAVLNGTSMATPHVAGAAALVKQKNPSWSNVQIRNHLKNTATSLGSTNEYGSGLVNAEAATR

SEQ ID NO: 12 - protein, 270 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMDVANLSLGSPSPSATLEQAVNSATSRGVLVVAASGNSGEPEISYPARYANAMAVGATDQNNNRASFSQYGPGLDIVAPGVDILSTWPGGTYAVLNGTSMATPHVAGAAALVKQKNPSWSNVQIRNHLKNTATSLGSTNEYGSGLVNAEAATRR

SEQ ID NO: 13 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGPGLDIVAPGVDILSTWPGGTYAVLNGTSMATPHVAGAAALVKQKNPSWSNVQIRNHLKNTATDLGSANEYGSGLVNAEAATR

SEQ ID NO: 14 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSDSPSATLEQAVNSATSRGVLVVAASGNSGAGSISYPARYANAMAVGATDQNNNRASFSQYGPGLDIVAPGVDILSTWPGGTYAVLNGTSMATPHVAGAAALVKQKNPSWSNVQIRNHLKNTATDLGSANEYGSGLVNAEAATR

SEQ ID NO: 15 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGEPEISYPARYANAMAVGATDQNNNRASFSQYGPGLDIVAPGVNVLSTWPGSTYASLNGTSMATPHVAGAAALVKQKNPSWSNVQIRNHLKNTATSLGDTNEYGSGLVNAEAATR

SEQ ID NO: 16 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGADEISYPARYANAMAVGATDQNNNRASFSQYGPGLDIVAPGVNVLSTWPGSTYASLNGTSMATPHVAGAAALVKQKNPSWSNVQIRNHLKNTATSLGDTNEYGSGLVNAEAATR

SEQ ID NO: 17 - protein, 271 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMDVANLSLGSPSPSATLEQAVNSATSRGVLVVAASGNSGEPEISYPARYANAMAVGATDQNNNRASFSQYGPGLDIVAPGVNVLSTWPGSTYASLNGTSMATPHVAGAAALVKQKNPSWSNVQIRNHLKNTATSLGDTNEYGSGLVNAEAATRHH

SEQ ID NO: 18 - protein, 270 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMDVANLSLGSPSPSATLEQAVNSATSRGVLVVAASGNSGEPEISYPARYANAMAVGATDQNNNRASFSQYGPGLDIVAPGVNVLSTWPGSTYASLNGTSMATPHVAGAAALVKQKNPSWSNVQIRNHLKNTATSLGDTNEYGSGLVNAEAATRR

SEQ ID NO: 19 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGPGLDIVAPGVNVLSTWPGSTYASLNGTSMATPHVAGAAALVKQKNPSWSNVQIRNHLKNTATSLGDANEYGSGLVNAEAATR

SEQ ID NO: 20 - protein, 269 aa, Artificial sequence
  Synthetic construct
AQSVPWGIERVQAPAAHNRGLTGSGVKVAVLDTGISTHPDLNIRGGASFVPGEPSTQDENGHGTHVAGTIAALDNSIGVLGVAPSAELYAVKVLGASGSGSVSSIAQGLEWAGNNGMHVANLSLGSPSPSATLEQAVNSATSRGVLVVAASGNSGEPEISYPARYANAMAVGATDQNNNRASFSQYGAGLDIVAPGVNVLSTYPGSTYASLNGTSMATPHVAGAAALVKQKNPSWSNVQIRNHLKNTATSLGSTNEYGSGLVNAEAATR

SEQ ID NO: 21 - protein, 270 aa, Artificial sequence
  Synthetic construct
AQSVPWGIERVQAPAAHNRGLTGSGVKVAVLDTGISTHPDLNIRGGASFVPGEPSTQDENGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVDVLSTWPGSTYASLNGTSMATPHVAGAAALVKQKNPSWSNVQIRNHLKNTATSLGSTNEYGSGLVNAEAATRR

SEQ ID NO: 22 - protein, 269 aa, Artificial sequence; identical to SEQ ID NO: 10
  Synthetic construct

SEQ ID NO: 23 - protein, 269 aa, Artificial sequence; identical to SEQ ID NO: 11
  Synthetic construct

SEQ ID NO: 24 - protein, 269 aa, Artificial sequence; identical to SEQ ID NO: 11
  Synthetic construct

SEQ ID NO: 25 - protein, 270 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGPELDIVAPGVDILSTWPGGTYAVLNGTSMATPHVAGAAALVKQKNPSWSNVQIRNHLKNTATSLGSTNEYGSGLVNAEAATRR

SEQ ID NO: 26 - protein, 269 aa, Artificial sequence
  Synthetic construct
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGPELDIVAPGVDILSTWPGGTYAVLNGTSMATPHVAGAAALVKQKNPSWSNVQIRNHLKNTATSLGSTNEYGSGLVNAEAATR

SEQ ID NO: 27 - protein, 269 aa, Artificial sequence; identical to SEQ ID NO: 26
  Synthetic construct

(19) United States
(12) Patent Application Publication
(10) Pub. No.: US 2017/0175098 A1
(43) Pub. Date: Jun. 22, 2017

(54) Enzyme Variants and Polynucleotides Encoding Same

(71) Applicant: Novozymes A/S, Bagsvaerd (DK)

(72) Inventors: Poul Erik Pedersen, Farum (DK); Jon Martin Persson, Bjaerred (SE); Esben Peter Friis, Herlev (DK); Simon Glanville, Kalundborg (DK)

(73) Assignee: Novozymes A/S, Bagsvaerd (DK)

(21) Appl. No.: 15/127,539

(22) PCT Filed: Mar. 30, 2015
(86) PCT No.: PCT/EP2015/056896

§ 371 Date: Sep. 20, 2016

(30) Foreign Application Priority Data
Mar. 28, 2014 (EP) 14162434.6

(51) Int. Cl. C12N 9/54; C11D 3/386

(52) U.S. Cl. CPC C12N 9/54; C12Y 304/00; C11D 3/386; C11D 3/38681

(57) ABSTRACT

The present invention relates to enzyme variants. The present invention also relates to polynucleotides encoding the variants; nucleic acid constructs, vectors, and host cells comprising the polynucleotides; methods of using the variants and composition comprising variants.

### REFERENCE TO A SEQUENCE LISTING

This application contains a Sequence Listing in computer readable form, which is incorporated herein by reference.

### BACKGROUND OF THE INVENTION

Field of the Invention

The present invention relates to protease variants, polynucleotides encoding the variants, methods of producing the variants, and methods of using the variants.

Description of the Related Art

Enzymes have many commercial applications in various industries and enzymes are widely used in the detergent industry. The most relevant detergent enzymes are enzymes such as proteases, amylases, lipases and cellulases, each having its unique stain removal properties e.g. proteases cleave proteinaceous stains. Thus these enzymes are essential for removal of stains and spotting on textiles and hard surfaces in laundry and dish wash processes. Many of enzymes used in the detergent industry are optimized to perform its unique stain removal actions usually by protein engineering of the enzyme altering the amino acid compositions of the enzyme. The protein engineered enzymes are optimized for increased performance in cleaning processes such as laundry and dish wash however the alterations in the primary sequence may reduce the solubility of the enzymes which could be a problem in production and purification processes. Also many wild type enzymes have low solubility.

The present invention provides variants of proteins such as proteases with improved solubility compared to its parent. Preferably these variants have retained or even improved performance, such as increased wash performance, increased substrate specific performance and/or improved stability, such as storage stability compared to a reference enzyme.

### SUMMARY OF THE INVENTION

The present invention relates to proteins comprising 2 to 6 histidines at positions corresponding to positions on the surface of the mature polypeptide of the protein, wherein the protein have increased solubility below pH 5 compared to a protein having identical amino acid sequence except the 2 to 6 histidines at positions on the surface of the mature polypeptide of the protein.

The present invention also relates to polynucleotides encoding the variants; nucleic acid constructs, vectors, and host cells comprising the polynucleotides; and methods of producing the variants.

The present invention also relates to methods of producing a protein having increasing the solubility below pH 5 compared to the parent protein comprising substituting histidines at 2 to 6 positions corresponding to positions on the surface of the mature polypeptide of the protein.

The present invention also relates to methods of producing a protein having increasing the solubility below pH 5 compared to the parent protein comprising insertion of 2 to 6 histidines adjacent to positions corresponding to positions on the surface of the mature polypeptide of the protein.

One aspect of the invention relates to a method of producing a variant of a parent protein wherein the variant has at least two additional histidines on the surface compared to the parent, the method comprising introducing into the parent protein alterations to obtain additional 2 to 6 histidines at the surface of the mature polypeptide of the parent protein, wherein each alteration is independently a substitution or insertion.

The invention further relates to a method of producing a variant of a parent protein, wherein the variant has at least two additional histidines on the surface compared to the parent protein comprising the steps of:
a) identifying amino acid positions at the surface of the mature polypeptide of the parent protein;
b) selecting at least one amino acid position among the positions identified in a) which is not occupied by a histidine in the parent protein; and
c) substituting amino acid selected in b) with histidine; and
d) expressing the modified nucleic acid sequence in a host cell to produce the variant.

The invention further relates to a method of producing a variant of a parent protein, wherein the variant has at least two additional histidines on the surface compared to the parent protein comprising the steps of:
a) identifying amino acid positions at the surface of the mature polypeptide of the parent enzyme;
b) selecting at least one amino acid position among the positions identified in a); and
c) inserting one or more histidine adjacent to the at least one amino acid position selected in b); and
d) expressing the modified nucleic acid sequence in a host cell to produce the variant.

One aspect of the invention relates a protein variant, which when compared to the parent protein comprises 2 to 6 additional histidines at positions corresponding to positions at the surface of the mature polypeptide of the parent.

One embodiment of the invention relates to protein variant, which when compared to the parent protein comprises 2 to 6 additional histidines at positions corresponding to positions at the surface of the mature polypeptide of SEQ ID NO: 2, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity to SEQ ID NO: 3. One aspect of the invention relates to a protease variant, wherein the variant compared to SEQ ID NO 3, comprises 2 to 6 of the following substitutions: A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H. In one embodiment the protease variant comprises one or more of the substitutions G20H, T22H, Y89H, N115H, Q135H, S139H, R143H, N232H, V238H, N242H or N246H. One aspect of the invention relates to a protease variant wherein the variant when compared to SEQ ID NO 3 comprises 2 to 6 of the following insertions: *1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH, *110aH,*111aH,*114aH,*115aH,*116aH,*118aH,*125aH,*126aH,*127aH,*128aH,*129aH, *131aH,*132aH, *134aH,*135aH,*138aH, *139aH, *142aH,*143aH, *144aH, *153aH,*154aH, *156aH,*157aH, *158aH,*161aH, *164aH,*166aH, *167aH,*175aH,*176aH,*177aH, *178aH, *179aH,*180aH, *182aH,*183aH,*186aH, *188aH, *189aH,*198aH, *200aH, *203aH,*204aH, *205aH, *206aH, *207aH, *208aH, *210aH, *211aH, *212aH, *229aH, *230aH, *231aH, *232aH, *233aH, *234aH, *235aH, *236aH, *238aH, *239aH,*241aH,*242aH,*243aH,*245aH,*246aH, *249aH, *250aH, *251aH, *252aH, *253aH, *254aH, *255aH, *256aH, *257aH, *259aH, *269aH.

In one aspect, the variant comprises one or more of the insertions *20aH, *22aH, *89aH, *115aH, *135H, *139aH, *143H, *232aH, *238aH, *242aH and *246aH. In one aspect the variant has increased solubility at pH 5 and/or improved wash performance compared to a SEQ ID NO 3. In one aspect of the invention the histidine modified protease variants or variants of a protease parent wherein the protease parent is a protease comprising at least e.g., at least 65%, at least 70%, at least 75%, at least 80%, at least 85%, at least 90%, at least 95%, at least 96%, at least 97%, at least 98%, or at least 99% sequence identity to SEQ ID NO: 3. In one aspect the protease parent comprises SEQ ID NO 3. In one aspect the variants further comprising one or more substitutions at positions selected from the group consisting of positions 3, 4, 9, 15, 24, 42, 59, 66, 74, 76, 97, 99, 101, 102, 118, 126, 127, 128, 154, 156, 157, 158, 161, 164, 176, 179, 182, 188, 198, 199, 200, 203, 210, 211, 212, 216, 239, 255 and 256, preferably positions 9, 15, 42, 66, 74, 97, 99, 154, 200, 203, 211, 212 and/or 256 (numbering according to SEQ ID NO: 3). The present invention further relates to detergent compositions and washing processing such as laundry or dish wash comprising a variant protein comprising 2 to 6 histidines at positions corresponding to positions on the surface of the mature polypeptide of the protein.

### DEFINITIONS

The term “allelic variant” means any of 2 to 6 alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant

The term “detergent composition” includes, unless otherwise indicated, all forms of detergent compositions such as gel, granulate, liquid, paste, powder, spray or tablet compositions including heavy-duty liquids (HDL), fine-fabric liquid detergents, liquid and/or solid laundry detergents and fine fabric detergents; hard surface cleaning formulations for e.g. glass, wood, ceramic and metal counter tops and windows; carpet cleaners; oven cleaners; fabric fresheners; fabric softeners; textile and laundry pre-spotters, as well as dish wash detergents such as hand dishwashing agents, light duty dishwashing agents, machine dishwashing agents; all-purpose or heavy-duty washing agents, liquid, gel or paste-form all-purpose washing agents, liquid cleaning and disinfecting agents, including antibacterial hand-wash types, cleaning bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types.

In addition to containing a histidine modified variant of the invention, the detergent formulation may contain one or more additional enzymes (such as proteases, amylases, lipases, cutinases, cellulases, endoglucanases, xyloglucanases, pectinases, pectin lyases, xanthanases, peroxidaes, haloperoxygenases, catalases and mannanases, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

The term “dish washing composition” refers to all forms of compositions for cleaning hard surfaces. The present invention is not restricted to any particular type of dish wash composition or any particular detergent.

The term “expression” includes any step involved in the production of a protein variant according to the invention including, but not limited to, transcription, post-transcriptional modification, translation, post-translational modification, and secretion.

The term “expression vector” means a linear or circular DNA molecule that comprises a polynucleotide encoding a variant and is operably linked to control sequences that provide for its expression.

The term “hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, and cutlery such as spoons, knives, and forks, serving utensils, ceramics, plastics such as melamine, metals, china, glass and acrylics.

The term “fragment” means a polypeptide having one or more (e.g., several) amino acids absent from the amino and/or carboxyl terminus of a mature polypeptide; wherein the fragment has the enzyme activity of the mature polypeptide. In one aspect, a fragment contains at least 269 amino acid residues (e.g., amino acids 1 to 269 of SEQ ID NO: 2), at least 200 amino acid residues (e.g., amino acids 1 to 200 of SEQ ID NO: 2), or at least 150 amino acid residues (e.g., amino acids 1 to 150 of SEQ ID NO: 2).

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

The term “improved property” means a characteristic associated with a variant that is improved compared to the parent. Such improved properties include, but are not limited to, catalytic efficiency, catalytic rate, chemical stability, oxidation stability, pH activity, pH stability, specific activity, stability under storage conditions, substrate binding, substrate cleavage, substrate specificity, substrate stability, surface properties, thermal activity, and thermostability.

The histidine modified protein variants of the invention preferably have in addition to increased solubility at least one improved property compared to the parent protein. In a particular aspect of the invention the variant has increased solubility compared to the parent at pH below 5 and improved wash performance and/or improved stability such as improved storage stability. In a preferred aspect the variant is a variant of a polypeptide having at least 60% identity to the mature polypeptide of SEQ ID NO 2 wherein the variant has increased solubility at pH below 5 and/or improved wash performance and/or improved stability compared to SEQ ID NO 3.

The term “solubility” is the amount of protein in solution or the maximum amount of protein in solution. The term “improved solubility” is defined herein as a variant protein displaying an alteration of the solubility relative to the parent protein (i.e. relative to a protein having the identical amino acid sequence of the variant but excluding the alterations in the variant), such as relative to the mature polypeptide of SEQ ID NO: 2. Solubility can be measured as described in Example 2.

The term “improved wash performance” is defined herein as a variant protein displaying an alteration of the wash performance relative to the parent protein (i.e. relative to a protein having the identical amino acid sequence of the variant but excluding the alterations in the variant), such as relative to the mature polypeptide of SEQ ID NO: 2 or relative to the mature polypeptide of SEQ ID NO: 3, e.g. by increased stain removal. The term “wash performance” includes wash performance in dish wash but also in laundry. The wash performance may be determined by calculating the so-called intensity value (Int) as defined in the Automatic Mechanical Stress Assay (AMSA) for Automatic Dish Wash in the Materials and Methods section herein.

The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “laundering” relates to both household laundering and industrial laundering and means the process of treating textiles and/or fabrics with a solution containing a detergent composition of the present invention. The laundering process can for example be carried out using e.g. a household or an industrial washing machine or can be carried out by hand.

The term “mature polypeptide” means a polypeptide in its final form following translation and any post-translational modifications, such as N-terminal processing, C-terminal truncation, glycosylation, phosphorylation, autocatalytic activation etc. In one aspect, the mature polypeptide is amino acids 1 to 269 of SEQ ID NO: 2 based on SignalP (Nielsen et al., 1997, Protein Engineering 10: 1-6)] that predicts amino acids −111 to −85 of SEQ ID NO: 2 are a signal peptide. It is known in the art that a host cell may produce a mixture of two of more different mature polypeptides (i.e., with a different C-terminal and/or N-terminal amino acid) expressed by the same polynucleotide. SEQ ID NO 2 is the deduced amino acid sequence of SEQ ID NO 1 the mature polypeptide of SEQ ID NO 2 is indicated as SEQ ID NO 3.

The term “mature polypeptide coding sequence” means a polynucleotide that encodes a mature polypeptide having enzyme activity. When the enzyme is a protease the mature polypeptide has protease activity. In one aspect, the mature polypeptide coding sequence is nucleotides 334 to 1140 of SEQ ID NO: 1.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “mutant” means a polynucleotide encoding a variant.

The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

The term “operably linked” means a configuration in which a control sequence is placed at an appropriate position relative to the coding sequence of a polynucleotide such that the control sequence directs expression of the coding sequence.

The term “parent” or “parent enzyme, such as parent protease means an enzyme such as protease to which an alteration is made to produce the enzyme variants, such as protease variants of the present invention. The parent is to be understood as the point of origin of the enzyme variant thus the parent of the present invention may be any enzyme not having the modifications of the variant of the present invention. In a particular aspect the variant is a protein comprising 2 to 6 histidines at positions corresponding to positions on the surface of the mature polypeptide of the protein and the parent is a protein having identical amino acid sequence except the 2 to 6 histidines at positions on the surface of the mature polypeptide. In a preferred aspect the variant is a protease comprising 2 to 6 histidines at positions corresponding to positions on the surface of the mature polypeptide of the protease and the parent is a protease having identical amino acid sequence except the 2 to 6 histidines at positions on the surface of the mature polypeptide. In an even more preferred aspect of the invention the variant is a protease comprising 2 to 6 histidines at positions corresponding to positions on the surface of the mature polypeptide of SEQ ID NO 2 or SEQ ID NO 3 and the parent is a protease having the amino acid sequence with SEQ ID NO 3.

The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The most widely used proteases in the detergent industry such as laundry and dish wash are the serine proteases or serine peptidases which is a subgroup of proteases characterised by having a serine in the active site, which forms a covalent adduct with the substrate. Further the subtilases (and the serine proteases) are characterised by having two active site amino acid residues apart from the serine, namely a histidine and an aspartic acid residue. Subtilase refer to a sub-group of serine protease according to Siezen et al., Protein Engng. 4 (1991) 719-737 and Siezen et al. Protein Science 6 (1997) 501-523. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Proteases usably in detergents are mainly endopeptidases (EC 3.4.21). There are several protease activity types: The three main activity types are: trypsin-like where there is cleavage of amide substrates following Arg or Lys at P1, chymotrypsin-like where cleavage occurs following one of the hydrophobic amino acids at P1, and elastase-like with cleavage following an Ala at P1. For purposes of the present invention, protein activity, such as protease activity is determined according to the procedure described in the Examples section below. In one aspect, the variants of the present invention have at least 20%, e.g., at least 40%, at least 50%, at least 60%, at least 70%, at least 80%, at least 90%, at least 95%, or at least 100% of the enzyme activity of the mature polypeptide of the parent enzyme. In one particular aspect the variants of the present invention have at least 20%, e.g., at least 40%, at least 50%, at least 60%, at least 70%, at least 80%, at least 90%, at least 95%, or at least 100% of the enzyme activity of the mature polypeptide of SEQ ID NO: 2.

The term “protease activity” means a proteolytic activity (EC 3.4). Proteases of the invention are endopeptidases (EC 3.4.21). There are several protease activity types: The three main activity types are: trypsin-like where there is cleavage of amide substrates following Arg or Lys at P1, chymotrypsin-like where cleavage occurs following one of the hydrophobic amino acids at P1, and elastase-like with cleavage following an Ala at P1. For purposes of the present invention, protease activity is determined according to the procedure described in “Materials and Methods” below. The histidine modified variants of the present invention preferably have at least 20%, e.g., at least 40%, at least 50%, at least 60%, at least 70%, at least 80%, at least 90%, at least 95%, and at least 100% of the protease activity of the mature polypeptide of SEQ ID NO: 2.

The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

The term “variant” means a polypeptide comprising an alteration, i.e., a substitution, insertion, and/or deletion, at one or more (e.g., several) positions. In the present context a “protease variant” is a variant of a protease, a “histidine modified variant” is a polypeptide which is modified by either inserting or substituting amino acids in the parent polypeptide to obtain a polypeptide variant comprising 2 to 6 additional histidines compared to the parent polypeptide and “a histidine modified protease variant” is a protease modified as described above to obtain a protease comprising 2 to 6 additional histidines compared to the parent protease. A substitution means replacement of the amino acid occupying a position with a different amino acid; a deletion means removal of the amino acid occupying a position; and an insertion means adding one or more amino acids adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the activity of the mature polypeptide of the parent enzyme.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “textile” means any textile material including yarns, yarn intermediates, fibers, non-woven materials, natural materials, synthetic materials, as well as fabrics made of these materials such as garments, cloths and other articles). When the term fabric or garment is used it is intend

The term “wash performance” is used as an enzyme's ability to remove stains present on the object to be cleaned during e.g. wash, such as laundry or hard surface cleaning. The improvement in the wash performance may be quantified by calculating the so-called intensity value (Int) defined in AMSA assay, as described in Materials and Methods in the present application.

The term “wild-type” enzyme means an enzyme e.g. protease expressed by a naturally occurring microorganism, such as a bacterium, yeast, or filamentous fungus found in nature.

### Conventions for Designation of Variants

The principles described below for proteases can be used for any protein. For purposes of the present invention, the mature polypeptide disclosed in SEQ ID NO: 3 is used to determine the corresponding amino acid residue in another protease. The amino acid sequence of another protease is aligned with the mature polypeptide disclosed in SEQ ID NO: 3, and based on the alignment, the amino acid position number corresponding to any amino acid residue in the mature polypeptide disclosed in SEQ ID NO: 3 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another protease can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 3 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. 2 to 6 protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of alanine at position 15 with threonine is designated as “Ala15Thr” or “A15T”. In the present context the original amino acid indicated is the amino acid present in SEQ ID NO 3. Thus in position 15 of SEQ ID NO 3 the amino acid is Alanine. It is clear the skilled artisan that if the substitution is made in another back bone e.g. another protease the original amino acid might be different from the amino acid in SEQ ID NO 3.

Deletions.

For an amino acid deletion, the following nomenclature is used: Original amino acid, position, *. Accordingly, the deletion of glycine at position 189 is designated as “Gly189*” or “G189*”. Multiple deletions are separated by addition marks (“+”), e.g., “Gly189*+Ser184*” or “G189*+S184*”.

Insertions:

The insertion of an additional amino acid residue such as e.g. a lysine after G189 may be indicated by: Gly189GlyLys or G189GK. Alternatively insertion of an additional amino acid residue such as lysine after G189 may be indicated by: *189aK. When more than one amino acid residue is inserted, such as e.g. a Lys and Ala after G189 this may be indicated as: Gly189GlyLysAla or G189GKA. In such cases, the inserted amino acid residue(s) may also be numbered by the addition of lower case letters to the position number of the amino acid residue preceding the inserted amino acid residue(s), in this example: *189aK *189bA. In the above example, the sequences 188 to 190 would thus be:

| | |
|---|---|
| | 188 189 190 |
| Savinase | A - G - L |
| | 188 189 189a 189b 190 |
| Variant | A - G - K - A - L |

In cases where a substitution and an insertion occur at the same position, this may be indicated as S97SD+S97A or in short S97AD. The same modification may also be indicated as S97A+*97aD.

In cases where an amino acid residue identical to the existing amino acid residue is inserted, it is clear that degeneracy in the nomenclature arises. If for example a glycine is inserted after the glycine in the above example this would be indicated by G189GG or *189aGbG. The same actual change could just as well be indicated as A188AG or *188aG for the change from:

| | |
|---|---|
| | 188 189 190 |
| Savinase | A - G - L |

### To:

| | |
|---|---|
| | 188 189 189a 190 |
| Variant | A - G - G - L |
| | 188 189a 189 190 |

Such instances will be apparent to the skilled person and the indication G189GG and corresponding indications for this type of insertions are thus meant to comprise such equivalent degenerate indications.

Multiple Alterations.

Variants comprising multiple alterations may be separated by addition marks (“+”), e.g., “Arg164Tyr+Gly189Glu” or “R164Y+G189E” representing a substitution of arginine and glycine at positions 164 and 189 with tyrosine and glutamic acid, respectively. Alternatively multiple alterations may be separated with space or a comma e.g. R164Y G189E or R164Y, G189E respectively.

Different Alterations.

Where different alterations can be introduced at a position, the different alterations may be separated by a comma, e.g., “Arg164Tyr,Glu” represents a substitution of arginine at position 164 with tyrosine or glutamic acid. Thus, “Tyr161Gly,Ala+Arg164Gly,Ala” designates the following variants:

### “Tyr161Gly+Arg164Gly”, “Tyr161Gly+Arg164Ala”, “Tyr161Ala+Arg164Gly”, and “Tyr161Ala+Arg164Ala”.

Alternatively different alterations or optional substitutions may be indicated in brackets e.g. Arg164 [Tyr, Glu] or Arg164 {Tyr, Glu} or in short R164 [Y, E] or R164 {Y, E}.

### DETAILED DESCRIPTION OF THE INVENTION

The present invention relates to a method of producing protein variants having at least two additional histidines at the surface compared to the parent protein the method comprising introducing into the parent protein alterations to obtain additional 2 to 6 histidines at the surface of the mature polypeptide of the parent protein, wherein each alteration is independently a substitution or insertion. The invention further relates to protein variants, which when compared to the parent protein comprises 2 to 6 additional histidines at positions corresponding to positions at the surface of the mature polypeptide of the parent protein. The inventors have found that proteins such as proteases comprising at least two additional histidines at the N or C-terminal or internally at the surface of the molecule increase the solubility and/or improve the re-solubilization kinetics of the protein. Without being bound by theory it is assumed that the histidine side-chains, which have pKa values around 6, and are positively charged at low pH increases the solubility by lowering the pH significantly below the pKa value. This property is beneficial, since proteins precipitating at a pH above the pKa of histidine can easily be re-solubilized by lowering the pH. Thus the histidine modified variants according to the invention comprising 2 to 6 additional histidines at the surface area have altered solubility compared to its parent having fewer histidines at the surface area. Thus in one aspect of the invention the histidine modified variants according to the invention have higher solubility below pH 5 compared to the parent. In another aspect the histidine modified variants according to the invention have a higher solubility at low pH, such as pH 5 than at a high pH such as pH 8 relative to the parent. Thus the ratio (solubility at pH 5/solubility pH 8) is higher for the variant than for the parent of the variant. Solubility is defined in the present context as the maximum amount of protein in solution. The amount of protein in solution can be measured in standard protein assays such as a Bradford assay wherein the protein concentration in the solution is determined (Bradford, M. M. (1976), “Rapid and sensitive method for the quantitation of microgram quantities of protein utilizing the principle of protein-dye binding”, Anal. Biochem. 72: 248-254). Proteins having high solubility at low pH are particularly advantageous when recovering proteins from processes running at low pH e.g. between 3.5 to 6.5. In a particular context of the present invention the variants have higher solubility when measured as described in “solubility assay” in example 2 herein.

In a particular embodiment, the 2 to 6 additional histidines situated at the surface of the protein e.g. SEQ ID NO 3 are not at the N- or C-terminal of the protein or at least one of the additional histidines (compared to histidines of the parent protein) are not at the N- or C-terminal. Thus, in one embodiment the additional histidines are not all at the N-terminal of C-terminal of the protein. One embodiment of the invention relates to a composition comprising at least one protein variant, such as a protease variant e.g. such as a protease variant having at least 60% sequence identity to SEQ ID NO 3, which, when compared to the parent protein, comprises 2 to 6 additional histidines at positions corresponding to positions at the surface of the mature polypeptide of the parent protein. In another embodiment the invention relates to a composition comprising at least one first histidine modified protein variant wherein the protein variant when compared to the parent protein comprises 2 to 6 additional histidines at positions corresponding to positions at the surface of the parent protein and at least one second protein variant which is identical to the first protein variant excluding the additional 2 to 6 histidines at positions corresponding to positions at the surface of the parent protein. The parent protein is preferably a protease having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO 3.

The composition may be a cleaning composition, such as a hard surface cleaning composition or a laundry composition; such compositions may also comprise additional enzymes such as amylases, lipases, mannanases etc. such as described below. The composition may also comprise components such as surfactants, builders, polymers e.g. polyols, bleaches. For the cleaning composition detergent components such as surfactants and builders are well known and are described in details in the “composition” section below. The histidines modified variants of the invention preferably have increased performance e.g. substrate performance on substrates such as EMPA117EH, PC-03 and PC-05 when measured in the relevant assay as described in Methods and Materials in the application.

In one aspect according to the invention, 2 to 6 histidines are substituted instead of amino acids at the surface area of the protein or added to the N- and/or C-terminal of the protein. Alternatively to substituting histidines for existing non-histidine amino acids the histidines can be inserted between amino acids at the surface of the protein. The protein is preferably a protease and even more preferably a protease having at least 60% identity to the mature polypeptide of SEQ ID NO 2.

The present invention relates to proteins comprising 2 to 6 additional histidines compared to the parent protein at positions corresponding to positions on the surface of the mature polypeptide of the protein, wherein the protein have increased solubility compared to an protein having identical amino acid sequence except the 2 to 6 additional histidines at positions on the surface of the mature polypeptide.

The term “additional histidines” is to be understood as the histidine modified variants having more histidines than its corresponding parent. Proteins may comprise one or more histidines, however the inventors have found that increasing the amount of histidines at the surface area provide a beneficial effect on the solubilization and re-solubilization kinetics of the protein as described above. In addition, many proteins are modified to optimize them for specific purposes e.g. proteins such as proteases are often modified to increase their stain removal capacity, however these modifications may lead to a modified protease which is less soluble, which can cause problems e.g. during the recovery process. The inventors have found that increasing the amount of histidines by 2 to 6 histidines on the surface of certain proteases increases the solubility of the protease. The terms “parent” or “parent protein” simply means starting or precursor protein or the protein into which the 2 to 6 additional histidines are inserted and/or substituted.

In a preferred embodiment, the invention relates to a histidine modified protease variant comprising 2 to 6 additional histidines at positions corresponding to positions on the surface of the mature polypeptide of the protease, wherein the protease have increased solubility at pH 5 compared to a protease having identical amino acid sequence except the 2 to 6 additional histidines at positions on the surface of the mature polypeptide. In a preferred embodiment, the histidine modified protease variants comprises an amino acid sequence with at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, identical to the mature polypeptide of SEQ ID NO 2 (or SEQ ID NO 3). Thus a preferred embodiment relates to a protease variant comprising 2 to 6 additional histidines at positions corresponding to positions on the surface of the mature polypeptide of the protease, wherein the protease have increased solubility compared to the mature polypeptide of SEQ ID NO 2 (or SEQ ID NO 3) and wherein the histidine modified protease variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the mature polypeptide of SEQ ID NO 2. In an even more preferred aspect the invention relates to a histidine modified protease variant having at least 60% identity with the amino acid sequence of SEQ ID NO 2 wherein the histidine modified protease variant comprises 2 to 6 additional histidines at positions corresponding to positions on the surface of the mature polypeptide of the protease, wherein the protease have increased solubility at pH 5 compared to the protease having identical amino acid sequence except the 2 to 6 additional histidines at positions on the surface of the mature polypeptide and wherein the protease has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the mature polypeptide of SEQ ID NO 2.

In one embodiment, the histidines are added to the N or C-terminal of a protein. In a particular embodiment 2 to 6 histidines are added to the N or C-terminal. In a preferred embodiment 2 to 6 histidines are added to the N or C-terminal of a protease having at least 60% identity to the mature polypeptide of SEQ ID NO 2, wherein the protease have increased solubility at pH 5 compared to the protease having identical amino acid sequence except the 2 to 6 additional histidines.

In one preferred embodiment, the histidines are added to the N or C-terminal of a protein. In a particular embodiment 2 to 5 histidines are added to the N or C-terminal. In a preferred embodiment 2 to 5 histidines are added to the N or C-terminal of a protease having at least 60% identity to the mature polypeptide of SEQ ID NO 2, wherein the protease have increased solubility at pH 5 compared to the protease having identical amino acid sequence except the 2 to 5 additional histidines.

In one embodiment, the histidines are added to the N or C-terminal of a protein. In a particular embodiment 2 to 4 histidines are added to the N or C-terminal. In a preferred embodiment 2 to 4 histidines are added to the N or C-terminal of a protease having at least 60% identity to the mature polypeptide of SEQ ID NO 2, wherein the protease have increased solubility at pH 5 compared to the protease having identical amino acid sequence except the 2 to 4 additional histidines.

In one embodiment, the histidines are added to the N or C-terminal of a protein. In a particular embodiment 2 to 3 histidines are added to the N or C-terminal. In a preferred embodiment 2 to 3 histidines are added to the N or C-terminal of a protease having at least 60% identity to the mature polypeptide of SEQ ID NO 2, wherein the protease have increased solubility at pH 5 compared to the protease having identical amino acid sequence except the 2 to 3 additional histidines.

In one embodiment, the histidines are added to the N or C-terminal of a protein. In a particular embodiment 3 to 4 histidines are added to the N or C-terminal. In a preferred embodiment 3 to 4 histidines are added to the N or C-terminal of a protease having at least 60% identity to the mature polypeptide of SEQ ID NO 2, wherein the protease have increased solubility at pH 5 compared to the protease having identical amino acid sequence except the 3 to 4 additional histidines.

In one embodiment, the histidines are added to the N or C-terminal of a protein. In a particular embodiment 3 to 5 histidines are added to the N or C-terminal. In a preferred embodiment 3 to 5 histidines are added to the N or C-terminal of a protease having at least 60% identity to the mature polypeptide of SEQ ID NO 2, wherein the protease have increased solubility at pH 5 compared to the protease having identical amino acid sequence except the 3 to 5 additional histidines.

In one embodiment, the histidines are added to the N or C-terminal of a protein. In a particular embodiment 4 to 5 histidines are added to the N or C-terminal. In a preferred embodiment 4 to 5 histidines are added to the N or C-terminal of a protease having at least 60% identity to the mature polypeptide of SEQ ID NO 2, wherein the protease have increased solubility at pH 5 compared to the protease having identical amino acid sequence except the 4 to 5 additional histidines.

In one embodiment, the histidines are added to the N or C-terminal of a protein. In a particular embodiment 2 histidines are added to the N or C-terminal. In a preferred embodiment 2 histidines are added to the N or C-terminal of a protease having at least 60% identity to the mature polypeptide of SEQ ID NO 2, wherein the protease have increased solubility at pH 5 compared to the protease having identical amino acid sequence except the 2 additional histidines.

In one embodiment, the histidines are added to the N or C-terminal of a protein. In a particular embodiment 3 histidines are added to the N or C-terminal. In a preferred embodiment 3 histidines are added to the N or C-terminal of a protease having at least 60% identity to the mature polypeptide of SEQ ID NO 2, wherein the protease have increased solubility at pH 5 compared to the protease having identical amino acid sequence except the 3 additional histidines.

In one embodiment, the histidines are added to the N or C-terminal of a protein. In a particular embodiment 4 histidines are added to the N or C-terminal. In a preferred embodiment 4 histidines are added to the N or C-terminal of a protease having at least 60% identity to the mature polypeptide of SEQ ID NO 2, wherein the protease have increased solubility at pH 5 compared to the protease having identical amino acid sequence except the 4 additional histidines.

In one embodiment, the histidines are added to the N or C-terminal of a protein. In a particular embodiment 5 histidines are added to the N or C-terminal. In a preferred embodiment 5 histidines are added to the N or C-terminal of a protease having at least 60% identity to the mature polypeptide of SEQ ID NO 2, wherein the protease have increased solubility at pH 5 compared to the protease having identical amino acid sequence except the 5 additional histidines.

In one embodiment, the histidines are added to the N or C-terminal of a protein. In a particular embodiment 6 histidines are added to the N or C-terminal. In a preferred embodiment 6 histidines are added to the N or C-terminal of a protease having at least 60% identity to the mature polypeptide of SEQ ID NO 2, wherein the protease have increased solubility at pH 5 compared to the protease having identical amino acid sequence except the 6 additional histidines.

One embodiment of the invention relates to a variant of a parent protein, which when compared to the parent protein comprise 2 to 6 additional histidines at positions corresponding to positions at the surface of the mature polypeptide of the parent protein.

One embodiment of the invention relates to a variant of a parent protease, which when compared to the parent protease comprise 2 to 6 additional histidines at positions corresponding to positions at the surface of the mature polypeptide of the parent protease.

In one embodiment of the invention, the parent protease is the polypeptide with SEQ ID NO 3. Thus One embodiment of the invention relates to a variant of a polypeptide with SEQ ID NO 3, which when compared to the polypeptide with SEQ ID NO 3 comprise 2 to 6 additional histidines at positions corresponding to positions at the surface of the polypeptide with SEQ ID NO: 3, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity the polypeptide with SEQ ID NO: 3.

In another embodiment of the invention, the parent protease is the polypeptide with SEQ ID NO 3. Thus One embodiment of the invention relates to a variant of a polypeptide with SEQ ID NO 3, which when compared to the polypeptide with SEQ ID NO 3 comprise 2 to 6 additional histidines at positions corresponding to positions at the surface of the polypeptide with SEQ ID NO: 3, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity the polypeptide with SEQ ID NO: 3 and wherein the variant has an increased solubility below pH 5 compared to the polypeptide with SEQ ID NO 3.

One embodiment of the invention relates to a protease variant of a parent protease, which when compared to the parent protease comprise 2 additional histidines at positions corresponding to positions at the surface of the mature polypeptide of the parent protease, wherein the positions are selected from the group consisting of A1, Q2, S3, V4, W6, S9, R10, Q12, P14, A15, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, Y89, A96, S97, G98, S99, G100, S101, V102, S103, Q107, E110, W111, N114, N115, G116, G125, S126, P127, S128, P129, A131, T132, E134, Q135, N138, S139, S142, R143, G144, N153, S154, G155, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, K245, N246, T247, T249, S250, L251, G252, S253, T254, N255, L256, Y257, S259, E265 and R269 (positions numbered according to SEQ ID NO 3).

In one embodiment of the invention, the parent protease is the polypeptide with SEQ ID NO 3. Thus One embodiment of the invention relates to a variant of a polypeptide with SEQ ID NO 3, which when compared to the polypeptide with SEQ ID NO 3 comprise 2 additional histidines at positions corresponding to positions at the surface of the polypeptide with SEQ ID NO: 3, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity the polypeptide with SEQ ID NO: 3.

One embodiment of the invention relates to a variant of a parent protein, which when compared to the parent protein comprise 3 additional histidines at positions corresponding to positions at the surface of the mature polypeptide of the parent protein.

In one embodiment of the invention, the parent protease is the polypeptide with SEQ ID NO 3. Thus One embodiment of the invention relates to a variant of a polypeptide with SEQ ID NO 3, which when compared to the polypeptide with SEQ ID NO 3 comprise 3 additional histidines at positions corresponding to positions at the surface of the polypeptide with SEQ ID NO: 3, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity the polypeptide with SEQ ID NO: 3.

One embodiment of the invention relates to a variant of a parent protein, which when compared to the parent protein comprise 4 additional histidines at positions corresponding to positions at the surface of the mature polypeptide of the parent protein.

In another embodiment of the invention, the parent protease is the polypeptide with SEQ ID NO 3. Thus One embodiment of the invention relates to a variant of a polypeptide with SEQ ID NO 3, which when compared to the polypeptide with SEQ ID NO 3 comprise 4 additional histidines at positions corresponding to positions at the surface of the polypeptide with SEQ ID NO: 3, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity the polypeptide with SEQ ID NO: 3.

One embodiment of the invention relates to a variant of a parent protein, which when compared to the parent protein comprise 5 additional histidines at positions corresponding to positions at the surface of the mature polypeptide of the parent protein.

In another embodiment of the invention, the parent protease is the polypeptide with SEQ ID NO 3. Thus, one embodiment of the invention relates to a variant of a polypeptide with SEQ ID NO 3, which when compared to the polypeptide with SEQ ID NO 3 comprise 5 additional histidines at positions corresponding to positions at the surface of the polypeptide with SEQ ID NO: 3, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity the polypeptide with SEQ ID NO: 3.

One embodiment of the invention relates to a variant of a parent protein, which when compared to the parent protein comprise 6 additional histidines at positions corresponding to positions at the surface of the mature polypeptide of the parent protein.

In another embodiment of the invention, the parent protease is the polypeptide with SEQ ID NO 3. Thus, one embodiment of the invention relates to a variant of a polypeptide with SEQ ID NO 3, which when compared to the polypeptide with SEQ ID NO 3 comprise 6 additional histidines at positions corresponding to positions at the surface of the polypeptide with SEQ ID NO: 3, wherein the variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity the polypeptide with SEQ ID NO: 3.

One embodiment of the invention relates to a method of producing a variant of a parent protein wherein the variant has at least two additional histidines on the surface compared to the parent, the method comprising introducing into the parent protein alterations to obtain additional 2 to 6 histidines at the surface of the mature polypeptide of the parent protein, wherein each alteration is independently a substitution or insertion.

One embodiment of the invention relates to a method of producing a variant of a parent protein wherein the variant has at least two additional histidines on the surface compared to the parent, the method comprising substituting 2 to 6 histidines at positions corresponding to positions at the surface of the mature polypeptide of the parent protein.

One embodiment of the invention relates to a method of producing a variant of a parent protein wherein the variant has at least two additional histidines on the surface compared to the parent, the method comprising substituting 2 to 6 histidines at positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising inserting 2 to 6 histidines at adjacent positions corresponding to positions at the surface of the mature polypeptide of the parent protein.

One embodiment of the invention relates to a method of producing a variant of a parent protein wherein the variant has at least two additional histidines on the surface compared to the parent, the method comprising inserting 2 to 6 histidines at adjacent positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising introducing into the parent protein alterations to obtain additional 2 histidines at the surface of the mature polypeptide of the parent protein, wherein each alteration is independently a substitution or insertion, wherein the variant has increased solubility below pH 5 compared to the parent protein.

One embodiment of the invention relates to a method of producing a variant of a parent protein wherein the variant has at least two additional histidines on the surface compared to the parent, the method comprising substituting 2 histidines at positions corresponding to positions at the surface of the mature polypeptide of the parent protein.

One embodiment of the invention relates to a method of producing a variant of a parent protein wherein the variant has at least two additional histidines on the surface compared to the parent, the method comprising substituting 2 histidines at positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising inserting 2 histidines adjacent positions corresponding to positions at the surface of the mature polypeptide of the parent protein.

One embodiment of the invention relates to a method of producing a variant of a parent protein wherein the variant has at least two additional histidines on the surface compared to the parent, the method comprising inserting 2 histidines adjacent positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising introducing into the parent protein alterations to obtain additional 3 histidines at the surface of the mature polypeptide of the parent protein, wherein each alteration is independently a substitution or insertion, wherein the variant has increased solubility below pH 5 compared to the parent protein.

One embodiment of the invention relates to a method of producing a variant of a parent protein wherein the variant has at least two additional histidines on the surface compared to the parent, the method comprising substituting 3 histidines at positions corresponding to positions at the surface of the mature polypeptide of the parent protein.

One embodiment of the invention relates to a method of producing a variant of a parent protein wherein the variant has at least two additional histidines on the surface compared to the parent, the method comprising substituting 3 histidines at positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising inserting 3 histidines adjacent positions corresponding to positions at the surface of the mature polypeptide of the parent protein.

One embodiment of the invention relates to a method of producing a variant of a parent protein wherein the variant has at least two additional histidines on the surface compared to the parent, the method comprising inserting 3 histidines adjacent positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising introducing into the parent protein alterations to obtain additional 4 histidines at the surface of the mature polypeptide of the parent protein, wherein each alteration is independently a substitution or insertion, wherein the variant has increased solubility below pH 5 compared to the parent protein.

One embodiment of the invention relates to a method of producing a variant of a parent protein wherein the variant has at least two additional histidines on the surface compared to the parent, the method comprising substituting 4 histidines at positions corresponding to positions at the surface of the mature polypeptide of the parent protein.

One embodiment of the invention relates to a method of producing a variant of a parent protein wherein the variant has at least two additional histidines on the surface compared to the parent, the method comprising substituting 4 histidines at positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising inserting 4 histidines adjacent positions corresponding to positions at the surface of the mature polypeptide of the parent protein.

One embodiment of the invention relates to a method of producing a variant of a parent protein wherein the variant has at least two additional histidines on the surface compared to the parent, the method comprising inserting 4 histidines adjacent positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising introducing into the parent protein alterations to obtain additional 5 histidines at the surface of the mature polypeptide of the parent protein, wherein each alteration is independently a substitution or insertion, wherein the variant has increased solubility below pH 5 compared to the parent protein.

One embodiment of the invention relates to a method of producing a variant of a parent protein wherein the variant has at least two additional histidines on the surface compared to the parent, the method comprising substituting 5 histidines at positions corresponding to positions at the surface of the mature polypeptide of the parent protein.

One embodiment of the invention relates to a method of producing a variant of a parent protein wherein the variant has at least two additional histidines on the surface compared to the parent, the method comprising substituting 5 histidines at positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising inserting 5 histidines adjacent positions corresponding to positions at the surface of the mature polypeptide of the parent protein.

One embodiment of the invention relates to a method of producing a variant of a parent protein wherein the variant has at least two additional histidines on the surface compared to the parent, the method comprising inserting 5 histidines adjacent positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising introducing into the parent protein alterations to obtain additional 6 histidines at the surface of the mature polypeptide of the parent protein, wherein each alteration is independently a substitution or insertion, wherein the variant has increased solubility below pH 5 compared to the parent protein.

One embodiment of the invention relates to a method of producing a variant of a parent protein wherein the variant has at least two additional histidines on the surface compared to the parent, the method comprising substituting 6 histidines at positions corresponding to positions at the surface of the mature polypeptide of the parent protein.

One embodiment of the invention relates to a method of producing a variant of a parent protein wherein the variant has at least two additional histidines on the surface compared to the parent, the method comprising substituting 6 histidines at positions corresponding to positions at the surface of the mature polypeptide of the parent protein, wherein the variant has increased solubility below pH 5 compared to the parent protein.

One embodiment of the invention relates to a method of producing a variant of a parent protein wherein the variant has at least two additional histidines on the surface compared to the parent, the method comprising inserting 6 histidines adjacent positions corresponding to positions at the surface of the mature polypeptide of the parent protein.

One embodiment of the invention relates to a method of producing a variant of a parent protein wherein the variant has at least two additional histidines on the surface compared to the parent, the method comprising inserting 6 histidines adjacent positions corresponding to positions at the surface of the mature polypeptide of the parent protein, wherein the variant has increased solubility below pH 5 compared to the parent protein.

One embodiment of the invention relates to a method of producing a variant of a parent protease wherein the variant has at least two additional histidines on the surface compared to the parent protease, the method comprising introducing into the parent protease alterations to obtain additional 2 to 6 histidines at the surface of the mature polypeptide of the parent protease, wherein each alteration is independently a substitution or insertion.

One embodiment of the invention relates to a method of producing a variant of a parent protease wherein the variant has at least two additional histidines on the surface compared to the parent protease, the method comprising introducing into the parent protease alterations to obtain additional 2 to 6 histidines at the surface of the mature polypeptide of the parent protease, wherein each alteration is independently a substitution or insertion, wherein the variant has increased solubility below pH 5 compared to the parent protease.

One embodiment of the invention relates to a method of producing a variant of a parent protease wherein the variant has at least two additional histidines on the surface compared to the parent protease, the method comprising substituting 2 to 6 histidines at positions corresponding to positions at the surface of the mature polypeptide of the parent protease.

One embodiment of the invention relates to a method of producing a variant of a parent protease wherein the variant has at least two additional histidines on the surface compared to the parent protease, the method comprising inserting 2 to 6 histidines at adjacent positions corresponding to positions at the surface of the mature polypeptide of the parent protease.

One embodiment of the invention relates to a method of producing a variant of a parent protease wherein the variant has at least two additional histidines on the surface compared to the parent protease, the method comprising inserting 2 to 6 histidines at adjacent positions corresponding to positions at the surface of the mature polypeptide of the parent protease, wherein the variant has increased solubility below pH 5 compared to the parent protease. In a particular preferred embodiment of the invention the parent protease is a protease having at least 60% sequence identity to the amino acid sequences of SEQ ID NO 3. In another embodiment of the invention the parent protease is a protease with SEQ ID NO 3.

One embodiment of the invention relates to a method of producing a variant of a protease with SEQ ID NO 3 wherein the protease variant has at least two additional histidines on the surface compared to SEQ ID NO 3, the method comprising introducing into a protease with SEQ ID NO 3 alterations to obtain additional 2 to 6 histidines at the surface of the protease with SEQ ID NO 3, wherein each alteration is independently a substitution or insertion, wherein the protease variant has increased solubility below pH 5 compared SEQ ID NO 3.

One embodiment of the invention relates to a method of producing a variant of a protease with SEQ ID NO 3 wherein the protease variant has at least two additional histidines on the surface compared to SEQ ID NO 3, the method comprising introducing into a protease with SEQ ID NO 3 alterations to obtain additional 2 to 6 histidines at the surface of the protease with SEQ ID NO 3, wherein each alteration is independently a substitution or insertion, wherein when the alteration is an insertions the histidines are not added to the C- or N-terminal and wherein the protease variant has increased solubility below pH 5 compared SEQ ID NO 3.

As described above the histidine modified variants of the invention comprises in addition to increased solubility as described above also increased performance on at least one substrate selected from, EnzChek Protease kit Red, DQ Red BSA, EMPA117EH, AZCL-Hemoglobin, PC-03 and PC-05 as shown in the examples below.

One embodiment of the invention relates to a method of producing a variant of a protease having at least 60% sequence identity to the mature polypeptide of SEQ ID NO 2 wherein the variant has at least two additional histidines on the surface compared to the mature polypeptide of SEQ ID NO 2, the method comprising substituting 2 to 6 histidines at positions corresponding to positions at the surface of the mature polypeptide of SEQ ID NO 2.

One embodiment of the invention relates to a method of producing a variant of a parent protease having at least 60% sequence identity to the mature polypeptide of SEQ ID NO 2 wherein the variant has at least two additional histidines on the surface compared to the mature polypeptide of SEQ ID NO 2, the method comprising inserting 2 to 6 histidines adjacent positions corresponding to positions at the surface of the mature polypeptide of SEQ ID NO 2.

One aspect of the invention relates to a method of producing a variant of a parent protein, wherein the variant has at least two additional histidines on the surface compared to the parent protein comprising the steps of:
a) identifying amino acid positions at the surface of the mature polypeptide of the parent protein;
b) selecting at least one amino acid position among the positions identified in a) which is not occupied by a histidine in the parent protein; and
c) substituting amino acid selected in b) with histidine; and
d) expressing the modified nucleic acid sequence in a host cell to produce the variant.

Another aspect relates to a method of producing a variant of a parent protein, wherein the variant has at least two additional histidines on the surface compared to the parent protein comprising the steps of:
a) identifying amino acid positions at the surface of the mature polypeptide of the parent enzyme;
b) selecting at least one amino acid position among the positions identified in a); and
c) inserting one or more histidine adjacent to the at least one amino acid position selected in b); and
d) expressing the modified nucleic acid sequence in a host cell to produce the variant.

Yet another embodiment relates to a method of producing a variant of a parent enzyme, wherein the wherein the variant has at least two additional histidines at the surface compared to the parent protein comprising the steps of:
a) adding 2 to 6 histidines at the N or C-terminal of an parent enzyme; and
b) expressing the modified nucleic acid sequence in a host cell to produce the variant.

In a preferred embodiment of the invention, step a) is performed using the method described in “solvent accessibility of residues in 3D model”.

The surface area of a protease having SEQ ID NO 3 is described below. This method can be applied for any protein.

### Solvent Accessibility of Residues in 3D Model

A method to identify amino acid positions at the surface of the mature polypeptide of the parent protein is described in the following. The surface area, the amino acids at the surface or the solvent accessible surface area is calculated for each residue using the DSSP software (W. Kabsch and C. Sander, Biopolymers 22 (1983) 2577-2637). Each solvent accessible surface area is divided by a standard value for the particular amino acid found in that position and multiplied by 100, thereby obtaining a percentage of the standard value for each residue.

The standard solvent accessible surface areas for the 20 different amino acids are defined as (using one-letter codes for the amino acids): A=62, C=92, D=69, E=156, F=123, G=50, H=130, 1=84, K=174, L=97, M=103, N=85, P=67, Q=127, R=211, S=64, T=80, V=81, W=126, Y=104.

Based on the location of the C-alpha atom of each residue in the 3D model, residues are selected which meet the following criteria.

### Accessibility >40, >45, >50, >60%, >70%, >80%, >90%, >100%, >110%, >120%, >130% or >140%.

The following lists some particular amino acid residues and combination of these selected by the criteria stated above.

Distances between residues were measured as C-alpha distances in the PDB:1SVN crystal structure (downloadable from http://wwwscsb.org/pdb/). Below is listed the positions corresponding to positions on the surface of the mature polypeptide of SEQ ID NO 2. The residues were renumbered to sequential numbering from 1 to 269 of SEQ ID NO 3 (the mature polypeptide of SEQ ID NO 2) throughout the application. Thus the numbering starts with A1 of SEQ ID NO: 2 as position 1.

### Accessibility >40%, Min Distance to Active Site 6 Angstroms

A1, Q2, S3, V4, W6, S9, R10, Q12, P14, A15, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, Y89, A96, S97, G98, S99, G100, S101, V102, S103, Q107, E110, W111, N114, N115, G116, G125, S126, P127, S128, P129, A131, T132, E134, Q135, N138, S139, S142, R143, G144, N153, S154, G155, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, K245, N246, T247, T249, S250, L251, G252, S253, T254, N255, L256, Y257, S259, E265, R269.

### Accessibility >45%, Min Distance to Active Site 6 Angstroms

A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259, R269.

### Accessibility >50%, Min Distance to Active Site 6 Angstroms

A1, Q2, S3, V4, W6, S9, R10, Q12, P14, A15, N18, R19, G20, S24, G25, K27, T37, P39, N42, 143, R44, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S142, R143, G144, N153, S154, A156, G157, S158, R164, A166, N167, D175, Q176, N177, N178, N179, R180, S182, F183, Y186, A188, G189, N198, Q200, Y203, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259, R269

### Accessibility >60%, Min Distance to Active Site 6 Angstroms

A1, Q2, S3, V4, W6, S9, R10, Q12, P14, N18, R19, G20, S24, G25, K27, T37, P39, N42, 143, R44, F49, P51, G52, E53, P54, S55, T56, Q57, G59, L73, N74, N75, S76, 177, S85, S97, S99, S101, V102, S103, Q107, E110, W111, N114, N115, G116, H118, G125, P127, S128, P129, A131, T132, E134, Q135, N138, S142, R143, N153, S154, A156, G157, S158, R164, A166, N167, Q176, N177, N178, N179, R180, S182, F183, Y186, A188, G189, N198, Q200, Y203, G205, S206, T207, Y208, S210, L211, N212, K229, Q230, K231, P233, S234, W235, S236, V238, Q239, R241, N242, H243, K245, N246, T249, S250, L251, G252, S253, T254, N255, L256, R269

### Accessibility >70%, Min Distance to Active Site 6 Angstroms

A1, Q2, S3, V4, W6, S9, R10, Q12, P14, N18, R19, G20, S24, G25, K27, T37, P39, N42, R44, F49, P51, G52, E53, P54, S55, T56, Q57, G59, L73, N74, N75, S76, 177, S85, S97, S99, S101, V102, S103, Q107, E110, W111, N114, N115, G116, H118, G125, P127, S128, P129, A131, T132, E134, Q135, N138, S142, R143, N153, S154, S158, R164, A166, N167, Q176, N177, N178, N179, R180, S182, F183, Y186, A188, G189, N198, Q200, Y203, G205, S206, T207, Y208, S210, L211, N212, Q230, K231, P233, S234, W235, S236, V238, Q239, N242, H243, K245, N246, T249, S250, L251, S253, T254, N255, L256, R269

### Accessibility >80%, Min Distance to Active Site 6 Angstroms

A1, S3, V4, S9, R10, Q12, N18, R19, S24, K27, T37, P39, N42, R44, P51, G52, P54, S55, Q57, G59, N74, N75, S76, 177, S85, S97, S99, S101, S103, Q107, E110, W111, N114, N115, H118, P127, S128, P129, A131, T132, E134, Q135, N138, S142, R143, N153, S154, S158, R164, A166, N167, Q176, N177, N179, R180, S182, Y186, A188, N198, Q200, Y203, G205, S206, T207, Y208, S210, L211, N212, Q230, K231, P233, S234, W235, S236, V238, Q239, N242, H243, N246, T249, S250, S253, T254, N255, L256, R269

### Accessibility >90%, Min Distance to Active Site 6 Angstroms

A1, S3, V4, S9, R10, Q12, N18, R19, S24, T37, P39, N42, R44, P51, G52, P54, S55, Q57, G59, N74, N75, S76, 177, S97, S99, S101, S103, Q107, E110, N114, N115, H118, P127, S128, P129, A131, T132, E134, Q135, S142, R143, N153, S154, S158, R164, A166, N167, Q176, N177, N179, R180, S182, A188, N198, Q200, Y203, G205, S206, T207, Y208, S210, N212, K231, P233, S234, W235, V238, Q239, N242, N246, T249, S250, S253, T254, N255, R269

### Accessibility >100%, Min Distance to Active Site 6 Angstroms

A1, S3, V4, S9, R10, Q12, N18, R19, S24, T37, P39, N42, R44, P51, G52, P54, Q57, N74, S76, S97, S99, S103, Q107, E110, N114, N115, P127, S128, P129, A131, T132, E134, Q135, S142, R143, N153, S154, R164, A166, Q176, N177, N179, S182, A188, N198, Q200, Y203, G205, S206, T207, Y208, N212, K231, P233, S234, V238, Q239, N242, N246, S250, T254, N255, R269

### Accessibility >110%, Min Distance to Active Site 6 Angstroms

A1, R10, Q12, N18, R19, T37, N42, R44, P51, G52, P54, Q57, N74, S76, S97, S99, Q107, N114, N115, P127, P129, T132, E134, Q135, S142, R143, N153, S154, R164, A166, Q176, N177, N179, S182, A188, N198, Q200, Y203, G205, S206, N212, K231, P233, S234, V238, Q239, N242, N246, S250, T254, N255, R269

### Accessibility >120%, Min Distance to Active Site 6 Angstroms

A1, R10, N18, R19, T37, N42, R44, P51, G52, P54, Q57, N74, S76, S97, S99, Q107, N114, N115, P127, P129, E134, Q135, S142, R143, N153, S154, R164, A166, Q176, N177, N179, S182, A188, N198, Q200, Y203, N212, K231, P233, S234, Q239, N246, S250, T254, N255, R269

### Accessibility >130%, Min Distance to Active Site 6 Angstroms

### A1, N18, R19, T37, N42, R44, P51, G52, P54, Q57, N74, S76, S97, Q107, N114, P127, P129, Q135, S142, R143, R164, Q176, N177, S182, Q200, Y203, N212, K231, P233, S234, Q239, N246, S250, T254, R269

### Accessibility >140%, Min Distance to Active Site 6 Angstroms

### A1, N18, R19, T37, N42, R44, P51, P54, Q57, N74, S76, S97, Q107, N114, P127, P129, Q135, S142, R164, N177, S182, N212, K231, P233, S234, Q239, N246, S250, T254, R269.

Introducing Histidines into the Surface Exposed Areas

2 to 6 histidines residues may be introduced into the surface exposed areas by 2 to 6 of the following substitutions.

A protein variant may comprise 2 to 6 of the following substitutions, such that the total number of introduced histidines is 2, 3, 4, 5 or 6. The residues that may be selected for substitution/insertion vary depending on the surface accessibility threshold chosen (here 40, 45, 50, 60, 70, 80, 90, 100, 110, 120, 130 or 140%)

### Accessibility >40%, Min Distance to Active Site 6 Angstroms

A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, Y89H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, G155H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T247H, T249H, S250H, L251H, G252H, S253H, T254H, N255H, L256H, Y257H, S259H, E265H, R269H

### Accessibility >45%, Min Distance to Active Site 6 Angstroms

A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H.

### Accessibility >50%, Min Distance to Active Site 6 Angstroms

A1H, Q2H, S3H, V4H, W6H, S9H, R10H, Q12H, P14H, A15H, N18H, R19H, G20H, S24H, G25H, K27H, T37H, P39H, N42H, 143H, R44H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S142H, R143H, G144H, N153H, S154H, A156H, G157H, S158H, R164H, A166H, N167H, D175H, Q176H, N177H, N178H, N179H, R180H, S182H, F183H, Y186H, A188H, G189H, N198H, Q200H, Y203H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H.

### Accessibility >60%, Min Distance to Active Site 6 Angstroms

A1H, Q2H, S3H, V4H, W6H, S9H, R10H, Q12H, P14H, N18H, R19H, G20H, S24H, G25H, K27H, T37H, P39H, N42H, 143H, R44H, F49H, P51H, G52H, E53H, P54H, S55H, T56H, Q57H, G59H, L73H, N74H, N75H, S76H, 177H, S85H, S97H, S99H, S101H, V102H, S103H, Q107H, E110H, W111H, N114H, N115H, G116H, G125H, P127H, S128H, P129H, A131H, T132H, E134H, Q135H, N138H, S142H, R143H, N153H, S154H, A156H, G157H, S158H, R164H, A166H, N167H, Q176H, N177H, N178H, N179H, R180H, S182H, F183H, Y186H, A188H, G189H, N198H, Q200H, Y203H, G205H, S206H, T207H, Y208H, S210H, L211H, N212H, K229H, Q230H, K231H, P233H, S234H, W235H, S236H, V238H, Q239H, R241H, N242H, K245H, N246H, T249H, S250H, L251H, G252H, S253H, T254H, N255H, L256H, R269H

### Accessibility >70%, Min Distance to Active Site 6 Angstroms

A1H, Q2H, S3H, V4H, W6H, S9H, R10H, Q12H, P14H, N18H, R19H, G20H, S24H, G25H, K27H, T37H, P39H, N42H, R44H, F49H, P51H, G52H, E53H, P54H, S55H, T56H, Q57H, G59H, L73H, N74H, N75H, S76H, 177H, S85H, S97H, S99H, S101H, V102H, S103H, Q107H, E110H, W111H, N114H, N115H, G116H, G125H, P127H, S128H, P129H, A131H, T132H, E134H, Q135H, N138H, S142H, R143H, N153H, S154H, S158H, R164H, A166H, N167H, Q176H, N177H, N178H, N179H, R180H, S182H, F183H, Y186H, A188H, G189H, N198H, Q200H, Y203H, G205H, S206H, T207H, Y208H, S210H, L211H, N212H, Q230H, K231H, P233H, S234H, W235H, S236H, V238H, Q239H, N242H, K245H, N246H, T249H, S250H, L251H, S253H, T254H, N255H, L256H, R269H

### Accessibility >80%, Min Distance to Active Site 6 Angstroms

A1H, S3H, V4H, S9H, R10H, Q12H, N18H, R19H, S24H, K27H, T37H, P39H, N42H, R44H, P51H, G52H, P54H, S55H, Q57H, G59H, N74H, N75H, S76H, 177H, S85H, S97H, S99H, S101H, S103H, Q107H, E110H, W111H, N114H, N115H, P127H, S128H, P129H, A131H, T132H, E134H, Q135H, N138H, S142H, R143H, N153H, S154H, S158H, R164H, A166H, N167H, Q176H, N177H, N179H, R180H, S182H, Y186H, A188H, N198H, Q200H, Y203H, G205H, S206H, T207H, Y208H, S210H, L211H, N212H, Q230H, K231H, P233H, S234H, W235H, S236H, V238H, Q239H, N242H, N246H, T249H, S250H, S253H, T254H, N255H, L256H, R269H

### Accessibility >90%, Min Distance to Active Site 6 Angstroms

A1H, S3H, V4H, S9H, R10H, Q12H, N18H, R19H, S24H, T37H, P39H, N42H, R44H, P51H, G52H, P54H, S55H, Q57H, G59H, N74H, N75H, S76H, 177H, S97H, S99H, S101H, S103H, Q107H, E110H, N114H, N115H, P127H, S128H, P129H, A131H, T132H, E134H, Q135H, S142H, R143H, N153H, S154H, S158H, R164H, A166H, N167H, Q176H, N177H, N179H, R180H, S182H, A188H, N198H, Q200H, Y203H, G205H, S206H, T207H, Y208H, S210H, N212H, K231H, P233H, S234H, W235H, V238H, Q239H, N242H, N246H, T249H, S250H, S253H, T254H, N255H, R269H

### Accessibility >100%, Min Distance to Active Site 6 Angstroms

A1H, S3H, V4H, S9H, R10H, Q12H, N18H, R19H, S24H, T37H, P39H, N42H, R44H, P51H, G52H, P54H, Q57H, N74H, S76H, S97H, S99H, S103H, Q107H, E110H, N114H, N115H, P127H, S128H, P129H, A131H, T132H, E134H, Q135H, S142H, R143H, N153H, S154H, R164H, A166H, Q176H, N177H, N179H, S182H, A188H, N198H, Q200H, Y203H, G205H, S206H, T207H, Y208H, N212H, K231H, P233H, S234H, V238H, Q239H, N242H, N246H, S250H, T254H, N255H, R269H

### Accessibility >110%, Min Distance to Active Site 6 Angstroms

A1H, R10H, Q12H, N18H, R19H, T37H, N42H, R44H, P51H, G52H, P54H, Q57H, N74H, S76H, S97H, S99H, Q107H, N114H, N115H, P127H, P129H, T132H, E134H, Q135H, S142H, R143H, N153H, S154H, R164H, A166H, Q176H, N177H, N179H, S182H, A188H, N198H, Q200H, Y203H, G205H, S206H, N212H, K231H, P233H, S234H, V238H, Q239H, N242H, N246H, S250H, T254H, N255H, R269H

### Accessibility >120%, Min Distance to Active Site 6 Angstroms

A1H, R10H, N18H, R19H, T37H, N42H, R44H, P51H, G52H, P54H, Q57H, N74H, S76H, S97H, S99H, Q107H, N114H, N115H, P127H, P129H, E134H, Q135H, S142H, R143H, N153H, S154H, R164H, A166H, Q176H, N177H, N179H, S182H, A188H, N198H, Q200H, Y203H, N212H, K231H, P233H, S234H, Q239H, N246H, S250H, T254H, N255H, R269H

### Accessibility >130%, Min Distance to Active Site 6 Angstroms

### A1H, N18H, R19H, T37H, N42H, R44H, P51H, G52H, P54H, Q57H, N74H, S76H, S97H, Q107H, N114H, P127H, P129H, Q135H, S142H, R143H, R164H, Q176H, N177H, S182H, Q200H, Y203H, N212H, K231H, P233H, S234H, Q239H, N246H, S250H, T254H, R269H

### Accessibility >140%, Min Distance to Active Site 6 Angstroms

### A1H, N18H, R19H, T37H, N42H, R44H, P51H, P54H, Q57H, N74H, S76H, S97H, Q107H, N114H, P127H, P129H, Q135H, S142H, R164H, N177H, S182H, N212H, K231H, P233H, S234H, Q239H, N246H, S250H, T254H, R269H

In a preferred embodiment, the histidine modified variant comprises any one or more of the following substitutions G20H, T22H, Y89H, N115H, Q135H, S139H, R143H, N232H, V238H, N242H or N246H.

A particular preferred embodiment relates to a protease variant comprising an amino acid sequence which is at least 60% identical to SEQ ID NO 3 and comprises 2 to 6 of the following substitutions: G20H, T22H, Y89H, N115H, Q135H, S139H, R143H, N232H, V238H, N242H or N246H compared to SEQ ID NO 3.

A particular preferred embodiment relates to a protease variant comprising an amino acid sequence which is at least 60% identical to SEQ ID NO 3 and comprises 2 to 6 of the following substitutions: G20H, T22H, Y89H, N115H, Q135H, S139H, R143H, N232H, V238H, N242H or N246H compared to SEQ ID NO 4.

In one particular embodiment, the histidine modified protease variant of the invention comprises the substitution A47H and according to a particular preferred embodiment relates to a protease variant comprising an amino acid sequence which is at least 60% identical to SEQ ID NO 3 and comprises 2 to 6 of the following substitutions: G20H, T22H, A47H, Y89H, N115H, Q135H, S139H, R143H, N232H, V238H, N242H or N246H compared to SEQ ID NO 3.

A particular preferred embodiment relates to a protease variant comprising an amino acid sequence which is at least 60% identical to SEQ ID NO 3 and comprises 2 to 6 of the following substitutions: G20H, T22H, A47H, Y89H, N115H, Q135H, S139H, R143H, N232H, V238H, N242H or N246H compared to SEQ ID NO 4.

Another preferred embodiment relates to a protease variant of a protease parent with at least 60% identity to SEQ ID 3, wherein the protease variant comprises 2 to 6 of the histidines which have a surface accessibility of 140% i.e. histidines at any of the positions selected from the list consisting of 1, 18, 19, 37, 42, 44, 51, 54, 57, 74, 76, 97, 107, 114, 127, 129, 135, 142, 164, 177, 182, 212, 231, 233, 234, 239, 246, 250, 254 and 269, wherein the positions corresponds to the positions in SEQ ID NO 3. One preferred embodiment relates to a protease variant of a protease parent with SEQ ID NO 3 or a protease parent having at least 60% identity hereto, wherein the variant comprises 2 to 6 of the substitutions selected from the group consisting of A1H, N18H, R19H, T37H, N42H, R44H, P51H, P54H, Q57H, N74H, S76H, S97H, Q107H, N114H, P127H, P129H, Q135H, S142H, R164H, N177H, S182H, N212H, K231H, P233H, S234H, Q239H, N246H, S250H, T254H and R269H, wherein the positions corresponds to the positions in SEQ ID NO 3. The positions is in the context of the present application written with the amino acid present in the position corresponding to the position in the protease with SEQ ID NO 3, which is also the protease used for numbering. It is clear to the skilled artisan that a protease parent within 60% sequence identity to SEQ ID NO 3 may have another amino acid at the specific position. The above substitutions could also be written as X1H, X18H, X19H, X37H, X42H, X44H, X51H, X54H, X57H, X74H, X76H, X97H, X107H, X114H, X127H, X129H, X135H, X142H, X164H, X177H, X182H, X212H, X231H, X233H, X234H, X239H, X246H, X250H, X254H and X269H, where X indicate any amino acid could be present at the position.

One embodiment of the invention relates to a method of producing a protease variant of a parent protease by introducing into the parent protease 2 to 6 of the following substitutions: A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H. A preferred embodiment of the invention relates to a method of producing a variant of a parent protease having at least 60% identity to SEQ ID NO 3 wherein the protease variant has at least two additional histidines on the surface compared to the parent protease, the method comprising introducing into the parent protease alterations at 2 to 6 positions selected from the list consisting of A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259, R269 to obtain additional 2 to 6 histidines at the surface of the mature polypeptide of the parent protease, wherein each alteration is independently a substitution or insertion.

Another preferred embodiment of the invention relates a method of producing a protease variant having increased solubility at pH below 5 the method comprising substituting 2 to 6 of the following A1H, N18H, R19H, T37H, N42H, R44H, P51H, P54H, Q57H, N74H, S76H, S97H, Q107H, N114H, P127H, P129H, Q135H, S142H, R164H, N177H, S182H, N212H, K231H, P233H, S234H, Q239H, N246H, S250H, T254H, R269H into a polypeptide having at least 60% identity to SEQ ID NO 3. The histidine modified variants having increased solubility may further comprise additional substitutions, deletions and/or insertions at one or more positions (e.g. several).

A particular preferred embodiment of the invention relates to a protease variant having an amino acid sequence which is at least 60% identical to the mature polypeptide with SEQ ID NO 2 and comprises histidines at 2 to 6 positions selected from the list consisting of: A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259 and R269. In one embodiment when the alteration is an insertion the histidines are not added to the C- or N-terminal. In a preferred embodiment the protease variant has an amino acid sequence which is at least 60% identical to the mature polypeptide with SEQ ID NO 2 and comprises 2 to 6 of the substitutions selected from the group consisting of A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H and R269H. In one embodiment the protease variant comprises at least one additional substitution at positions selected from the group consisting of positions: 3, 4, 9, 15, 24, 42, 59, 66, 74, 76, 97, 99, 101, 102, 118, 126, 127, 128, 154, 156, 157, 158, 161, 164, 176, 179, 182, 188, 198, 199, 200, 203, 210, 211, 212, 216, 239, 255 and 256, preferably positions 9, 15, 42, 66, 74, 97, 99, 154, 200, 203, 211, 212 and/or 256 (numbering according to SEQ ID NO: 3). It will be clear to the skilled artisan that if a position has already been altered once, then it will not be altered a second time. In a preferred embodiment, the additional substitution at one or more positions (e.g. several) is selected from the group consisting of S3T, V41, S9[E,D,K,R], A15T, S24G, N42[R,K], G59E, V66[G,A,S;T], N74[D,E], S76[N,Q], S97[D,E,A], S99[R,K,N,M,E,D,L,I], S101A, V1021, H118[D,E], S126[L,I,V], P127[N,Q] S128A, S154[E,D], A156[D,E], G157P, S158[E,D], Y161A, R164S, Q176 [D,E], N179[D,E], S182[D,E], A188P, V199M, N198[D,E], V1991, Q200L, Y203W, S210V, L211[D,E], N212[D,E], M216S, Q239[K,R], N255[D,E] and L256[D,E] (numbering according to SEQ ID NO: 3). In an even more preferred embodiment, the histidine modified variant further comprises one or more substitutions selected from the group consisting of V41, S9E, N42R, V66A, N74D, S97D, S97A, S99L, S99M, S99D, S99E, S99N, R164S, S154D, Y161A, N179E, S182E, V1991, Q200L, Y203W, L211D, L211E, Q239R, and/or L256E.

In one particular embodiment, the histidine modified protease variant of the invention comprises the amino acid sequence of SEQ ID NO 4. In one embodiment the histidine modified protease of the invention comprises an amino acid sequence which is at least 60% identical to SEQ ID NO 4 and comprises histidines at 2 to 6 positions selected from the list consisting of: A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259 and R269 when compared to SEQ ID NO 4. In one embodiment the histidine modified protease variant consist of SEQ ID NO 4 and histidines at 2 to 6 positions selected from the list consisting of: A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259 and R269 when compared to SEQ ID NO 4

One embodiment of the invention relates to protease variants which compared to SEQ ID NO 3 comprises any of the following substitutions:

A1H+Q2H, A1H+S3H, A1H+V4H, A1H+W6H, A1H+S9H, A1H+R10H, A1H+Q12H, A1H+P14H, A1H+A15H, A1H+N18H, A1H+R19H, A1H+G20H, A1H+T22H, A1H+S24H, A1H+G25H, A1H+K27H, A1H+T37H, A1H+P39H, A1H+N42H, A1H+143H, A1H+R44H, A1H+G45H, A1H+G46H, A1H+S48H, A1H+F49H, A1H+P51H, A1H+G52H, A1H+E53H, A1H+P54H, A1H+S55H, A1H+T56H, A1H+Q57H, A1H+G59H, A1H+L73H, A1H+N74H, A1H+N75H, A1H+S76H, A1H+177H, A1H+S85H, A1H+E87H, A1H+Y89H, A1H+A96H, A1H+S97H, A1H+G98H, A1H+S99H, A1H+G100H, A1H+S101H, A1H+V102H, A1H+S103H, A1H+Q107H, A1H+E110H, A1H+W111H, A1H+N114H, A1H+N115H, A1H+G116H, A1H+G125H, A1H+S126H, A1H+P127H, A1H+S128H, A1H+P129H, A1H+A131H, A1H+T132H, A1H+E134H, A1H+Q135H, A1H+N138H, A1H+S139H, A1H+S142H, A1H+R143H, A1H+G144H, A1H+N153H, A1H+S154H, A1H+G155H, A1H+A156H, A1H+G157H, A1H+S158H, A1H+Y161H, A1H+R164H, A1H+A166H, A1H+N167H, A1H+D175H, A1H+Q176H, A1H+N177H, A1H+N178H, A1H+N179H, A1H+R180H, A1H+S182H, A1H+F183H, A1H+Y186H, A1H+A188H, A1H+G189H, A1H+N198H, A1H+Q200H, A1H+Y203H, A1H+P204H, A1H+G205H, A1H+S206H, A1H+T207H, A1H+Y208H, A1H+S210H, A1H+L211H, A1H+N212H, A1H+K229H, A1H+Q230H, A1H+K231H, A1H+N232H, A1H+P233H, A1H+S234H, A1H+W235H, A1H+S236H, A1H+V238H, A1H+Q239H, A1H+R241H, A1H+N242H, A1H+K245H, A1H+N246H, A1H+T247H, A1H+T249H, A1H+S250H, A1H+L251H, A1H+G252H, A1H+S253H, A1H+T254H, A1H+N255H, A1H+L256H, A1H+Y257H, A1H+S259H, A1H+E265H, A1H+R269H, Q2H+S3H, Q2H+V4H, Q2H+W6H, Q2H+S9H, Q2H+R10H, Q2H+Q12H, Q2H+P14H, Q2H+A15H, Q2H+N18H, Q2H+R19H, Q2H+G20H, Q2H+T22H, Q2H+S24H, Q2H+G25H, Q2H+K27H, Q2H+T37H, Q2H+P39H, Q2H+N42H, Q2H+I43H, Q2H+R44H, Q2H+G45H, Q2H+G46H, Q2H+S48H, Q2H+F49H, Q2H+P51H, Q2H+G52H, Q2H+E53H, Q2H+P54H, Q2H+S55H, Q2H+T56H, Q2H+Q57H, Q2H+G59H, Q2H+L73H, Q2H+N74H, Q2H+N75H, Q2H+S76H, Q2H+177H, Q2H+S85H, Q2H+E87H, Q2H+Y89H, Q2H+A96H, Q2H+S97H, Q2H+G98H, Q2H+S99H, Q2H+G100H, Q2H+S101H, Q2H+V102H, Q2H+S103H, Q2H+Q107H, Q2H+E110H, Q2H+W111H, Q2H+N114H, Q2H+N115H, Q2H+G116H, Q2H+G125H, Q2H+S126H, Q2H+P127H, Q2H+S128H, Q2H+P129H, Q2H+A131H, Q2H+T132H, Q2H+E134H, Q2H+Q135H, Q2H+N138H, Q2H+S139H, Q2H+S142H, Q2H+R143H, Q2H+G144H, Q2H+N153H, Q2H+S154H, Q2H+G155H, Q2H+A156H, Q2H+G157H, Q2H+S158H, Q2H+Y161H, Q2H+R164H, Q2H+A166H, Q2H+N167H, Q2H+D175H, Q2H+Q176H, Q2H+N177H, Q2H+N178H, Q2H+N179H, Q2H+R180H, Q2H+S182H, Q2H+F183H, Q2H+Y186H, Q2H+A188H, Q2H+G189H, Q2H+N198H, Q2H+Q200H, Q2H+Y203H, Q2H+P204H, Q2H+G205H, Q2H+S206H, Q2H+T207H, Q2H+Y208H, Q2H+S210H, Q2H+L211H, Q2H+N212H, Q2H+K229H, Q2H+Q230H, Q2H+K231H, Q2H+N232H, Q2H+P233H, Q2H+S234H, Q2H+W235H, Q2H+S236H, Q2H+V238H, Q2H+Q239H, Q2H+R241H, Q2H+N242H, Q2H+K245H, Q2H+N246H, Q2H+T247H, Q2H+T249H, Q2H+S250H, Q2H+L251H, Q2H+G252H, Q2H+S253H, Q2H+T254H, Q2H+N255H, Q2H+L256H, Q2H+Y257H, Q2H+S259H, Q2H+E265H, Q2H+R269H, S3H+V4H, S3H+W6H, S3H+S9H, S3H+R10H, S3H+Q12H, S3H+P14H, S3H+A15H, S3H+N18H, S3H+R19H, S3H+G20H, S3H+T22H, S3H+S24H, S3H+G25H, S3H+K27H, S3H+T37H, S3H+P39H, S3H+N42H, S3H+I43H, S3H+R44H, S3H+G45H, S3H+G46H, S3H+S48H, S3H+F49H, S3H+P51H, S3H+G52H, S3H+E53H, S3H+P54H, S3H+S55H, S3H+T56H, S3H+Q57H, S3H+G59H, S3H+L73H, S3H+N74H, S3H+N75H, S3H+S76H, S3H+177H, S3H+S85H, S3H+E87H, S3H+Y89H, S3H+A96H, S3H+S97H, S3H+G98H, S3H+S99H, S3H+G100H, S3H+S101H, S3H+V102H, S3H+S103H, S3H+Q107H, S3H+E110H, S3H+W111H, S3H+N114H, S3H+N115H, S3H+G116H, S3H+G125H, S3H+S126H, S3H+P127H, S3H+S128H, S3H+P129H, S3H+A131H, S3H+T132H, S3H+E134H, S3H+Q135H, S3H+N138H, S3H+S139H, S3H+S142H, S3H+R143H, S3H+G144H, S3H+N153H, S3H+S154H, S3H+G155H, S3H+A156H, S3H+G157H, S3H+S158H, S3H+Y161H, S3H+R164H, S3H+A166H, S3H+N167H, S3H+D175H, S3H+Q176H, S3H+N177H, S3H+N178H, S3H+N179H, S3H+R180H, S3H+S182H, S3H+F183H, S3H+Y186H, S3H+A188H, S3H+G189H, S3H+N198H, S3H+Q200H, S3H+Y203H, S3H+P204H, S3H+G205H, S3H+S206H, S3H+T207H, S3H+Y208H, S3H+S210H, S3H+L211H, S3H+N212H, S3H+K229H, S3H+Q230H, S3H+K231H, S3H+N232H, S3H+P233H, S3H+S234H, S3H+W235H, S3H+S236H, S3H+V238H, S3H+Q239H, S3H+R241H, S3H+N242H, S3H+K245H, S3H+N246H, S3H+T247H, S3H+T249H, S3H+S250H, S3H+L251H, S3H+G252H, S3H+S253H, S3H+T254H, S3H+N255H, S3H+L256H, S3H+Y257H, S3H+S259H, S3H+E265H, S3H+R269H, V4H+W6H, V4H+S9H, V4H+R10H, V4H+Q12H, V4H+P14H, V4H+A15H, V4H+N18H, V4H+R19H, V4H+G20H, V4H+T22H, V4H+S24H, V4H+G25H, V4H+K27H, V4H+T37H, V4H+P39H, V4H+N42H, V4H+I43H, V4H+R44H, V4H+G45H, V4H+G46H, V4H+S48H, V4H+F49H, V4H+P51H, V4H+G52H, V4H+E53H, V4H+P54H, V4H+S55H, V4H+T56H, V4H+Q57H, V4H+G59H, V4H+L73H, V4H+N74H, V4H+N75H, V4H+S76H, V4H+I77H, V4H+S85H, V4H+E87H, V4H+Y89H, V4H+A96H, V4H+S97H, V4H+G98H, V4H+S99H, V4H+G100H, V4H+S101H, V4H+V102H, V4H+S103H, V4H+Q107H, V4H+E110H, V4H+W111H, V4H+N114H, V4H+N115H, V4H+G116H, V4H+G125H, V4H+S126H, V4H+P127H, V4H+S128H, V4H+P129H, V4H+A131H, V4H+T132H, V4H+E134H, V4H+Q135H, V4H+N138H, V4H+S139H, V4H+S142H, V4H+R143H, V4H+G144H, V4H+N153H, V4H+S154H, V4H+G155H, V4H+A156H, V4H+G157H, V4H+S158H, V4H+Y161H, V4H+R164H, V4H+A166H, V4H+N167H, V4H+D175H, V4H+Q176H, V4H+N177H, V4H+N178H, V4H+N179H, V4H+R180H, V4H+S182H, V4H+F183H, V4H+Y186H, V4H+A188H, V4H+G189H, V4H+N198H, V4H+Q200H, V4H+Y203H, V4H+P204H, V4H+G205H, V4H+S206H, V4H+T207H, V4H+Y208H, V4H+S210H, V4H+L211H, V4H+N212H, V4H+K229H, V4H+Q230H, V4H+K231H, V4H+N232H, V4H+P233H, V4H+S234H, V4H+W235H, V4H+S236H, V4H+V238H, V4H+Q239H, V4H+R241H, V4H+N242H, V4H+K245H, V4H+N246H, V4H+T247H, V4H+T249H, V4H+S250H, V4H+L251H, V4H+G252H, V4H+S253H, V4H+T254H, V4H+N255H, V4H+L256H, V4H+Y257H, V4H+S259H, V4H+E265H, V4H+R269H, W6H+S9H, W6H+R10H, W6H+Q12H, W6H+P14H, W6H+A15H, W6H+N18H, W6H+R19H, W6H+G20H, W6H+T22H, W6H+S24H, W6H+G25H, W6H+K27H, W6H+T37H, W6H+P39H, W6H+N42H, W6H+I43H, W6H+R44H, W6H+G45H, W6H+G46H, W6H+S48H, W6H+F49H, W6H+P51H, W6H+G52H, W6H+E53H, W6H+P54H, W6H+S55H, W6H+T56H, W6H+Q57H, W6H+G59H, W6H+L73H, W6H+N74H, W6H+N75H, W6H+S76H, W6H+I77H, W6H+S85H, W6H+E87H, W6H+Y89H, W6H+A96H, W6H+S97H, W6H+G98H, W6H+S99H, W6H+G100H, W6H+S101H, W6H+V102H, W6H+S103H, W6H+Q107H, W6H+E110H, W6H+W111H, W6H+N114H, W6H+N115H, W6H+G116H, W6H+G125H, W6H+S126H, W6H+P127H, W6H+S128H, W6H+P129H, W6H+A131H, W6H+T132H, W6H+E134H, W6H+Q135H, W6H+N138H, W6H+S139H, W6H+S142H, W6H+R143H, W6H+G144H, W6H+N153H, W6H+S154H, W6H+G155H, W6H+A156H, W6H+G157H, W6H+S158H, W6H+Y161H, W6H+R164H, W6H+A166H, W6H+N167H, W6H+D175H, W6H+Q176H, W6H+N177H, W6H+N178H, W6H+N179H, W6H+R180H, W6H+S182H, W6H+F183H, W6H+Y186H, W6H+A188H, W6H+G189H, W6H+N198H, W6H+Q200H, W6H+Y203H, W6H+P204H, W6H+G205H, W6H+S206H, W6H+T207H, W6H+Y208H, W6H+S210H, W6H+L211H, W6H+N212H, W6H+K229H, W6H+Q230H, W6H+K231H, W6H+N232H, W6H+P233H, W6H+S234H, W6H+W235H, W6H+S236H, W6H+V238H, W6H+Q239H, W6H+R241H, W6H+N242H, W6H+K245H, W6H+N246H, W6H+T247H, W6H+T249H, W6H+S250H, W6H+L251H, W6H+G252H, W6H+S253H, W6H+T254H, W6H+N255H, W6H+L256H, W6H+Y257H, W6H+S259H, W6H+E265H, W6H+R269H, S9H+R10H, S9H+Q12H, S9H+P14H, S9H+A15H, S9H+N18H, S9H+R19H, S9H+G20H, S9H+T22H, S9H+S24H, S9H+G25H, S9H+K27H, S9H+T37H, S9H+P39H, S9H+N42H, S9H+I43H, S9H+R44H, S9H+G45H, S9H+G46H, S9H+S48H, S9H+F49H, S9H+P51H, S9H+G52H, S9H+E53H, S9H+P54H, S9H+S55H, S9H+T56H, S9H+Q57H, S9H+G59H, S9H+L73H, S9H+N74H, S9H+N75H, S9H+S76H, S9H+I77H, S9H+S85H, S9H+E87H, S9H+Y89H, S9H+A96H, S9H+S97H, S9H+G98H, S9H+S99H, S9H+G100H, S9H+S101H, S9H+V102H, S9H+S103H, S9H+Q107H, S9H+E110H, S9H+W111H, S9H+N114H, S9H+N115H, S9H+G116H, S9H+G125H, S9H+S126H, S9H+P127H, S9H+S128H, S9H+P129H, S9H+A131H, S9H+T132H, S9H+E134H, S9H+Q135H, S9H+N138H, S9H+S139H, S9H+S142H, S9H+R143H, S9H+G144H, S9H+N153H, S9H+S154H, S9H+G155H, S9H+A156H, S9H+G157H, S9H+S158H, S9H+Y161H, S9H+R164H, S9H+A166H, S9H+N167H, S9H+D175H, S9H+Q176H, S9H+N177H, S9H+N178H, S9H+N179H, S9H+R180H, S9H+S182H, S9H+F183H, S9H+Y186H, S9H+A188H, S9H+G189H, S9H+N198H, S9H+Q200H, S9H+Y203H, S9H+P204H, S9H+G205H, S9H+S206H, S9H+T207H, S9H+Y208H, S9H+S210H, S9H+L211H, S9H+N212H, S9H+K229H, S9H+Q230H, S9H+K231H, S9H+N232H, S9H+P233H, S9H+S234H, S9H+W235H, S9H+S236H, S9H+V238H, S9H+Q239H, S9H+R241H, S9H+N242H, S9H+K245H, S9H+N246H, S9H+T247H, S9H+T249H, S9H+S250H, S9H+L251H, S9H+G252H, S9H+S253H, S9H+T254H, S9H+N255H, S9H+L256H, S9H+Y257H, S9H+S259H, S9H+E265H, S9H+R269H, R10H+Q12H, R10H+P14H, R10H+A15H, R10H+N18H, R10H+R19H, R10H+G20H, R10H+T22H, R10H+S24H, R10H+G25H, R10H+K27H, R10H+T37H, R10H+P39H, R10H+N42H, R10H+143H, R10H+R44H, R10H+G45H, R10H+G46H, R10H+S48H, R10H+F49H, R10H+P51H, R10H+G52H, R10H+E53H, R10H+P54H, R10H+S55H, R10H+T56H, R10H+Q57H, R10H+G59H, R10H+L73H, R10H+N74H, R10H+N75H, R10H+S76H, R10H+177H, R10H+S85H, R10H+E87H, R10H+Y89H, R10H+A96H, R10H+S97H, R10H+G98H, R10H+S99H, R10H+G100H, R10H+S101H, R10H+V102H, R10H+S103H, R10H+Q107H, R10H+E110H, R10H+W111H, R10H+N114H, R10H+N115H, R10H+G116H, R10H+G125H, R10H+S126H, R10H+P127H, R10H+S128H, R10H+P129H, R10H+A131H, R10H+T132H, R10H+E134H, R10H+Q135H, R10H+N138H, R10H+S139H, R10H+S142H, R10H+R143H, R10H+G144H, R10H+N153H, R10H+S154H, R10H+G155H, R10H+A156H, R10H+G157H, R10H+S158H, R10H+Y161H, R10H+R164H, R10H+A166H, R10H+N167H, R10H+D175H, R10H+Q176H, R10H+N177H, R10H+N178H, R10H+N179H, R10H+R180H, R10H+S182H, R10H+F183H, R10H+Y186H, R10H+A188H, R10H+G189H, R10H+N198H, R10H+Q200H, R10H+Y203H, R10H+P204H, R10H+G205H, R10H+S206H, R10H+T207H, R10H+Y208H, R10H+S210H, R10H+L211H, R10H+N212H, R10H+K229H, R10H+Q230H, R10H+K231H, R10H+N232H, R10H+P233H, R10H+S234H, R10H+W235H, R10H+S236H, R10H+V238H, R10H+Q239H, R10H+R241H, R10H+N242H, R10H+K245H, R10H+N246H, R10H+T247H, R10H+T249H, R10H+S250H, R10H+L251H, R10H+G252H, R10H+S253H, R10H+T254H, R10H+N255H, R10H+L256H, R10H+Y257H, R10H+S259H, R10H+E265H, R10H+R269H, Q12H+P14H, Q12H+A15H, Q12H+N18H, Q12H+R19H, Q12H+G20H, Q12H+T22H, Q12H+S24H, Q12H+G25H, Q12H+K27H, Q12H+T37H, Q12H+P39H, Q12H+N42H, Q12H+I43H, Q12H+R44H, Q12H+G45H, Q12H+G46H, Q12H+S48H, Q12H+F49H, Q12H+P51H, Q12H+G52H, Q12H+E53H, Q12H+P54H, Q12H+S55H, Q12H+T56H, Q12H+Q57H, Q12H+G59H, Q12H+L73H, Q12H+N74H, Q12H+N75H, Q12H+S76H, Q12H+177H, Q12H+S85H, Q12H+E87H, Q12H+Y89H, Q12H+A96H, Q12H+S97H, Q12H+G98H, Q12H+S99H, Q12H+G100H, Q12H+S101H, Q12H+V102H, Q12H+S103H, Q12H+Q107H, Q12H+E110H, Q12H+W111H, Q12H+N114H, Q12H+N115H, Q12H+G116H, Q12H+G125H, Q12H+S126H, Q12H+P127H, Q12H+S128H, Q12H+P129H, Q12H+A131H, Q12H+T132H, Q12H+E134H, Q12H+Q135H, Q12H+N138H, Q12H+S139H, Q12H+S142H, Q12H+R143H, Q12H+G144H, Q12H+N153H, Q12H+S154H, Q12H+G155H, Q12H+A156H, Q12H+G157H, Q12H+S158H, Q12H+Y161H, Q12H+R164H, Q12H+A166H, Q12H+N167H, Q12H+D175H, Q12H+Q176H, Q12H+N177H, Q12H+N178H, Q12H+N179H, Q12H+R180H, Q12H+S182H, Q12H+F183H, Q12H+Y186H, Q12H+A188H, Q12H+G189H, Q12H+N198H, Q12H+Q200H, Q12H+Y203H, Q12H+P204H, Q12H+G205H, Q12H+S206H, Q12H+T207H, Q12H+Y208H, Q12H+S210H, Q12H+L211H, Q12H+N212H, Q12H+K229H, Q12H+Q230H, Q12H+K231H, Q12H+N232H, Q12H+P233H, Q12H+S234H, Q12H+W235H, Q12H+S236H, Q12H+V238H, Q12H+Q239H, Q12H+R241H, Q12H+N242H, Q12H+K245H, Q12H+N246H, Q12H+T247H, Q12H+T249H, Q12H+S250H, Q12H+L251H, Q12H+G252H, Q12H+S253H, Q12H+T254H, Q12H+N255H, Q12H+L256H, Q12H+Y257H, Q12H+S259H, Q12H+E265H, Q12H+R269H, P14H+A15H, P14H+N18H, P14H+R19H, P14H+G20H, P14H+T22H, P14H+S24H, P14H+G25H, P14H+K27H, P14H+T37H, P14H+P39H, P14H+N42H, P14H+I43H, P14H+R44H, P14H+G45H, P14H+G46H, P14H+S48H, P14H+F49H, P14H+P51H, P14H+G52H, P14H+E53H, P14H+P54H, P14H+S55H, P14H+T56H, P14H+Q57H, P14H+G59H, P14H+L73H, P14H+N74H, P14H+N75H, P14H+S76H, P14H+177H, P14H+S85H, P14H+E87H, P14H+Y89H, P14H+A96H, P14H+S97H, P14H+G98H, P14H+S99H, P14H+G100H, P14H+S101H, P14H+V102H, P14H+S103H, P14H+Q107H, P14H+E110H, P14H+W111H, P14H+N114H, P14H+N115H, P14H+G116H, P14H+G125H, P14H+S126H, P14H+P127H, P14H+S128H, P14H+P129H, P14H+A131H, P14H+T132H, P14H+E134H, P14H+Q135H, P14H+N138H, P14H+S139H, P14H+S142H, P14H+R143H, P14H+G144H, P14H+N153H, P14H+S154H, P14H+G155H, P14H+A156H, P14H+G157H, P14H+S158H, P14H+Y161H, P14H+R164H, P14H+A166H, P14H+N167H, P14H+D175H, P14H+Q176H, P14H+N177H, P14H+N178H, P14H+N179H, P14H+R180H, P14H+S182H, P14H+F183H, P14H+Y186H, P14H+A188H, P14H+G189H, P14H+N198H, P14H+Q200H, P14H+Y203H, P14H+P204H, P14H+G205H, P14H+S206H, P14H+T207H, P14H+Y208H, P14H+S210H, P14H+L211H, P14H+N212H, P14H+K229H, P14H+Q230H, P14H+K231H, P14H+N232H, P14H+P233H, P14H+S234H, P14H+W235H, P14H+S236H, P14H+V238H, P14H+Q239H, P14H+R241H, P14H+N242H, P14H+K245H, P14H+N246H, P14H+T247H, P14H+T249H, P14H+S250H, P14H+L251H, P14H+G252H, P14H+S253H, P14H+T254H, P14H+N255H, P14H+L256H, P14H+Y257H, P14H+S259H, P14H+E265H, P14H+R269H, A15H+N18H, A15H+R19H, A15H+G20H, A15H+T22H, A15H+S24H, A15H+G25H, A15H+K27H, A15H+T37H, A15H+P39H, A15H+N42H, A15H+I43H, A15H+R44H, A15H+G45H, A15H+G46H, A15H+S48H, A15H+F49H, A15H+P51H, A15H+G52H, A15H+E53H, A15H+P54H, A15H+S55H, A15H+T56H, A15H+Q57H, A15H+G59H, A15H+L73H, A15H+N74H, P15H+N75H, P15H+S76H, P15H+177H, A15H+S85H, A15H+E87H, P15H+Y89H, A15H+A96H, A15H+S97H, A15H+G98H, A15H+S99H, A15H+G100H, A15H+S101H, A15H+V102H, A15H+S103H, A15H+Q107H, A15H+E110H, A15H+W111H, A15H+N114H, A15H+N115H, A15H+G116H, A15H+G125H, A15H+S126H, A15H+P127H, A15H+S128H, A15H+P129H, A15H+A131H, A15H+T132H, A15H+E134H, A15H+Q135H, A15H+N138H, A15H+S139H, A15H+S142H, A15H+R143H, A15H+G144H, A15H+N153H, A15H+S154H, A15H+G155H, A15H+A156H, A15H+G157H, A15H+S158H, A15H+Y161H, A15H+R164H, A15H+A166H, A15H+N167H, A15H+D175H, A15H+Q176H, A15H+N177H, A15H+N178H, A15H+N179H, A15H+R180H, A15H+S182H, A15H+F183H, A15H+Y186H, A15H+A188H, A15H+G189H, A15H+N198H, A15H+Q200H, A15H+Y203H, A15H+P204H, A15H+G205H, A15H+S206H, A15H+T207H, A15H+Y208H, A15H+S210H, A15H+L211H, A15H+N212H, A15H+K229H, A15H+Q230H, A15H+K231H, A15H+N232H, A15H+P233H, A15H+S234H, A15H+W235H, A15H+S236H, A15H+V238H, A15H+Q239H, A15H+R241H, A15H+N242H, A15H+K245H, A15H+N246H, A15H+T247H, A15H+T249H, A15H+S250H, A15H+L251H, A15H+G252H, A15H+S253H, A15H+T254H, A15H+N255H, A15H+L256H, A15H+Y257H, A15H+S259H, A15H+E265H, A15H+R269H, N18H+R19H, N18H+G20H, N18H+T22H, N18H+S24H, N18H+G25H, N18H+K27H, N18H+T37H, N18H+P39H, N18H+N42H, N18H+I43H, N18H+R44H, N18H+G45H, N18H+G46H, N18H+S48H, N18H+F49H, N18H+P51H, N18H+G52H, N18H+E53H, N18H+P54H, N18H+S55H, N18H+T56H, N18H+Q57H, N18H+G59H, N18H+L73H, N18H+N74H, N18H+N75H, N18H+S76H, N18H+177H, N18H+S85H, N18H+E87H, N18H+Y89H, N18H+A96H, N18H+S97H, N18H+G98H, N18H+S99H, N18H+G100H, N18H+S101H, N18H+V102H, N18H+S103H, N18H+Q107H, N18H+E110H, N18H+W111H, N18H+N114H, N18H+N115H, N18H+G116H, N18H+G125H, N18H+S126H, N18H+P127H, N18H+S128H, N18H+P129H, N18H+A131H, N18H+T132H, N18H+E134H, N18H+Q135H, N18H+N138H, N18H+S139H, N18H+S142H, N18H+R143H, N18H+G144H, N18H+N153H, N18H+S154H, N18H+G155H, N18H+A156H, N18H+G157H, N18H+S158H, N18H+Y161H, N18H+R164H, N18H+A166H, N18H+N167H, N18H+D175H, N18H+Q176H, N18H+N177H, N18H+N178H, N18H+N179H, N18H+R180H, N18H+S182H, N18H+F183H, N18H+Y186H, N18H+A188H, N18H+G189H, N18H+N198H, N18H+Q200H, N18H+Y203H, N18H+P204H, N18H+G205H, N18H+S206H, N18H+T207H, N18H+Y208H, N18H+S210H, N18H+L211H, N18H+N212H, N18H+K229H, N18H+Q230H, N18H+K231H, N18H+N232H, N18H+P233H, N18H+S234H, N18H+W235H, N18H+S236H, N18H+V238H, N18H+Q239H, N18H+R241H, N18H+N242H, N18H+K245H, N18H+N246H, N18H+T247H, N18H+T249H, N18H+S250H, N18H+L251H, N18H+G252H, N18H+S253H, N18H+T254H, N18H+N255H, N18H+L256H, N18H+Y257H, N18H+S259H, N18H+E265H, N18H+R269H, R19H+G20H, R19H+T22H, R19H+S24H, R19H+G25H, R19H+K27H, R19H+T37H, R19H+P39H, R19H+N42H, R19H+I43H, R19H+R44H, R19H+G45H, R19H+G46H, R19H+S48H, R19H+F49H, R19H+P51H, R19H+G52H, R19H+E53H, R19H+P54H, R19H+S55H, R19H+T56H, R19H+Q57H, R19H+G59H, R19H+L73H, R19H+N74H, R19H+N75H, R19H+S76H, R19H+177H, R19H+S85H, R19H+E87H, R19H+Y89H, R19H+A96H, R19H+S97H, R19H+G98H, R19H+S99H, R19H+G100H, R19H+S101H, R19H+V102H, R19H+S103H, R19H+Q107H, R19H+E110H, R19H+W111H, R19H+N114H, R19H+N115H, R19H+G116H, R19H+G125H, R19H+S126H, R19H+P127H, R19H+S128H, R19H+P129H, R19H+A131H, R19H+T132H, R19H+E134H, R19H+Q135H, R19H+N138H, R19H+S139H, R19H+S142H, R19H+R143H, R19H+G144H, R19H+N153H, R19H+S154H, R19H+G155H, R19H+A156H, R19H+G157H, R19H+S158H, R19H+Y161H, R19H+R164H, R19H+A166H, R19H+N167H, R19H+D175H, R19H+Q176H, R19H+N177H, R19H+N178H, R19H+N179H, R19H+R180H, R19H+S182H, R19H+F183H, R19H+Y186H, R19H+A188H, R19H+G189H, R19H+N198H, R19H+Q200H, R19H+Y203H, R19H+P204H, R19H+G205H, R19H+S206H, R19H+T207H, R19H+Y208H, R19H+S210H, R19H+L211H, R19H+N212H, R19H+K229H, R19H+Q230H, R19H+K231H, R19H+N232H, R19H+P233H, R19H+S234H, R19H+W235H, R19H+S236H, R19H+V238H, R19H+Q239H, R19H+R241H, R19H+N242H, R19H+K245H, R19H+N246H, R19H+T247H, R19H+T249H, R19H+S250H, R19H+L251H, R19H+G252H, R19H+S253H, R19H+T254H, R19H+N255H, R19H+L256H, R19H+Y257H, R19H+S259H, R19H+E265H, R19H+R269H, G20H+T22H, G20H+S24H, G20H+G25H, G20H+K27H, G20H+T37H, G20H+P39H, G20H+N42H, G20H+143H, G20H+R44H, G20H+G45H, G20H+G46H, G20H+S48H, G20H+F49H, G20H+P51H, G20H+G52H, G20H+E53H, G20H+P54H, G20H+S55H, G20H+T56H, G20H+Q57H, G20H+G59H, G20H+L73H, G20H+N74H, G20H+N75H, G20H+S76H, G20H+177H, G20H+S85H, G20H+E87H, G20H+Y89H, G20H+A96H, G20H+S97H, G20H+G98H, G20H+S99H, G20H+G100H, G20H+S101H, G20H+V102H, G20H+S103H, G20H+Q107H, G20H+E110H, G20H+W111H, G20H+N114H, G20H+N115H, G20H+G116H, G20H+G125H, G20H+S126H, G20H+P127H, G20H+S128H, G20H+P129H, G20H+A131H, G20H+T132H, G20H+E134H, G20H+Q135H, G20H+N138H, G20H+S139H, G20H+S142H, G20H+R143H, G20H+G144H, G20H+N153H, G20H+S154H, G20H+G155H, G20H+A156H, G20H+G157H, G20H+S158H, G20H+Y161H, G20H+R164H, G20H+A166H, G20H+N167H, G20H+D175H, G20H+Q176H, G20H+N177H, G20H+N178H, G20H+N179H, G20H+R180H, G20H+S182H, G20H+F183H, G20H+Y186H, G20H+A188H, G20H+G189H, G20H+N198H, G20H+Q200H, G20H+Y203H, G20H+P204H, G20H+G205H, G20H+S206H, G20H+T207H, G20H+Y208H, G20H+S210H, G20H+L211H, G20H+N212H, G20H+K229H, G20H+Q230H, G20H+K231H, G20H+N232H, G20H+P233H, G20H+S234H, G20H+W235H, G20H+S236H, G20H+V238H, G20H+Q239H, G20H+R241H, G20H+N242H, G20H+K245H, G20H+N246H, G20H+T247H, G20H+T249H, G20H+S250H, G20H+L251H, G20H+G252H, G20H+S253H, G20H+T254H, G20H+N255H, G20H+L256H, G20H+Y257H, G20H+S259H, G20H+E265H, G20H+R269H, T22H+S24H, T22H+G25H, T22H+K27H, T22H+T37H, T22H+P39H, T22H+N42H, T22H+I43H, T22H+R44H, T22H+G45H, T22H+G46H, T22H+S48H, T22H+F49H, T22H+P51H, T22H+G52H, T22H+E53H, T22H+P54H, T22H+S55H, T22H+T56H, T22H+Q57H, T22H+G59H, T22H+L73H, T22H+N74H, T22H+N75H, T22H+S76H, T22H+177H, T22H+S85H, T22H+E87H, T22H+Y89H, T22H+A96H, T22H+S97H, T22H+G98H, T22H+S99H, T22H+G100H, T22H+S101H, T22H+V102H, T22H+S103H, T22H+Q107H, T22H+E110H, T22H+W111H, T22H+N114H, T22H+N115H, T22H+G116H, T22H+G125H, T22H+S126H, T22H+P127H, T22H+S128H, T22H+P129H, T22H+A131H, T22H+T132H, T22H+E134H, T22H+Q135H, T22H+N138H, T22H+S139H, T22H+S142H, T22H+R143H, T22H+G144H, T22H+N153H, T22H+S154H, T22H+G155H, T22H+A156H, T22H+G157H, T22H+S158H, T22H+Y161H, T22H+R164H, T22H+A166H, T22H+N167H, T22H+D175H, T22H+Q176H, T22H+N177H, T22H+N178H, T22H+N179H, T22H+R180H, T22H+S182H, T22H+F183H, T22H+Y186H, T22H+A188H, T22H+G189H, T22H+N198H, T22H+Q200H, T22H+Y203H, T22H+P204H, T22H+G205H, T22H+S206H, T22H+T207H, T22H+Y208H, T22H+S210H, T22H+L211H, T22H+N212H, T22H+K229H, T22H+Q230H, T22H+K231H, T22H+N232H, T22H+P233H, T22H+S234H, T22H+W235H, T22H+S236H, T22H+V238H, T22H+Q239H, T22H+R241H, T22H+N242H, T22H+K245H, T22H+N246H, T22H+T247H, T22H+T249H, T22H+S250H, T22H+L251H, T22H+G252H, T22H+S253H, T22H+T254H, T22H+N255H, T22H+L256H, T22H+Y257H, T22H+S259H, T22H+E265H, T22H+R269H, S24H+G25H, S24H+K27H, S24H+T37H, S24H+P39H, S24H+N42H, S24H+I43H, S24H+R44H, S24H+G45H, S24H+G46H, S24H+S48H, S24H+F49H, S24H+P51H, S24H+G52H, S24H+E53H, S24H+P54H, S24H+S55H, S24H+T56H, S24H+Q57H, S24H+G59H, S24H+L73H, S24H+N74H, S24H+N75H, S24H+S76H, S24H+177H, S24H+S85H, S24H+E87H, S24H+Y89H, S24H+A96H, S24H+S97H, S24H+G98H, S24H+S99H, S24H+G100H, S24H+S101H, S24H+V102H, S24H+S103H, S24H+Q107H, S24H+E110H, S24H+W111H, S24H+N114H, S24H+N115H, S24H+G116H, S24H+G125H, S24H+S126H, S24H+P127H, S24H+S128H, S24H+P129H, S24H+A131H, S24H+T132H, S24H+E134H, S24H+Q135H, S24H+N138H, S24H+S139H, S24H+S142H, S24H+R143H, S24H+G144H, S24H+N153H, S24H+S154H, S24H+G155H, S24H+A156H, S24H+G157H, S24H+S158H, S24H+Y161H, S24H+R164H, S24H+A166H, S24H+N167H, S24H+D175H, S24H+Q176H, S24H+N177H, S24H+N178H, S24H+N179H, S24H+R180H, S24H+S182H, S24H+F183H, S24H+Y186H, S24H+A188H, S24H+G189H, S24H+N198H, S24H+Q200H, S24H+Y203H, S24H+P204H, S24H+G205H, S24H+S206H, S24H+T207H, S24H+Y208H, S24H+S210H, S24H+L211H, S24H+N212H, S24H+K229H, S24H+Q230H, S24H+K231H, S24H+N232H, S24H+P233H, S24H+S234H, S24H+W235H, S24H+S236H, S24H+V238H, S24H+Q239H, S24H+R241H, S24H+N242H, S24H+K245H, S24H+N246H, S24H+T247H, S24H+T249H, S24H+S250H, S24H+L251H, S24H+G252H, S24H+S253H, S24H+T254H, S24H+N255H, S24H+L256H, S24H+Y257H, S24H+S259H, S24H+E265H, S24H+R269H, G25H+K27H, G25H+T37H, G25H+P39H, G25H+N42H, G25H+I43H, G25H+R44H, G25H+G45H, G25H+G46H, G25H+S48H, G25H+F49H, G25H+P51H, G25H+G52H, G25H+E53H, G25H+P54H, G25H+S55H, G25H+T56H, G25H+Q57H, G25H+G59H, G25H+L73H, G25H+N74H, G25H+N75H, G25H+S76H, G25H+177H, G25H+S85H, G25H+E87H, G25H+Y89H, G25H+A96H, G25H+S97H, G25H+G98H, G25H+S99H, G25H+G100H, G25H+S101H, G25H+V102H, G25H+S103H, G25H+Q107H, G25H+E110H, G25H+W111H, G25H+N114H, G25H+N115H, G25H+G116H, G25H+G125H, G25H+S126H, G25H+P127H, G25H+S128H, G25H+P129H, G25H+A131H, G25H+T132H, G25H+E134H, G25H+Q135H, G25H+N138H, G25H+S139H, G25H+S142H, G25H+R143H, G25H+G144H, G25H+N153H, G25H+S154H, G25H+G155H, G25H+A156H, G25H+G157H, G25H+S158H, G25H+Y161H, G25H+R164H, G25H+A166H, G25H+N167H, G25H+D175H, G25H+Q176H, G25H+N177H, G25H+N178H, G25H+N179H, G25H+R180H, G25H+S182H, G25H+F183H, G25H+Y186H, G25H+A188H, G25H+G189H, G25H+N198H, G25H+Q200H, G25H+Y203H, G25H+P204H, G25H+G205H, G25H+S206H, G25H+T207H, G25H+Y208H, G25H+S210H, G25H+L211H, G25H+N212H, G25H+K229H, G25H+Q230H, G25H+K231H, G25H+N232H, G25H+P233H, G25H+S234H, G25H+W235H, G25H+S236H, G25H+V238H, G25H+Q239H, G25H+R241H, G25H+N242H, G25H+K245H, G25H+N246H, G25H+T247H, G25H+T249H, G25H+S250H, G25H+L251H, G25H+G252H, G25H+S253H, G25H+T254H, G25H+N255H, G25H+L256H, G25H+Y257H, G25H+S259H, G25H+E265H, G25H+R269H, K27H+T37H, K27H+P39H, K27H+N42H, K27H+I43H, K27H+R44H, K27H+G45H, K27H+G46H, K27H+S48H, K27H+F49H, K27H+P51H, K27H+G52H, K27H+E53H, K27H+P54H, K27H+S55H, K27H+T56H, K27H+Q57H, K27H+G59H, K27H+L73H, K27H+N74H, K27H+N75H, K27H+S76H, K27H+177H, K27H+S85H, K27H+E87H, K27H+Y89H, K27H+A96H, K27H+S97H, K27H+G98H, K27H+S99H, K27H+G100H, K27H+S101H, K27H+V102H, K27H+S103H, K27H+Q107H, K27H+E110H, K27H+W111H, K27H+N114H, K27H+N115H, K27H+G116H, K27H+G125H, K27H+S126H, K27H+P127H, K27H+S128H, K27H+P129H, K27H+A131H, K27H+T132H, K27H+E134H, K27H+Q135H, K27H+N138H, K27H+S139H, K27H+S142H, K27H+R143H, K27H+G144H, K27H+N153H, K27H+S154H, K27H+G155H, K27H+A156H, K27H+G157H, K27H+S158H, K27H+Y161H, K27H+R164H, K27H+A166H, K27H+N167H, K27H+D175H, K27H+Q176H, K27H+N177H, K27H+N178H, K27H+N179H, K27H+R180H, K27H+S182H, K27H+F183H, K27H+Y186H, K27H+A188H, K27H+G189H, K27H+N198H, K27H+Q200H, K27H+Y203H, K27H+P204H, K27H+G205H, K27H+S206H, K27H+T207H, K27H+Y208H, K27H+S210H, K27H+L211H, K27H+N212H, K27H+K229H, K27H+Q230H, K27H+K231H, K27H+N232H, K27H+P233H, K27H+S234H, K27H+W235H, K27H+S236H, K27H+V238H, K27H+Q239H, K27H+R241H, K27H+N242H, K27H+K245H, K27H+N246H, K27H+T247H, K27H+T249H, K27H+S250H, K27H+L251H, K27H+G252H, K27H+S253H, K27H+T254H, K27H+N255H, K27H+L256H, K27H+Y257H, K27H+S259H, K27H+E265H, K27H+R269H, T37H+P39H, T37H+N42H, T37H+I43H, T37H+R44H, T37H+G45H, T37H+G46H, T37H+S48H, T37H+F49H, T37H+P51H, T37H+G52H, T37H+E53H, T37H+P54H, T37H+S55H, T37H+T56H, T37H+Q57H, T37H+G59H, T37H+L73H, T37H+N74H, T37H+N75H, T37H+S76H, T37H+177H, T37H+S85H, T37H+E87H, T37H+Y89H, T37H+A96H, T37H+S97H, T37H+G98H, T37H+S99H, T37H+G100H, T37H+S101H, T37H+V102H, T37H+S103H, T37H+Q107H, T37H+E110H, T37H+W111H, T37H+N114H, T37H+N115H, T37H+G116H, T37H+G125H, T37H+S126H, T37H+P127H, T37H+S128H, T37H+P129H, T37H+A131H, T37H+T132H, T37H+E134H, T37H+Q135H, T37H+N138H, T37H+S139H, T37H+S142H, T37H+R143H, T37H+G144H, T37H+N153H, T37H+S154H, T37H+G155H, T37H+A156H, T37H+G157H, T37H+S158H, T37H+Y161H, T37H+R164H, T37H+A166H, T37H+N167H, T37H+D175H, T37H+Q176H, T37H+N177H, T37H+N178H, T37H+N179H, T37H+R180H, T37H+S182H, T37H+F183H, T37H+Y186H, T37H+A188H, T37H+G189H, T37H+N198H, T37H+Q200H, T37H+Y203H, T37H+P204H, T37H+G205H, T37H+S206H, T37H+T207H, T37H+Y208H, T37H+S210H, T37H+L211H, T37H+N212H, T37H+K229H, T37H+Q230H, T37H+K231H, T37H+N232H, T37H+P233H, T37H+S234H, T37H+W235H, T37H+S236H, T37H+V238H, T37H+Q239H, T37H+R241H, T37H+N242H, T37H+K245H, T37H+N246H, T37H+T247H, T37H+T249H, T37H+S250H, T37H+L251H, T37H+G252H, T37H+S253H, T37H+T254H, T37H+N255H, T37H+L256H, T37H+Y257H, T37H+S259H, T37H+E265H, T37H+R269H, P39H+N42H, P39H+I43H, P39H+R44H, P39H+G45H, P39H+G46H, P39H+S48H, P39H+F49H, P39H+P51H, P39H+G52H, P39H+E53H, P39H+P54H, P39H+S55H, P39H+T56H, P39H+Q57H, P39H+G59H, P39H+L73H, P39H+N74H, P39H+N75H, P39H+S76H, P39H+177H, P39H+S85H, P39H+E87H, P39H+Y89H, P39H+A96H, P39H+S97H, P39H+G98H, P39H+S99H, P39H+G100H, P39H+S101H, P39H+V102H, P39H+S103H, P39H+Q107H, P39H+E110H, P39H+W111H, P39H+N114H, P39H+N115H, P39H+G116H, P39H+G125H, P39H+S126H, P39H+P127H, P39H+S128H, P39H+P129H, P39H+A131H, P39H+T132H, P39H+E134H, P39H+Q135H, P39H+N138H, P39H+S139H, P39H+S142H, P39H+R143H, P39H+G144H, P39H+N153H, P39H+S154H, P39H+G155H, P39H+A156H, P39H+G157H, P39H+S158H, P39H+Y161H, P39H+R164H, P39H+A166H, P39H+N167H, P39H+D175H, P39H+Q176H, P39H+N177H, P39H+N178H, P39H+N179H, P39H+R180H, P39H+S182H, P39H+F183H, P39H+Y186H, P39H+A188H, P39H+G189H, P39H+N198H, P39H+Q200H, P39H+Y203H, P39H+P204H, P39H+G205H, P39H+S206H, P39H+T207H, P39H+Y208H, P39H+S210H, P39H+L211H, P39H+N212H, P39H+K229H, P39H+Q230H, P39H+K231H, P39H+N232H, P39H+P233H, P39H+S234H, P39H+W235H, P39H+S236H, P39H+V238H, P39H+Q239H, P39H+R241H, P39H+N242H, P39H+K245H, P39H+N246H, P39H+T247H, P39H+T249H, P39H+S250H, P39H+L251H, P39H+G252H, P39H+S253H, P39H+T254H, P39H+N255H, P39H+L256H, P39H+Y257H, P39H+S259H, P39H+E265H, P39H+R269H, N42H+I43H, N42H+R44H, N42H+G45H, N42H+G46H, N42H+S48H, N42H+F49H, N42H+P51H, N42H+G52H, N42H+E53H, N42H+P54H, N42H+S55H, N42H+T56H, N42H+Q57H, N42H+G59H, N42H+L73H, N42H+N74H, N42H+N75H, N42H+S76H, N42H+177H, N42H+S85H, N42H+E87H, N42H+Y89H, N42H+A96H, N42H+S97H, N42H+G98H, N42H+S99H, N42H+G100H, N42H+S101H, N42H+V102H, N42H+S103H, N42H+Q107H, N42H+E110H, N42H+W111H, N42H+N114H, N42H+N115H, N42H+G116H, N42H+G125H, N42H+S126H, N42H+P127H, N42H+S128H, N42H+P129H, N42H+A131H, N42H+T132H, N42H+E134H, N42H+Q135H, N42H+N138H, N42H+S139H, N42H+S142H, N42H+R143H, N42H+G144H, N42H+N153H, N42H+S154H, N42H+G155H, N42H+A156H, N42H+G157H, N42H+S158H, N42H+Y161H, N42H+R164H, N42H+A166H, N42H+N167H, N42H+D175H, N42H+Q176H, N42H+N177H, N42H+N178H, N42H+N179H, N42H+R180H, N42H+S182H, N42H+F183H, N42H+Y186H, N42H+A188H, N42H+G189H, N42H+N198H, N42H+Q200H, N42H+Y203H, N42H+P204H, N42H+G205H, N42H+S206H, N42H+T207H, N42H+Y208H, N42H+S210H, N42H+L211H, N42H+N212H, N42H+K229H, N42H+Q230H, N42H+K231H, N42H+N232H, N42H+P233H, N42H+S234H, N42H+W235H, N42H+S236H, N42H+V238H, N42H+Q239H, N42H+R241H, N42H+N242H, N42H+K245H, N42H+N246H, N42H+T247H, N42H+T249H, N42H+S250H, N42H+L251H, N42H+G252H, N42H+S253H, N42H+T254H, N42H+N255H, N42H+L256H, N42H+Y257H, N42H+S259H, N42H+E265H, N42H+R269H, I43H+R44H, I43H+G45H, I43H+G46H, I43H+S48H, I43H+F49H, I43H+P51H, I43H+G52H, I43H+E53H, I43H+P54H, I43H+S55H, I43H+T56H, I43H+Q57H, I43H+G59H, I43H+L73H, I43H+N74H, I43H+N75H, I43H+S76H, 143H+177H, I43H+S85H, I43H+E87H, I43H+Y89H, I43H+A96H, I43H+S97H, I43H+G98H, I43H+S99H, I43H+G100H, 143H+S101H, 143H+V102H, 143H+S103H, 143H+Q107H, 143H+E110H, I43H+W111H, I43H+N114H, I43H+N115H, I43H+G116H, I43H+G125H, I43H+S126H, I43H+P127H, I43H+S128H, I43H+P129H, I43H+A131H, I43H+T132H, I43H+E134H, I43H+Q135H, I43H+N138H, I43H+S139H, I43H+S142H, I43H+R143H, I43H+G144H, I43H+N153H, I43H+S154H, I43H+G155H, I43H+A156H, I43H+G157H, I43H+S158H, I43H+Y161H, I43H+R164H, I43H+A166H, I43H+N167H, I43H+D175H, I43H+Q176H, I43H+N177H, I43H+N178H, I43H+N179H, 143H+R180H, I43H+S182H, I43H+F183H, I43H+Y186H, I43H+A188H, I43H+G189H, I43H+N198H, 143H+Q200H, I43H+Y203H, I43H+P204H, I43H+G205H, I43H+S206H, I43H+T207H, I43H+Y208H, 143H+S210H, I43H+L211H, I43H+N212H, I43H+K229H, 143H+Q230H, I43H+K231H, I43H+N232H, I43H+P233H, I43H+S234H, I43H+W235H, I43H+S236H, I43H+V238H, 143H+Q239H, I43H+R241H, I43H+N242H, I43H+K245H, I43H+N246H, I43H+T247H, I43H+T249H, I43H+S250H, I43H+L251H, I43H+G252H, I43H+S253H, I43H+T254H, I43H+N255H, I43H+L256H, I43H+Y257H, I43H+S259H, I43H+E265H, I43H+R269H, R44H+G45H, R44H+G46H, R44H+S48H, R44H+F49H, R44H+P51H, R44H+G52H, R44H+E53H, R44H+P54H, R44H+S55H, R44H+T56H, R44H+Q57H, R44H+G59H, R44H+L73H, R44H+N74H, R44H+N75H, R44H+S76H, R44H+177H, R44H+S85H, R44H+E87H, R44H+Y89H, R44H+A96H, R44H+S97H, R44H+G98H, R44H+S99H, R44H+G100H, R44H+S101H, R44H+V102H, R44H+S103H, R44H+Q107H, R44H+E110H, R44H+W111H, R44H+N114H, R44H+N115H, R44H+G116H, R44H+G125H, R44H+S126H, R44H+P127H, R44H+S128H, R44H+P129H, R44H+A131H, R44H+T132H, R44H+E134H, R44H+Q135H, R44H+N138H, R44H+S139H, R44H+S142H, R44H+R143H, R44H+G144H, R44H+N153H, R44H+S154H, R44H+G155H, R44H+A156H, R44H+G157H, R44H+S158H, R44H+Y161H, R44H+R164H, R44H+A166H, R44H+N167H, R44H+D175H, R44H+Q176H, R44H+N177H, R44H+N178H, R44H+N179H, R44H+R180H, R44H+S182H, R44H+F183H, R44H+Y186H, R44H+A188H, R44H+G189H, R44H+N198H, R44H+Q200H, R44H+Y203H, R44H+P204H, R44H+G205H, R44H+S206H, R44H+T207H, R44H+Y208H, R44H+S210H, R44H+L211H, R44H+N212H, R44H+K229H, R44H+Q230H, R44H+K231H, R44H+N232H, R44H+P233H, R44H+S234H, R44H+W235H, R44H+S236H, R44H+V238H, R44H+Q239H, R44H+R241H, R44H+N242H, R44H+K245H, R44H+N246H, R44H+T247H, R44H+T249H, R44H+S250H, R44H+L251H, R44H+G252H, R44H+S253H, R44H+T254H, R44H+N255H, R44H+L256H, R44H+Y257H, R44H+S259H, R44H+E265H, R44H+R269H, G45H+G46H, G45H+S48H, G45H+F49H, G45H+P51H, G45H+G52H, G45H+E53H, G45H+P54H, G45H+S55H, G45H+T56H, G45H+Q57H, G45H+G59H, G45H+L73H, G45H+N74H, G45H+N75H, G45H+S76H, G45H+177H, G45H+S85H, G45H+E87H, G45H+Y89H, G45H+A96H, G45H+S97H, G45H+G98H, G45H+S99H, G45H+G100H, G45H+S101H, G45H+V102H, G45H+S103H, G45H+Q107H, G45H+E110H, G45H+W111H, G45H+N114H, G45H+N115H, G45H+G116H, G45H+G125H, G45H+S126H, G45H+P127H, G45H+S128H, G45H+P129H, G45H+A131H, G45H+T132H, G45H+E134H, G45H+Q135H, G45H+N138H, G45H+S139H, G45H+S142H, G45H+R143H, G45H+G144H, G45H+N153H, G45H+S154H, G45H+G155H, G45H+A156H, G45H+G157H, G45H+S158H, G45H+Y161H, G45H+R164H, G45H+A166H, G45H+N167H, G45H+D175H, G45H+Q176H, G45H+N177H, G45H+N178H, G45H+N179H, G45H+R180H, G45H+S182H, G45H+F183H, G45H+Y186H, G45H+A188H, G45H+G189H, G45H+N198H, G45H+Q200H, G45H+Y203H, G45H+P204H, G45H+G205H, G45H+S206H, G45H+T207H, G45H+Y208H, G45H+S210H, G45H+L211H, G45H+N212H, G45H+K229H, G45H+Q230H, G45H+K231H, G45H+N232H, G45H+P233H, G45H+S234H, G45H+W235H, G45H+S236H, G45H+V238H, G45H+Q239H, G45H+R241H, G45H+N242H, G45H+K245H, G45H+N246H, G45H+T247H, G45H+T249H, G45H+S250H, G45H+L251H, G45H+G252H, G45H+S253H, G45H+T254H, G45H+N255H, G45H+L256H, G45H+Y257H, G45H+S259H, G45H+E265H, G45H+R269H, G46H+S48H, G46H+F49H, G46H+P51H, G46H+G52H, G46H+E53H, G46H+P54H, G46H+S55H, G46H+T56H, G46H+Q57H, G46H+G59H, G46H+L73H, G46H+N74H, G46H+N75H, G46H+S76H, G46H+177H, G46H+S85H, G46H+E87H, G46H+Y89H, G46H+A96H, G46H+S97H, G46H+G98H, G46H+S99H, G46H+G100H, G46H+S101H, G46H+V102H, G46H+S103H, G46H+Q107H, G46H+E110H, G46H+W111H, G46H+N114H, G46H+N115H, G46H+G116H, G46H+G125H, G46H+S126H, G46H+P127H, G46H+S128H, G46H+P129H, G46H+A131H, G46H+T132H, G46H+E134H, G46H+Q135H, G46H+N138H, G46H+S139H, G46H+S142H, G46H+R143H, G46H+G144H, G46H+N153H, G46H+S154H, G46H+G155H, G46H+A156H, G46H+G157H, G46H+S158H, G46H+Y161H, G46H+R164H, G46H+A166H, G46H+N167H, G46H+D175H, G46H+Q176H, G46H+N177H, G46H+N178H, G46H+N179H, G46H+R180H, G46H+S182H, G46H+F183H, G46H+Y186H, G46H+A188H, G46H+G189H, G46H+N198H, G46H+Q200H, G46H+Y203H, G46H+P204H, G46H+G205H, G46H+S206H, G46H+T207H, G46H+Y208H, G46H+S210H, G46H+L211H, G46H+N212H, G46H+K229H, G46H+Q230H, G46H+K231H, G46H+N232H, G46H+P233H, G46H+S234H, G46H+W235H, G46H+S236H, G46H+V238H, G46H+Q239H, G46H+R241H, G46H+N242H, G46H+K245H, G46H+N246H, G46H+T247H, G46H+T249H, G46H+S250H, G46H+L251H, G46H+G252H, G46H+S253H, G46H+T254H, G46H+N255H, G46H+L256H, G46H+Y257H, G46H+S259H, G46H+E265H, G46H+R269H, S48H+F49H, S48H+P51H, S48H+G52H, S48H+E53H, S48H+P54H, S48H+S55H, S48H+T56H, S48H+Q57H, S48H+G59H, S48H+L73H, S48H+N74H, S48H+N75H, S48H+S76H, S48H+177H, S48H+S85H, S48H+E87H, S48H+Y89H, S48H+A96H, S48H+S97H, S48H+G98H, S48H+S99H, S48H+G100H, S48H+S101H, S48H+V102H, S48H+S103H, S48H+Q107H, S48H+E110H, S48H+W111H, S48H+N114H, S48H+N115H, S48H+G116H, S48H+G125H, S48H+S126H, S48H+P127H, S48H+S128H, S48H+P129H, S48H+A131H, S48H+T132H, S48H+E134H, S48H+Q135H, S48H+N138H, S48H+S139H, S48H+S142H, S48H+R143H, S48H+G144H, S48H+N153H, S48H+S154H, S48H+G155H, S48H+A156H, S48H+G157H, S48H+S158H, S48H+Y161H, S48H+R164H, S48H+A166H, S48H+N167H, S48H+D175H, S48H+Q176H, S48H+N177H, S48H+N178H, S48H+N179H, S48H+R180H, S48H+S182H, S48H+F183H, S48H+Y186H, S48H+A188H, S48H+G189H, S48H+N198H, S48H+Q200H, S48H+Y203H, S48H+P204H, S48H+G205H, S48H+S206H, S48H+T207H, S48H+Y208H, S48H+S210H, S48H+L211H, S48H+N212H, S48H+K229H, S48H+Q230H, S48H+K231H, S48H+N232H, S48H+P233H, S48H+S234H, S48H+W235H, S48H+S236H, S48H+V238H, S48H+Q239H, S48H+R241H, S48H+N242H, S48H+K245H, S48H+N246H, S48H+T247H, S48H+T249H, S48H+S250H, S48H+L251H, S48H+G252H, S48H+S253H, S48H+T254H, S48H+N255H, S48H+L256H, S48H+Y257H, S48H+S259H, S48H+E265H, S48H+R269H, F49H+P51H, F49H+G52H, F49H+E53H, F49H+P54H, F49H+S55H, F49H+T56H, F49H+Q57H, F49H+G59H, F49H+L73H, F49H+N74H, F49H+N75H, F49H+S76H, F49H+177H, F49H+S85H, F49H+E87H, F49H+Y89H, F49H+A96H, F49H+S97H, F49H+G98H, F49H+S99H, F49H+G100H, F49H+S101H, F49H+V102H, F49H+S103H, F49H+Q107H, F49H+E110H, F49H+W111H, F49H+N114H, F49H+N115H, F49H+G116H, F49H+G125H, F49H+S126H, F49H+P127H, F49H+S128H, F49H+P129H, F49H+A131H, F49H+T132H, F49H+E134H, F49H+Q135H, F49H+N138H, F49H+S139H, F49H+S142H, F49H+R143H, F49H+G144H, F49H+N153H, F49H+S154H, F49H+G155H, F49H+A156H, F49H+G157H, F49H+S158H, F49H+Y161H, F49H+R164H, F49H+A166H, F49H+N167H, F49H+D175H, F49H+Q176H, F49H+N177H, F49H+N178H, F49H+N179H, F49H+R180H, F49H+S182H, F49H+F183H, F49H+Y186H, F49H+A188H, F49H+G189H, F49H+N198H, F49H+Q200H, F49H+Y203H, F49H+P204H, F49H+G205H, F49H+S206H, F49H+T207H, F49H+Y208H, F49H+S210H, F49H+L211H, F49H+N212H, F49H+K229H, F49H+Q230H, F49H+K231H, F49H+N232H, F49H+P233H, F49H+S234H, F49H+W235H, F49H+S236H, F49H+V238H, F49H+Q239H, F49H+R241H, F49H+N242H, F49H+K245H, F49H+N246H, F49H+T247H, F49H+T249H, F49H+S250H, F49H+L251H, F49H+G252H, F49H+S253H, F49H+T254H, F49H+N255H, F49H+L256H, F49H+Y257H, F49H+S259H, F49H+E265H, F49H+R269H, P51H+G52H, P51H+E53H, P51H+P54H, P51H+S55H, P51H+T56H, P51H+Q57H, P51H+G59H, P51H+L73H, P51H+N74H, P51H+N75H, P51H+S76H, P51H+177H, P51H+S85H, P51H+E87H, P51H+Y89H, P51H+A96H, P51H+S97H, P51H+G98H, P51H+S99H, P51H+G100H, P51H+S101H, P51H+V102H, P51H+S103H, P51H+Q107H, P51H+E110H, P51H+W111H, P51H+N114H, P51H+N115H, P51H+G116H, P51H+G125H, P51H+S126H, P51H+P127H, P51H+S128H, P51H+P129H, P51H+A131H, P51H+T132H, P51H+E134H, P51H+Q135H, P51H+N138H, P51H+S139H, P51H+S142H, P51H+R143H, P51H+G144H, P51H+N153H, P51H+S154H, P51H+G155H, P51H+A156H, P51H+G157H, P51H+S158H, P51H+Y161H, P51H+R164H, P51H+A166H, P51H+N167H, P51H+D175H, P51H+Q176H, P51H+N177H, P51H+N178H, P51H+N179H, P51H+R180H, P51H+S182H, P51H+F183H, P51H+Y186H, P51H+A188H, P51H+G189H, P51H+N198H, P51H+Q200H, P51H+Y203H, P51H+P204H, P51H+G205H, P51H+S206H, P51H+T207H, P51H+Y208H, P51H+S210H, P51H+L211H, P51H+N212H, P51H+K229H, P51H+Q230H, P51H+K231H, P51H+N232H, P51H+P233H, P51H+S234H, P51H+W235H, P51H+S236H, P51H+V238H, P51H+Q239H, P51H+R241H, P51H+N242H, P51H+K245H, P51H+N246H, P51H+T247H, P51H+T249H, P51H+S250H, P51H+L251H, P51H+G252H, P51H+S253H, P51H+T254H, P51H+N255H, P51H+L256H, P51H+Y257H, P51H+S259H, P51H+E265H, P51H+R269H, G52H+E53H, G52H+P54H, G52H+S55H, G52H+T56H, G52H+Q57H, G52H+G59H, G52H+L73H, G52H+N74H, G52H+N75H, G52H+S76H, G52H+177H, G52H+S85H, G52H+E87H, G52H+Y89H, G52H+A96H, G52H+S97H, G52H+G98H, G52H+S99H, G52H+G100H, G52H+S101H, G52H+V102H, G52H+S103H, G52H+Q107H, G52H+E110H, G52H+W111H, G52H+N114H, G52H+N115H, G52H+G116H, G52H+G125H, G52H+S126H, G52H+P127H, G52H+S128H, G52H+P129H, G52H+A131H, G52H+T132H, G52H+E134H, G52H+Q135H, G52H+N138H, G52H+S139H, G52H+S142H, G52H+R143H, G52H+G144H, G52H+N153H, G52H+S154H, G52H+G155H, G52H+A156H, G52H+G157H, G52H+S158H, G52H+Y161H, G52H+R164H, G52H+A166H, G52H+N167H, G52H+D175H, G52H+Q176H, G52H+N177H, G52H+N178H, G52H+N179H, G52H+R180H, G52H+S182H, G52H+F183H, G52H+Y186H, G52H+A188H, G52H+G189H, G52H+N198H, G52H+Q200H, G52H+Y203H, G52H+P204H, G52H+G205H, G52H+S206H, G52H+T207H, G52H+Y208H, G52H+S210H, G52H+L211H, G52H+N212H, G52H+K229H, G52H+Q230H, G52H+K231H, G52H+N232H, G52H+P233H, G52H+S234H, G52H+W235H, G52H+S236H, G52H+V238H, G52H+Q239H, G52H+R241H, G52H+N242H, G52H+K245H, G52H+N246H, G52H+T247H, G52H+T249H, G52H+S250H, G52H+L251H, G52H+G252H, G52H+S253H, G52H+T254H, G52H+N255H, G52H+L256H, G52H+Y257H, G52H+S259H, G52H+E265H, G52H+R269H, E53H+P54H, E53H+S55H, E53H+T56H, E53H+Q157H, E53H+G59H, E53H+L73H, E53H+N74H, E53H+N75H, E53H+S76H, E53H+177H, E53H+S85H, E53H+E87H, E53H+Y89H, E53H+A96H, E53H+S97H, E53H+G98H, E53H+S99H, E53H+G100H, E53H+S101H, E53H+V102H, E53H+S103H, E53H+Q107H, E53H+E110H, E53H+W111H, E53H+N114H, E53H+N115H, E53H+G116H, E53H+G125H, E53H+S126H, E53H+P127H, E53H+S128H, E53H+P129H, E53H+A131H, E53H+T132H, E53H+E134H, E53H+Q135H, E53H+N138H, E53H+S139H, E53H+S142H, E53H+R143H, E53H+G144H, E53H+N153H, E53H+S154H, E53H+G155H, E53H+A156H, E53H+G157H, E53H+S158H, E53H+Y161H, E53H+R164H, E53H+A166H, E53H+N167H, E53H+D175H, E53H+Q176H, E53H+N177H, E53H+N178H, E53H+N179H, E53H+R180H, E53H+S182H, E53H+F183H, E53H+Y186H, E53H+A188H, E53H+G189H, E53H+N198H, E53H+Q200H, E53H+Y203H, E53H+P204H, E53H+G205H, E53H+S206H, E53H+T207H, E53H+Y208H, E53H+S210H, E53H+L211H, E53H+N212H, E53H+K229H, E53H+Q230H, E53H+K231H, E53H+N232H, E53H+P233H, E53H+S234H, E53H+W235H, E53H+S236H, E53H+V238H, E53H+Q239H, E53H+R241H, E53H+N242H, E53H+K245H, E53H+N246H, E53H+T247H, E53H+T249H, E53H+S250H, E53H+L251H, E53H+G252H, E53H+S253H, E53H+T254H, E53H+N255H, E53H+L256H, E53H+Y257H, E53H+S259H, E53H+E265H, E53H+R269H, P54H+S55H, P54H+T56H, P54H+Q57H, P54H+G59H, P54H+L73H, P54H+N74H, P54H+N75H, P54H+S76H, P54H+177H, P54H+S85H, P54H+E87H, P54H+Y89H, P54H+A96H, P54H+S97H, P54H+G98H, P54H+S99H, P54H+G100H, P54H+S101H, P54H+V102H, P54H+S103H, P54H+Q107H, P54H+E110H, P54H+W111H, P54H+N114H, P54H+N115H, P54H+G116H, P54H+G125H, P54H+S126H, P54H+P127H, P54H+S128H, P54H+P129H, P54H+A131H, P54H+T132H, P54H+E134H, P54H+Q135H, P54H+N138H, P54H+S139H, P54H+S142H, P54H+R143H, P54H+G144H, P54H+N153H, P54H+S154H, P54H+G155H, P54H+A156H, P54H+G157H, P54H+S158H, P54H+Y161H, P54H+R164H, P54H+A166H, P54H+N167H, P54H+D175H, P54H+Q176H, P54H+N177H, P54H+N178H, P54H+N179H, P54H+R180H, P54H+S182H, P54H+F183H, P54H+Y186H, P54H+A188H, P54H+G189H, P54H+N198H, P54H+Q200H, P54H+Y203H, P54H+P204H, P54H+G205H, P54H+S206H, P54H+T207H, P54H+Y208H, P54H+S210H, P54H+L211H, P54H+N212H, P54H+K229H, P54H+Q230H, P54H+K231H, P54H+N232H, P54H+P233H, P54H+S234H, P54H+W235H, P54H+S236H, P54H+V238H, P54H+Q239H, P54H+R241H, P54H+N242H, P54H+K245H, P54H+N246H, P54H+T247H, P54H+T249H, P54H+S250H, P54H+L251H, P54H+G252H, P54H+S253H, P54H+T254H, P54H+N255H, P54H+L256H, P54H+Y257H, P54H+S259H, P54H+E265H, P54H+R269H, S55H+T56H, S55H+Q57H, S55H+G59H, S55H+L73H, S55H+N74H, S55H+N75H, S55H+S76H, S55H+177H, S55H+S85H, S55H+E87H, S55H+Y89H, S55H+A96H, S55H+S97H, S55H+G98H, S55H+S99H, S55H+G100H, S55H+S101H, S55H+V102H, S55H+S103H, S55H+Q107H, S55H+E110H, S55H+W111H, S55H+N114H, S55H+N115H, S55H+G116H, S55H+G125H, S55H+S126H, S55H+P127H, S55H+S128H, S55H+P129H, S55H+A131H, S55H+T132H, S55H+E134H, S55H+Q135H, S55H+N138H, S55H+S139H, S55H+S142H, S55H+R143H, S55H+G144H, S55H+N153H, S55H+S154H, S55H+G155H, S55H+A156H, S55H+G157H, S55H+S158H, S55H+Y161H, S55H+R164H, S55H+A166H, S55H+N167H, S55H+D175H, S55H+Q176H, S55H+N177H, S55H+N178H, S55H+N179H, S55H+R180H, S55H+S182H, S55H+F183H, S55H+Y186H, S55H+A188H, S55H+G189H, S55H+N198H, S55H+Q200H, S55H+Y203H, S55H+P204H, S55H+G205H, S55H+S206H, S55H+T207H, S55H+Y208H, S55H+S210H, S55H+L211H, S55H+N212H, S55H+K229H, S55H+Q230H, S55H+K231H, S55H+N232H, S55H+P233H, S55H+S234H, S55H+W235H, S55H+S236H, S55H+V238H, S55H+Q239H, S55H+R241H, S55H+N242H, S55H+K245H, S55H+N246H, S55H+T247H, S55H+T249H, S55H+S250H, S55H+L251H, S55H+G252H, S55H+S253H, S55H+T254H, S55H+N255H, S55H+L256H, S55H+Y257H, S55H+S259H, S55H+E265H, S55H+R269H, T56H+Q57H, T56H+G59H, T56H+L73H, T56H+N74H, T56H+N75H, T56H+S76H, T56H+177H, T56H+S85H, T56H+E87H, T56H+Y89H, T56H+A96H, T56H+S97H, T56H+G98H, T56H+S99H, T56H+G100H, T56H+S101H, T56H+V102H, T56H+S103H, T56H+Q107H, T56H+E110H, T56H+W111H, T56H+N114H, T56H+N115H, T56H+G116H, T56H+G125H, T56H+S126H, T56H+P127H, T56H+S128H, T56H+P129H, T56H+A131H, T56H+T132H, T56H+E134H, T56H+Q135H, T56H+N138H, T56H+S139H, T56H+S142H, T56H+R143H, T56H+G144H, T56H+N153H, T56H+S154H, T56H+G155H, T56H+A156H, T56H+G157H, T56H+S158H, T56H+Y161H, T56H+R164H, T56H+A166H, T56H+N167H, T56H+D175H, T56H+Q176H, T56H+N177H, T56H+N178H, T56H+N179H, T56H+R180H, T56H+S182H, T56H+F183H, T56H+Y186H, T56H+A188H, T56H+G189H, T56H+N198H, T56H+Q200H, T56H+Y203H, T56H+P204H, T56H+G205H, T56H+S206H, T56H+T207H, T56H+Y208H, T56H+S210H, T56H+L211H, T56H+N212H, T56H+K229H, T56H+Q230H, T56H+K231H, T56H+N232H, T56H+P233H, T56H+S234H, T56H+W235H, T56H+S236H, T56H+V238H, T56H+Q239H, T56H+R241H, T56H+N242H, T56H+K245H, T56H+N246H, T56H+T247H, T56H+T249H, T56H+S250H, T56H+L251H, T56H+G252H, T56H+S253H, T56H+T254H, T56H+N255H, T56H+L256H, T56H+Y257H, T56H+S259H, T56H+E265H, T56H+R269H, Q57H+G59H, Q57H+L73H, Q57H+N74H, Q57H+N75H, Q57H+S76H, Q57H+I77H, Q57H+S85H, Q57H+E87H, Q57H+Y89H, Q57H+A96H, Q57H+S97H, Q57H+G98H, Q57H+S99H, Q57H+G100H, Q57H+S101H, Q57H+V102H, Q57H+S103H, Q57H+Q107H, Q57H+E110H, Q57H+W111H, Q57H+N114H, Q57H+N115H, Q57H+G116H, Q57H+G125H, Q57H+S126H, Q57H+P127H, Q57H+S128H, Q57H+P129H, Q57H+A131H, Q57H+T132H, Q57H+E134H, Q57H+Q135H, Q57H+N138H, Q57H+S139H, Q57H+S142H, Q57H+R143H, Q57H+G144H, Q57H+N153H, Q57H+S154H, Q57H+G155H, Q57H+A156H, Q57H+G157H, Q57H+S158H, Q57H+Y161H, Q57H+R164H, Q57H+A166H, Q57H+N167H, Q57H+D175H, Q57H+Q176H, Q57H+N177H, Q57H+N178H, Q57H+N179H, Q57H+R180H, Q57H+S182H, Q57H+F183H, Q57H+Y186H, Q57H+A188H, Q57H+G189H, Q57H+N198H, Q57H+Q200H, Q57H+Y203H, Q57H+P204H, Q57H+G205H, Q57H+S206H, Q57H+T207H, Q57H+Y208H, Q57H+S210H, Q57H+L211H, Q57H+N212H, Q57H+K229H, Q57H+Q230H, Q57H+K231H, Q57H+N232H, Q57H+P233H, Q57H+S234H, Q57H+W235H, Q57H+S236H, Q57H+V238H, Q57H+Q239H, Q57H+R241H, Q57H+N242H, Q57H+K245H, Q57H+N246H, Q57H+T247H, Q57H+T249H, Q57H+S250H, Q57H+L251H, Q57H+G252H, Q57H+S253H, Q57H+T254H, Q57H+N255H, Q57H+L256H, Q57H+Y257H, Q57H+S259H, Q57H+E265H, Q57H+R269H, G59H+L73H, G59H+N74H, G59H+N75H, G59H+S76H, G59H+I77H, G59H+S85H, G59H+E87H, G59H+Y89H, G59H+A96H, G59H+S97H, G59H+G98H, G59H+S99H, G59H+G100H, G59H+S101H, G59H+V102H, G59H+S103H, G59H+Q107H, G59H+E110H, G59H+W111H, G59H+N114H, G59H+N115H, G59H+G116H, G59H+G125H, G59H+S126H, G59H+P127H, G59H+S128H, G59H+P129H, G59H+A131H, G59H+T132H, G59H+E134H, G59H+Q135H, G59H+N138H, G59H+S139H, G59H+S142H, G59H+R143H, G59H+G144H, G59H+N153H, G59H+S154H, G59H+G155H, G59H+A156H, G59H+G157H, G59H+S158H, G59H+Y161H, G59H+R164H, G59H+A166H, G59H+N167H, G59H+D175H, G59H+Q176H, G59H+N177H, G59H+N178H, G59H+N179H, G59H+R180H, G59H+S182H, G59H+F183H, G59H+Y186H, G59H+A188H, G59H+G189H, G59H+N198H, G59H+Q200H, G59H+Y203H, G59H+P204H, G59H+G205H, G59H+S206H, G59H+T207H, G59H+Y208H, G59H+S210H, G59H+L211H, G59H+N212H, G59H+K229H, G59H+Q230H, G59H+K231H, G59H+N232H, G59H+P233H, G59H+S234H, G59H+W235H, G59H+S236H, G59H+V238H, G59H+Q239H, G59H+R241H, G59H+N242H, G59H+K245H, G59H+N246H, G59H+T247H, G59H+T249H, G59H+S250H, G59H+L251H, G59H+G252H, G59H+S253H, G59H+T254H, G59H+N255H, G59H+L256H, G59H+Y257H, G59H+S259H, G59H+E265H, G59H+R269H, L73H+N74H, L73H+N75H, L73H+S76H, L73H+177H, L73H+S85H, L73H+E87H, L73H+Y89H, L73H+A96H, L73H+S97H, L73H+G98H, L73H+S99H, L73H+G100H, L73H+S101H, L73H+V102H, L73H+S103H, L73H+Q107H, L73H+E110H, L73H+W111H, L73H+N114H, L73H+N115H, L73H+G116H, L73H+G125H, L73H+S126H, L73H+P127H, L73H+S128H, L73H+P129H, L73H+A131H, L73H+T132H, L73H+E134H, L73H+Q135H, L73H+N138H, L73H+S139H, L73H+S142H, L73H+R143H, L73H+G144H, L73H+N153H, L73H+S154H, L73H+G155H, L73H+A156H, L73H+G157H, L73H+S158H, L73H+Y161H, L73H+R164H, L73H+A166H, L73H+N167H, L73H+D175H, L73H+Q176H, L73H+N177H, L73H+N178H, L73H+N179H, L73H+R180H, L73H+S182H, L73H+F183H, L73H+Y186H, L73H+A188H, L73H+G189H, L73H+N198H, L73H+Q200H, L73H+Y203H, L73H+P204H, L73H+G205H, L73H+S206H, L73H+T207H, L73H+Y208H, L73H+S210H, L73H+L211H, L73H+N212H, L73H+K229H, L73H+Q230H, L73H+K231H, L73H+N232H, L73H+P233H, L73H+S234H, L73H+W235H, L73H+S236H, L73H+V238H, L73H+Q239H, L73H+R241H, L73H+N242H, L73H+K245H, L73H+N246H, L73H+T247H, L73H+T249H, L73H+S250H, L73H+L251H, L73H+G252H, L73H+S253H, L73H+T254H, L73H+N255H, L73H+L256H, L73H+Y257H, L73H+S259H, L73H+E265H, L73H+R269H, N74H+N75H, N74H+S76H, N74H+177H, N74H+S85H, N74H+E87H, N74H+Y89H, N74H+A96H, N74H+S97H, N74H+G98H, N74H+S99H, N74H+G100H, N74H+S101H, N74H+V102H, N74H+S103H, N74H+Q107H, N74H+E110H, N74H+W111H, N74H+N114H, N74H+N115H, N74H+G116H, N74H+G125H, N74H+S126H, N74H+P127H, N74H+S128H, N74H+P129H, N74H+A131H, N74H+T132H, N74H+E134H, N74H+Q135H, N74H+N138H, N74H+S139H, N74H+S142H, N74H+R143H, N74H+G144H, N74H+N153H, N74H+S154H, N74H+G155H, N74H+A156H, N74H+G157H, N74H+S158H, N74H+Y161H, N74H+R164H, N74H+A166H, N74H+N167H, N74H+D175H, N74H+Q176H, N74H+N177H, N74H+N178H, N74H+N179H, N74H+R180H, N74H+S182H, N74H+F183H, N74H+Y186H, N74H+A188H, N74H+G189H, N74H+N198H, N74H+Q200H, N74H+Y203H, N74H+P204H, N74H+G205H, N74H+S206H, N74H+T207H, N74H+Y208H, N74H+S210H, N74H+L211H, N74H+N212H, N74H+K229H, N74H+Q230H, N74H+K231H, N74H+N232H, N74H+P233H, N74H+S234H, N74H+W235H, N74H+S236H, N74H+V238H, N74H+Q239H, N74H+R241H, N74H+N242H, N74H+K245H, N74H+N246H, N74H+T247H, N74H+T249H, N74H+S250H, N74H+L251H, N74H+G252H, N74H+S253H, N74H+T254H, N74H+N255H, N74H+L256H, N74H+Y257H, N74H+S259H, N74H+E265H, N74H+R269H, N75H+S76H, N75H+177H, N75H+S85H, N75H+E87H, N75H+Y89H, N75H+A96H, N75H+S97H, N75H+G98H, N75H+S99H, N75H+G100H, N75H+S101H, N75H+V102H, N75H+S103H, N75H+Q107H, N75H+E110H, N75H+W111H, N75H+N114H, N75H+N115H, N75H+G116H, N75H+G125H, N75H+S126H, N75H+P127H, N75H+S128H, N75H+P129H, N75H+A131H, N75H+T132H, N75H+E134H, N75H+Q135H, N75H+N138H, N75H+S139H, N75H+S142H, N75H+R143H, N75H+G144H, N75H+N153H, N75H+S154H, N75H+G155H, N75H+A156H, N75H+G157H, N75H+S158H, N75H+Y161H, N75H+R164H, N75H+A166H, N75H+N167H, N75H+D175H, N75H+Q176H, N75H+N177H, N75H+N178H, N75H+N179H, N75H+R180H, N75H+S182H, N75H+F183H, N75H+Y186H, N75H+A188H, N75H+G189H, N75H+N198H, N75H+Q200H, N75H+Y203H, N75H+P204H, N75H+G205H, N75H+S206H, N75H+T207H, N75H+Y208H, N75H+S210H, N75H+L211H, N75H+N212H, N75H+K229H, N75H+Q230H, N75H+K231H, N75H+N232H, N75H+P233H, N75H+S234H, N75H+W235H, N75H+S236H, N75H+V238H, N75H+Q239H, N75H+R241H, N75H+N242H, N75H+K245H, N75H+N246H, N75H+T247H, N75H+T249H, N75H+S250H, N75H+L251H, N75H+G252H, N75H+S253H, N75H+T254H, N75H+N255H, N75H+L256H, N75H+Y257H, N75H+S259H, N75H+E265H, N75H+R269H, S76H+177H, S76H+S85H, S76H+E87H, S76H+Y89H, S76H+A96H, S76H+S97H, S76H+G98H, S76H+S99H, S76H+G100H, S76H+S101H, S76H+V102H, S76H+S103H, S76H+Q107H, S76H+E110H, S76H+W111H, S76H+N114H, S76H+N115H, S76H+G116H, S76H+G125H, S76H+S126H, S76H+P127H, S76H+S128H, S76H+P129H, S76H+A131H, S76H+T132H, S76H+E134H, S76H+Q135H, S76H+N138H, S76H+S139H, S76H+S142H, S76H+R143H, S76H+G144H, S76H+N153H, S76H+S154H, S76H+G155H, S76H+A156H, S76H+G157H, S76H+S158H, S76H+Y161H, S76H+R164H, S76H+A166H, S76H+N167H, S76H+D175H, S76H+Q176H, S76H+N177H, S76H+N178H, S76H+N179H, S76H+R180H, S76H+S182H, S76H+F183H, S76H+Y186H, S76H+A188H, S76H+G189H, S76H+N198H, S76H+Q200H, S76H+Y203H, S76H+P204H, S76H+G205H, S76H+S206H, S76H+T207H, S76H+Y208H, S76H+S210H, S76H+L211H, S76H+N212H, S76H+K229H, S76H+Q230H, S76H+K231H, S76H+N232H, S76H+P233H, S76H+S234H, S76H+W235H, S76H+S236H, S76H+V238H, S76H+Q239H, S76H+R241H, S76H+N242H, S76H+K245H, S76H+N246H, S76H+T247H, S76H+T249H, S76H+S250H, S76H+L251H, S76H+G252H, S76H+S253H, S76H+T254H, S76H+N255H, S76H+L256H, S76H+Y257H, S76H+S259H, S76H+E265H, S76H+R269H, 177H+S85H, 177H+E87H, 177H+Y89H, 177H+A96H, I77H+S97H, I77H+G98H, I77H+S99H, I77H+G100H, I77H+S101H, I77H+V102H, 177H+S103H, 177H+Q107H, 177H+E110H, 177H+W111H, 177H+N114H, 177H+N115H, 177H+G116H, 177H+G125H, 177H+S126H, 177H+P127H, 177H+S128H, 177H+P129H, 177H+A131H, 177H+T132H, 177H+E134H, 177H+Q135H, 177H+N138H, 177H+S139H, 177H+S142H, 177H+R143H, 177H+G144H, 177H+N153H, 177H+S154H, 177H+G155H, 177H+A156H, 177H+G157H, 177H+S158H, 177H+Y161H, 177H+R164H, 177H+A166H, 177H+N167H, 177H+D175H, 177H+Q176H, 177H+N177H, 177H+N178H, 177H+N179H, 177H+R180H, 177H+S182H, 177H+F183H, 177H+Y186H, 177H+A188H, 177H+G189H, 177H+N198H, 177H+Q200H, 177H+Y203H, 177H+P204H, 177H+G205H, 177H+S206H, 177H+T207H, 177H+Y208H, 177H+S210H, 177H+L211H, 177H+N212H, 177H+K229H, 177H+Q230H, 177H+K231H, I77H+N232H, I77H+P233H, I77H+S234H, I77H+W235H, I77H+S236H, I77H+V238H, 177H+Q239H, 177H+R241H, I77H+N242H, I77H+K245H, I77H+N246H, I77H+T247H, I77H+T249H, I77H+S250H, 177H+L251H, I77H+G252H, I77H+S253H, I77H+T254H, I77H+N255H, I77H+L256H, I77H+Y257H, I77H+S259H, I77H+E265H, I77H+R269H, S85H+E87H, S85H+Y89H, S85H+A96H, S85H+S97H, S85H+G98H, S85H+S99H, S85H+G100H, S85H+S101H, S85H+V102H, S85H+S103H, S85H+Q107H, S85H+E110H, S85H+W111H, S85H+N114H, S85H+N115H, S85H+G116H, S85H+G125H, S85H+S126H, S85H+P127H, S85H+S128H, S85H+P129H, S85H+A131H, S85H+T132H, S85H+E134H, S85H+Q135H, S85H+N138H, S85H+S139H, S85H+S142H, S85H+R143H, S85H+G144H, S85H+N153H, S85H+S154H, S85H+G155H, S85H+A156H, S85H+G157H, S85H+S158H, S85H+Y161H, S85H+R164H, S85H+A166H, S85H+N167H, S85H+D175H, S85H+Q176H, S85H+N177H, S85H+N178H, S85H+N179H, S85H+R180H, S85H+S182H, S85H+F183H, S85H+Y186H, S85H+A188H, S85H+G189H, S85H+N198H, S85H+Q200H, S85H+Y203H, S85H+P204H, S85H+G205H, S85H+S206H, S85H+T207H, S85H+Y208H, S85H+S210H, S85H+L211H, S85H+N212H, S85H+K229H, S85H+Q230H, S85H+K231H, S85H+N232H, S85H+P233H, S85H+S234H, S85H+W235H, S85H+S236H, S85H+V238H, S85H+Q239H, S85H+R241H, S85H+N242H, S85H+K245H, S85H+N246H, S85H+T247H, S85H+T249H, S85H+S250H, S85H+L251H, S85H+G252H, S85H+S253H, S85H+T254H, S85H+N255H, S85H+L256H, S85H+Y257H, S85H+S259H, S85H+E265H, S85H+R269H, E87H+Y89H, E87H+A96H, E87H+S97H, E87H+G98H, E87H+S99H, E87H+G100H, E87H+S101H, E87H+V102H, E87H+S103H, E87H+Q107H, E87H+E110H, E87H+W111H, E87H+N114H, E87H+N115H, E87H+G116H, E87H+G125H, E87H+S126H, E87H+P127H, E87H+S128H, E87H+P129H, E87H+A131H, E87H+T132H, E87H+E134H, E87H+Q135H, E87H+N138H, E87H+S139H, E87H+S142H, E87H+R143H, E87H+G144H, E87H+N153H, E87H+S154H, E87H+G155H, E87H+A156H, E87H+G157H, E87H+S158H, E87H+Y161H, E87H+R164H, E87H+A166H, E87H+N167H, E87H+D175H, E87H+Q176H, E87H+N177H, E87H+N178H, E87H+N179H, E87H+R180H, E87H+S182H, E87H+F183H, E87H+Y186H, E87H+A188H, E87H+G189H, E87H+N198H, E87H+Q200H, E87H+Y203H, E87H+P204H, E87H+G205H, E87H+S206H, E87H+T207H, E87H+Y208H, E87H+S210H, E87H+L211H, E87H+N212H, E87H+K229H, E87H+Q230H, E87H+K231H, E87H+N232H, E87H+P233H, E87H+S234H, E87H+W235H, E87H+S236H, E87H+V238H, E87H+Q239H, E87H+R241H, E87H+N242H, E87H+K245H, E87H+N246H, E87H+T247H, E87H+T249H, E87H+S250H, E87H+L251H, E87H+G252H, E87H+S253H, E87H+T254H, E87H+N255H, E87H+L256H, E87H+Y257H, E87H+S259H, E87H+E265H, E87H+R269H, Y89H+A96H, Y89H+S97H, Y89H+G98H, Y89H+S99H, Y89H+G100H, Y89H+S101H, Y89H+V102H, Y89H+S103H, Y89H+Q107H, Y89H+E110H, Y89H+W111H, Y89H+N114H, Y89H+N115H, Y89H+G116H, Y89H+G125H, Y89H+S126H, Y89H+P127H, Y89H+S128H, Y89H+P129H, Y89H+A131H, Y89H+T132H, Y89H+E134H, Y89H+Q135H, Y89H+N138H, Y89H+S139H, Y89H+S142H, Y89H+R143H, Y89H+G144H, Y89H+N153H, Y89H+S154H, Y89H+G155H, Y89H+A156H, Y89H+G157H, Y89H+S158H, Y89H+Y161H, Y89H+R164H, Y89H+A166H, Y89H+N167H, Y89H+D175H, Y89H+Q176H, Y89H+N177H, Y89H+N178H, Y89H+N179H, Y89H+R180H, Y89H+S182H, Y89H+F183H, Y89H+Y186H, Y89H+A188H, Y89H+G189H, Y89H+N198H, Y89H+Q200H, Y89H+Y203H, Y89H+P204H, Y89H+G205H, Y89H+S206H, Y89H+T207H, Y89H+Y208H, Y89H+S210H, Y89H+L211H, Y89H+N212H, Y89H+K229H, Y89H+Q230H, Y89H+K231H, Y89H+N232H, Y89H+P233H, Y89H+S234H, Y89H+W235H, Y89H+S236H, Y89H+V238H, Y89H+Q239H, Y89H+R241H, Y89H+N242H, Y89H+K245H, Y89H+N246H, Y89H+T247H, Y89H+T249H, Y89H+S250H, Y89H+L251H, Y89H+G252H, Y89H+S253H, Y89H+T254H, Y89H+N255H, Y89H+L256H, Y89H+Y257H, Y89H+S259H, Y89H+E265H, Y89H+R269H, A96H+S97H, A96H+G98H, A96H+S99H, A96H+G100H, A96H+S101H, A96H+V102H, A96H+S103H, A96H+Q107H, A96H+E110H, A96H+W111H, A96H+N114H, A96H+N115H, A96H+G116H, A96H+G125H, A96H+S126H, A96H+P127H, A96H+S128H, A96H+P129H, A96H+A131H, A96H+T132H, A96H+E134H, A96H+Q135H, A96H+N138H, A96H+S139H, A96H+S142H, A96H+R143H, A96H+G144H, A96H+N153H, A96H+S154H, A96H+G155H, A96H+A156H, A96H+G157H, A96H+S158H, A96H+Y161H, A96H+R164H, A96H+A166H, A96H+N167H, A96H+D175H, A96H+Q176H, A96H+N177H, A96H+N178H, A96H+N179H, A96H+R180H, A96H+S182H, A96H+F183H, A96H+Y186H, A96H+A188H, A96H+G189H, A96H+N198H, A96H+Q200H, A96H+Y203H, A96H+P204H, A96H+G205H, A96H+S206H, A96H+T207H, A96H+Y208H, A96H+S210H, A96H+L211H, A96H+N212H, A96H+K229H, A96H+Q230H, A96H+K231H, A96H+N232H, A96H+P233H, A96H+S234H, A96H+W235H, A96H+S236H, A96H+V238H, A96H+Q239H, A96H+R241H, A96H+N242H, A96H+K245H, A96H+N246H, A96H+T247H, A96H+T249H, A96H+S250H, A96H+L251H, A96H+G252H, A96H+S253H, A96H+T254H, A96H+N255H, A96H+L256H, A96H+Y257H, A96H+S259H, A96H+E265H, A96H+R269H, S97H+G98H, S97H+S99H, S97H+G100H, S97H+S101H, S97H+V102H, S97H+S103H, S97H+Q107H, S97H+E110H, S97H+W111H, S97H+N114H, S97H+N115H, S97H+G116H, S97H+G125H, S97H+S126H, S97H+P127H, S97H+S128H, S97H+P129H, S97H+A131H, S97H+T132H, S97H+E134H, S97H+Q135H, S97H+N138H, S97H+S139H, S97H+S142H, S97H+R143H, S97H+G144H, S97H+N153H, S97H+S154H, S97H+G155H, S97H+A156H, S97H+G157H, S97H+S158H, S97H+Y161H, S97H+R164H, S97H+A166H, S97H+N167H, S97H+D175H, S97H+Q176H, S97H+N177H, S97H+N178H, S97H+N179H, S97H+R180H, S97H+S182H, S97H+F183H, S97H+Y186H, S97H+A188H, S97H+G189H, S97H+N198H, S97H+Q200H, S97H+Y203H, S97H+P204H, S97H+G205H, S97H+S206H, S97H+T207H, S97H+Y208H, S97H+S210H, S97H+L211H, S97H+N212H, S97H+K229H, S97H+Q230H, S97H+K231H, S97H+N232H, S97H+P233H, S97H+S234H, S97H+W235H, S97H+S236H, S97H+V238H, S97H+Q239H, S97H+R241H, S97H+N242H, S97H+K245H, S97H+N246H, S97H+T247H, S97H+T249H, S97H+S250H, S97H+L251H, S97H+G252H, S97H+S253H, S97H+T254H, S97H+N255H, S97H+L256H, S97H+Y257H, S97H+S259H, S97H+E265H, S97H+R269H, G98H+S99H, G98H+G100H, G98H+S101H, G98H+V102H, G98H+S103H, G98H+Q107H, G98H+E110H, G98H+W111H, G98H+N114H, G98H+N115H, G98H+G116H, G98H+G125H, G98H+S126H, G98H+P127H, G98H+S128H, G98H+P129H, G98H+A131H, G98H+T132H, G98H+E134H, G98H+Q135H, G98H+N138H, G98H+S139H, G98H+S142H, G98H+R143H, G98H+G144H, G98H+N153H, G98H+S154H, G98H+G155H, G98H+A156H, G98H+G157H, G98H+S158H, G98H+Y161H, G98H+R164H, G98H+A166H, G98H+N167H, G98H+D175H, G98H+Q176H, G98H+N177H, G98H+N178H, G98H+N179H, G98H+R180H, G98H+S182H, G98H+F183H, G98H+Y186H, G98H+A188H, G98H+G189H, G98H+N198H, G98H+Q200H, G98H+Y203H, G98H+P204H, G98H+G205H, G98H+S206H, G98H+T207H, G98H+Y208H, G98H+S210H, G98H+L211H, G98H+N212H, G98H+K229H, G98H+Q230H, G98H+K231H, G98H+N232H, G98H+P233H, G98H+S234H, G98H+W235H, G98H+S236H, G98H+V238H, G98H+Q239H, G98H+R241H, G98H+N242H, G98H+K245H, G98H+N246H, G98H+T247H, G98H+T249H, G98H+S250H, G98H+L251H, G98H+G252H, G98H+S253H, G98H+T254H, G98H+N255H, G98H+L256H, G98H+Y257H, G98H+S259H, G98H+E265H, G98H+R269H, S99H+G100H, S99H+S101H, S99H+V102H, S99H+S103H, S99H+Q107H, S99H+E110H, S99H+W111H, S99H+N114H, S99H+N115H, S99H+G116H, S99H+G125H, S99H+S126H, S99H+P127H, S99H+S128H, S99H+P129H, S99H+A131H, S99H+T132H, S99H+E134H, S99H+Q135H, S99H+N138H, S99H+S139H, S99H+S142H, S99H+R143H, S99H+G144H, S99H+N153H, S99H+S154H, S99H+G155H, S99H+A156H, S99H+G157H, S99H+S158H, S99H+Y161H, S99H+R164H, S99H+A166H, S99H+N167H, S99H+D175H, S99H+Q176H, S99H+N177H, S99H+N178H, S99H+N179H, S99H+R180H, S99H+S182H, S99H+F183H, S99H+Y186H, S99H+A188H, S99H+G189H, S99H+N198H, S99H+Q200H, S99H+Y203H, S99H+P204H, S99H+G205H, S99H+S206H, S99H+T207H, S99H+Y208H, S99H+S210H, S99H+L211H, S99H+N212H, S99H+K229H, S99H+Q230H, S99H+K231H, S99H+N232H, S99H+P233H, S99H+S234H, S99H+W235H, S99H+S236H, S99H+V238H, S99H+Q239H, S99H+R241H, S99H+N242H, S99H+K245H, S99H+N246H, S99H+T247H, S99H+T249H, S99H+S250H, S99H+L251H, S99H+G252H, S99H+S253H, S99H+T254H, S99H+N255H, S99H+L256H, S99H+Y257H, S99H+S259H, S99H+E265H, S99H+R269H, G100H+S101H, G100H+V102H, G100H+S103H, G100H+Q107H, G100H+E110H, G100H+W111H, G100H+N114H, G100H+N115H, G100H+G116H, G100H+G125H, G100H+S126H, G100H+P127H, G100H+S128H, G100H+P129H, G100H+A131H, G100H+T132H, G100H+E134H, G100H+Q135H, G100H+N138H, G100H+S139H, G100H+S142H, G100H+R143H, G100H+G144H, G100H+N153H, G100H+S154H, G100H+G155H, G100H+A156H, G100H+G157H, G100H+S158H, G100H+Y161H, G100H+R164H, G100H+A166H, G100H+N167H, G100H+D175H, G100H+Q176H, G100H+N177H, G100H+N178H, G100H+N179H, G100H+R180H, G100H+S182H, G100H+F183H, G100H+Y186H, G100H+A188H, G100H+G189H, G100H+N198H, G100H+Q200H, G100H+Y203H, G100H+P204H, G100H+G205H, G100H+S206H, G100H+T207H, G100H+Y208H, G100H+S210H, G100H+L211H, G100H+N212H, G100H+K229H, G100H+Q230H, G100H+K231H, G100H+N232H, G100H+P233H, G100H+S234H, G100H+W235H, G100H+S236H, G100H+V238H, G100H+Q239H, G100H+R241H, G100H+N242H, G100H+K245H, G100H+N246H, G100H+T247H, G100H+T249H, G100H+S250H, G100H+L251H, G100H+G252H, G100H+S253H, G100H+T254H, G100H+N255H, G100H+L256H, G100H+Y257H, G100H+S259H, G100H+E265H, G100H+R269H, S101H+V102H, S101H+S103H, S101H+Q107H, S101H+E110H, S101H+W111H, S101H+N114H, S101H+N115H, S101H+G116H, S101H+G125H, S101H+S126H, S101H+P127H, S101H+S128H, S101H+P129H, S101H+A131H, S101H+T132H, S101H+E134H, S101H+Q135H, S101H+N138H, S101H+S139H, S101H+S142H, S101H+R143H, S101H+G144H, S101H+N153H, S101H+S154H, S101H+G155H, S101H+A156H, S101H+G157H, S101H+S158H, S101H+Y161H, S101H+R164H, S101H+A166H, S101H+N167H, S101H+D175H, S101H+Q176H, S101H+N177H, S101H+N178H, S101H+N179H, S101H+R180H, S101H+S182H, S101H+F183H, S101H+Y186H, S101H+A188H, S101H+G189H, S101H+N198H, S101H+Q200H, S101H+Y203H, S101H+P204H, S101H+G205H, S101H+S206H, S101H+T207H, S101H+Y208H, S101H+S210H, S101H+L211H, S101H+N212H, S101H+K229H, S101H+Q230H, S101H+K231H, S101H+N232H, S101H+P233H, S101H+S234H, S101H+W235H, S101H+S236H, S101H+V238H, S101H+Q239H, S101H+R241H, S101H+N242H, S101H+K245H, S101H+N246H, S101H+T247H, S101H+T249H, S101H+S250H, S101H+L251H, S101H+G252H, S101H+S253H, S101H+T254H, S101H+N255H, S101H+L256H, S101H+Y257H, S101H+S259H, S101H+E265H, S101H+R269H, V102H+S103H, V102H+Q107H, V102H+E110H, V102H+W111H, V102H+N114H, V102H+N115H, V102H+G116H, V102H+G125H, V102H+S126H, V102H+P127H, V102H+S128H, V102H+P129H, V102H+A131H, V102H+T132H, V102H+E134H, V102H+Q135H, V102H+N138H, V102H+S139H, V102H+S142H, V102H+R143H, V102H+G144H, V102H+N153H, V102H+S154H, V102H+G155H, V102H+A156H, V102H+G157H, V102H+S158H, V102H+Y161H, V102H+R164H, V102H+A166H, V102H+N167H, V102H+D175H, V102H+Q176H, V102H+N177H, V102H+N178H, V102H+N179H, V102H+R180H, V102H+S182H, V102H+F183H, V102H+Y186H, V102H+A188H, V102H+G189H, V102H+N198H, V102H+Q200H, V102H+Y203H, V102H+P204H, V102H+G205H, V102H+S206H, V102H+T207H, V102H+Y208H, V102H+S210H, V102H+L211H, V102H+N212H, V102H+K229H, V102H+Q230H, V102H+K231H, V102H+N232H, V102H+P233H, V102H+S234H, V102H+W235H, V102H+S236H, V102H+V238H, V102H+Q239H, V102H+R241H, V102H+N242H, V102H+K245H, V102H+N246H, V102H+T247H, V102H+T249H, V102H+S250H, V102H+L251H, V102H+G252H, V102H+S253H, V102H+T254H, V102H+N255H, V102H+L256H, V102H+Y257H, V102H+S259H, V102H+E265H, V102H+R269H, S103H+Q107H, S103H+E110H, S103H+W111H, S103H+N114H, S103H+N115H, S103H+G116H, S103H+G125H, S103H+S126H, S103H+P127H, S103H+S128H, S103H+P129H, S103H+A131H, S103H+T132H, S103H+E134H, S103H+Q135H, S103H+N138H, S103H+S139H, S103H+S142H, S103H+R143H, S103H+G144H, S103H+N153H, S103H+S154H, S103H+G155H, S103H+A156H, S103H+G157H, S103H+S158H, S103H+Y161H, S103H+R164H, S103H+A166H, S103H+N167H, S103H+D175H, S103H+Q176H, S103H+N177H, S103H+N178H, S103H+N179H, S103H+R180H, S103H+S182H, S103H+F183H, S103H+Y186H, S103H+A188H, S103H+G189H, S103H+N198H, S103H+Q200H, S103H+Y203H, S103H+P204H, S103H+G205H, S103H+S206H, S103H+T207H, S103H+Y208H, S103H+S210H, S103H+L211H, S103H+N212H, S103H+K229H, S103H+Q230H, S103H+K231H, S103H+N232H, S103H+P233H, S103H+S234H, S103H+W235H, S103H+S236H, S103H+V238H, S103H+Q239H, S103H+R241H, S103H+N242H, S103H+K245H, S103H+N246H, S103H+T247H, S103H+T249H, S103H+S250H, S103H+L251H, S103H+G252H, S103H+S253H, S103H+T254H, S103H+N255H, S103H+L256H, S103H+Y257H, S103H+S259H, S103H+E265H, S103H+R269H, Q107H+E110H, Q107H+W111H, Q107H+N114H, Q107H+N115H, Q107H+G116H, Q107H+G125H, Q107H+S126H, Q107H+P127H, Q107H+S128H, Q107H+P129H, Q107H+A131H, Q107H+T132H, Q107H+E134H, Q107H+Q135H, Q107H+N138H, Q107H+S139H, Q107H+S142H, Q107H+R143H, Q107H+G144H, Q107H+N153H, Q107H+S154H, Q107H+G155H, Q107H+A156H, Q107H+G157H, Q107H+S158H, Q107H+Y161H, Q107H+R164H, Q107H+A166H, Q107H+N167H, Q107H+D175H, Q107H+Q176H, Q107H+N177H, Q107H+N178H, Q107H+N179H, Q107H+R180H, Q107H+S182H, Q107H+F183H, Q107H+Y186H, Q107H+A188H, Q107H+G189H, Q107H+N198H, Q107H+Q200H, Q107H+Y203H, Q107H+P204H, Q107H+G205H, Q107H+S206H, Q107H+T207H, Q107H+Y208H, Q107H+S210H, Q107H+L211H, Q107H+N212H, Q107H+K229H, Q107H+Q230H, Q107H+K231H, Q107H+N232H, Q107H+P233H, Q107H+S234H, Q107H+W235H, Q107H+S236H, Q107H+V238H, Q107H+Q239H, Q107H+R241H, Q107H+N242H, Q107H+K245H, Q107H+N246H, Q107H+T247H, Q107H+T249H, Q107H+S250H, Q107H+L251H, Q107H+G252H, Q107H+S253H, Q107H+T254H, Q107H+N255H, Q107H+L256H, Q107H+Y257H, Q107H+S259H, Q107H+E265H, Q107H+R269H, E110H+W111H, E110H+N114H, E110H+N115H, E110H+G116H, E110H+G125H, E110H+S126H, E110H+P127H, E110H+S128H, E110H+P129H, E110H+A131H, E110H+T132H, E110H+E134H, E110H+Q135H, E110H+N138H, E110H+S139H, E110H+S142H, E110H+R143H, E110H+G144H, E110H+N153H, E110H+S154H, E110H+G155H, E110H+A156H, E110H+G157H, E110H+S158H, E110H+Y161H, E110H+R164H, E110H+A166H, E110H+N167H, E110H+D175H, E110H+Q176H, E110H+N177H, E110H+N178H, E110H+N179H, E110H+R180H, E110H+S182H, E110H+F183H, E110H+Y186H, E110H+A188H, E110H+G189H, E110H+N198H, E110H+Q200H, E110H+Y203H, E110H+P204H, E110H+G205H, E110H+S206H, E110H+T207H, E110H+Y208H, E110H+S210H, E110H+L211H, E110H+N212H, E110H+K229H, E110H+Q230H, E110H+K231H, E110H+N232H, E110H+P233H, E110H+S234H, E110H+W235H, E110H+S236H, E110H+V238H, E110H+Q239H, E110H+R241H, E110H+N242H, E110H+K245H, E110H+N246H, E110H+T247H, E110H+T249H, E110H+S250H, E110H+L251H, E110H+G252H, E110H+S253H, E110H+T254H, E110H+N255H, E110H+L256H, E110H+Y257H, E110H+S259H, E110H+E265H, E110H+R269H, W111H+N114H, W111H+N115H, W111H+G116H, W111H+G125H, W111H+S126H, W111H+P127H, W111H+S128H, W111H+P129H, W111H+A131H, W111H+T132H, W111H+E134H, W111H+Q135H, W111H+N138H, W111H+S139H, W111H+S142H, W111H+R143H, W111H+G144H, W111H+N153H, W111H+S154H, W111H+G155H, W111H+A156H, W111H+G157H, W111H+S158H, W111H+Y161H, W111H+R164H, W111H+A166H, W111H+N167H, W111H+D175H, W111H+Q176H, W111H+N177H, W111H+N178H, W111H+N179H, W111H+R180H, W111H+S182H, W111H+F183H, W111H+Y186H, W111H+A188H, W111H+G189H, W111H+N198H, W111H+Q200H, W111H+Y203H, W111H+P204H, W111H+G205H, W111H+S206H, W111H+T207H, W111H+Y208H, W111H+S210H, W111H+L211H, W111H+N212H, W111H+K229H, W111H+Q230H, W111H+K231H, W111H+N232H, W111H+P233H, W111H+S234H, W111H+W235H, W111H+S236H, W111H+V238H, W111H+Q239H, W111H+R241H, W111H+N242H, W111H+K245H, W111H+N246H, W111H+T247H, W111H+T249H, W111H+S250H, W111H+L251H, W111H+G252H, W111H+S253H, W111H+T254H, W111H+N255H, W111H+L256H, W111H+Y257H, W111H+S259H, W111H+E265H, W111H+R269H, N114H+N115H, N114H+G116H, N114H+G125H, N114H+S126H, N114H+P127H, N114H+S128H, N114H+P129H, N114H+A131H, N114H+T132H, N114H+E134H, N114H+Q135H, N114H+N138H, N114H+S139H, N114H+S142H, N114H+R143H, N114H+G144H, N114H+N153H, N114H+S154H, N114H+G155H, N114H+A156H, N114H+G157H, N114H+S158H, N114H+Y161H, N114H+R164H, N114H+A166H, N114H+N167H, N114H+D175H, N114H+Q176H, N114H+N177H, N114H+N178H, N114H+N179H, N114H+R180H, N114H+S182H, N114H+F183H, N114H+Y186H, N114H+A188H, N114H+G189H, N114H+N198H, N114H+Q200H, N114H+Y203H, N114H+P204H, N114H+G205H, N114H+S206H, N114H+T207H, N114H+Y208H, N114H+S210H, N114H+L211H, N114H+N212H, N114H+K229H, N114H+Q230H, N114H+K231H, N114H+N232H, N114H+P233H, N114H+S234H, N114H+W235H, N114H+S236H, N114H+V238H, N114H+Q239H, N114H+R241H, N114H+N242H, N114H+K245H, N114H+N246H, N114H+T247H, N114H+T249H, N114H+S250H, N114H+L251H, N114H+G252H, N114H+S253H, N114H+T254H, N114H+N255H, N114H+L256H, N114H+Y257H, N114H+S259H, N114H+E265H, N114H+R269H, N115H+G116H, N115H+G125H, N115H+S126H, N115H+P127H, N115H+S128H, N115H+P129H, N115H+A131H, N115H+T132H, N115H+E134H, N115H+Q135H, N115H+N138H, N115H+S139H, N115H+S142H, N115H+R143H, N115H+G144H, N115H+N153H, N115H+S154H, N115H+G155H, N115H+A156H, N115H+G157H, N115H+S158H, N115H+Y161H, N115H+R164H, N115H+A166H, N115H+N167H, N115H+D175H, N115H+Q176H, N115H+N177H, N115H+N178H, N115H+N179H, N115H+R180H, N115H+S182H, N115H+F183H, N115H+Y186H, N115H+A188H, N115H+G189H, N115H+N198H, N115H+Q200H, N115H+Y203H, N115H+P204H, N115H+G205H, N115H+S206H, N115H+T207H, N115H+Y208H, N115H+S210H, N115H+L211H, N115H+N212H, N115H+K229H, N115H+Q230H, N115H+K231H, N115H+N232H, N115H+P233H, N115H+S234H, N115H+W235H, N115H+S236H, N115H+V238H, N115H+Q239H, N115H+R241H, N115H+N242H, N115H+K245H, N115H+N246H, N115H+T247H, N115H+T249H, N115H+S250H, N115H+L251H, N115H+G252H, N115H+S253H, N115H+T254H, N115H+N255H, N115H+L256H, N115H+Y257H, N115H+S259H, N115H+E265H, N115H+R269H, G116H+G125H, G116H+S126H, G116H+P127H, G116H+S128H, G116H+P129H, G116H+A131H, G116H+T132H, G116H+E134H, G116H+Q135H, G116H+N138H, G116H+S139H, G116H+S142H, G116H+R143H, G116H+G144H, G116H+N153H, G116H+S154H, G116H+G155H, G116H+A156H, G116H+G157H, G116H+S158H, G116H+Y161H, G116H+R164H, G116H+A166H, G116H+N167H, G116H+D175H, G116H+Q176H, G116H+N177H, G116H+N178H, G116H+N179H, G116H+R180H, G116H+S182H, G116H+F183H, G116H+Y186H, G116H+A188H, G116H+G189H, G116H+N198H, G116H+Q200H, G116H+Y203H, G116H+P204H, G116H+G205H, G116H+S206H, G116H+T207H, G116H+Y208H, G116H+S210H, G116H+L211H, G116H+N212H, G116H+K229H, G116H+Q230H, G116H+K231H, G116H+N232H, G116H+P233H, G116H+S234H, G116H+W235H, G116H+S236H, G116H+V238H, G116H+Q239H, G116H+R241H, G116H+N242H, G116H+K245H, G116H+N246H, G116H+T247H, G116H+T249H, G116H+S250H, G116H+L251H, G116H+G252H, G116H+S253H, G116H+T254H, G116H+N255H, G116H+L256H, G116H+Y257H, G116H+S259H, G116H+E265H, G116H+R269H, G125H+S126H, G125H+P127H, G125H+S128H, G125H+P129H, G125H+A131H, G125H+T132H, G125H+E134H, G125H+Q135H, G125H+N138H, G125H+S139H, G125H+S142H, G125H+R143H, G125H+G144H, G125H+N153H, G125H+S154H, G125H+G155H, G125H+A156H, G125H+G157H, G125H+S158H, G125H+Y161H, G125H+R164H, G125H+A166H, G125H+N167H, G125H+D175H, G125H+Q176H, G125H+N177H, G125H+N178H, G125H+N179H, G125H+R180H, G125H+S182H, G125H+F183H, G125H+Y186H, G125H+A188H, G125H+G189H, G125H+N198H, G125H+Q200H, G125H+Y203H, G125H+P204H, G125H+G205H, G125H+S206H, G125H+T207H, G125H+Y208H, G125H+S210H, G125H+L211H, G125H+N212H, G125H+K229H, G125H+Q230H, G125H+K231H, G125H+N232H, G125H+P233H, G125H+S234H, G125H+W235H, G125H+S236H, G125H+V238H, G125H+Q239H, G125H+R241H, G125H+N242H, G125H+K245H, G125H+N246H, G125H+T247H, G125H+T249H, G125H+S250H, G125H+L251H, G125H+G252H, G125H+S253H, G125H+T254H, G125H+N255H, G125H+L256H, G125H+Y257H, G125H+S259H, G125H+E265H, G125H+R269H, S126H+P127H, S126H+S128H, S126H+P129H, S126H+A131H, S126H+T132H, S126H+E134H, S126H+Q135H, S126H+N138H, S126H+S139H, S126H+S142H, S126H+R143H, S126H+G144H, S126H+N153H, S126H+S154H, S126H+G155H, S126H+A156H, S126H+G157H, S126H+S158H, S126H+Y161H, S126H+R164H, S126H+A166H, S126H+N167H, S126H+D175H, S126H+Q176H, S126H+N177H, S126H+N178H, S126H+N179H, S126H+R180H, S126H+S182H, S126H+F183H, S126H+Y186H, S126H+A188H, S126H+G189H, S126H+N198H, S126H+Q200H, S126H+Y203H, S126H+P204H, S126H+G205H, S126H+S206H, S126H+T207H, S126H+Y208H, S126H+S210H, S126H+L211H, S126H+N212H, S126H+K229H, S126H+Q230H, S126H+K231H, S126H+N232H, S126H+P233H, S126H+S234H, S126H+W235H, S126H+S236H, S126H+V238H, S126H+Q239H, S126H+R241H, S126H+N242H, S126H+K245H, S126H+N246H, S126H+T247H, S126H+T249H, S126H+S250H, S126H+L251H, S126H+G252H, S126H+S253H, S126H+T254H, S126H+N255H, S126H+L256H, S126H+Y257H, S126H+S259H, S126H+E265H, S126H+R269H, P127H+S128H, P127H+P129H, P127H+A131H, P127H+T132H, P127H+E134H, P127H+Q135H, P127H+N138H, P127H+S139H, P127H+S142H, P127H+R143H, P127H+G144H, P127H+N153H, P127H+S154H, P127H+G155H, P127H+A156H, P127H+G157H, P127H+S158H, P127H+Y161H, P127H+R164H, P127H+A166H, P127H+N167H, P127H+D175H, P127H+Q176H, P127H+N177H, P127H+N178H, P127H+N179H, P127H+R180H, P127H+S182H, P127H+F183H, P127H+Y186H, P127H+A188H, P127H+G189H, P127H+N198H, P127H+Q200H, P127H+Y203H, P127H+P204H, P127H+G205H, P127H+S206H, P127H+T207H, P127H+Y208H, P127H+S210H, P127H+L211H, P127H+N212H, P127H+K229H, P127H+Q230H, P127H+K231H, P127H+N232H, P127H+P233H, P127H+S234H, P127H+W235H, P127H+S236H, P127H+V238H, P127H+Q239H, P127H+R241H, P127H+N242H, P127H+K245H, P127H+N246H, P127H+T247H, P127H+T249H, P127H+S250H, P127H+L251H, P127H+G252H, P127H+S253H, P127H+T254H, P127H+N255H, P127H+L256H, P127H+Y257H, P127H+S259H, P127H+E265H, P127H+R269H, S128H+P129H, S128H+A131H, S128H+T132H, S128H+E134H, S128H+Q135H, S128H+N138H, S128H+S139H, S128H+S142H, S128H+R143H, S128H+G144H, S128H+N153H, S128H+S154H, S128H+G155H, S128H+A156H, S128H+G157H, S128H+S158H, S128H+Y161H, S128H+R164H, S128H+A166H, S128H+N167H, S128H+D175H, S128H+Q176H, S128H+N177H, S128H+N178H, S128H+N179H, S128H+R180H, S128H+S182H, S128H+F183H, S128H+Y186H, S128H+A188H, S128H+G189H, S128H+N198H, S128H+Q200H, S128H+Y203H, S128H+P204H, S128H+G205H, S128H+S206H, S128H+T207H, S128H+Y208H, S128H+S210H, S128H+L211H, S128H+N212H, S128H+K229H, S128H+Q230H, S128H+K231H, S128H+N232H, S128H+P233H, S128H+S234H, S128H+W235H, S128H+S236H, S128H+V238H, S128H+Q239H, S128H+R241H, S128H+N242H, S128H+K245H, S128H+N246H, S128H+T247H, S128H+T249H, S128H+S250H, S128H+L251H, S128H+G252H, S128H+S253H, S128H+T254H, S128H+N255H, S128H+L256H, S128H+Y257H, S128H+S259H, S128H+E265H, S128H+R269H, P129H+A131H, P129H+T132H, P129H+E134H, P129H+Q135H, P129H+N138H, P129H+S139H, P129H+S142H, P129H+R143H, P129H+G144H, P129H+N153H, P129H+S154H, P129H+G155H, P129H+A156H, P129H+G157H, P129H+S158H, P129H+Y161H, P129H+R164H, P129H+A166H, P129H+N167H, P129H+D175H, P129H+Q176H, P129H+N177H, P129H+N178H, P129H+N179H, P129H+R180H, P129H+S182H, P129H+F183H, P129H+Y186H, P129H+A188H, P129H+G189H, P129H+N198H, P129H+Q200H, P129H+Y203H, P129H+P204H, P129H+G205H, P129H+S206H, P129H+T207H, P129H+Y208H, P129H+S210H, P129H+L211H, P129H+N212H, P129H+K229H, P129H+Q230H, P129H+K231H, P129H+N232H, P129H+P233H, P129H+S234H, P129H+W235H, P129H+S236H, P129H+V238H, P129H+Q239H, P129H+R241H, P129H+N242H, P129H+K245H, P129H+N246H, P129H+T247H, P129H+T249H, P129H+S250H, P129H+L251H, P129H+G252H, P129H+S253H, P129H+T254H, P129H+N255H, P129H+L256H, P129H+Y257H, P129H+S259H, P129H+E265H, P129H+R269H, A131H+T132H, A131H+E134H, A131H+Q135H, A131H+N138H, A131H+S139H, A131H+S142H, A131H+R143H, A131H+G144H, A131H+N153H, A131H+S154H, A131H+G155H, A131H+A156H, A131H+G157H, A131H+S158H, A131H+Y161H, A131H+R164H, A131H+A166H, A131H+N167H, A131H+D175H, A131H+Q176H, A131H+N177H, A131H+N178H, A131H+N179H, A131H+R180H, A131H+S182H, A131H+F183H, A131H+Y186H, A131H+A188H, A131H+G189H, A131H+N198H, A131H+Q200H, A131H+Y203H, A131H+P204H, A131H+G205H, A131H+S206H, A131H+T207H, A131H+Y208H, A131H+S210H, A131H+L211H, A131H+N212H, A131H+K229H, A131H+Q230H, A131H+K231H, A131H+N232H, A131H+P233H, A131H+S234H, A131H+W235H, A131H+S236H, A131H+V238H, A131H+Q239H, A131H+R241H, A131H+N242H, A131H+K245H, A131H+N246H, A131H+T247H, A131H+T249H, A131H+S250H, A131H+L251H, A131H+G252H, A131H+S253H, A131H+T254H, A131H+N255H, A131H+L256H, A131H+Y257H, A131H+S259H, A131H+E265H, A131H+R269H, T132H+E134H, T132H+Q135H, T132H+N138H, T132H+S139H, T132H+S142H, T132H+R143H, T132H+G144H, T132H+N153H, T132H+S154H, T132H+G155H, T132H+A156H, T132H+G157H, T132H+S158H, T132H+Y161H, T132H+R164H, T132H+A166H, T132H+N167H, T132H+D175H, T132H+Q176H, T132H+N177H, T132H+N178H, T132H+N179H, T132H+R180H, T132H+S182H, T132H+F183H, T132H+Y186H, T132H+A188H, T132H+G189H, T132H+N198H, T132H+Q200H, T132H+Y203H, T132H+P204H, T132H+G205H, T132H+S206H, T132H+T207H, T132H+Y208H, T132H+S210H, T132H+L211H, T132H+N212H, T132H+K229H, T132H+Q230H, T132H+K231H, T132H+N232H, T132H+P233H, T132H+S234H, T132H+W235H, T132H+S236H, T132H+V238H, T132H+Q239H, T132H+R241H, T132H+N242H, T132H+K245H, T132H+N246H, T132H+T247H, T132H+T249H, T132H+S250H, T132H+L251H, T132H+G252H, T132H+S253H, T132H+T254H, T132H+N255H, T132H+L256H, T132H+Y257H, T132H+S259H, T132H+E265H, T132H+R269H, E134H+Q135H, E134H+N138H, E134H+S139H, E134H+S142H, E134H+R143H, E134H+G144H, E134H+N153H, E134H+S154H, E134H+G155H, E134H+A156H, E134H+G157H, E134H+S158H, E134H+Y161H, E134H+R164H, E134H+A166H, E134H+N167H, E134H+D175H, E134H+Q176H, E134H+N177H, E134H+N178H, E134H+N179H, E134H+R180H, E134H+S182H, E134H+F183H, E134H+Y186H, E134H+A188H, E134H+G189H, E134H+N198H, E134H+Q200H, E134H+Y203H, E134H+P204H, E134H+G205H, E134H+S206H, E134H+T207H, E134H+Y208H, E134H+S210H, E134H+L211H, E134H+N212H, E134H+K229H, E134H+Q230H, E134H+K231H, E134H+N232H, E134H+P233H, E134H+S234H, E134H+W235H, E134H+S236H, E134H+V238H, E134H+Q239H, E134H+R241H, E134H+N242H, E134H+K245H, E134H+N246H, E134H+T247H, E134H+T249H, E134H+S250H, E134H+L251H, E134H+G252H, E134H+S253H, E134H+T254H, E134H+N255H, E134H+L256H, E134H+Y257H, E134H+S259H, E134H+E265H, E134H+R269H, Q135H+N138H, Q135H+S139H, Q135H+S142H, Q135H+R143H, Q135H+G144H, Q135H+N153H, Q135H+S154H, Q135H+G155H, Q135H+A156H, Q135H+G157H, Q135H+S158H, Q135H+Y161H, Q135H+R164H, Q135H+A166H, Q135H+N167H, Q135H+D175H, Q135H+Q176H, Q135H+N177H, Q135H+N178H, Q135H+N179H, Q135H+R180H, Q135H+S182H, Q135H+F183H, Q135H+Y186H, Q135H+A188H, Q135H+G189H, Q135H+N198H, Q135H+Q200H, Q135H+Y203H, Q135H+P204H, Q135H+G205H, Q135H+S206H, Q135H+T207H, Q135H+Y208H, Q135H+S210H, Q135H+L211H, Q135H+N212H, Q135H+K229H, Q135H+Q230H, Q135H+K231H, Q135H+N232H, Q135H+P233H, Q135H+S234H, Q135H+W235H, Q135H+S236H, Q135H+V238H, Q135H+Q239H, Q135H+R241H, Q135H+N242H, Q135H+K245H, Q135H+N246H, Q135H+T247H, Q135H+T249H, Q135H+S250H, Q135H+L251H, Q135H+G252H, Q135H+S253H, Q135H+T254H, Q135H+N255H, Q135H+L256H, Q135H+Y257H, Q135H+S259H, Q135H+E265H, Q135H+R269H, N138H+S139H, N138H+S142H, N138H+R143H, N138H+G144H, N138H+N153H, N138H+S154H, N138H+G155H, N138H+A156H, N138H+G157H, N138H+S158H, N138H+Y161H, N138H+R164H, N138H+A166H, N138H+N167H, N138H+D175H, N138H+Q176H, N138H+N177H, N138H+N178H, N138H+N179H, N138H+R180H, N138H+S182H, N138H+F183H, N138H+Y186H, N138H+A188H, N138H+G189H, N138H+N198H, N138H+Q200H, N138H+Y203H, N138H+P204H, N138H+G205H, N138H+S206H, N138H+T207H, N138H+Y208H, N138H+S210H, N138H+L211H, N138H+N212H, N138H+K229H, N138H+Q230H, N138H+K231H, N138H+N232H, N138H+P233H, N138H+S234H, N138H+W235H, N138H+S236H, N138H+V238H, N138H+Q239H, N138H+R241H, N138H+N242H, N138H+K245H, N138H+N246H, N138H+T247H, N138H+T249H, N138H+S250H, N138H+L251H, N138H+G252H, N138H+S253H, N138H+T254H, N138H+N255H, N138H+L256H, N138H+Y257H, N138H+S259H, N138H+E265H, N138H+R269H, S139H+S142H, S139H+R143H, S139H+G144H, S139H+N153H, S139H+S154H, S139H+G155H, S139H+A156H, S139H+G157H, S139H+S158H, S139H+Y161H, S139H+R164H, S139H+A166H, S139H+N167H, S139H+D175H, S139H+Q176H, S139H+N177H, S139H+N178H, S139H+N179H, S139H+R180H, S139H+S182H, S139H+F183H, S139H+Y186H, S139H+A188H, S139H+G189H, S139H+N198H, S139H+Q200H, S139H+Y203H, S139H+P204H, S139H+G205H, S139H+S206H, S139H+T207H, S139H+Y208H, S139H+S210H, S139H+L211H, S139H+N212H, S139H+K229H, S139H+Q230H, S139H+K231H, S139H+N232H, S139H+P233H, S139H+S234H, S139H+W235H, S139H+S236H, S139H+V238H, S139H+Q239H, S139H+R241H, S139H+N242H, S139H+K245H, S139H+N246H, S139H+T247H, S139H+T249H, S139H+S250H, S139H+L251H, S139H+G252H, S139H+S253H, S139H+T254H, S139H+N255H, S139H+L256H, S139H+Y257H, S139H+S259H, S139H+E265H, S139H+R269H, S142H+R143H, S142H+G144H, S142H+N153H, S142H+S154H, S142H+G155H, S142H+A156H, S142H+G157H, S142H+S158H, S142H+Y161H, S142H+R164H, S142H+A166H, S142H+N167H, S142H+D175H, S142H+Q176H, S142H+N177H, S142H+N178H, S142H+N179H, S142H+R180H, S142H+S182H, S142H+F183H, S142H+Y186H, S142H+A188H, S142H+G189H, S142H+N198H, S142H+Q200H, S142H+Y203H, S142H+P204H, S142H+G205H, S142H+S206H, S142H+T207H, S142H+Y208H, S142H+S210H, S142H+L211H, S142H+N212H, S142H+K229H, S142H+Q230H, S142H+K231H, S142H+N232H, S142H+P233H, S142H+S234H, S142H+W235H, S142H+S236H, S142H+V238H, S142H+Q239H, S142H+R241H, S142H+N242H, S142H+K245H, S142H+N246H, S142H+T247H, S142H+T249H, S142H+S250H, S142H+L251H, S142H+G252H, S142H+S253H, S142H+T254H, S142H+N255H, S142H+L256H, S142H+Y257H, S142H+S259H, S142H+E265H, S142H+R269H, R143H+G144H, R143H+N153H, R143H+S154H, R143H+G155H, R143H+A156H, R143H+G157H, R143H+S158H, R143H+Y161H, R143H+R164H, R143H+A166H, R143H+N167H, R143H+D175H, R143H+Q176H, R143H+N177H, R143H+N178H, R143H+N179H, R143H+R180H, R143H+S182H, R143H+F183H, R143H+Y186H, R143H+A188H, R143H+G189H, R143H+N198H, R143H+Q200H, R143H+Y203H, R143H+P204H, R143H+G205H, R143H+S206H, R143H+T207H, R143H+Y208H, R143H+S210H, R143H+L211H, R143H+N212H, R143H+K229H, R143H+Q230H, R143H+K231H, R143H+N232H, R143H+P233H, R143H+S234H, R143H+W235H, R143H+S236H, R143H+V238H, R143H+Q239H, R143H+R241H, R143H+N242H, R143H+K245H, R143H+N246H, R143H+T247H, R143H+T249H, R143H+S250H, R143H+L251H, R143H+G252H, R143H+S253H, R143H+T254H, R143H+N255H, R143H+L256H, R143H+Y257H, R143H+S259H, R143H+E265H, R143H+R269H, G144H+N153H, G144H+S154H, G144H+G155H, G144H+A156H, G144H+G157H, G144H+S158H, G144H+Y161H, G144H+R164H, G144H+A166H, G144H+N167H, G144H+D175H, G144H+Q176H, G144H+N177H, G144H+N178H, G144H+N179H, G144H+R180H, G144H+S182H, G144H+F183H, G144H+Y186H, G144H+A188H, G144H+G189H, G144H+N198H, G144H+Q200H, G144H+Y203H, G144H+P204H, G144H+G205H, G144H+S206H, G144H+T207H, G144H+Y208H, G144H+S210H, G144H+L211H, G144H+N212H, G144H+K229H, G144H+Q230H, G144H+K231H, G144H+N232H, G144H+P233H, G144H+S234H, G144H+W235H, G144H+S236H, G144H+V238H, G144H+Q239H, G144H+R241H, G144H+N242H, G144H+K245H, G144H+N246H, G144H+T247H, G144H+T249H, G144H+S250H, G144H+L251H, G144H+G252H, G144H+S253H, G144H+T254H, G144H+N255H, G144H+L256H, G144H+Y257H, G144H+S259H, G144H+E265H, G144H+R269H, N153H+S154H, N153H+G155H, N153H+A156H, N153H+G157H, N153H+S158H, N153H+Y161H, N153H+R164H, N153H+A166H, N153H+N167H, N153H+D175H, N153H+Q176H, N153H+N177H, N153H+N178H, N153H+N179H, N153H+R180H, N153H+S182H, N153H+F183H, N153H+Y186H, N153H+A188H, N153H+G189H, N153H+N198H, N153H+Q200H, N153H+Y203H, N153H+P204H, N153H+G205H, N153H+S206H, N153H+T207H, N153H+Y208H, N153H+S210H, N153H+L211H, N153H+N212H, N153H+K229H, N153H+Q230H, N153H+K231H, N153H+N232H, N153H+P233H, N153H+S234H, N153H+W235H, N153H+S236H, N153H+V238H, N153H+Q239H, N153H+R241H, N153H+N242H, N153H+K245H, N153H+N246H, N153H+T247H, N153H+T249H, N153H+S250H, N153H+L251H, N153H+G252H, N153H+S253H, N153H+T254H, N153H+N255H, N153H+L256H, N153H+Y257H, N153H+S259H, N153H+E265H, N153H+R269H, S154H+G155H, S154H+A156H, S154H+G157H, S154H+S158H, S154H+Y161H, S154H+R164H, S154H+A166H, S154H+N167H, S154H+D175H, S154H+Q176H, S154H+N177H, S154H+N178H, S154H+N179H, S154H+R180H, S154H+S182H, S154H+F183H, S154H+Y186H, S154H+A188H, S154H+G189H, S154H+N198H, S154H+Q200H, S154H+Y203H, S154H+P204H, S154H+G205H, S154H+S206H, S154H+T207H, S154H+Y208H, S154H+S210H, S154H+L211H, S154H+N212H, S154H+K229H, S154H+Q230H, S154H+K231H, S154H+N232H, S154H+P233H, S154H+S234H, S154H+W235H, S154H+S236H, S154H+V238H, S154H+Q239H, S154H+R241H, S154H+N242H, S154H+K245H, S154H+N246H, S154H+T247H, S154H+T249H, S154H+S250H, S154H+L251H, S154H+G252H, S154H+S253H, S154H+T254H, S154H+N255H, S154H+L256H, S154H+Y257H, S154H+S259H, S154H+E265H, S154H+R269H, G155H+A156H, G155H+G157H, G155H+S158H, G155H+Y161H, G155H+R164H, G155H+A166H, G155H+N167H, G155H+D175H, G155H+Q176H, G155H+N177H, G155H+N178H, G155H+N179H, G155H+R180H, G155H+S182H, G155H+F183H, G155H+Y186H, G155H+A188H, G155H+G189H, G155H+N198H, G155H+Q200H, G155H+Y203H, G155H+P204H, G155H+G205H, G155H+S206H, G155H+T207H, G155H+Y208H, G155H+S210H, G155H+L211H, G155H+N212H, G155H+K229H, G155H+Q230H, G155H+K231H, G155H+N232H, G155H+P233H, G155H+S234H, G155H+W235H, G155H+S236H, G155H+V238H, G155H+Q239H, G155H+R241H, G155H+N242H, G155H+K245H, G155H+N246H, G155H+T247H, G155H+T249H, G155H+S250H, G155H+L251H, G155H+G252H, G155H+S253H, G155H+T254H, G155H+N255H, G155H+L256H, G155H+Y257H, G155H+S259H, G155H+E265H, G155H+R269H, A156H+G157H, A156H+S158H, A156H+Y161H, A156H+R164H, A156H+A166H, A156H+N167H, A156H+D175H, A156H+Q176H, A156H+N177H, A156H+N178H, A156H+N179H, A156H+R180H, A156H+S182H, A156H+F183H, A156H+Y186H, A156H+A188H, A156H+G189H, A156H+N198H, A156H+Q200H, A156H+Y203H, A156H+P204H, A156H+G205H, A156H+S206H, A156H+T207H, A156H+Y208H, A156H+S210H, A156H+L211H, A156H+N212H, A156H+K229H, A156H+Q230H, A156H+K231H, A156H+N232H, A156H+P233H, A156H+S234H, A156H+W235H, A156H+S236H, A156H+V238H, A156H+Q239H, A156H+R241H, A156H+N242H, A156H+K245H, A156H+N246H, A156H+T247H, A156H+T249H, A156H+S250H, A156H+L251H, A156H+G252H, A156H+S253H, A156H+T254H, A156H+N255H, A156H+L256H, A156H+Y257H, A156H+S259H, A156H+E265H, A156H+R269H, G157H+S158H, G157H+Y161H, G157H+R164H, G157H+A166H, G157H+N167H, G157H+D175H, G157H+Q176H, G157H+N177H, G157H+N178H, G157H+N179H, G157H+R180H, G157H+S182H, G157H+F183H, G157H+Y186H, G157H+A188H, G157H+G189H, G157H+N198H, G157H+Q200H, G157H+Y203H, G157H+P204H, G157H+G205H, G157H+S206H, G157H+T207H, G157H+Y208H, G157H+S210H, G157H+L211H, G157H+N212H, G157H+K229H, G157H+Q230H, G157H+K231H, G157H+N232H, G157H+P233H, G157H+S234H, G157H+W235H, G157H+S236H, G157H+V238H, G157H+Q239H, G157H+R241H, G157H+N242H, G157H+K245H, G157H+N246H, G157H+T247H, G157H+T249H, G157H+S250H, G157H+L251H, G157H+G252H, G157H+S253H, G157H+T254H, G157H+N255H, G157H+L256H, G157H+Y257H, G157H+S259H, G157H+E265H, G157H+R269H, S158H+Y161H, S158H+R164H, S158H+A166H, S158H+N167H, S158H+D175H, S158H+Q176H, S158H+N177H, S158H+N178H, S158H+N179H, S158H+R180H, S158H+S182H, S158H+F183H, S158H+Y186H, S158H+A188H, S158H+G189H, S158H+N198H, S158H+Q200H, S158H+Y203H, S158H+P204H, S158H+G205H, S158H+S206H, S158H+T207H, S158H+Y208H, S158H+S210H, S158H+L211H, S158H+N212H, S158H+K229H, S158H+Q230H, S158H+K231H, S158H+N232H, S158H+P233H, S158H+S234H, S158H+W235H, S158H+S236H, S158H+V238H, S158H+Q239H, S158H+R241H, S158H+N242H, S158H+K245H, S158H+N246H, S158H+T247H, S158H+T249H, S158H+S250H, S158H+L251H, S158H+G252H, S158H+S253H, S158H+T254H, S158H+N255H, S158H+L256H, S158H+Y257H, S158H+S259H, S158H+E265H, S158H+R269H, Y161H+R164H, Y161H+A166H, Y161H+N167H, Y161H+D175H, Y161H+Q176H, Y161H+N177H, Y161H+N178H, Y161H+N179H, Y161H+R180H, Y161H+S182H, Y161H+F183H, Y161H+Y186H, Y161H+A188H, Y161H+G189H, Y161H+N198H, Y161H+Q200H, Y161H+Y203H, Y161H+P204H, Y161H+G205H, Y161H+S206H, Y161H+T207H, Y161H+Y208H, Y161H+S210H, Y161H+L211H, Y161H+N212H, Y161H+K229H, Y161H+Q230H, Y161H+K231H, Y161H+N232H, Y161H+P233H, Y161H+S234H, Y161H+W235H, Y161H+S236H, Y161H+V238H, Y161H+Q239H, Y161H+R241H, Y161H+N242H, Y161H+K245H, Y161H+N246H, Y161H+T247H, Y161H+T249H, Y161H+S250H, Y161H+L251H, Y161H+G252H, Y161H+S253H, Y161H+T254H, Y161H+N255H, Y161H+L256H, Y161H+Y257H, Y161H+S259H, Y161H+E265H, Y161H+R269H, R164H+A166H, R164H+N167H, R164H+D175H, R164H+Q176H, R164H+N177H, R164H+N178H, R164H+N179H, R164H+R180H, R164H+S182H, R164H+F183H, R164H+Y186H, R164H+A188H, R164H+G189H, R164H+N198H, R164H+Q200H, R164H+Y203H, R164H+P204H, R164H+G205H, R164H+S206H, R164H+T207H, R164H+Y208H, R164H+S210H, R164H+L211H, R164H+N212H, R164H+K229H, R164H+Q230H, R164H+K231H, R164H+N232H, R164H+P233H, R164H+S234H, R164H+W235H, R164H+S236H, R164H+V238H, R164H+Q239H, R164H+R241H, R164H+N242H, R164H+K245H, R164H+N246H, R164H+T247H, R164H+T249H, R164H+S250H, R164H+L251H, R164H+G252H, R164H+S253H, R164H+T254H, R164H+N255H, R164H+L256H, R164H+Y257H, R164H+S259H, R164H+E265H, R164H+R269H, A166H+N167H, A166H+D175H, A166H+Q176H, A166H+N177H, A166H+N178H, A166H+N179H, A166H+R180H, A166H+S182H, A166H+F183H, A166H+Y186H, A166H+A188H, A166H+G189H, A166H+N198H, A166H+Q200H, A166H+Y203H, A166H+P204H, A166H+G205H, A166H+S206H, A166H+T207H, A166H+Y208H, A166H+S210H, A166H+L211H, A166H+N212H, A166H+K229H, A166H+Q230H, A166H+K231H, A166H+N232H, A166H+P233H, A166H+S234H, A166H+W235H, A166H+S236H, A166H+V238H, A166H+Q239H, A166H+R241H, A166H+N242H, A166H+K245H, A166H+N246H, A166H+T247H, A166H+T249H, A166H+S250H, A166H+L251H, A166H+G252H, A166H+S253H, A166H+T254H, A166H+N255H, A166H+L256H, A166H+Y257H, A166H+S259H, A166H+E265H, A166H+R269H, N167H+D175H, N167H+Q176H, N167H+N177H, N167H+N178H, N167H+N179H, N167H+R180H, N167H+S182H, N167H+F183H, N167H+Y186H, N167H+A188H, N167H+G189H, N167H+N198H, N167H+Q200H, N167H+Y203H, N167H+P204H, N167H+G205H, N167H+S206H, N167H+T207H, N167H+Y208H, N167H+S210H, N167H+L211H, N167H+N212H, N167H+K229H, N167H+Q230H, N167H+K231H, N167H+N232H, N167H+P233H, N167H+S234H, N167H+W235H, N167H+S236H, N167H+V238H, N167H+Q239H, N167H+R241H, N167H+N242H, N167H+K245H, N167H+N246H, N167H+T247H, N167H+T249H, N167H+S250H, N167H+L251H, N167H+G252H, N167H+S253H, N167H+T254H, N167H+N255H, N167H+L256H, N167H+Y257H, N167H+S259H, N167H+E265H, N167H+R269H, D175H+Q176H, D175H+N177H, D175H+N178H, D175H+N179H, D175H+R180H, D175H+S182H, D175H+F183H, D175H+Y186H, D175H+A188H, D175H+G189H, D175H+N198H, D175H+Q200H, D175H+Y203H, D175H+P204H, D175H+G205H, D175H+S206H, D175H+T207H, D175H+Y208H, D175H+S210H, D175H+L211H, D175H+N212H, D175H+K229H, D175H+Q230H, D175H+K231H, D175H+N232H, D175H+P233H, D175H+S234H, D175H+W235H, D175H+S236H, D175H+V238H, D175H+Q239H, D175H+R241H, D175H+N242H, D175H+K245H, D175H+N246H, D175H+T247H, D175H+T249H, D175H+S250H, D175H+L251H, D175H+G252H, D175H+S253H, D175H+T254H, D175H+N255H, D175H+L256H, D175H+Y257H, D175H+S259H, D175H+E265H, D175H+R269H, Q176H+N177H, Q176H+N178H, Q176H+N179H, Q176H+R180H, Q176H+S182H, Q176H+F183H, Q176H+Y186H, Q176H+A188H, Q176H+G189H, Q176H+N198H, Q176H+Q200H, Q176H+Y203H, Q176H+P204H, Q176H+G205H, Q176H+S206H, Q176H+T207H, Q176H+Y208H, Q176H+S210H, Q176H+L211H, Q176H+N212H, Q176H+K229H, Q176H+Q230H, Q176H+K231H, Q176H+N232H, Q176H+P233H, Q176H+S234H, Q176H+W235H, Q176H+S236H, Q176H+V238H, Q176H+Q239H, Q176H+R241H, Q176H+N242H, Q176H+K245H, Q176H+N246H, Q176H+T247H, Q176H+T249H, Q176H+S250H, Q176H+L251H, Q176H+G252H, Q176H+S253H, Q176H+T254H, Q176H+N255H, Q176H+L256H, Q176H+Y257H, Q176H+S259H, Q176H+E265H, Q176H+R269H, N177H+N178H, N177H+N179H, N177H+R180H, N177H+S182H, N177H+F183H, N177H+Y186H, N177H+A188H, N177H+G189H, N177H+N198H, N177H+Q200H, N177H+Y203H, N177H+P204H, N177H+G205H, N177H+S206H, N177H+T207H, N177H+Y208H, N177H+S210H, N177H+L211H, N177H+N212H, N177H+K229H, N177H+Q230H, N177H+K231H, N177H+N232H, N177H+P233H, N177H+S234H, N177H+W235H, N177H+S236H, N177H+V238H, N177H+Q239H, N177H+R241H, N177H+N242H, N177H+K245H, N177H+N246H, N177H+T247H, N177H+T249H, N177H+S250H, N177H+L251H, N177H+G252H, N177H+S253H, N177H+T254H, N177H+N255H, N177H+L256H, N177H+Y257H, N177H+S259H, N177H+E265H, N177H+R269H, N178H+N179H, N178H+R180H, N178H+S182H, N178H+F183H, N178H+Y186H, N178H+A188H, N178H+G189H, N178H+N198H, N178H+Q200H, N178H+Y203H, N178H+P204H, N178H+G205H, N178H+S206H, N178H+T207H, N178H+Y208H, N178H+S210H, N178H+L211H, N178H+N212H, N178H+K229H, N178H+Q230H, N178H+K231H, N178H+N232H, N178H+P233H, N178H+S234H, N178H+W235H, N178H+S236H, N178H+V238H, N178H+Q239H, N178H+R241H, N178H+N242H, N178H+K245H, N178H+N246H, N178H+T247H, N178H+T249H, N178H+S250H, N178H+L251H, N178H+G252H, N178H+S253H, N178H+T254H, N178H+N255H, N178H+L256H, N178H+Y257H, N178H+S259H, N178H+E265H, N178H+R269H, N179H+R180H, N179H+S182H, N179H+F183H, N179H+Y186H, N179H+A188H, N179H+G189H, N179H+N198H, N179H+Q200H, N179H+Y203H, N179H+P204H, N179H+G205H, N179H+S206H, N179H+T207H, N179H+Y208H, N179H+S210H, N179H+L211H, N179H+N212H, N179H+K229H, N179H+Q230H, N179H+K231H, N179H+N232H, N179H+P233H, N179H+S234H, N179H+W235H, N179H+S236H, N179H+V238H, N179H+Q239H, N179H+R241H, N179H+N242H, N179H+K245H, N179H+N246H, N179H+T247H, N179H+T249H, N179H+S250H, N179H+L251H, N179H+G252H, N179H+S253H, N179H+T254H, N179H+N255H, N179H+L256H, N179H+Y257H, N179H+S259H, N179H+E265H, N179H+R269H, R180H+S182H, R180H+F183H, R180H+Y186H, R180H+A188H, R180H+G189H, R180H+N198H, R180H+Q200H, R180H+Y203H, R180H+P204H, R180H+G205H, R180H+S206H, R180H+T207H, R180H+Y208H, R180H+S210H, R180H+L211H, R180H+N212H, R180H+K229H, R180H+Q230H, R180H+K231H, R180H+N232H, R180H+P233H, R180H+S234H, R180H+W235H, R180H+S236H, R180H+V238H, R180H+Q239H, R180H+R241H, R180H+N242H, R180H+K245H, R180H+N246H, R180H+T247H, R180H+T249H, R180H+S250H, R180H+L251H, R180H+G252H, R180H+S253H, R180H+T254H, R180H+N255H, R180H+L256H, R180H+Y257H, R180H+S259H, R180H+E265H, R180H+R269H, S182H+F183H, S182H+Y186H, S182H+A188H, S182H+G189H, S182H+N198H, S182H+Q200H, S182H+Y203H, S182H+P204H, S182H+G205H, S182H+S206H, S182H+T207H, S182H+Y208H, S182H+S210H, S182H+L211H, S182H+N212H, S182H+K229H, S182H+Q230H, S182H+K231H, S182H+N232H, S182H+P233H, S182H+S234H, S182H+W235H, S182H+S236H, S182H+V238H, S182H+Q239H, S182H+R241H, S182H+N242H, S182H+K245H, S182H+N246H, S182H+T247H, S182H+T249H, S182H+S250H, S182H+L251H, S182H+G252H, S182H+S253H, S182H+T254H, S182H+N255H, S182H+L256H, S182H+Y257H, S182H+S259H, S182H+E265H, S182H+R269H, F183H+Y186H, F183H+A188H, F183H+G189H, F183H+N198H, F183H+Q200H, F183H+Y203H, F183H+P204H, F183H+G205H, F183H+S206H, F183H+T207H, F183H+Y208H, F183H+S210H, F183H+L211H, F183H+N212H, F183H+K229H, F183H+Q230H, F183H+K231H, F183H+N232H, F183H+P233H, F183H+S234H, F183H+W235H, F183H+S236H, F183H+V238H, F183H+Q239H, F183H+R241H, F183H+N242H, F183H+K245H, F183H+N246H, F183H+T247H, F183H+T249H, F183H+S250H, F183H+L251H, F183H+G252H, F183H+S253H, F183H+T254H, F183H+N255H, F183H+L256H, F183H+Y257H, F183H+S259H, F183H+E265H, F183H+R269H, Y186H+A188H, Y186H+G189H, Y186H+N198H, Y186H+Q200H, Y186H+Y203H, Y186H+P204H, Y186H+G205H, Y186H+S206H, Y186H+T207H, Y186H+Y208H, Y186H+S210H, Y186H+L211H, Y186H+N212H, Y186H+K229H, Y186H+Q230H, Y186H+K231H, Y186H+N232H, Y186H+P233H, Y186H+S234H, Y186H+W235H, Y186H+S236H, Y186H+V238H, Y186H+Q239H, Y186H+R241H, Y186H+N242H, Y186H+K245H, Y186H+N246H, Y186H+T247H, Y186H+T249H, Y186H+S250H, Y186H+L251H, Y186H+G252H, Y186H+S253H, Y186H+T254H, Y186H+N255H, Y186H+L256H, Y186H+Y257H, Y186H+S259H, Y186H+E265H, Y186H+R269H, A188H+G189H, A188H+N198H, A188H+Q200H, A188H+Y203H, A188H+P204H, A188H+G205H, A188H+S206H, A188H+T207H, A188H+Y208H, A188H+S210H, A188H+L211H, A188H+N212H, A188H+K229H, A188H+Q230H, A188H+K231H, A188H+N232H, A188H+P233H, A188H+S234H, A188H+W235H, A188H+S236H, A188H+V238H, A188H+Q239H, A188H+R241H, A188H+N242H, A188H+K245H, A188H+N246H, A188H+T247H, A188H+T249H, A188H+S250H, A188H+L251H, A188H+G252H, A188H+S253H, A188H+T254H, A188H+N255H, A188H+L256H, A188H+Y257H, A188H+S259H, A188H+E265H, A188H+R269H, G189H+N198H, G189H+Q200H, G189H+Y203H, G189H+P204H, G189H+G205H, G189H+S206H, G189H+T207H, G189H+Y208H, G189H+S210H, G189H+L211H, G189H+N212H, G189H+K229H, G189H+Q230H, G189H+K231H, G189H+N232H, G189H+P233H, G189H+S234H, G189H+W235H, G189H+S236H, G189H+V238H, G189H+Q239H, G189H+R241H, G189H+N242H, G189H+K245H, G189H+N246H, G189H+T247H, G189H+T249H, G189H+S250H, G189H+L251H, G189H+G252H, G189H+S253H, G189H+T254H, G189H+N255H, G189H+L256H, G189H+Y257H, G189H+S259H, G189H+E265H, G189H+R269H, N198H+Q200H, N198H+Y203H, N198H+P204H, N198H+G205H, N198H+S206H, N198H+T207H, N198H+Y208H, N198H+S210H, N198H+L211H, N198H+N212H, N198H+K229H, N198H+Q230H, N198H+K231H, N198H+N232H, N198H+P233H, N198H+S234H, N198H+W235H, N198H+S236H, N198H+V238H, N198H+Q239H, N198H+R241H, N198H+N242H, N198H+K245H, N198H+N246H, N198H+T247H, N198H+T249H, N198H+S250H, N198H+L251H, N198H+G252H, N198H+S253H, N198H+T254H, N198H+N255H, N198H+L256H, N198H+Y257H, N198H+S259H, N198H+E265H, N198H+R269H, Q200H+Y203H, Q200H+P204H, Q200H+G205H, Q200H+S206H, Q200H+T207H, Q200H+Y208H, Q200H+S210H, Q200H+L211H, Q200H+N212H, Q200H+K229H, Q200H+Q230H, Q200H+K231H, Q200H+N232H, Q200H+P233H, Q200H+S234H, Q200H+W235H, Q200H+S236H, Q200H+V238H, Q200H+Q239H, Q200H+R241H, Q200H+N242H, Q200H+K245H, Q200H+N246H, Q200H+T247H, Q200H+T249H, Q200H+S250H, Q200H+L251H, Q200H+G252H, Q200H+S253H, Q200H+T254H, Q200H+N255H, Q200H+L256H, Q200H+Y257H, Q200H+S259H, Q200H+E265H, Q200H+R269H, Y203H+P204H, Y203H+G205H, Y203H+S206H, Y203H+T207H, Y203H+Y208H, Y203H+S210H, Y203H+L211H, Y203H+N212H, Y203H+K229H, Y203H+Q230H, Y203H+K231H, Y203H+N232H, Y203H+P233H, Y203H+S234H, Y203H+W235H, Y203H+S236H, Y203H+V238H, Y203H+Q239H, Y203H+R241H, Y203H+N242H, Y203H+K245H, Y203H+N246H, Y203H+T247H, Y203H+T249H, Y203H+S250H, Y203H+L251H, Y203H+G252H, Y203H+S253H, Y203H+T254H, Y203H+N255H, Y203H+L256H, Y203H+Y257H, Y203H+S259H, Y203H+E265H, Y203H+R269H, P204H+G205H, P204H+S206H, P204H+T207H, P204H+Y208H, P204H+S210H, P204H+L211H, P204H+N212H, P204H+K229H, P204H+Q230H, P204H+K231H, P204H+N232H, P204H+P233H, P204H+S234H, P204H+W235H, P204H+S236H, P204H+V238H, P204H+Q239H, P204H+R241H, P204H+N242H, P204H+K245H, P204H+N246H, P204H+T247H, P204H+T249H, P204H+S250H, P204H+L251H, P204H+G252H, P204H+S253H, P204H+T254H, P204H+N255H, P204H+L256H, P204H+Y257H, P204H+S259H, P204H+E265H, P204H+R269H, G205H+S206H, G205H+T207H, G205H+Y208H, G205H+S210H, G205H+L211H, G205H+N212H, G205H+K229H, G205H+Q230H, G205H+K231H, G205H+N232H, G205H+P233H, G205H+S234H, G205H+W235H, G205H+S236H, G205H+V238H, G205H+Q239H, G205H+R241H, G205H+N242H, G205H+K245H, G205H+N246H, G205H+T247H, G205H+T249H, G205H+S250H, G205H+L251H, G205H+G252H, G205H+S253H, G205H+T254H, G205H+N255H, G205H+L256H, G205H+Y257H, G205H+S259H, G205H+E265H, G205H+R269H, S206H+T207H, S206H+Y208H, S206H+S210H, S206H+L211H, S206H+N212H, S206H+K229H, S206H+Q230H, S206H+K231H, S206H+N232H, S206H+P233H, S206H+S234H, S206H+W235H, S206H+S236H, S206H+V238H, S206H+Q239H, S206H+R241H, S206H+N242H, S206H+K245H, S206H+N246H, S206H+T247H, S206H+T249H, S206H+S250H, S206H+L251H, S206H+G252H, S206H+S253H, S206H+T254H, S206H+N255H, S206H+L256H, S206H+Y257H, S206H+S259H, S206H+E265H, S206H+R269H, T207H+Y208H, T207H+S210H, T207H+L211H, T207H+N212H, T207H+K229H, T207H+Q230H, T207H+K231H, T207H+N232H, T207H+P233H, T207H+S234H, T207H+W235H, T207H+S236H, T207H+V238H, T207H+Q239H, T207H+R241H, T207H+N242H, T207H+K245H, T207H+N246H, T207H+T247H, T207H+T249H, T207H+S250H, T207H+L251H, T207H+G252H, T207H+S253H, T207H+T254H, T207H+N255H, T207H+L256H, T207H+Y257H, T207H+S259H, T207H+E265H, T207H+R269H, Y208H+S210H, Y208H+L211H, Y208H+N212H, Y208H+K229H, Y208H+Q230H, Y208H+K231H, Y208H+N232H, Y208H+P233H, Y208H+S234H, Y208H+W235H, Y208H+S236H, Y208H+V238H, Y208H+Q239H, Y208H+R241H, Y208H+N242H, Y208H+K245H, Y208H+N246H, Y208H+T247H, Y208H+T249H, Y208H+S250H, Y208H+L251H, Y208H+G252H, Y208H+S253H, Y208H+T254H, Y208H+N255H, Y208H+L256H, Y208H+Y257H, Y208H+S259H, Y208H+E265H, Y208H+R269H, S210H+L211H, S210H+N212H, S210H+K229H, S210H+Q230H, S210H+K231H, S210H+N232H, S210H+P233H, S210H+S234H, S210H+W235H, S210H+S236H, S210H+V238H, S210H+Q239H, S210H+R241H, S210H+N242H, S210H+K245H, S210H+N246H, S210H+T247H, S210H+T249H, S210H+S250H, S210H+L251H, S210H+G252H, S210H+S253H, S210H+T254H, S210H+N255H, S210H+L256H, S210H+Y257H, S210H+S259H, S210H+E265H, S210H+R269H, L211H+N212H, L211H+K229H, L211H+Q230H, L211H+K231H, L211H+N232H, L211H+P233H, L211H+S234H, L211H+W235H, L211H+S236H, L211H+V238H, L211H+Q239H, L211H+R241H, L211H+N242H, L211H+K245H, L211H+N246H, L211H+T247H, L211H+T249H, L211H+S250H, L211H+L251H, L211H+G252H, L211H+S253H, L211H+T254H, L211H+N255H, L211H+L256H, L211H+Y257H, L211H+S259H, L211H+E265H, L211H+R269H, N212H+K229H, N212H+Q230H, N212H+K231H, N212H+N232H, N212H+P233H, N212H+S234H, N212H+W235H, N212H+S236H, N212H+V238H, N212H+Q239H, N212H+R241H, N212H+N242H, N212H+K245H, N212H+N246H, N212H+T247H, N212H+T249H, N212H+S250H, N212H+L251H, N212H+G252H, N212H+S253H, N212H+T254H, N212H+N255H, N212H+L256H, N212H+Y257H, N212H+S259H, N212H+E265H, N212H+R269H, K229H+Q230H, K229H+K231H, K229H+N232H, K229H+P233H, K229H+S234H, K229H+W235H, K229H+S236H, K229H+V238H, K229H+Q239H, K229H+R241H, K229H+N242H, K229H+K245H, K229H+N246H, K229H+T247H, K229H+T249H, K229H+S250H, K229H+L251H, K229H+G252H, K229H+S253H, K229H+T254H, K229H+N255H, K229H+L256H, K229H+Y257H, K229H+S259H, K229H+E265H, K229H+R269H, Q230H+K231H, Q230H+N232H, Q230H+P233H, Q230H+S234H, Q230H+W235H, Q230H+S236H, Q230H+V238H, Q230H+Q239H, Q230H+R241H, Q230H+N242H, Q230H+K245H, Q230H+N246H, Q230H+T247H, Q230H+T249H, Q230H+S250H, Q230H+L251H, Q230H+G252H, Q230H+S253H, Q230H+T254H, Q230H+N255H, Q230H+L256H, Q230H+Y257H, Q230H+S259H, Q230H+E265H, Q230H+R269H, K231H+N232H, K231H+P233H, K231H+S234H, K231H+W235H, K231H+S236H, K231H+V238H, K231H+Q239H, K231H+R241H, K231H+N242H, K231H+K245H, K231H+N246H, K231H+T247H, K231H+T249H, K231H+S250H, K231H+L251H, K231H+G252H, K231H+S253H, K231H+T254H, K231H+N255H, K231H+L256H, K231H+Y257H, K231H+S259H, K231H+E265H, K231H+R269H, N232H+P233H, N232H+S234H, N232H+W235H, N232H+S236H, N232H+V238H, N232H+Q239H, N232H+R241H, N232H+N242H, N232H+K245H, N232H+N246H, N232H+T247H, N232H+T249H, N232H+S250H, N232H+L251H, N232H+G252H, N232H+S253H, N232H+T254H, N232H+N255H, N232H+L256H, N232H+Y257H, N232H+S259H, N232H+E265H, N232H+R269H, P233H+S234H, P233H+W235H, P233H+S236H, P233H+V238H, P233H+Q239H, P233H+R241H, P233H+N242H, P233H+K245H, P233H+N246H, P233H+T247H, P233H+T249H, P233H+S250H, P233H+L251H, P233H+G252H, P233H+S253H, P233H+T254H, P233H+N255H, P233H+L256H, P233H+Y257H, P233H+S259H, P233H+E265H, P233H+R269H, S234H+W235H, S234H+S236H, S234H+V238H, S234H+Q239H, S234H+R241H, S234H+N242H, S234H+K245H, S234H+N246H, S234H+T247H, S234H+T249H, S234H+S250H, S234H+L251H, S234H+G252H, S234H+S253H, S234H+T254H, S234H+N255H, S234H+L256H, S234H+Y257H, S234H+S259H, S234H+E265H, S234H+R269H, W235H+S236H, W235H+V238H, W235H+Q239H, W235H+R241H, W235H+N242H, W235H+K245H, W235H+N246H, W235H+T247H, W235H+T249H, W235H+S250H, W235H+L251H, W235H+G252H, W235H+S253H, W235H+T254H, W235H+N255H, W235H+L256H, W235H+Y257H, W235H+S259H, W235H+E265H, W235H+R269H, S236H+V238H, S236H+Q239H, S236H+R241H, S236H+N242H, S236H+K245H, S236H+N246H, S236H+T247H, S236H+T249H, S236H+S250H, S236H+L251H, S236H+G252H, S236H+S253H, S236H+T254H, S236H+N255H, S236H+L256H, S236H+Y257H, S236H+S259H, S236H+E265H, S236H+R269H, V238H+Q239H, V238H+R241H, V238H+N242H, V238H+K245H, V238H+N246H, V238H+T247H, V238H+T249H, V238H+S250H, V238H+L251H, V238H+G252H, V238H+S253H, V238H+T254H, V238H+N255H, V238H+L256H, V238H+Y257H, V238H+S259H, V238H+E265H, V238H+R269H, Q239H+R241H, Q239H+N242H, Q239H+K245H, Q239H+N246H, Q239H+T247H, Q239H+T249H, Q239H+S250H, Q239H+L251H, Q239H+G252H, Q239H+S253H, Q239H+T254H, Q239H+N255H, Q239H+L256H, Q239H+Y257H, Q239H+S259H, Q239H+E265H, Q239H+R269H, R241H+N242H, R241H+K245H, R241H+N246H, R241H+T247H, R241H+T249H, R241H+S250H, R241H+L251H, R241H+G252H, R241H+S253H, R241H+T254H, R241H+N255H, R241H+L256H, R241H+Y257H, R241H+S259H, R241H+E265H, R241H+R269H, N242H+K245H, N242H+N246H, N242H+T247H, N242H+T249H, N242H+S250H, N242H+L251H, N242H+G252H, N242H+S253H, N242H+T254H, N242H+N255H, N242H+L256H, N242H+Y257H, N242H+S259H, N242H+E265H, N242H+R269H, K245H+N246H, K245H+T247H, K245H+T249H, K245H+S250H, K245H+L251H, K245H+G252H, K245H+S253H, K245H+T254H, K245H+N255H, K245H+L256H, K245H+Y257H, K245H+S259H, K245H+E265H, K245H+R269H, N246H+T247H, N246H+T249H, N246H+S250H, N246H+L251H, N246H+G252H, N246H+S253H, N246H+T254H, N246H+N255H, N246H+L256H, N246H+Y257H, N246H+S259H, N246H+E265H, N246H+R269H, T247H+T249H, T247H+S250H, T247H+L251H, T247H+G252H, T247H+S253H, T247H+T254H, T247H+N255H, T247H+L256H, T247H+Y257H, T247H+S259H, T247H+E265H, T247H+R269H, T249H+S250H, T249H+L251H, T249H+G252H, T249H+S253H, T249H+T254H, T249H+N255H, T249H+L256H, T249H+Y257H, T249H+S259H, T249H+E265H, T249H+R269H, S250H+L251H, S250H+G252H, S250H+S253H, S250H+T254H, S250H+N255H, S250H+L256H, S250H+Y257H, S250H+S259H, S250H+E265H, S250H+R269H, L251H+G252H, L251H+S253H, L251H+T254H, L251H+N255H, L251H+L256H, L251H+Y257H, L251H+S259H, L251H+E265H, L251H+R269H, G252H+S253H, G252H+T254H, G252H+N255H, G252H+L256H, G252H+Y257H, G252H+S259H, G252H+E265H, G252H+R269H, S253H+T254H, S253H+N255H, S253H+L256H, S253H+Y257H, S253H+S259H, S253H+E265H, S253H+R269H, T254H+N255H, T254H+L256H, T254H+Y257H, T254H+S259H, T254H+E265H, T254H+R269H, N255H+L256H, N255H+Y257H, N255H+S259H, N255H+E265H, N255H+R269H, L256H+Y257H, L256H+S259H, L256H+E265H, L256H+R269H, Y257H+S259H, Y257H+E265H, Y257H+R269H, S259H+E265H, S259H+R269H, E265H+R269H.

In one embodiment of the invention, the histidines are introduced into the surface of the protein such as a protease by insertion of 2 to 6 histidines at any of the surface positions described in the solvent accessibility section above. In a preferred embodiment the histidines are inserted in the surface of a protease having at least 60% identity to the mature polypeptide of SEQ ID NO 2. A particular preferred embodiment relates to a method of producing a protease variant having at least 60% identity to SEQ ID NO 3 wherein the method comprising insertion of a histidine amino acid at 2 to 6 of the positions selected from the group consisting of: A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259, R269, wherein the positions corresponds to the positions of SEQ ID NO 3.

One embodiment of the invention relates to protease variants which compared to SEQ ID NO 3 comprises any of the following insertions:

### Accessibility >40%, Min Distance to Active Site 6 Angstroms

*1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *89aH, *96aH, *97aH, *98aH, *99aH, *100aH, *101aH, *102aH,*103aH,*107aH,*110aH,*111aH,*114aH,*115aH,*116aH,*118aH,*125aH,*126aH, *127aH,*128aH,*129aH,*131aH,*132aH,*134aH,*135aH,*138aH,*139aH,*142aH,*143aH, *144aH, *153aH, *154aH, *155aH, *156aH, *157aH, *158aH, *161aH, *164aH, *166aH, *167aH, *175aH,*176aH,*177aH,*178aH,*179aH,*180aH,*182aH,*183aH,*186aH,*188aH,*189aH, *198aH,*200aH,*203aH,*204aH,*205aH,*206aH,*207aH,*208aH,*210aH,*211aH,*212aH, *229aH,*230aH,*231aH,*232aH,*233aH,*234aH,*235aH,*236aH,*238aH,*239aH,*241aH, *242aH, *243aH, *245aH, *246aH, *247aH, *249aH, *250aH, *251aH, *252aH, *253aH, *254aH, *255aH, *256aH, *257aH, *259aH, *265aH, *269aH

### Accessibility >45%, Min Distance to Active Site 6 Angstroms

*1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH,*107aH,*110aH,*111aH,*114aH,*115aH,*116aH,*118aH,*125aH,*126aH,*127aH, *128aH,*129aH,*131aH,*132aH,*134aH,*135aH,*138aH,*139aH,*142aH,*143aH,*144aH, *153aH, *154aH, *156aH, *157aH, *158aH, *161aH, *164aH, *166aH, *167aH, *175aH, *176aH, *177aH,*178aH,*179aH,*180aH,*182aH,*183aH,*186aH,*188aH,*189aH,*198aH,*200aH, *203aH,*204aH,*205aH,*206aH,*207aH,*208aH,*210aH,*211aH,*212aH,*229aH,*230aH, *231aH, *232aH, *233aH, *234aH, *235aH, *236aH, *238aH, *239aH, *241aH, *242aH, *243aH, *245aH, *246aH, *249aH, *250aH, *251aH, *252aH, *253aH, *254aH, *255aH, *256aH, *257aH, *259aH, *269aH

### Accessibility >50%, Min Distance to Active Site 6 Angstroms

*1aH, *2aH, *3aH, *4aH, *6aH, *9aH, *10aH, *12aH, *14aH, *15aH, *18aH, *19aH, *20aH, *24aH, *25aH, *27aH, *37aH, *39aH, *42aH, *43aH, *44aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH, *110aH, *111aH, *114aH, *115aH, *116aH, *118aH, *125aH, *126aH, *127aH, *128aH, *129aH, *131aH, *132aH, *134aH, *135aH,*138aH, *142aH,*143aH, *144aH,*153aH, *154aH,*156aH,*157aH, *158aH, *164aH,*166aH, *167aH,*175aH,*176aH, *177aH, *178aH,*179aH, *180aH, *182aH,*183aH, *186aH,*188aH, *189aH,*198aH,*200aH, *203aH, *205aH,*206aH, *207aH, *208aH,*210aH, *211aH,*212aH, *229aH,*230aH, *231aH,*232aH, *233aH, *234aH, *235aH, *236aH, *238aH, *239aH, *241aH, *242aH, *243aH, *245aH, *246aH, *249aH, *250aH, *251aH, *252aH, *253aH, *254aH, *255aH, *256aH, *257aH, *259aH, *269aH

### Accessibility >60%, Min Distance to Active Site 6 Angstroms

*1aH, *2aH, *3aH, *4aH, *6aH, *9aH, *10aH, *12aH, *14aH, *18aH, *19aH, *20aH, *24aH, *25aH, *27aH, *37aH, *39aH, *42aH, *43aH, *44aH, *49aH, *51aH, *52aH, *53aH, *54aH, *55aH, *56aH, *57aH, *59aH, *73aH, *74aH, *75aH, *76aH, *77aH, *85aH, *97aH, *99aH, *101aH,*102aH,*103aH,*107aH,*110aH,*111aH,*114aH,*115aH,*116aH,*118aH,*125aH, *127aH, *128aH, *129aH,*131aH,*132aH, *134aH, *135aH,*138aH, *142aH, *143aH,*153aH, *154aH,*156aH, *157aH,*158aH,*164aH, *166aH, *167aH,*176aH, *177aH, *178aH,*179aH, *180aH,*182aH, *183aH,*186aH,*188aH,*189aH,*198aH, *200aH,*203aH,*205aH,*206aH, *207aH,*208aH, *210aH,*211aH,*212aH, *229aH, *230aH,*231aH, *233aH, *234aH, *235aH, *236aH, *238aH, *239aH,*241aH,*242aH, *243aH, *245aH, *246aH, *249aH, *250aH,*251aH, *252aH, *253aH, *254aH, *255aH, *256aH, *269aH

### Accessibility >70%, Min Distance to Active Site 6 Angstroms

*1aH, *2aH, *3aH, *4aH, *6aH, *9aH, *10aH, *12aH, *14aH, *18aH, *19aH, *20aH, *24aH, *25aH, *27aH, *37aH, *39aH, *42aH, *44aH, *49aH, *51aH, *52aH, *53aH, *54aH, *55aH, *56aH, *57aH, *59aH, *73aH, *74aH, *75aH, *76aH, *77aH, *85aH, *97aH, *99aH, *101aH, *102aH,*103aH,*107aH,*110aH,*111aH,*114aH,*115aH,*116aH,*118aH,*125aH,*127aH, *128aH, *129aH, *131aH,*132aH, *134aH,*135aH,*138aH, *142aH,*143aH, *153aH, *154aH, *158aH,*164aH, *166aH,*167aH,*176aH, *177aH, *178aH,*179aH, *180aH, *182aH,*183aH, *186aH,*188aH, *189aH,*198aH,*200aH, *203aH, *205aH,*206aH, *207aH, *208aH,*210aH, *211aH, *212aH,*230aH, *231aH,*233aH, *234aH, *235aH, *236aH, *238aH, *239aH, *242aH, *243aH, *245aH, *246aH, *249aH, *250aH, *251aH, *253aH, *254aH, *255aH, *256aH, *269aH

### Accessibility >80%, Min Distance to Active Site 6 Angstroms

*1aH, *3aH, *4aH, *9aH, *10aH, *12aH, *18aH, *19aH, *24aH, *27aH, *37aH, *39aH, *42aH, *44aH, *51aH, *52aH, *54aH, *55aH, *57aH, *59aH, *74aH, *75aH, *76aH, *77aH, *85aH, *97aH, *99aH, *101aH, *103aH, *107aH, *110aH, *111aH, *114aH, *115aH, *118aH, *127aH, *128aH, *129aH, *131aH, *132aH, *134aH, *135aH, *138aH, *142aH, *143aH, *153aH, *154aH, *158aH, *164aH, *166aH,*167aH,*176aH, *177aH,*179aH,*180aH, *182aH,*186aH,*188aH, *198aH,*200aH, *203aH,*205aH,*206aH, *207aH, *208aH,*210aH, *211aH, *212aH,*230aH, *231aH, *233aH, *234aH, *235aH, *236aH, *238aH, *239aH, *242aH, *243aH, *246aH, *249aH, *250aH, *253aH, *254aH, *255aH, *256aH, *269aH

### Accessibility >90%, Min Distance to Active Site 6 Angstroms

*1aH, *3aH, *4aH, *9aH, *10aH, *12aH, *18aH, *19aH, *24aH, *37aH, *39aH, *42aH, *44aH, *51aH, *52aH, *54aH, *55aH, *57aH, *59aH, *74aH, *75aH, *76aH, *77aH, *97aH, *99aH, *101aH,*103aH, *107aH,*110aH,*114aH, *115aH, *118aH,*127aH, *128aH, *129aH,*131aH, *132aH, *134aH, *135aH, *142aH, *143aH, *153aH, *154aH, *158aH, *164aH, *166aH, *167aH, *176aH, *177aH, *179aH, *180aH, *182aH, *188aH, *198aH, *200aH, *203aH, *205aH, *206aH, *207aH, *208aH, *210aH, *212aH, *231aH, *233aH, *234aH, *235aH, *238aH, *239aH, *242aH, *246aH, *249aH, *250aH, *253aH, *254aH, *255aH, *269aH

### Accessibility >100%, Min Distance to Active Site 6 Angstroms

*1aH, *3aH, *4aH, *9aH, *10aH, *12aH, *18aH, *19aH, *24aH, *37aH, *39aH, *42aH, *44aH, *51aH, *52aH, *54aH, *57aH, *74aH, *76aH, *97aH, *99aH, *103aH, *107aH, *110aH, *114aH, *115aH, *127aH, *128aH, *129aH, *131aH, *132aH, *134aH, *135aH, *142aH, *143aH, *153aH, *154aH, *164aH, *166aH, *176aH, *177aH, *179aH, *182aH, *188aH, *198aH, *200aH, *203aH, *205aH, *206aH, *207aH, *208aH, *212aH, *231aH, *233aH, *234aH, *238aH, *239aH, *242aH, *246aH, *250aH, *254aH, *255aH, *269aH

### Accessibility >110%, Min Distance to Active Site 6 Angstroms

*1aH, *10aH, *12aH, *18aH, *19aH, *37aH, *42aH, *44aH, *51aH, *52aH, *54aH, *57aH, *74aH, *76aH, *97aH, *99aH, *107aH, *114aH, *115aH, *127aH, *129aH, *132aH, *134aH, *135aH, *142aH, *143aH, *153aH, *154aH, *164aH, *166aH, *176aH, *177aH, *179aH, *182aH, *188aH, *198aH, *200aH, *203aH, *205aH, *206aH, *212aH, *231aH, *233aH, *234aH, *238aH, *239aH, *242aH, *246aH, *250aH, *254aH, *255aH, *269aH

### Accessibility >120%, Min Distance to Active Site 6 Angstroms

*1aH, *10aH, *18aH, *19aH, *37aH, *42aH, *44aH, *51aH, *52aH, *54aH, *57aH, *74aH, *76aH, *97aH, *99aH, *107aH, *114aH, *115aH, *127aH, *129aH, *134aH, *135aH, *142aH, *143aH, *153aH, *154aH, *164aH, *166aH, *176aH, *177aH, *179aH, *182aH, *188aH, *198aH, *200aH, *203aH, *212aH, *231aH, *233aH, *234aH, *239aH, *246aH, *250aH, *254aH, *255aH, *269aH

### Accessibility >130%, Min Distance to Active Site 6 Angstroms

*1aH, *18aH, *19aH, *37aH, *42aH, *44aH, *51aH, *52aH, *54aH, *57aH, *74aH, *76aH, *97aH, *107aH, *114aH, *127aH, *129aH, *135aH, *142aH, *143aH, *164aH, *176aH, *177aH, *182aH, *200aH, *203aH, *212aH, *231aH, *233aH, *234aH, *239aH, *246aH, *250aH, *254aH, *269aH

### Accessibility >140%, Min Distance to Active Site 6 Angstroms

*1aH, *18aH, *19aH, *37aH, *42aH, *44aH, *51aH, *54aH, *57aH, *74aH, *76aH, *97aH, *107aH, *114aH, *127aH,*129aH,*135aH, *142aH, *164aH, *177aH, *182aH, *212aH,*231aH, *233aH, *234aH, *239aH, *246aH, *250aH, *254aH, *269aH

### Accessibility >40%, Min Distance to Active Site 6 Angstroms

*1aH+*1bH, *2aH+*2bH, *3aH+*3bH, *4aH+*4bH, *6aH+*6bH, *9aH+*9bH, *10aH+*10bH, *12aH+*12bH, *14aH+*14bH, *15aH+*15bH, *17aH+*17bH, *18aH+*18bH, *19aH+*19bH, *20aH+*20bH, *22aH+*22bH, *24aH+*24bH, *25aH+*25bH, *27aH+*27bH, *37aH+*37bH, *39aH+*39bH, *42aH+*42bH, *43aH+*43bH, *44aH+*44bH, *45aH+*45bH, *46aH+*46bH, *48aH+*48bH, *49aH+*49bH, *51aH+*51bH, *52aH+*52bH, *53aH+*53bH, *54aH+*54bH, *55aH+*55bH, *56aH+*56bH, *57aH+*57bH, *59aH+*59bH, *73aH+*73bH, *74aH+*74bH, *75aH+*75bH, *76aH+*76bH, *77aH+*77bH, *85aH+*85bH, *87aH+*87bH, *89aH+*89bH, *96aH+*96bH, *97aH+*97bH, *98aH+*98bH, *99aH+*99bH, *100aH+*100bH, *101aH+*101bH, *102aH+*102bH, *103aH+*103bH, *107aH+*107bH, *110aH+*110bH, *111aH+*111bH, *114aH+*114bH, *115aH+*115bH, *116aH+*116bH, *118aH+*118bH, *125aH+*125bH, *126aH+*126bH, *127aH+*127bH, *128aH+*128bH, *129aH+*129bH, *131aH+*131bH, *132aH+*132bH, *134aH+*134bH, *135aH+*135bH, *138aH+*138bH, *139aH+*139bH, *142aH+*142bH, *143aH+*143bH, *144aH+*144bH, *153aH+*153bH, *154aH+*154bH, *155aH+*155bH, *156aH+*156bH, *157aH+*157bH, *158aH+*158bH, *161aH+*161bH, *164aH+*164bH, *166aH+*166bH, *167aH+*167bH, *175aH+*175bH, *176aH+*176bH, *177aH+*177bH, *178aH+*178bH, *179aH+*179bH, *180aH+*180bH, *182aH+*182bH, *183aH+*183bH, *186aH+*186bH, *188aH+*188bH, *189aH+*189bH, *198aH+*198bH, *200aH+*200bH, *203aH+*203bH, *204aH+*204bH, *205aH+*205bH, *206aH+*206bH, *207aH+*207bH, *208aH+*208bH, *210aH+*210bH, *211aH+*211bH, *212aH+*212bH, *229aH+*229bH, *230aH+*230bH, *231aH+*231bH, *232aH+*232bH, *233aH+*233bH, *234aH+*234bH, *235aH+*235bH, *236aH+*236bH, *238aH+*238bH, *239aH+*239bH, *241aH+*241bH, *242aH+*242bH, *243aH+*243bH, *245aH+*245bH, *246aH+*246bH, *247aH+*247bH, *249aH+*249bH, *250aH+*250bH, *251aH+*251bH, *252aH+*252bH, *253aH+*253bH, *254aH+*254bH, *255aH+*255bH, *256aH+*256bH, *257aH+*257bH, *259aH+*259bH, *265aH+*265bH, *269aH+*269bH

### Accessibility >45%, Min Distance to Active Site 6 Angstroms

*1aH+*1bH, *2aH+*2bH, *3aH+*3bH, *4aH+*4bH, *6aH+*6bH, *9aH+*9bH, *10aH+*10bH, *12aH+*12bH, *14aH+*14bH, *15aH+*15bH, *17aH+*17bH, *18aH+*18bH, *19aH+*19bH, *20aH+*20bH, *22aH+*22bH, *24aH+*24bH, *25aH+*25bH, *27aH+*27bH, *37aH+*37bH, *39aH+*39bH, *42aH+*42bH, *43aH+*43bH, *44aH+*44bH, *45aH+*45bH, *46aH+*46bH, *48aH+*48bH, *49aH+*49bH, *51aH+*51bH, *52aH+*52bH, *53aH+*53bH, *54aH+*54bH, *55aH+*55bH, *56aH+*56bH, *57aH+*57bH, *59aH+*59bH, *73aH+*73bH, *74aH+*74bH, *75aH+*75bH, *76aH+*76bH, *77aH+*77bH, *85aH+*85bH, *87aH+*87bH, *96aH+*96bH, *97aH+*97bH, *98aH+*98bH, *99aH+*99bH, *100aH+*100bH, *101aH+*101bH, *102aH+*102bH, *103aH+*103bH, *107aH+*107bH, *110aH+*110bH, *111aH+*111bH, *114aH+*114bH, *115aH+*115bH, *116aH+*116bH, *118aH+*118bH, *125aH+*125bH, *126aH+*126bH, *127aH+*127bH, *128aH+*128bH, *129aH+*129bH, *131aH+*131bH, *132aH+*132bH, *134aH+*134bH, *135aH+*135bH, *138aH+*138bH, *139aH+*139bH, *142aH+*142bH, *143aH+*143bH, *144aH+*144bH, *153aH+*153bH, *154aH+*154bH, *156aH+*156bH, *157aH+*157bH, *158aH+*158bH, *161aH+*161bH, *164aH+*164bH, *166aH+*166bH, *167aH+*167bH, *175aH+*175bH, *176aH+*176bH, *177aH+*177bH, *178aH+*178bH, *179aH+*179bH, *180aH+*180bH, *182aH+*182bH, *183aH+*183bH, *186aH+*186bH, *188aH+*188bH, *189aH+*189bH, *198aH+*198bH, *200aH+*200bH, *203aH+*203bH, *204aH+*204bH, *205aH+*205bH, *206aH+*206bH, *207aH+*207bH, *208aH+*208bH, *210aH+*210bH, *211aH+*211bH, *212aH+*212bH, *229aH+*229bH, *230aH+*230bH, *231aH+*231bH, *232aH+*232bH, *233aH+*233bH, *234aH+*234bH, *235aH+*235bH, *236aH+*236bH, *238aH+*238bH, *239aH+*239bH, *241aH+*241bH, *242aH+*242bH, *243aH+*243bH, *245aH+*245bH, *246aH+*246bH, *249aH+*249bH, *250aH+*250bH, *251aH+*251bH, *252aH+*252bH, *253aH+*253bH, *254aH+*254bH, *255aH+*255bH, *256aH+*256bH, *257aH+*257bH, *259aH+*259bH, *269aH+*269bH

### Accessibility >50%, Min Distance to Active Site 6 Angstroms

*1aH+*1bH, *2aH+*2bH, *3aH+*3bH, *4aH+*4bH, *6aH+*6bH, *9aH+*9bH, *10aH+*10bH, *12aH+*12bH, *14aH+*14bH, *15aH+*15bH, *18aH+*18bH, *19aH+*19bH, *20aH+*20bH, *24aH+*24bH, *25aH+*25bH, *27aH+*27bH, *37aH+*37bH, *39aH+*39bH, *42aH+*42bH, *43aH+*43bH, *44aH+*44bH, *49aH+*49bH, *51aH+*51bH, *52aH+*52bH, *53aH+*53bH, *54aH+*54bH, *55aH+*55bH, *56aH+*56bH, *57aH+*57bH, *59aH+*59bH, *73aH+*73bH, *74aH+*74bH, *75aH+*75bH, *76aH+*76bH, *77aH+*77bH, *85aH+*85bH, *87aH+*87bH, *96aH+*96bH, *97aH+*97bH, *98aH+*98bH, *99aH+*99bH, *100aH+*100bH, *101aH+*101bH, *102aH+*102bH, *103aH+*103bH, *107aH+*107bH, *110aH+*110bH, *111aH+*111bH, *114aH+*114bH, *115aH+*115bH, *116aH+*116bH, *118aH+*118bH, *125aH+*125bH, *126aH+*126bH, *127aH+*127bH, *128aH+*128bH, *129aH+*129bH, *131aH+*131bH, *132aH+*132bH, *134aH+*134bH, *135aH+*135bH, *138aH+*138bH, *142aH+*142bH, *143aH+*143bH, *144aH+*144bH, *153aH+*153bH, *154aH+*154bH, *156aH+*156bH, *157aH+*157bH, *158aH+*158bH, *164aH+*164bH, *166aH+*166bH, *167aH+*167bH, *175aH+*175bH, *176aH+*176bH, *177aH+*177bH, *178aH+*178bH, *179aH+*179bH, *180aH+*180bH, *182aH+*182bH, *183aH+*183bH, *186aH+*186bH, *188aH+*188bH, *189aH+*189bH, *198aH+*198bH, *200aH+*200bH, *203aH+*203bH, *205aH+*205bH, *206aH+*206bH, *207aH+*207bH, *208aH+*208bH, *210aH+*210bH, *211aH+*211bH, *212aH+*212bH, *229aH+*229bH, *230aH+*230bH, *231aH+*231bH, *232aH+*232bH, *233aH+*233bH, *234aH+*234bH, *235aH+*235bH, *236aH+*236bH, *238aH+*238bH, *239aH+*239bH, *241aH+*241bH, *242aH+*242bH, *243aH+*243bH, *245aH+*245bH, *246aH+*246bH, *249aH+*249bH, *250aH+*250bH, *251aH+*251bH, *252aH+*252bH, *253aH+*253bH, *254aH+*254bH, *255aH+*255bH, *256aH+*256bH, *257aH+*257bH, *259aH+*259bH, *269aH+*269bH

### Accessibility >60%, Min Distance to Active Site 6 Angstroms

*1aH+*1bH, *2aH+*2bH, *3aH+*3bH, *4aH+*4bH, *6aH+*6bH, *9aH+*9bH, *10aH+*10bH, *12aH+*12bH, *14aH+*14bH, *18aH+*18bH, *19aH+*19bH, *20aH+*20bH, *24aH+*24bH, *25aH+*25bH, *27aH+*27bH, *37aH+*37bH, *39aH+*39bH, *42aH+*42bH, *43aH+*43bH, *44aH+*44bH, *49aH+*49bH, *51aH+*51bH, *52aH+*52bH, *53aH+*53bH, *54aH+*54bH, *55aH+*55bH, *56aH+*56bH, *57aH+*57bH, *59aH+*59bH, *73aH+*73bH, *74aH+*74bH, *75aH+*75bH, *76aH+*76bH, *77aH+*77bH, *85aH+*85bH, *97aH+*97bH, *99aH+*99bH, *101aH+*101bH, *102aH+*102bH, *103aH+*103bH, *107aH+*107bH, *110aH+*110bH, *111aH+*111bH, *114aH+*114bH, *115aH+*115bH, *116aH+*116bH, *118aH+*118bH, *125aH+*125bH, *127aH+*127bH, *128aH+*128bH, *129aH+*129bH, *131aH+*131bH, *132aH+*132bH, *134aH+*134bH, *135aH+*135bH, *138aH+*138bH, *142aH+*142bH, *143aH+*143bH, *153aH+*153bH, *154aH+*154bH, *156aH+*156bH, *157aH+*157bH, *158aH+*158bH, *164aH+*164bH, *166aH+*166bH, *167aH+*167bH, *176aH+*176bH, *177aH+*177bH, *178aH+*178bH, *179aH+*179bH, *180aH+*180bH, *182aH+*182bH, *183aH+*183bH, *186aH+*186bH, *188aH+*188bH, *189aH+*189bH, *198aH+*198bH, *200aH+*200bH, *203aH+*203bH, *205aH+*205bH, *206aH+*206bH, *207aH+*207bH, *208aH+*208bH, *210aH+*210bH, *211aH+*211bH, *212aH+*212bH, *229aH+*229bH, *230aH+*230bH, *231aH+*231bH, *233aH+*233bH, *234aH+*234bH, *235aH+*235bH, *236aH+*236bH, *238aH+*238bH, *239aH+*239bH, *241aH+*241bH, *242aH+*242bH, *243aH+*243bH, *245aH+*245bH, *246aH+*246bH, *249aH+*249bH, *250aH+*250bH, *251aH+*251bH, *252aH+*252bH, *253aH+*253bH, *254aH+*254bH, *255aH+*255bH, *256aH+*256bH, *269aH+*269bH

### Accessibility >70%, Min Distance to Active Site 6 Angstroms

*1aH+*1bH, *2aH+*2bH, *3aH+*3bH, *4aH+*4bH, *6aH+*6bH, *9aH+*9bH, *10aH+*10bH, *12aH+*12bH, *14aH+*14bH, *18aH+*18bH, *19aH+*19bH, *20aH+*20bH, *24aH+*24bH, *25aH+*25bH, *27aH+*27bH, *37aH+*37bH, *39aH+*39bH, *42aH+*42bH, *44aH+*44bH, *49aH+*49bH, *51aH+*51bH, *52aH+*52bH, *53aH+*53bH, *54aH+*54bH, *55aH+*55bH, *56aH+*56bH, *57aH+*57bH, *59aH+*59bH, *73aH+*73bH, *74aH+*74bH, *75aH+*75bH, *76aH+*76bH, *77aH+*77bH, *85aH+*85bH, *97aH+*97bH, *99aH+*99bH, *101aH+*101bH, *102aH+*102bH, *103aH+*103bH, *107aH+*107bH, *110aH+*110bH, *111aH+*111bH, *114aH+*114bH, *115aH+*115bH, *116aH+*116bH, *118aH+*118bH, *125aH+*125bH, *127aH+*127bH, *128aH+*128bH, *129aH+*129bH, *131aH+*131bH, *132aH+*132bH, *134aH+*134bH, *135aH+*135bH, *138aH+*138bH, *142aH+*142bH, *143aH+*143bH, *153aH+*153bH, *154aH+*154bH, *158aH+*158bH, *164aH+*164bH, *166aH+*166bH, *167aH+*167bH, *176aH+*176bH, *177aH+*177bH, *178aH+*178bH, *179aH+*179bH, *180aH+*180bH, *182aH+*182bH, *183aH+*183bH, *186aH+*186bH, *188aH+*188bH, *189aH+*189bH, *198aH+*198bH, *200aH+*200bH, *203aH+*203bH, *205aH+*205bH, *206aH+*206bH, *207aH+*207bH, *208aH+*208bH, *210aH+*210bH, *211aH+*211bH, *212aH+*212bH, *230aH+*230bH, *231aH+*231bH, *233aH+*233bH, *234aH+*234bH, *235aH+*235bH, *236aH+*236bH, *238aH+*238bH, *239aH+*239bH, *242aH+*242bH, *243aH+*243bH, *245aH+*245bH, *246aH+*246bH, *249aH+*249bH, *250aH+*250bH, *251aH+*251bH, *253aH+*253bH, *254aH+*254bH, *255aH+*255bH, *256aH+*256bH, *269aH+*269bH

### Accessibility >80%, Min Distance to Active Site 6 Angstroms

*1aH+*1bH, *3aH+*3bH, *4aH+*4bH, *9aH+*9bH, *10aH+*10bH, *12aH+*12bH, *18aH+*18bH, *19aH+*19bH, *24aH+*24bH, *27aH+*27bH, *37aH+*37bH, *39aH+*39bH, *42aH+*42bH, *44aH+*44bH, *51aH+*51bH, *52aH+*52bH, *54aH+*54bH, *55aH+*55bH, *57aH+*57bH, *59aH+*59bH, *74aH+*74bH, *75aH+*75bH, *76aH+*76bH, *77aH+*77bH, *85aH+*85bH, *97aH+*97bH, *99aH+*99bH, *101aH+*101bH, *103aH+*103bH, *107aH+*107bH, *110aH+*110bH, *111aH+*111bH, *114aH+*114bH, *115aH+*115bH, *118aH+*118bH, *127aH+*127bH, *128aH+*128bH, *129aH+*129bH, *131aH+*131bH, *132aH+*132bH, *134aH+*134bH, *135aH+*135bH, *138aH+*138bH, *142aH+*142bH, *143aH+*143bH, *153aH+*153bH, *154aH+*154bH, *158aH+*158bH, *164aH+*164bH, *166aH+*166bH, *167aH+*167bH, *176aH+*176bH, *177aH+*177bH, *179aH+*179bH, *180aH+*180bH, *182aH+*182bH, *186aH+*186bH, *188aH+*188bH, *198aH+*198bH, *200aH+*200bH, *203aH+*203bH, *205aH+*205bH, *206aH+*206bH, *207aH+*207bH, *208aH+*208bH, *210aH+*210bH, *211aH+*211bH, *212aH+*212bH, *230aH+*230bH, *231aH+*231bH, *233aH+*233bH, *234aH+*234bH, *235aH+*235bH, *236aH+*236bH, *238aH+*238bH, *239aH+*239bH, *242aH+*242bH, *243aH+*243bH, *246aH+*246bH, *249aH+*249bH, *250aH+*250bH, *253aH+*253bH, *254aH+*254bH, *255aH+*255bH, *256aH+*256bH, *269aH+*269bH

### Accessibility >90%, Min Distance to Active Site 6 Angstroms

*1aH+*1bH, *3aH+*3bH, *4aH+*4bH, *9aH+*9bH, *10aH+*10bH, *12aH+*12bH, *18aH+*18bH, *19aH+*19bH, *24aH+*24bH, *37aH+*37bH, *39aH+*39bH, *42aH+*42bH, *44aH+*44bH, *51aH+*51bH, *52aH+*52bH, *54aH+*54bH, *55aH+*55bH, *57aH+*57bH, *59aH+*59bH, *74aH+*74bH, *75aH+*75bH, *76aH+*76bH, *77aH+*77bH, *97aH+*97bH, *99aH+*99bH, *101aH+*101bH, *103aH+*103bH, *107aH+*107bH, *110aH+*110bH, *114aH+*114bH, *115aH+*115bH, *118aH+*118bH, *127aH+*127bH, *128aH+*128bH, *129aH+*129bH, *131aH+*131bH, *132aH+*132bH, *134aH+*134bH, *135aH+*135bH, *142aH+*142bH, *143aH+*143bH, *153aH+*153bH, *154aH+*154bH, *158aH+*158bH, *164aH+*164bH, *166aH+*166bH, *167aH+*167bH, *176aH+*176bH, *177aH+*177bH, *179aH+*179bH, *180aH+*180bH, *182aH+*182bH, *188aH+*188bH, *198aH+*198bH, *200aH+*200bH, *203aH+*203bH, *205aH+*205bH, *206aH+*206bH, *207aH+*207bH, *208aH+*208bH, *210aH+*210bH, *212aH+*212bH, *231aH+*231bH, *233aH+*233bH, *234aH+*234bH, *235aH+*235bH, *238aH+*238bH, *239aH+*239bH, *242aH+*242bH, *246aH+*246bH, *249aH+*249bH, *250aH+*250bH, *253aH+*253bH, *254aH+*254bH, *255aH+*255bH, *269aH+*269bH

### Accessibility >100%, Min Distance to Active Site 6 Angstroms

*1aH+*1bH, *3aH+*3bH, *4aH+*4bH, *9aH+*9bH, *10aH+*10bH, *12aH+*12bH, *18aH+*18bH, *19aH+*19bH, *24aH+*24bH, *37aH+*37bH, *39aH+*39bH, *42aH+*42bH, *44aH+*44bH, *51aH+*51bH, *52aH+*52bH, *54aH+*54bH, *57aH+*57bH, *74aH+*74bH, *76aH+*76bH, *97aH+*97bH, *99aH+*99bH, *103aH+*103bH, *107aH+*107bH, *110aH+*110bH, *114aH+*114bH, *115aH+*115bH, *127aH+*127bH, *128aH+*128bH, *129aH+*129bH, *131aH+*131bH, *132aH+*132bH, *134aH+*134bH, *135aH+*135bH, *142aH+*142bH, *143aH+*143bH, *153aH+*153bH, *154aH+*154bH, *164aH+*164bH, *166aH+*166bH, *176aH+*176bH, *177aH+*177bH, *179aH+*179bH, *182aH+*182bH, *188aH+*188bH, *198aH+*198bH, *200aH+*200bH, *203aH+*203bH, *205aH+*205bH, *206aH+*206bH, *207aH+*207bH, *208aH+*208bH, *212aH+*212bH, *231aH+*231bH, *233aH+*233bH, *234aH+*234bH, *238aH+*238bH, *239aH+*239bH, *242aH+*242bH, *246aH+*246bH, *250aH+*250bH, *254aH+*254bH, *255aH+*255bH, *269aH+*269bH

### Accessibility >110%, Min Distance to Active Site 6 Angstroms

*1aH+*1bH, *10aH+*10bH, *12aH+*12bH, *18aH+*18bH, *19aH+*19bH, *37aH+*37bH, *42aH+*42bH, *44aH+*44bH, *51aH+*51bH, *52aH+*52bH, *54aH+*54bH, *57aH+*57bH, *74aH+*74bH, *76aH+*76bH, *97aH+*97bH, *99aH+*99bH, *107aH+*107bH, *114aH+*114bH, *115aH+*115bH, *127aH+*127bH, *129aH+*129bH, *132aH+*132bH, *134aH+*134bH, *135aH+*135bH, *142aH+*142bH, *143aH+*143bH, *153aH+*153bH, *154aH+*154bH, *164aH+*164bH, *166aH+*166bH, *176aH+*176bH, *177aH+*177bH, *179aH+*179bH, *182aH+*182bH, *188aH+*188bH, *198aH+*198bH, *200aH+*200bH, *203aH+*203bH, *205aH+*205bH, *206aH+*206bH, *212aH+*212bH, *231aH+*231bH, *233aH+*233bH, *234aH+*234bH, *238aH+*238bH, *239aH+*239bH, *242aH+*242bH, *246aH+*246bH, *250aH+*250bH, *254aH+*254bH, *255aH+*255bH, *269aH+*269bH

### Accessibility >120%, Min Distance to Active Site 6 Angstroms

*1aH+*1bH, *10aH+*10bH, *18aH+*18bH, *19aH+*19bH, *37aH+*37bH, *42aH+*42bH, *44aH+*44bH, *51aH+*51bH, *52aH+*52bH, *54aH+*54bH, *57aH+*57bH, *74aH+*74bH, *76aH+*76bH, *97aH+*97bH, *99aH+*99bH, *107aH+*107bH, *114aH+*114bH, *115aH+*115bH, *127aH+*127bH, *129aH+*129bH, *134aH+*134bH, *135aH+*135bH, *142aH+*142bH, *143aH+*143bH, *153aH+*153bH, *154aH+*154bH, *164aH+*164bH, *166aH+*166bH, *176aH+*176bH, *177aH+*177bH, *179aH+*179bH, *182aH+*182bH, *188aH+*188bH, *198aH+*198bH, *200aH+*200bH, *203aH+*203bH, *212aH+*212bH, *231aH+*231bH, *233aH+*233bH, *234aH+*234bH, *239aH+*239bH, *246aH+*246bH, *250aH+*250bH, *254aH+*254bH, *255aH+*255bH, *269aH+*269bH

### Accessibility >130%, Min Distance to Active Site 6 Angstroms

*1aH+*1bH, *18aH+*18bH, *19aH+*19bH, *37aH+*37bH, *42aH+*42bH, *44aH+*44bH, *51aH+*51bH, *52aH+*52bH, *54aH+*54bH, *57aH+*57bH, *74aH+*74bH, *76aH+*76bH, *97aH+*97bH, *107aH+*107bH, *114aH+*114bH, *127aH+*127bH, *129aH+*129bH, *135aH+*135bH, *142aH+*142bH, *143aH+*143bH, *164aH+*164bH, *176aH+*176bH, *177aH+*177bH, *182aH+*182bH, *200aH+*200bH, *203aH+*203bH, *212aH+*212bH, *231aH+*231bH, *233aH+*233bH, *234aH+*234bH, *239aH+*239bH, *246aH+*246bH, *250aH+*250bH, *254aH+*254bH, *269aH+*269bH

### Accessibility >140%, Min Distance to Active Site 6 Angstroms

*1aH+*1bH, *18aH+*18bH, *19aH+*19bH, *37aH+*37bH, *42aH+*42bH, *44aH+*44bH, *51aH+*51bH, *54aH+*54bH, *57aH+*57bH, *74aH+*74bH, *76aH+*76bH, *97aH+*97bH, *107aH+*107bH, *114aH+*114bH, *127aH+*127bH, *129aH+*129bH, *135aH+*135bH, *142aH+*142bH, *164aH+*164bH, *177aH+*177bH, *182aH+*182bH, *212aH+*212bH, *231aH+*231bH, *233aH+*233bH, *234aH+*234bH, *239aH+*239bH, *246aH+*246bH, *250aH+*250bH, *254aH+*254bH, *269aH+*269bH

### Accessibility >40%, Min Distance to Active Site 6 Angstroms

*1aH+*1bH+*1cH, *2aH+*2bH+*2cH, *3aH+*3bH+*3cH, *4aH+*4bH+*4cH, *6aH+*6bH+*6cH, *9aH+*9bH+*9cH, *10aH+*10bH+*10cH, *12aH+*12bH+*12cH, *14aH+*14bH+*14cH, *15aH+*15bH+*15cH, *17aH+*17bH+*17cH, *18aH+*18bH+*18cH, *19aH+*19bH+*19cH, *20aH+*20bH+*20cH, *22aH+*22bH+*22cH, *24aH+*24bH+*24cH, *25aH+*25bH+*25cH, *27aH+*27bH+*27cH, *37aH+*37bH+*37cH, *39aH+*39bH+*39cH, *42aH+*42bH+*42cH, *43aH+*43bH+*43cH, *44aH+*44bH+*44cH, *45aH+*45bH+*45cH, *46aH+*46bH+*46cH, *48aH+*48bH+*48cH, *49aH+*49bH+*49cH, *51aH+*51bH+*51cH, *52aH+*52bH+*52cH, *53aH+*53bH+*53cH, *54aH+*54bH+*54cH, *55aH+*55bH+*55cH, *56aH+*56bH+*56cH, *57aH+*57bH+*57cH, *59aH+*59bH+*59cH, *73aH+*73bH+*73cH, *74aH+*74bH+*74cH, *75aH+*75bH+*75cH, *76aH+*76bH+*76cH, *77aH+*77bH+*77cH, *85aH+*85bH+*85cH, *87aH+*87bH+*87cH, *89aH+*89bH+*89cH, *96aH+*96bH+*96cH, *97aH+*97bH+*97cH, *98aH+*98bH+*98cH, *99aH+*99bH+*99cH, *100aH+*100bH+*100cH, *101aH+*101bH+*101cH, *102aH+*102bH+*102cH, *103aH+*103bH+*103cH, *107aH+*107bH+*107cH, *110aH+*110bH+*110cH, *111aH+*111bH+*111cH, *114aH+*114bH+*114cH, *115aH+*115bH+*115cH, *116aH+*116bH+*116cH, *118aH+*118bH+*118cH, *125aH+*125bH+*125cH, *126aH+*126bH+*126cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *138aH+*138bH+*138cH, *139aH+*139bH+*139cH, *142aH+*142bH+*142cH, *143aH+*143bH+*143cH, *144aH+*144bH+*144cH, *153aH+*153bH+*153cH, *154aH+*154bH+*154cH, *155aH+*155bH+*155cH, *156aH+*156bH+*156cH, *157aH+*157bH+*157cH, *158aH+*158bH+*158cH, *161aH+*161bH+*161cH, *164aH+*164bH+*164cH, *166aH+*166bH+*166cH, *167aH+*167bH+*167cH, *175aH+*175bH+*175cH, *176aH+*176bH+*176cH, *177aH+*177bH+*177cH, *178aH+*178bH+*178cH, *179aH+*179bH+*179cH, *180aH+*180bH+*180cH, *182aH+*182bH+*182cH, *183aH+*183bH+*183cH, *186aH+*186bH+*186cH, *188aH+*188bH+*188cH, *189aH+*189bH+*189cH, *198aH+*198bH+*198cH, *200aH+*200bH+*200cH, *203aH+*203bH+*203cH, *204aH+*204bH+*204cH, *205aH+*205bH+*205cH, *206aH+*206bH+*206cH, *207aH+*207bH+*207cH, *208aH+*208bH+*208cH, *210aH+*210bH+*210cH, *211aH+*211bH+*211cH, *212aH+*212bH+*212cH, *229aH+*229bH+*229cH, *230aH+*230bH+*230cH, *231aH+*231bH+*231cH, *232aH+*232bH+*232cH, *233aH+*233bH+*233cH, *234aH+*234bH+*234cH, *235aH+*235bH+*235cH, *236aH+*236bH+*236cH, *238aH+*238bH+*238cH, *239aH+*239bH+*239cH, *241aH+*241bH+*241cH, *242aH+*242bH+*242cH, *243aH+*243bH+*243cH, *245aH+*245bH+*245cH, *246aH+*246bH+*246cH, *247aH+*247bH+*247cH, *249aH+*249bH+*249cH, *250aH+*250bH+*250cH, *251aH+*251bH+*251cH, *252aH+*252bH+*252cH, *253aH+*253bH+*253cH, *254aH+*254bH+*254cH, *255aH+*255bH+*255cH, *256aH+*256bH+*256cH, *257aH+*257bH+*257cH, *259aH+*259bH+*259cH, *265aH+*265bH+*265cH, *269aH+*269bH+*269cH.

### Accessibility >45%, Min Distance to Active Site 6 Angstroms

*1aH+*1bH+*1cH, *2aH+*2bH+*2cH, *3aH+*3bH+*3cH, *4aH+*4bH+*4cH, *6aH+*6bH+*6cH, *9aH+*9bH+*9cH, *10aH+*10bH+*10cH, *12aH+*12bH+*12cH, *14aH+*14bH+*14cH, *15aH+*15bH+*15cH, *17aH+*17bH+*17cH, *18aH+*18bH+*18cH, *19aH+*19bH+*19cH, *20aH+*20bH+*20cH, *22aH+*22bH+*22cH, *24aH+*24bH+*24cH, *25aH+*25bH+*25cH, *27aH+*27bH+*27cH, *37aH+*37bH+*37cH, *39aH+*39bH+*39cH, *42aH+*42bH+*42cH, *43aH+*43bH+*43cH, *44aH+*44bH+*44cH, *45aH+*45bH+*45cH, *46aH+*46bH+*46cH, *48aH+*48bH+*48cH, *49aH+*49bH+*49cH, *51aH+*51bH+*51cH, *52aH+*52bH+*52cH, *53aH+*53bH+*53cH, *54aH+*54bH+*54cH, *55aH+*55bH+*55cH, *56aH+*56bH+*56cH, *57aH+*57bH+*57cH, *59aH+*59bH+*59cH, *73aH+*73bH+*73cH, *74aH+*74bH+*74cH, *75aH+*75bH+*75cH, *76aH+*76bH+*76cH, *77aH+*77bH+*77cH, *85aH+*85bH+*85cH, *87aH+*87bH+*87cH, *96aH+*96bH+*96cH, *97aH+*97bH+*97cH, *98aH+*98bH+*98cH, *99aH+*99bH+*99cH, *100aH+*100bH+*100cH, *101aH+*101bH+*101cH, *102aH+*102bH+*102cH, *103aH+*103bH+*103cH, *107aH+*107bH+*107cH, *110aH+*110bH+*110cH, *111aH+*111bH+*111cH, *114aH+*114bH+*114cH, *115aH+*115bH+*115cH, *116aH+*116bH+*116cH, *118aH+*118bH+*118cH, *125aH+*125bH+*125cH, *126aH+*126bH+*126cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *138aH+*138bH+*138cH, *139aH+*139bH+*139cH, *142aH+*142bH+*142cH, *143aH+*143bH+*143cH, *144aH+*144bH+*144cH, *153aH+*153bH+*153cH, *154aH+*154bH+*154cH, *156aH+*156bH+*156cH, *157aH+*157bH+*157cH, *158aH+*158bH+*158cH, *161aH+*161bH+*161cH, *164aH+*164bH+*164cH, *166aH+*166bH+*166cH, *167aH+*167bH+*167cH, *175aH+*175bH+*175cH, *176aH+*176bH+*176cH, *177aH+*177bH+*177cH, *178aH+*178bH+*178cH, *179aH+*179bH+*179cH, *180aH+*180bH+*180cH, *182aH+*182bH+*182cH, *183aH+*183bH+*183cH, *186aH+*186bH+*186cH, *188aH+*188bH+*188cH, *189aH+*189bH+*189cH, *198aH+*198bH+*198cH, *200aH+*200bH+*200cH, *203aH+*203bH+*203cH, *204aH+*204bH+*204cH, *205aH+*205bH+*205cH, *206aH+*206bH+*206cH, *207aH+*207bH+*207cH, *208aH+*208bH+*208cH, *210aH+*210bH+*210cH, *211aH+*211bH+*211cH, *212aH+*212bH+*212cH, *229aH+*229bH+*229cH, *230aH+*230bH+*230cH, *231aH+*231bH+*231cH, *232aH+*232bH+*232cH, *233aH+*233bH+*233cH, *234aH+*234bH+*234cH, *235aH+*235bH+*235cH, *236aH+*236bH+*236cH, *238aH+*238bH+*238cH, *239aH+*239bH+*239cH, *241aH+*241bH+*241cH, *242aH+*242bH+*242cH, *243aH+*243bH+*243cH, *245aH+*245bH+*245cH, *246aH+*246bH+*246cH, *249aH+*249bH+*249cH, *250aH+*250bH+*250cH, *251aH+*251bH+*251cH, *252aH+*252bH+*252cH, *253aH+*253bH+*253cH, *254aH+*254bH+*254cH, *255aH+*255bH+*255cH, *256aH+*256bH+*256cH, *257aH+*257bH+*257cH, *259aH+*259bH+*259cH, *269aH+*269bH+*269cH

### Accessibility >50%, Min Distance to Active Site 6 Angstroms

*1aH+*1bH+*1cH, *2aH+*2bH+*2cH, *3aH+*3bH+*3cH, *4aH+*4bH+*4cH, *6aH+*6bH+*6cH, *9aH+*9bH+*9cH, *10aH+*10bH+*10cH, *12aH+*12bH+*12cH, *14aH+*14bH+*14cH, *15aH+*15bH+*15cH, *18aH+*18bH+*18cH, *19aH+*19bH+*19cH, *20aH+*20bH+*20cH, *24aH+*24bH+*24cH, *25aH+*25bH+*25cH, *27aH+*27bH+*27cH, *37aH+*37bH+*37cH, *39aH+*39bH+*39cH, *42aH+*42bH+*42cH, *43aH+*43bH+*43cH, *44aH+*44bH+*44cH, *49aH+*49bH+*49cH, *51aH+*51bH+*51cH, *52aH+*52bH+*52cH, *53aH+*53bH+*53cH, *54aH+*54bH+*54cH, *55aH+*55bH+*55cH, *56aH+*56bH+*56cH, *57aH+*57bH+*57cH, *59aH+*59bH+*59cH, *73aH+*73bH+*73cH, *74aH+*74bH+*74cH, *75aH+*75bH+*75cH, *76aH+*76bH+*76cH, *77aH+*77bH+*77cH, *85aH+*85bH+*85cH, *87aH+*87bH+*87cH, *96aH+*96bH+*96cH, *97aH+*97bH+*97cH, *98aH+*98bH+*98cH, *99aH+*99bH+*99cH, *100aH+*100bH+*100cH, *101aH+*101bH+*101cH, *102aH+*102bH+*102cH, *103aH+*103bH+*103cH, *107aH+*107bH+*107cH, *110aH+*110bH+*110cH, *111aH+*111bH+*111cH, *114aH+*114bH+*114cH, *115aH+*115bH+*115cH, *116aH+*116bH+*116cH, *118aH+*118bH+*118cH, *125aH+*125bH+*125cH, *126aH+*126bH+*126cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *138aH+*138bH+*138cH, *142aH+*142bH+*142cH, *143aH+*143bH+*143cH, *144aH+*144bH+*144cH, *153aH+*153bH+*153cH, *154aH+*154bH+*154cH, *156aH+*156bH+*156cH, *157aH+*157bH+*157cH, *158aH+*158bH+*158cH, *164aH+*164bH+*164cH, *166aH+*166bH+*166cH, *167aH+*167bH+*167cH, *175aH+*175bH+*175cH, *176aH+*176bH+*176cH, *177aH+*177bH+*177cH, *178aH+*178bH+*178cH, *179aH+*179bH+*179cH, *180aH+*180bH+*180cH, *182aH+*182bH+*182cH, *183aH+*183bH+*183cH, *186aH+*186bH+*186cH, *188aH+*188bH+*188cH, *189aH+*189bH+*189cH, *198aH+*198bH+*198cH, *200aH+*200bH+*200cH, *203aH+*203bH+*203cH, *205aH+*205bH+*205cH, *206aH+*206bH+*206cH, *207aH+*207bH+*207cH, *208aH+*208bH+*208cH, *210aH+*210bH+*210cH, *211aH+*211bH+*211cH, *212aH+*212bH+*212cH, *229aH+*229bH+*229cH, *230aH+*230bH+*230cH, *231aH+*231bH+*231cH, *232aH+*232bH+*232cH, *233aH+*233bH+*233cH, *234aH+*234bH+*234cH, *235aH+*235bH+*235cH, *236aH+*236bH+*236cH, *238aH+*238bH+*238cH, *239aH+*239bH+*239cH, *241aH+*241bH+*241cH, *242aH+*242bH+*242cH, *243aH+*243bH+*243cH, *245aH+*245bH+*245cH, *246aH+*246bH+*246cH, *249aH+*249bH+*249cH, *250aH+*250bH+*250cH, *251aH+*251bH+*251cH, *252aH+*252bH+*252cH, *253aH+*253bH+*253cH, *254aH+*254bH+*254cH, *255aH+*255bH+*255cH, *256aH+*256bH+*256cH, *257aH+*257bH+*257cH, *259aH+*259bH+*259cH, *269aH+*269bH+*269cH

### Accessibility >60%, Min Distance to Active Site 6 Angstroms

*1aH+*1bH+*1cH, *2aH+*2bH+*2cH, *3aH+*3bH+*3cH, *4aH+*4bH+*4cH, *6aH+*6bH+*6cH, *9aH+*9bH+*9cH, *10aH+*10bH+*10cH, *12aH+*12bH+*12cH, *14aH+*14bH+*14cH, *18aH+*18bH+*18cH, *19aH+*19bH+*19cH, *20aH+*20bH+*20cH, *24aH+*24bH+*24cH, *25aH+*25bH+*25cH, *27aH+*27bH+*27cH, *37aH+*37bH+*37cH, *39aH+*39bH+*39cH, *42aH+*42bH+*42cH, *43aH+*43bH+*43cH, *44aH+*44bH+*44cH, *49aH+*49bH+*49cH, *51aH+*51bH+*51cH, *52aH+*52bH+*52cH, *53aH+*53bH+*53cH, *54aH+*54bH+*54cH, *55aH+*55bH+*55cH, *56aH+*56bH+*56cH, *57aH+*57bH+*57cH, *59aH+*59bH+*59cH, *73aH+*73bH+*73cH, *74aH+*74bH+*74cH, *75aH+*75bH+*75cH, *76aH+*76bH+*76cH, *77aH+*77bH+*77cH, *85aH+*85bH+*85cH, *97aH+*97bH+*97cH, *99aH+*99bH+*99cH, *101aH+*101bH+*101cH, *102aH+*102bH+*102cH, *103aH+*103bH+*103cH, *107aH+*107bH+*107cH, *110aH+*110bH+*110cH, *111aH+*111bH+*111cH, *114aH+*114bH+*114cH, *115aH+*115bH+*115cH, *116aH+*116bH+*116cH, *118aH+*118bH+*118cH, *125aH+*125bH+*125cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *138aH+*138bH+*138cH, *142aH+*142bH+*142cH, *143aH+*143bH+*143cH, *153aH+*153bH+*153cH, *154aH+*154bH+*154cH, *156aH+*156bH+*156cH, *157aH+*157bH+*157cH, *158aH+*158bH+*158cH, *164aH+*164bH+*164cH, *166aH+*166bH+*166cH, *167aH+*167bH+*167cH, *176aH+*176bH+*176cH, *177aH+*177bH+*177cH, *178aH+*178bH+*178cH, *179aH+*179bH+*179cH, *180aH+*180bH+*180cH, *182aH+*182bH+*182cH, *183aH+*183bH+*183cH, *186aH+*186bH+*186cH, *188aH+*188bH+*188cH, *189aH+*189bH+*189cH, *198aH+*198bH+*198cH, *200aH+*200bH+*200cH, *203aH+*203bH+*203cH, *205aH+*205bH+*205cH, *206aH+*206bH+*206cH, *207aH+*207bH+*207cH, *208aH+*208bH+*208cH, *210aH+*210bH+*210cH, *211aH+*211bH+*211cH, *212aH+*212bH+*212cH, *229aH+*229bH+*229cH, *230aH+*230bH+*230cH, *231aH+*231bH+*231cH, *233aH+*233bH+*233cH, *234aH+*234bH+*234cH, *235aH+*235bH+*235cH, *236aH+*236bH+*236cH, *238aH+*238bH+*238cH, *239aH+*239bH+*239cH, *241aH+*241bH+*241cH, *242aH+*242bH+*242cH, *243aH+*243bH+*243cH, *245aH+*245bH+*245cH, *246aH+*246bH+*246cH, *249aH+*249bH+*249cH, *250aH+*250bH+*250cH, *251aH+*251bH+*251cH, *252aH+*252bH+*252cH, *253aH+*253bH+*253cH, *254aH+*254bH+*254cH, *255aH+*255bH+*255cH, *256aH+*256bH+*256cH, *269aH+*269bH+*269cH

### Accessibility >70%, Min Distance to Active Site 6 Angstroms

*1aH+*1bH+*1cH, *2aH+*2bH+*2cH, *3aH+*3bH+*3cH, *4aH+*4bH+*4cH, *6aH+*6bH+*6cH, *9aH+*9bH+*9cH, *10aH+*10bH+*10cH, *12aH+*12bH+*12cH, *14aH+*14bH+*14cH, *18aH+*18bH+*18cH, *19aH+*19bH+*19cH, *20aH+*20bH+*20cH, *24aH+*24bH+*24cH, *25aH+*25bH+*25cH, *27aH+*27bH+*27cH, *37aH+*37bH+*37cH, *39aH+*39bH+*39cH, *42aH+*42bH+*42cH, *44aH+*44bH+*44cH, *49aH+*49bH+*49cH, *51aH+*51bH+*51cH, *52aH+*52bH+*52cH, *53aH+*53bH+*53cH, *54aH+*54bH+*54cH, *55aH+*55bH+*55cH, *56aH+*56bH+*56cH, *57aH+*57bH+*57cH, *59aH+*59bH+*59cH, *73aH+*73bH+*73cH, *74aH+*74bH+*74cH, *75aH+*75bH+*75cH, *76aH+*76bH+*76cH, *77aH+*77bH+*77cH, *85aH+*85bH+*85cH, *97aH+*97bH+*97cH, *99aH+*99bH+*99cH, *101aH+*101bH+*101cH, *102aH+*102bH+*102cH, *103aH+*103bH+*103cH, *107aH+*107bH+*107cH, *110aH+*110bH+*110cH, *111aH+*111bH+*111cH, *114aH+*114bH+*114cH, *115aH+*115bH+*115cH, *116aH+*116bH+*116cH, *118aH+*118bH+*118cH, *125aH+*125bH+*125cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *138aH+*138bH+*138cH, *142aH+*142bH+*142cH, *143aH+*143bH+*143cH, *153aH+*153bH+*153cH, *154aH+*154bH+*154cH, *158aH+*158bH+*158cH, *164aH+*164bH+*164cH, *166aH+*166bH+*166cH, *167aH+*167bH+*167cH, *176aH+*176bH+*176cH, *177aH+*177bH+*177cH, *178aH+*178bH+*178cH, *179aH+*179bH+*179cH, *180aH+*180bH+*180cH, *182aH+*182bH+*182cH, *183aH+*183bH+*183cH, *186aH+*186bH+*186cH, *188aH+*188bH+*188cH, *189aH+*189bH+*189cH, *198aH+*198bH+*198cH, *200aH+*200bH+*200cH, *203aH+*203bH+*203cH, *205aH+*205bH+*205cH, *206aH+*206bH+*206cH, *207aH+*207bH+*207cH, *208aH+*208bH+*208cH, *210aH+*210bH+*210cH, *211aH+*211bH+*211cH, *212aH+*212bH+*212cH, *230aH+*230bH+*230cH, *231aH+*231bH+*231cH, *233aH+*233bH+*233cH, *234aH+*234bH+*234cH, *235aH+*235bH+*235cH, *236aH+*236bH+*236cH, *238aH+*238bH+*238cH, *239aH+*239bH+*239cH, *242aH+*242bH+*242cH, *243aH+*243bH+*243cH, *245aH+*245bH+*245cH, *246aH+*246bH+*246cH, *249aH+*249bH+*249cH, *250aH+*250bH+*250cH, *251aH+*251bH+*251cH, *253aH+*253bH+*253cH, *254aH+*254bH+*254cH, *255aH+*255bH+*255cH, *256aH+*256bH+*256cH, *269aH+*269bH+*269cH

### Accessibility >80%, Min Distance to Active Site 6 Angstroms

*1aH+*1bH+*1cH, *3aH+*3bH+*3cH, *4aH+*4bH+*4cH, *9aH+*9bH+*9cH, *10aH+*10bH+*10cH, *12aH+*12bH+*12cH, *18aH+*18bH+*18cH, *19aH+*19bH+*19cH, *24aH+*24bH+*24cH, *27aH+*27bH+*27cH, *37aH+*37bH+*37cH, *39aH+*39bH+*39cH, *42aH+*42bH+*42cH, *44aH+*44bH+*44cH, *51aH+*51bH+*51cH, *52aH+*52bH+*52cH, *54aH+*54bH+*54cH, *55aH+*55bH+*55cH, *57aH+*57bH+*57cH, *59aH+*59bH+*59cH, *74aH+*74bH+*74cH, *75aH+*75bH+*75cH, *76aH+*76bH+*76cH, *77aH+*77bH+*77cH, *85aH+*85bH+*85cH, *97aH+*97bH+*97cH, *99aH+*99bH+*99cH, *101aH+*101bH+*101cH, *103aH+*103bH+*103cH, *107aH+*107bH+*107cH, *110aH+*110bH+*110cH, *111aH+*111bH+*111cH, *114aH+*114bH+*114cH, *115aH+*115bH+*115cH, *118aH+*118bH+*118cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *138aH+*138bH+*138cH, *142aH+*142bH+*142cH, *143aH+*143bH+*143cH, *153aH+*153bH+*153cH, *154aH+*154bH+*154cH, *158aH+*158bH+*158cH, *164aH+*164bH+*164cH, *166aH+*166bH+*166cH, *167aH+*167bH+*167cH, *176aH+*176bH+*176cH, *177aH+*177bH+*177cH, *179aH+*179bH+*179cH, *180aH+*180bH+*180cH, *182aH+*182bH+*182cH, *186aH+*186bH+*186cH, *188aH+*188bH+*188cH, *198aH+*198bH+*198cH, *200aH+*200bH+*200cH, *203aH+*203bH+*203cH, *205aH+*205bH+*205cH, *206aH+*206bH+*206cH, *207aH+*207bH+*207cH, *208aH+*208bH+*208cH, *210aH+*210bH+*210cH, *211aH+*211bH+*211cH, *212aH+*212bH+*212cH, *230aH+*230bH+*230cH, *231aH+*231bH+*231cH, *233aH+*233bH+*233cH, *234aH+*234bH+*234cH, *235aH+*235bH+*235cH, *236aH+*236bH+*236cH, *238aH+*238bH+*238cH, *239aH+*239bH+*239cH, *242aH+*242bH+*242cH, *243aH+*243bH+*243cH, *246aH+*246bH+*246cH, *249aH+*249bH+*249cH, *250aH+*250bH+*250cH, *253aH+*253bH+*253cH, *254aH+*254bH+*254cH, *255aH+*255bH+*255cH, *256aH+*256bH+*256cH, *269aH+*269bH+*269cH

### Accessibility >90%, Min Distance to Active Site 6 Angstroms

*1aH+*1bH+*1cH, *3aH+*3bH+*3cH, *4aH+*4bH+*4cH, *9aH+*9bH+*9cH, *10aH+*10bH+*10cH, *12aH+*12bH+*12cH, *18aH+*18bH+*18cH, *19aH+*19bH+*19cH, *24aH+*24bH+*24cH, *37aH+*37bH+*37cH, *39aH+*39bH+*39cH, *42aH+*42bH+*42cH, *44aH+*44bH+*44cH, *51aH+*51bH+*51cH, *52aH+*52bH+*52cH, *54aH+*54bH+*54cH, *55aH+*55bH+*55cH, *57aH+*57bH+*57cH, *59aH+*59bH+*59cH, *74aH+*74bH+*74cH, *75aH+*75bH+*75cH, *76aH+*76bH+*76cH, *77aH+*77bH+*77cH, *97aH+*97bH+*97cH, *99aH+*99bH+*99cH, *101aH+*101bH+*101cH, *103aH+*103bH+*103cH, *107aH+*107bH+*107cH, *110aH+*110bH+*110cH, *114aH+*114bH+*114cH, *115aH+*115bH+*115cH, *118aH+*118bH+*118cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *142aH+*142bH+*142cH, *143aH+*143bH+*143cH, *153aH+*153bH+*153cH, *154aH+*154bH+*154cH, *158aH+*158bH+*158cH, *164aH+*164bH+*164cH, *166aH+*166bH+*166cH, *167aH+*167bH+*167cH, *176aH+*176bH+*176cH, *177aH+*177bH+*177cH, *179aH+*179bH+*179cH, *180aH+*180bH+*180cH, *182aH+*182bH+*182cH, *188aH+*188bH+*188cH, *198aH+*198bH+*198cH, *200aH+*200bH+*200cH, *203aH+*203bH+*203cH, *205aH+*205bH+*205cH, *206aH+*206bH+*206cH, *207aH+*207bH+*207cH, *208aH+*208bH+*208cH, *210aH+*210bH+*210cH, *212aH+*212bH+*212cH, *231aH+*231bH+*231cH, *233aH+*233bH+*233cH, *234aH+*234bH+*234cH, *235aH+*235bH+*235cH, *238aH+*238bH+*238cH, *239aH+*239bH+*239cH, *242aH+*242bH+*242cH, *246aH+*246bH+*246cH, *249aH+*249bH+*249cH, *250aH+*250bH+*250cH, *253aH+*253bH+*253cH, *254aH+*254bH+*254cH, *255aH+*255bH+*255cH, *269aH+*269bH+*269cH

### Accessibility >100%, Min Distance to Active Site 6 Angstroms

*1aH+*1bH+*1cH, *3aH+*3bH+*3cH, *4aH+*4bH+*4cH, *9aH+*9bH+*9cH, *10aH+*10bH+*10cH, *12aH+*12bH+*12cH, *18aH+*18bH+*18cH, *19aH+*19bH+*19cH, *24aH+*24bH+*24cH, *37aH+*37bH+*37cH, *39aH+*39bH+*39cH, *42aH+*42bH+*42cH, *44aH+*44bH+*44cH, *51aH+*51bH+*51cH, *52aH+*52bH+*52cH, *54aH+*54bH+*54cH, *57aH+*57bH+*57cH, *74aH+*74bH+*74cH, *76aH+*76bH+*76cH, *97aH+*97bH+*97cH, *99aH+*99bH+*99cH, *103aH+*103bH+*103cH, *107aH+*107bH+*107cH, *110aH+*110bH+*110cH, *114aH+*114bH+*114cH, *115aH+*115bH+*115cH, *127aH+*127bH+*127cH, *128aH+*128bH+*128cH, *129aH+*129bH+*129cH, *131aH+*131bH+*131cH, *132aH+*132bH+*132cH, *134aH+*134bH+*134cH, *135aH+*135bH+*135cH, *142aH+*142bH+*142cH, *143aH+*143bH+*143cH, *153aH+*153bH+*153cH, *154aH+*154bH+*154cH, *164aH+*164bH+*164cH, *166aH+*166bH+*166cH, *176aH+*176bH+*176cH, *177aH+*177bH+*177cH, *179aH+*179bH+*179cH, *182aH+*182bH+*182cH, *188aH+*188bH+*188cH, *198aH+*198bH+*198cH, *200aH+*200bH+*200cH, *203aH+*203bH+*203cH, *205aH+*205bH+*205cH, *206aH+*206bH+*206cH, *207aH+*207bH+*207cH, *208aH+*208bH+*208cH, *212aH+*212bH+*212cH, *231aH+*231bH+*231cH, *233aH+*233bH+*233cH, *234aH+*234bH+*234cH, *238aH+*238bH+*238cH, *239aH+*239bH+*239cH, *242aH+*242bH+*242cH, *246aH+*246bH+*246cH, *250aH+*250bH+*250cH, *254aH+*254bH+*254cH, *255aH+*255bH+*255cH, *269aH+*269bH+*269cH

### Accessibility >110%, Min Distance to Active Site 6 Angstroms

*1aH+*1bH+*1cH, *10aH+*10bH+*10cH, *12aH+*12bH+*12cH, *18aH+*18bH+*18cH, *19aH+*19bH+*19cH, *37aH+*37bH+*37cH, *42aH+*42bH+*42cH, *44aH+*44bH+*44cH, *51aH+*51bH+*51cH, *52aH+*52bH+*52cH, *54aH+*54bH+*54cH, *57aH+*57bH+*57cH, *74aH+*74bH+*74cH, *76aH+*76bH+*76cH, *97aH+*97bH+*97cH, *99aH+*99bH+*99cH, *107aH+*107bH+*107cH, *114aH+*114bH+*114cH, *115aH+*115bH+*115cH, *127aH+*127bH+*127cH, *129aH+*129bH+*129cH, *132aH+*132bH+*132cH, *134aH+*134bH+*134cH, *135aH+*135bH+*135cH, *142aH+*142bH+*142cH, *143aH+*143bH+*143cH, *153aH+*153bH+*153cH, *154aH+*154bH+*154cH, *164aH+*164bH+*164cH, *166aH+*166bH+*166cH, *176aH+*176bH+*176cH, *177aH+*177bH+*177cH, *179aH+*179bH+*179cH, *182aH+*182bH+*182cH, *188aH+*188bH+*188cH, *198aH+*198bH+*198cH, *200aH+*200bH+*200cH, *203aH+*203bH+*203cH, *205aH+*205bH+*205cH, *206aH+*206bH+*206cH, *212aH+*212bH+*212cH, *231aH+*231bH+*231cH, *233aH+*233bH+*233cH, *234aH+*234bH+*234cH, *238aH+*238bH+*238cH, *239aH+*239bH+*239cH, *242aH+*242bH+*242cH, *246aH+*246bH+*246cH, *250aH+*250bH+*250cH, *254aH+*254bH+*254cH, *255aH+*255bH+*255cH, *269aH+*269bH+*269cH

### Accessibility >120%, Min Distance to Active Site 6 Angstroms

*1aH+*1bH+*1cH, *10aH+*10bH+*10cH, *18aH+*18bH+*18cH, *19aH+*19bH+*19cH, *37aH+*37bH+*37cH, *42aH+*42bH+*42cH, *44aH+*44bH+*44cH, *51aH+*51bH+*51cH, *52aH+*52bH+*52cH, *54aH+*54bH+*54cH, *57aH+*57bH+*57cH, *74aH+*74bH+*74cH, *76aH+*76bH+*76cH, *97aH+*97bH+*97cH, *99aH+*99bH+*99cH, *107aH+*107bH+*107cH, *114aH+*114bH+*114cH, *115aH+*115bH+*115cH, *127aH+*127bH+*127cH, *129aH+*129bH+*129cH, *134aH+*134bH+*134cH, *135aH+*135bH+*135cH, *142aH+*142bH+*142cH, *143aH+*143bH+*143cH, *153aH+*153bH+*153cH, *154aH+*154bH+*154cH, *164aH+*164bH+*164cH, *166aH+*166bH+*166cH, *176aH+*176bH+*176cH, *177aH+*177bH+*177cH, *179aH+*179bH+*179cH, *182aH+*182bH+*182cH, *188aH+*188bH+*188cH, *198aH+*198bH+*198cH, *200aH+*200bH+*200cH, *203aH+*203bH+*203cH, *212aH+*212bH+*212cH, *231aH+*231bH+*231cH, *233aH+*233bH+*233cH, *234aH+*234bH+*234cH, *239aH+*239bH+*239cH, *246aH+*246bH+*246cH, *250aH+*250bH+*250cH, *254aH+*254bH+*254cH, *255aH+*255bH+*255cH, *269aH+*269bH+*269cH

### Accessibility >130%, Min Distance to Active Site 6 Angstroms

*1aH+*1bH+*1cH, *18aH+*18bH+*18cH, *19aH+*19bH+*19cH, *37aH+*37bH+*37cH, *42aH+*42bH+*42cH, *44aH+*44bH+*44cH, *51aH+*51bH+*51cH, *52aH+*52bH+*52cH, *54aH+*54bH+*54cH, *57aH+*57bH+*57cH, *74aH+*74bH+*74cH, *76aH+*76bH+*76cH, *97aH+*97bH+*97cH, *107aH+*107bH+*107cH, *114aH+*114bH+*114cH, *127aH+*127bH+*127cH, *129aH+*129bH+*129cH, *135aH+*135bH+*135cH, *142aH+*142bH+*142cH, *143aH+*143bH+*143cH, *164aH+*164bH+*164cH, *176aH+*176bH+*176cH, *177aH+*177bH+*177cH, *182aH+*182bH+*182cH, *200aH+*200bH+*200cH, *203aH+*203bH+*203cH, *212aH+*212bH+*212cH, *231aH+*231bH+*231cH, *233aH+*233bH+*233cH, *234aH+*234bH+*234cH, *239aH+*239bH+*239cH, *246aH+*246bH+*246cH, *250aH+*250bH+*250cH, *254aH+*254bH+*254cH, *269aH+*269bH+*269cH

### Accessibility >140%, Min Distance to Active Site 6 Angstroms

*1aH+*1bH+*1cH, *18aH+*18bH+*18cH, *19aH+*19bH+*19cH, *37aH+*37bH+*37cH, *42aH+*42bH+*42cH, *44aH+*44bH+*44cH, *51aH+*51bH+*51cH, *54aH+*54bH+*54cH, *57aH+*57bH+*57cH, *74aH+*74bH+*74cH, *76aH+*76bH+*76cH, *97aH+*97bH+*97cH, *107aH+*107bH+*107cH, *114aH+*114bH+*114cH, *127aH+*127bH+*127cH, *129aH+*129bH+*129cH, *135aH+*135bH+*135cH, *142aH+*142bH+*142cH, *164aH+*164bH+*164cH, *177aH+*177bH+*177cH, *182aH+*182bH+*182cH, *212aH+*212bH+*212cH, *231aH+*231bH+*231cH, *233aH+*233bH+*233cH, *234aH+*234bH+*234cH, *239aH+*239bH+*239cH, *246aH+*246bH+*246cH, *250aH+*250bH+*250cH, *254aH+*254bH+*254cH, *269aH+*269bH+*269cH

### Accessibility >40%, Min Distance to Active Site 6 Angstroms

*1aH+*1bH+*1cH+*1dH, *2aH+*2bH+*2cH+*2dH, *3aH+*3bH+*3cH+*3dH, *4aH+*4bH+*4cH+*4dH, *6aH+*6bH+*6cH+*6dH, *9aH+*9bH+*9cH+*9dH, *10aH+*10bH+*10cH+*10dH, *12aH+*12bH+*12cH+*12dH, *14aH+*14bH+*14cH+*14dH, *15aH+*15bH+*15cH+*15dH, *17aH+*17bH+*17cH+*17dH, *18aH+*18bH+*18cH+*18dH, *19aH+*19bH+*19cH+*19dH, *20aH+*20bH+*20cH+*20dH, *22aH+*22bH+*22cH+*22dH, *24aH+*24bH+*24cH+*24dH, *25aH+*25bH+*25cH+*25dH, *27aH+*27bH+*27cH+*27dH, *37aH+*37bH+*37cH+*37dH, *39aH+*39bH+*39cH+*39dH, *42aH+*42bH+*42cH+*42dH, *43aH+*43bH+*43cH+*43dH, *44aH+*44bH+*44cH+*44dH, *45aH+*45bH+*45cH+*45dH, *46aH+*46bH+*46cH+*46dH, *48aH+*48bH+*48cH+*48dH, *49aH+*49bH+*49cH+*49dH, *51aH+*51bH+*51cH+*51dH, *52aH+*52bH+*52cH+*52dH, *53aH+*53bH+*53cH+*53dH, *54aH+*54bH+*54cH+*54dH, *55aH+*55bH+*55cH+*55dH, *56aH+*56bH+*56cH+*56dH, *57aH+*57bH+*57cH+*57dH, *59aH+*59bH+*59cH+*59dH, *73aH+*73bH+*73cH+*73dH, *74aH+*74bH+*74cH+*74dH, *75aH+*75bH+*75cH+*75dH, *76aH+*76bH+*76cH+*76dH, *77aH+*77bH+*77cH+*77dH, *85aH+*85bH+*85cH+*85dH, *87aH+*87bH+*87cH+*87dH, *89aH+*89bH+*89cH+*89dH, *96aH+*96bH+*96cH+*96dH, *97aH+*97bH+*97cH+*97dH, *98aH+*98bH+*98cH+*98dH, *99aH+*99bH+*99cH+*99dH, *100aH+*100bH+*100cH+*100dH, *101aH+*101bH+*101cH+*101dH, *102aH+*102bH+*102cH+*102dH, *103aH+*103bH+*103cH+*103dH, *107aH+*107bH+*107cH+*107dH, *110aH+*110bH+*110cH+*110dH, *111aH+*111bH+*111cH+*111dH, *114aH+*114bH+*114cH+*114dH, *115aH+*115bH+*115cH+*115dH, *116aH+*116bH+*116cH+*116dH, *118aH+*118bH+*118cH+*118dH, *125aH+*125bH+*125cH+*125dH, *126aH+*126bH+*126cH+*126dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *138aH+*138bH+*138cH+*138dH, *139aH+*139bH+*139cH+*139dH, *142aH+*142bH+*142cH+*142dH, *143aH+*143bH+*143cH+*143dH, *144aH+*144bH+*144cH+*144dH, *153aH+*153bH+*153cH+*153dH, *154aH+*154bH+*154cH+*154dH, *155aH+*155bH+*155cH+*155dH, *156aH+*156bH+*156cH+*156dH, *157aH+*157bH+*157cH+*157dH, *158aH+*158bH+*158cH+*158dH, *161aH+*161bH+*161cH+*161dH, *164aH+*164bH+*164cH+*164dH, *166aH+*166bH+*166cH+*166dH, *167aH+*167bH+*167cH+*167dH, *175aH+*175bH+*175cH+*175dH, *176aH+*176bH+*176cH+*176dH, *177aH+*177bH+*177cH+*177dH, *178aH+*178bH+*178cH+*178dH, *179aH+*179bH+*179cH+*179dH, *180aH+*180bH+*180cH+*180dH, *182aH+*182bH+*182cH+*182dH, *183aH+*183bH+*183cH+*183dH, *186aH+*186bH+*186cH+*186dH, *188aH+*188bH+*188cH+*188dH, *189aH+*189bH+*189cH+*189dH, *198aH+*198bH+*198cH+*198dH, *200aH+*200bH+*200cH+*200dH, *203aH+*203bH+*203cH+*203dH, *204aH+*204bH+*204cH+*204dH, *205aH+*205bH+*205cH+*205dH, *206aH+*206bH+*206cH+*206dH, *207aH+*207bH+*207cH+*207dH, *208aH+*208bH+*208cH+*208dH, *210aH+*210bH+*210cH+*210dH, *211aH+*211bH+*211cH+*211dH, *212aH+*212bH+*212cH+*212dH, *229aH+*229bH+*229cH+*229dH, *230aH+*230bH+*230cH+*230dH, *231aH+*231bH+*231cH+*231dH, *232aH+*232bH+*232cH+*232dH, *233aH+*233bH+*233cH+*233dH, *234aH+*234bH+*234cH+*234dH, *235aH+*235bH+*235cH+*235dH, *236aH+*236bH+*236cH+*236dH, *238aH+*238bH+*238cH+*238dH, *239aH+*239bH+*239cH+*239dH, *241aH+*241bH+*241cH+*241dH, *242aH+*242bH+*242cH+*242dH, *243aH+*243bH+*243cH+*243dH, *245aH+*245bH+*245cH+*245dH, *246aH+*246bH+*246cH+*246dH, *247aH+*247bH+*247cH+*247dH, *249aH+*249bH+*249cH+*249dH, *250aH+*250bH+*250cH+*250dH, *251aH+*251bH+*251cH+*251dH, *252aH+*252bH+*252cH+*252dH, *253aH+*253bH+*253cH+*253dH, *254aH+*254bH+*254cH+*254dH, *255aH+*255bH+*255cH+*255dH, *256aH+*256bH+*256cH+*256dH, *257aH+*257bH+*257cH+*257dH, *259aH+*259bH+*259cH+*259dH, *265aH+*265bH+*265cH+*265dH, *269aH+*269bH+*269cH+*269dH

### Accessibility >45%, Min Distance to Active Site 6 Angstroms

*1aH+*1bH+*1cH+*1dH, *2aH+*2bH+*2cH+*2dH, *3aH+*3bH+*3cH+*3dH, *4aH+*4bH+*4cH+*4dH, *6aH+*6bH+*6cH+*6dH, *9aH+*9bH+*9cH+*9dH, *10aH+*10bH+*10cH+*10dH, *12aH+*12bH+*12cH+*12dH, *14aH+*14bH+*14cH+*14dH, *15aH+*15bH+*15cH+*15dH, *17aH+*17bH+*17cH+*17dH, *18aH+*18bH+*18cH+*18dH, *19aH+*19bH+*19cH+*19dH, *20aH+*20bH+*20cH+*20dH, *22aH+*22bH+*22cH+*22dH, *24aH+*24bH+*24cH+*24dH, *25aH+*25bH+*25cH+*25dH, *27aH+*27bH+*27cH+*27dH, *37aH+*37bH+*37cH+*37dH, *39aH+*39bH+*39cH+*39dH, *42aH+*42bH+*42cH+*42dH, *43aH+*43bH+*43cH+*43dH, *44aH+*44bH+*44cH+*44dH, *45aH+*45bH+*45cH+*45dH, *46aH+*46bH+*46cH+*46dH, *48aH+*48bH+*48cH+*48dH, *49aH+*49bH+*49cH+*49dH, *51aH+*51bH+*51cH+*51dH, *52aH+*52bH+*52cH+*52dH, *53aH+*53bH+*53cH+*53dH, *54aH+*54bH+*54cH+*54dH, *55aH+*55bH+*55cH+*55dH, *56aH+*56bH+*56cH+*56dH, *57aH+*57bH+*57cH+*57dH, *59aH+*59bH+*59cH+*59dH, *73aH+*73bH+*73cH+*73dH, *74aH+*74bH+*74cH+*74dH, *75aH+*75bH+*75cH+*75dH, *76aH+*76bH+*76cH+*76dH, *77aH+*77bH+*77cH+*77dH, *85aH+*85bH+*85cH+*85dH, *87aH+*87bH+*87cH+*87dH, *96aH+*96bH+*96cH+*96dH, *97aH+*97bH+*97cH+*97dH, *98aH+*98bH+*98cH+*98dH, *99aH+*99bH+*99cH+*99dH, *100aH+*100bH+*100cH+*100dH, *101aH+*101bH+*101cH+*101dH, *102aH+*102bH+*102cH+*102dH, *103aH+*103bH+*103cH+*103dH, *107aH+*107bH+*107cH+*107dH, *110aH+*110bH+*110cH+*110dH, *111aH+*111bH+*111cH+*111dH, *114aH+*114bH+*114cH+*114dH, *115aH+*115bH+*115cH+*115dH, *116aH+*116bH+*116cH+*116dH, *118aH+*118bH+*118cH+*118dH, *125aH+*125bH+*125cH+*125dH, *126aH+*126bH+*126cH+*126dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *138aH+*138bH+*138cH+*138dH, *139aH+*139bH+*139cH+*139dH, *142aH+*142bH+*142cH+*142dH, *143aH+*143bH+*143cH+*143dH, *144aH+*144bH+*144cH+*144dH, *153aH+*153bH+*153cH+*153dH, *154aH+*154bH+*154cH+*154dH, *156aH+*156bH+*156cH+*156dH, *157aH+*157bH+*157cH+*157dH, *158aH+*158bH+*158cH+*158dH, *161aH+*161bH+*161cH+*161dH, *164aH+*164bH+*164cH+*164dH, *166aH+*166bH+*166cH+*166dH, *167aH+*167bH+*167cH+*167dH, *175aH+*175bH+*175cH+*175dH, *176aH+*176bH+*176cH+*176dH, *177aH+*177bH+*177cH+*177dH, *178aH+*178bH+*178cH+*178dH, *179aH+*179bH+*179cH+*179dH, *180aH+*180bH+*180cH+*180dH, *182aH+*182bH+*182cH+*182dH, *183aH+*183bH+*183cH+*183dH, *186aH+*186bH+*186cH+*186dH, *188aH+*188bH+*188cH+*188dH, *189aH+*189bH+*189cH+*189dH, *198aH+*198bH+*198cH+*198dH, *200aH+*200bH+*200cH+*200dH, *203aH+*203bH+*203cH+*203dH, *204aH+*204bH+*204cH+*204dH, *205aH+*205bH+*205cH+*205dH, *206aH+*206bH+*206cH+*206dH, *207aH+*207bH+*207cH+*207dH, *208aH+*208bH+*208cH+*208dH, *210aH+*210bH+*210cH+*210dH, *211aH+*211bH+*211cH+*211dH, *212aH+*212bH+*212cH+*212dH, *229aH+*229bH+*229cH+*229dH, *230aH+*230bH+*230cH+*230dH, *231aH+*231bH+*231cH+*231dH, *232aH+*232bH+*232cH+*232dH, *233aH+*233bH+*233cH+*233dH, *234aH+*234bH+*234cH+*234dH, *235aH+*235bH+*235cH+*235dH, *236aH+*236bH+*236cH+*236dH, *238aH+*238bH+*238cH+*238dH, *239aH+*239bH+*239cH+*239dH, *241aH+*241bH+*241cH+*241dH, *242aH+*242bH+*242cH+*242dH, *243aH+*243bH+*243cH+*243dH, *245aH+*245bH+*245cH+*245dH, *246aH+*246bH+*246cH+*246dH, *249aH+*249bH+*249cH+*249dH, *250aH+*250bH+*250cH+*250dH, *251aH+*251bH+*251cH+*251dH, *252aH+*252bH+*252cH+*252dH, *253aH+*253bH+*253cH+*253dH, *254aH+*254bH+*254cH+*254dH, *255aH+*255bH+*255cH+*255dH, *256aH+*256bH+*256cH+*256dH, *257aH+*257bH+*257cH+*257dH, *259aH+*259bH+*259cH+*259dH, *269aH+*269bH+*269cH+*269dH

### Accessibility >50%, Min Distance to Active Site 6 Angstroms

*1aH+*1bH+*1cH+*1dH, *2aH+*2bH+*2cH+*2dH, *3aH+*3bH+*3cH+*3dH, *4aH+*4bH+*4cH+*4dH, *6aH+*6bH+*6cH+*6dH, *9aH+*9bH+*9cH+*9dH, *10aH+*10bH+*10cH+*10dH, *12aH+*12bH+*12cH+*12dH, *14aH+*14bH+*14cH+*14dH, *15aH+*15bH+*15cH+*15dH, *18aH+*18bH+*18cH+*18dH, *19aH+*19bH+*19cH+*19dH, *20aH+*20bH+*20cH+*20dH, *24aH+*24bH+*24cH+*24dH, *25aH+*25bH+*25cH+*25dH, *27aH+*27bH+*27cH+*27dH, *37aH+*37bH+*37cH+*37dH, *39aH+*39bH+*39cH+*39dH, *42aH+*42bH+*42cH+*42dH, *43aH+*43bH+*43cH+*43dH, *44aH+*44bH+*44cH+*44dH, *49aH+*49bH+*49cH+*49dH, *51aH+*51bH+*51cH+*51dH, *52aH+*52bH+*52cH+*52dH, *53aH+*53bH+*53cH+*53dH, *54aH+*54bH+*54cH+*54dH, *55aH+*55bH+*55cH+*55dH, *56aH+*56bH+*56cH+*56dH, *57aH+*57bH+*57cH+*57dH, *59aH+*59bH+*59cH+*59dH, *73aH+*73bH+*73cH+*73dH, *74aH+*74bH+*74cH+*74dH, *75aH+*75bH+*75cH+*75dH, *76aH+*76bH+*76cH+*76dH, *77aH+*77bH+*77cH+*77dH, *85aH+*85bH+*85cH+*85dH, *87aH+*87bH+*87cH+*87dH, *96aH+*96bH+*96cH+*96dH, *97aH+*97bH+*97cH+*97dH, *98aH+*98bH+*98cH+*98dH, *99aH+*99bH+*99cH+*99dH, *100aH+*100bH+*100cH+*100dH, *101aH+*101bH+*101cH+*101dH, *102aH+*102bH+*102cH+*102dH, *103aH+*103bH+*103cH+*103dH, *107aH+*107bH+*107cH+*107dH, *110aH+*110bH+*110cH+*110dH, *111aH+*111bH+*111cH+*111dH, *114aH+*114bH+*114cH+*114dH, *115aH+*115bH+*115cH+*115dH, *116aH+*116bH+*116cH+*116dH, *118aH+*118bH+*118cH+*118dH, *125aH+*125bH+*125cH+*125dH, *126aH+*126bH+*126cH+*126dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *138aH+*138bH+*138cH+*138dH, *142aH+*142bH+*142cH+*142dH, *143aH+*143bH+*143cH+*143dH, *144aH+*144bH+*144cH+*144dH, *153aH+*153bH+*153cH+*153dH, *154aH+*154bH+*154cH+*154dH, *156aH+*156bH+*156cH+*156dH, *157aH+*157bH+*157cH+*157dH, *158aH+*158bH+*158cH+*158dH, *164aH+*164bH+*164cH+*164dH, *166aH+*166bH+*166cH+*166dH, *167aH+*167bH+*167cH+*167dH, *175aH+*175bH+*175cH+*175dH, *176aH+*176bH+*176cH+*176dH, *177aH+*177bH+*177cH+*177dH, *178aH+*178bH+*178cH+*178dH, *179aH+*179bH+*179cH+*179dH, *180aH+*180bH+*180cH+*180dH, *182aH+*182bH+*182cH+*182dH, *183aH+*183bH+*183cH+*183dH, *186aH+*186bH+*186cH+*186dH, *188aH+*188bH+*188cH+*188dH, *189aH+*189bH+*189cH+*189dH, *198aH+*198bH+*198cH+*198dH, *200aH+*200bH+*200cH+*200dH, *203aH+*203bH+*203cH+*203dH, *205aH+*205bH+*205cH+*205dH, *206aH+*206bH+*206cH+*206dH, *207aH+*207bH+*207cH+*207dH, *208aH+*208bH+*208cH+*208dH, *210aH+*210bH+*210cH+*210dH, *211aH+*211bH+*211cH+*211dH, *212aH+*212bH+*212cH+*212dH, *229aH+*229bH+*229cH+*229dH, *230aH+*230bH+*230cH+*230dH, *231aH+*231bH+*231cH+*231dH, *232aH+*232bH+*232cH+*232dH, *233aH+*233bH+*233cH+*233dH, *234aH+*234bH+*234cH+*234dH, *235aH+*235bH+*235cH+*235dH, *236aH+*236bH+*236cH+*236dH, *238aH+*238bH+*238cH+*238dH, *239aH+*239bH+*239cH+*239dH, *241aH+*241bH+*241cH+*241dH, *242aH+*242bH+*242cH+*242dH, *243aH+*243bH+*243cH+*243dH, *245aH+*245bH+*245cH+*245dH, *246aH+*246bH+*246cH+*246dH, *249aH+*249bH+*249cH+*249dH, *250aH+*250bH+*250cH+*250dH, *251aH+*251bH+*251cH+*251dH, *252aH+*252bH+*252cH+*252dH, *253aH+*253bH+*253cH+*253dH, *254aH+*254bH+*254cH+*254dH, *255aH+*255bH+*255cH+*255dH, *256aH+*256bH+*256cH+*256dH, *257aH+*257bH+*257cH+*257dH, *259aH+*259bH+*259cH+*259dH, *269aH+*269bH+*269cH+*269dH

### Accessibility >60%, Min Distance to Active Site 6 Angstroms

*1aH+*1bH+*1cH+*1dH, *2aH+*2bH+*2cH+*2dH, *3aH+*3bH+*3cH+*3dH, *4aH+*4bH+*4cH+*4dH, *6aH+*6bH+*6cH+*6dH, *9aH+*9bH+*9cH+*9dH, *10aH+*10bH+*10cH+*10dH, *12aH+*12bH+*12cH+*12dH, *14aH+*14bH+*14cH+*14dH, *18aH+*18bH+*18cH+*18dH, *19aH+*19bH+*19cH+*19dH, *20aH+*20bH+*20cH+*20dH, *24aH+*24bH+*24cH+*24dH, *25aH+*25bH+*25cH+*25dH, *27aH+*27bH+*27cH+*27dH, *37aH+*37bH+*37cH+*37dH, *39aH+*39bH+*39cH+*39dH, *42aH+*42bH+*42cH+*42dH, *43aH+*43bH+*43cH+*43dH, *44aH+*44bH+*44cH+*44dH, *49aH+*49bH+*49cH+*49dH, *51aH+*51bH+*51cH+*51dH, *52aH+*52bH+*52cH+*52dH, *53aH+*53bH+*53cH+*53dH, *54aH+*54bH+*54cH+*54dH, *55aH+*55bH+*55cH+*55dH, *56aH+*56bH+*56cH+*56dH, *57aH+*57bH+*57cH+*57dH, *59aH+*59bH+*59cH+*59dH, *73aH+*73bH+*73cH+*73dH, *74aH+*74bH+*74cH+*74dH, *75aH+*75bH+*75cH+*75dH, *76aH+*76bH+*76cH+*76dH, *77aH+*77bH+*77cH+*77dH, *85aH+*85bH+*85cH+*85dH, *97aH+*97bH+*97cH+*97dH, *99aH+*99bH+*99cH+*99dH, *101aH+*101bH+*101cH+*101dH, *102aH+*102bH+*102cH+*102dH, *103aH+*103bH+*103cH+*103dH, *107aH+*107bH+*107cH+*107dH, *110aH+*110bH+*110cH+*110dH, *111aH+*111bH+*111cH+*111dH, *114aH+*114bH+*114cH+*114dH, *115aH+*115bH+*115cH+*115dH, *116aH+*116bH+*116cH+*116dH, *118aH+*118bH+*118cH+*118dH, *125aH+*125bH+*125cH+*125dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *138aH+*138bH+*138cH+*138dH, *142aH+*142bH+*142cH+*142dH, *143aH+*143bH+*143cH+*143dH, *153aH+*153bH+*153cH+*153dH, *154aH+*154bH+*154cH+*154dH, *156aH+*156bH+*156cH+*156dH, *157aH+*157bH+*157cH+*157dH, *158aH+*158bH+*158cH+*158dH, *164aH+*164bH+*164cH+*164dH, *166aH+*166bH+*166cH+*166dH, *167aH+*167bH+*167cH+*167dH, *176aH+*176bH+*176cH+*176dH, *177aH+*177bH+*177cH+*177dH, *178aH+*178bH+*178cH+*178dH, *179aH+*179bH+*179cH+*179dH, *180aH+*180bH+*180cH+*180dH, *182aH+*182bH+*182cH+*182dH, *183aH+*183bH+*183cH+*183dH, *186aH+*186bH+*186cH+*186dH, *188aH+*188bH+*188cH+*188dH, *189aH+*189bH+*189cH+*189dH, *198aH+*198bH+*198cH+*198dH, *200aH+*200bH+*200cH+*200dH, *203aH+*203bH+*203cH+*203dH, *205aH+*205bH+*205cH+*205dH, *206aH+*206bH+*206cH+*206dH, *207aH+*207bH+*207cH+*207dH, *208aH+*208bH+*208cH+*208dH, *210aH+*210bH+*210cH+*210dH, *211aH+*211bH+*211cH+*211dH, *212aH+*212bH+*212cH+*212dH, *229aH+*229bH+*229cH+*229dH, *230aH+*230bH+*230cH+*230dH, *231aH+*231bH+*231cH+*231dH, *233aH+*233bH+*233cH+*233dH, *234aH+*234bH+*234cH+*234dH, *235aH+*235bH+*235cH+*235dH, *236aH+*236bH+*236cH+*236dH, *238aH+*238bH+*238cH+*238dH, *239aH+*239bH+*239cH+*239dH, *241aH+*241bH+*241cH+*241dH, *242aH+*242bH+*242cH+*242dH, *243aH+*243bH+*243cH+*243dH, *245aH+*245bH+*245cH+*245dH, *246aH+*246bH+*246cH+*246dH, *249aH+*249bH+*249cH+*249dH, *250aH+*250bH+*250cH+*250dH, *251aH+*251bH+*251cH+*251dH, *252aH+*252bH+*252cH+*252dH, *253aH+*253bH+*253cH+*253dH, *254aH+*254bH+*254cH+*254dH, *255aH+*255bH+*255cH+*255dH, *256aH+*256bH+*256cH+*256dH, *269aH+*269bH+*269cH+*269dH

### Accessibility >70%, Min Distance to Active Site 6 Angstroms

*1aH+*1bH+*1cH+*1dH, *2aH+*2bH+*2cH+*2dH, *3aH+*3bH+*3cH+*3dH, *4aH+*4bH+*4cH+*4dH, *6aH+*6bH+*6cH+*6dH, *9aH+*9bH+*9cH+*9dH, *10aH+*10bH+*10cH+*10dH, *12aH+*12bH+*12cH+*12dH, *14aH+*14bH+*14cH+*14dH, *18aH+*18bH+*18cH+*18dH, *19aH+*19bH+*19cH+*19dH, *20aH+*20bH+*20cH+*20dH, *24aH+*24bH+*24cH+*24dH, *25aH+*25bH+*25cH+*25dH, *27aH+*27bH+*27cH+*27dH, *37aH+*37bH+*37cH+*37dH, *39aH+*39bH+*39cH+*39dH, *42aH+*42bH+*42cH+*42dH, *44aH+*44bH+*44cH+*44dH, *49aH+*49bH+*49cH+*49dH, *51aH+*51bH+*51cH+*51dH, *52aH+*52bH+*52cH+*52dH, *53aH+*53bH+*53cH+*53dH, *54aH+*54bH+*54cH+*54dH, *55aH+*55bH+*55cH+*55dH, *56aH+*56bH+*56cH+*56dH, *57aH+*57bH+*57cH+*57dH, *59aH+*59bH+*59cH+*59dH, *73aH+*73bH+*73cH+*73dH, *74aH+*74bH+*74cH+*74dH, *75aH+*75bH+*75cH+*75dH, *76aH+*76bH+*76cH+*76dH, *77aH+*77bH+*77cH+*77dH, *85aH+*85bH+*85cH+*85dH, *97aH+*97bH+*97cH+*97dH, *99aH+*99bH+*99cH+*99dH, *101aH+*101bH+*101cH+*101dH, *102aH+*102bH+*102cH+*102dH, *103aH+*103bH+*103cH+*103dH, *107aH+*107bH+*107cH+*107dH, *110aH+*110bH+*110cH+*110dH, *111aH+*111bH+*111cH+*111dH, *114aH+*114bH+*114cH+*114dH, *115aH+*115bH+*115cH+*115dH, *116aH+*116bH+*116cH+*116dH, *118aH+*118bH+*118cH+*118dH, *125aH+*125bH+*125cH+*125dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *138aH+*138bH+*138cH+*138dH, *142aH+*142bH+*142cH+*142dH, *143aH+*143bH+*143cH+*143dH, *153aH+*153bH+*153cH+*153dH, *154aH+*154bH+*154cH+*154dH, *158aH+*158bH+*158cH+*158dH, *164aH+*164bH+*164cH+*164dH, *166aH+*166bH+*166cH+*166dH, *167aH+*167bH+*167cH+*167dH, *176aH+*176bH+*176cH+*176dH, *177aH+*177bH+*177cH+*177dH, *178aH+*178bH+*178cH+*178dH, *179aH+*179bH+*179cH+*179dH, *180aH+*180bH+*180cH+*180dH, *182aH+*182bH+*182cH+*182dH, *183aH+*183bH+*183cH+*183dH, *186aH+*186bH+*186cH+*186dH, *188aH+*188bH+*188cH+*188dH, *189aH+*189bH+*189cH+*189dH, *198aH+*198bH+*198cH+*198dH, *200aH+*200bH+*200cH+*200dH, *203aH+*203bH+*203cH+*203dH, *205aH+*205bH+*205cH+*205dH, *206aH+*206bH+*206cH+*206dH, *207aH+*207bH+*207cH+*207dH, *208aH+*208bH+*208cH+*208dH, *210aH+*210bH+*210cH+*210dH, *211aH+*211bH+*211cH+*211dH, *212aH+*212bH+*212cH+*212dH, *230aH+*230bH+*230cH+*230dH, *231aH+*231bH+*231cH+*231dH, *233aH+*233bH+*233cH+*233dH, *234aH+*234bH+*234cH+*234dH, *235aH+*235bH+*235cH+*235dH, *236aH+*236bH+*236cH+*236dH, *238aH+*238bH+*238cH+*238dH, *239aH+*239bH+*239cH+*239dH, *242aH+*242bH+*242cH+*242dH, *243aH+*243bH+*243cH+*243dH, *245aH+*245bH+*245cH+*245dH, *246aH+*246bH+*246cH+*246dH, *249aH+*249bH+*249cH+*249dH, *250aH+*250bH+*250cH+*250dH, *251aH+*251bH+*251cH+*251dH, *253aH+*253bH+*253cH+*253dH, *254aH+*254bH+*254cH+*254dH, *255aH+*255bH+*255cH+*255dH, *256aH+*256bH+*256cH+*256dH, *269aH+*269bH+*269cH+*269dH

### Accessibility >80%, Min Distance to Active Site 6 Angstroms

*1aH+*1bH+*1cH+*1dH, *3aH+*3bH+*3cH+*3dH, *4aH+*4bH+*4cH+*4dH, *9aH+*9bH+*9cH+*9dH, *10aH+*10bH+*10cH+*10dH, *12aH+*12bH+*12cH+*12dH, *18aH+*18bH+*18cH+*18dH, *19aH+*19bH+*19cH+*19dH, *24aH+*24bH+*24cH+*24dH, *27aH+*27bH+*27cH+*27dH, *37aH+*37bH+*37cH+*37dH, *39aH+*39bH+*39cH+*39dH, *42aH+*42bH+*42cH+*42dH, *44aH+*44bH+*44cH+*44dH, *51aH+*51bH+*51cH+*51dH, *52aH+*52bH+*52cH+*52dH, *54aH+*54bH+*54cH+*54dH, *55aH+*55bH+*55cH+*55dH, *57aH+*57bH+*57cH+*57dH, *59aH+*59bH+*59cH+*59dH, *74aH+*74bH+*74cH+*74dH, *75aH+*75bH+*75cH+*75dH, *76aH+*76bH+*76cH+*76dH, *77aH+*77bH+*77cH+*77dH, *85aH+*85bH+*85cH+*85dH, *97aH+*97bH+*97cH+*97dH, *99aH+*99bH+*99cH+*99dH, *101aH+*101bH+*101cH+*101dH, *103aH+*103bH+*103cH+*103dH, *107aH+*107bH+*107cH+*107dH, *110aH+*110bH+*110cH+*110dH, *111aH+*111bH+*111cH+*111dH, *114aH+*114bH+*114cH+*114dH, *115aH+*115bH+*115cH+*115dH, *118aH+*118bH+*118cH+*118dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *138aH+*138bH+*138cH+*138dH, *142aH+*142bH+*142cH+*142dH, *143aH+*143bH+*143cH+*143dH, *153aH+*153bH+*153cH+*153dH, *154aH+*154bH+*154cH+*154dH, *158aH+*158bH+*158cH+*158dH, *164aH+*164bH+*164cH+*164dH, *166aH+*166bH+*166cH+*166dH, *167aH+*167bH+*167cH+*167dH, *176aH+*176bH+*176cH+*176dH, *177aH+*177bH+*177cH+*177dH, *179aH+*179bH+*179cH+*179dH, *180aH+*180bH+*180cH+*180dH, *182aH+*182bH+*182cH+*182dH, *186aH+*186bH+*186cH+*186dH, *188aH+*188bH+*188cH+*188dH, *198aH+*198bH+*198cH+*198dH, *200aH+*200bH+*200cH+*200dH, *203aH+*203bH+*203cH+*203dH, *205aH+*205bH+*205cH+*205dH, *206aH+*206bH+*206cH+*206dH, *207aH+*207bH+*207cH+*207dH, *208aH+*208bH+*208cH+*208dH, *210aH+*210bH+*210cH+*210dH, *211aH+*211bH+*211cH+*211dH, *212aH+*212bH+*212cH+*212dH, *230aH+*230bH+*230cH+*230dH, *231aH+*231bH+*231cH+*231dH, *233aH+*233bH+*233cH+*233dH, *234aH+*234bH+*234cH+*234dH, *235aH+*235bH+*235cH+*235dH, *236aH+*236bH+*236cH+*236dH, *238aH+*238bH+*238cH+*238dH, *239aH+*239bH+*239cH+*239dH, *242aH+*242bH+*242cH+*242dH, *243aH+*243bH+*243cH+*243dH, *246aH+*246bH+*246cH+*246dH, *249aH+*249bH+*249cH+*249dH, *250aH+*250bH+*250cH+*250dH, *253aH+*253bH+*253cH+*253dH, *254aH+*254bH+*254cH+*254dH, *255aH+*255bH+*255cH+*255dH, *256aH+*256bH+*256cH+*256dH, *269aH+*269bH+*269cH+*269dH

### Accessibility >90%, Min Distance to Active Site 6 Angstroms

*1aH+*1bH+*1cH+*1dH, *3aH+*3bH+*3cH+*3dH, *4aH+*4bH+*4cH+*4dH, *9aH+*9bH+*9cH+*9dH, *10aH+*10bH+*10cH+*10dH, *12aH+*12bH+*12cH+*12dH, *18aH+*18bH+*18cH+*18dH, *19aH+*19bH+*19cH+*19dH, *24aH+*24bH+*24cH+*24dH, *37aH+*37bH+*37cH+*37dH, *39aH+*39bH+*39cH+*39dH, *42aH+*42bH+*42cH+*42dH, *44aH+*44bH+*44cH+*44dH, *51aH+*51bH+*51cH+*51dH, *52aH+*52bH+*52cH+*52dH, *54aH+*54bH+*54cH+*54dH, *55aH+*55bH+*55cH+*55dH, *57aH+*57bH+*57cH+*57dH, *59aH+*59bH+*59cH+*59dH, *74aH+*74bH+*74cH+*74dH, *75aH+*75bH+*75cH+*75dH, *76aH+*76bH+*76cH+*76dH, *77aH+*77bH+*77cH+*77dH, *97aH+*97bH+*97cH+*97dH, *99aH+*99bH+*99cH+*99dH, *101aH+*101bH+*101cH+*101dH, *103aH+*103bH+*103cH+*103dH, *107aH+*107bH+*107cH+*107dH, *110aH+*110bH+*110cH+*110dH, *114aH+*114bH+*114cH+*114dH, *115aH+*115bH+*115cH+*115dH, *118aH+*118bH+*118cH+*118dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *142aH+*142bH+*142cH+*142dH, *143aH+*143bH+*143cH+*143dH, *153aH+*153bH+*153cH+*153dH, *154aH+*154bH+*154cH+*154dH, *158aH+*158bH+*158cH+*158dH, *164aH+*164bH+*164cH+*164dH, *166aH+*166bH+*166cH+*166dH, *167aH+*167bH+*167cH+*167dH, *176aH+*176bH+*176cH+*176dH, *177aH+*177bH+*177cH+*177dH, *179aH+*179bH+*179cH+*179dH, *180aH+*180bH+*180cH+*180dH, *182aH+*182bH+*182cH+*182dH, *188aH+*188bH+*188cH+*188dH, *198aH+*198bH+*198cH+*198dH, *200aH+*200bH+*200cH+*200dH, *203aH+*203bH+*203cH+*203dH, *205aH+*205bH+*205cH+*205dH, *206aH+*206bH+*206cH+*206dH, *207aH+*207bH+*207cH+*207dH, *208aH+*208bH+*208cH+*208dH, *210aH+*210bH+*210cH+*210dH, *212aH+*212bH+*212cH+*212dH, *231aH+*231bH+*231cH+*231dH, *233aH+*233bH+*233cH+*233dH, *234aH+*234bH+*234cH+*234dH, *235aH+*235bH+*235cH+*235dH, *238aH+*238bH+*238cH+*238dH, *239aH+*239bH+*239cH+*239dH, *242aH+*242bH+*242cH+*242dH, *246aH+*246bH+*246cH+*246dH, *249aH+*249bH+*249cH+*249dH, *250aH+*250bH+*250cH+*250dH, *253aH+*253bH+*253cH+*253dH, *254aH+*254bH+*254cH+*254dH, *255aH+*255bH+*255cH+*255dH, *269aH+*269bH+*269cH+*269dH

### Accessibility >100%, Min Distance to Active Site 6 Angstroms

*1aH+*1bH+*1cH+*1dH, *3aH+*3bH+*3cH+*3dH, *4aH+*4bH+*4cH+*4dH, *9aH+*9bH+*9cH+*9dH, *10aH+*10bH+*10cH+*10dH, *12aH+*12bH+*12cH+*12dH, *18aH+*18bH+*18cH+*18dH, *19aH+*19bH+*19cH+*19dH, *24aH+*24bH+*24cH+*24dH, *37aH+*37bH+*37cH+*37dH, *39aH+*39bH+*39cH+*39dH, *42aH+*42bH+*42cH+*42dH, *44aH+*44bH+*44cH+*44dH, *51aH+*51bH+*51cH+*51dH, *52aH+*52bH+*52cH+*52dH, *54aH+*54bH+*54cH+*54dH, *57aH+*57bH+*57cH+*57dH, *74aH+*74bH+*74cH+*74dH, *76aH+*76bH+*76cH+*76dH, *97aH+*97bH+*97cH+*97dH, *99aH+*99bH+*99cH+*99dH, *103aH+*103bH+*103cH+*103dH, *107aH+*107bH+*107cH+*107dH, *110aH+*110bH+*110cH+*110dH, *114aH+*114bH+*114cH+*114dH, *115aH+*115bH+*115cH+*115dH, *127aH+*127bH+*127cH+*127dH, *128aH+*128bH+*128cH+*128dH, *129aH+*129bH+*129cH+*129dH, *131aH+*131bH+*131cH+*131dH, *132aH+*132bH+*132cH+*132dH, *134aH+*134bH+*134cH+*134dH, *135aH+*135bH+*135cH+*135dH, *142aH+*142bH+*142cH+*142dH, *143aH+*143bH+*143cH+*143dH, *153aH+*153bH+*153cH+*153dH, *154aH+*154bH+*154cH+*154dH, *164aH+*164bH+*164cH+*164dH, *166aH+*166bH+*166cH+*166dH, *176aH+*176bH+*176cH+*176dH, *177aH+*177bH+*177cH+*177dH, *179aH+*179bH+*179cH+*179dH, *182aH+*182bH+*182cH+*182dH, *188aH+*188bH+*188cH+*188dH, *198aH+*198bH+*198cH+*198dH, *200aH+*200bH+*200cH+*200dH, *203aH+*203bH+*203cH+*203dH, *205aH+*205bH+*205cH+*205dH, *206aH+*206bH+*206cH+*206dH, *207aH+*207bH+*207cH+*207dH, *208aH+*208bH+*208cH+*208dH, *212aH+*212bH+*212cH+*212dH, *231aH+*231bH+*231cH+*231dH, *233aH+*233bH+*233cH+*233dH, *234aH+*234bH+*234cH+*234dH, *238aH+*238bH+*238cH+*238dH, *239aH+*239bH+*239cH+*239dH, *242aH+*242bH+*242cH+*242dH, *246aH+*246bH+*246cH+*246dH, *250aH+*250bH+*250cH+*250dH, *254aH+*254bH+*254cH+*254dH, *255aH+*255bH+*255cH+*255dH, *269aH+*269bH+*269cH+*269dH

### Accessibility >110%, Min Distance to Active Site 6 Angstroms

*1aH+*1bH+*1cH+*1dH, *10aH+*10bH+*10cH+*10dH, *12aH+*12bH+*12cH+*12dH, *18aH+*18bH+*18cH+*18dH, *19aH+*19bH+*19cH+*19dH, *37aH+*37bH+*37cH+*37dH, *42aH+*42bH+*42cH+*42dH, *44aH+*44bH+*44cH+*44dH, *51aH+*51bH+*51cH+*51dH, *52aH+*52bH+*52cH+*52dH, *54aH+*54bH+*54cH+*54dH, *57aH+*57bH+*57cH+*57dH, *74aH+*74bH+*74cH+*74dH, *76aH+*76bH+*76cH+*76dH, *97aH+*97bH+*97cH+*97dH, *99aH+*99bH+*99cH+*99dH, *107aH+*107bH+*107cH+*107dH, *114aH+*114bH+*114cH+*114dH, *115aH+*115bH+*115cH+*115dH, *127aH+*127bH+*127cH+*127dH, *129aH+*129bH+*129cH+*129dH, *132aH+*132bH+*132cH+*132dH, *134aH+*134bH+*134cH+*134dH, *135aH+*135bH+*135cH+*135dH, *142aH+*142bH+*142cH+*142dH, *143aH+*143bH+*143cH+*143dH, *153aH+*153bH+*153cH+*153dH, *154aH+*154bH+*154cH+*154dH, *164aH+*164bH+*164cH+*164dH, *166aH+*166bH+*166cH+*166dH, *176aH+*176bH+*176cH+*176dH, *177aH+*177bH+*177cH+*177dH, *179aH+*179bH+*179cH+*179dH, *182aH+*182bH+*182cH+*182dH, *188aH+*188bH+*188cH+*188dH, *198aH+*198bH+*198cH+*198dH, *200aH+*200bH+*200cH+*200dH, *203aH+*203bH+*203cH+*203dH, *205aH+*205bH+*205cH+*205dH, *206aH+*206bH+*206cH+*206dH, *212aH+*212bH+*212cH+*212dH, *231aH+*231bH+*231cH+*231dH, *233aH+*233bH+*233cH+*233dH, *234aH+*234bH+*234cH+*234dH, *238aH+*238bH+*238cH+*238dH, *239aH+*239bH+*239cH+*239dH, *242aH+*242bH+*242cH+*242dH, *246aH+*246bH+*246cH+*246dH, *250aH+*250bH+*250cH+*250dH, *254aH+*254bH+*254cH+*254dH, *255aH+*255bH+*255cH+*255dH, *269aH+*269bH+*269cH+*269dH

### Accessibility >120%, Min Distance to Active Site 6 Angstroms

*1aH+*1bH+*1cH+*1dH, *10aH+*10bH+*10cH+*10dH, *18aH+*18bH+*18cH+*18dH, *19aH+*19bH+*19cH+*19dH, *37aH+*37bH+*37cH+*37dH, *42aH+*42bH+*42cH+*42dH, *44aH+*44bH+*44cH+*44dH, *51aH+*51bH+*51cH+*51dH, *52aH+*52bH+*52cH+*52dH, *54aH+*54bH+*54cH+*54dH, *57aH+*57bH+*57cH+*57dH, *74aH+*74bH+*74cH+*74dH, *76aH+*76bH+*76cH+*76dH, *97aH+*97bH+*97cH+*97dH, *99aH+*99bH+*99cH+*99dH, *107aH+*107bH+*107cH+*107dH, *114aH+*114bH+*114cH+*114dH, *115aH+*115bH+*115cH+*115dH, *127aH+*127bH+*127cH+*127dH, *129aH+*129bH+*129cH+*129dH, *134aH+*134bH+*134cH+*134dH, *135aH+*135bH+*135cH+*135dH, *142aH+*142bH+*142cH+*142dH, *143aH+*143bH+*143cH+*143dH, *153aH+*153bH+*153cH+*153dH, *154aH+*154bH+*154cH+*154dH, *164aH+*164bH+*164cH+*164dH, *166aH+*166bH+*166cH+*166dH, *176aH+*176bH+*176cH+*176dH, *177aH+*177bH+*177cH+*177dH, *179aH+*179bH+*179cH+*179dH, *182aH+*182bH+*182cH+*182dH, *188aH+*188bH+*188cH+*188dH, *198aH+*198bH+*198cH+*198dH, *200aH+*200bH+*200cH+*200dH, *203aH+*203bH+*203cH+*203dH, *212aH+*212bH+*212cH+*212dH, *231aH+*231bH+*231cH+*231dH, *233aH+*233bH+*233cH+*233dH, *234aH+*234bH+*234cH+*234dH, *239aH+*239bH+*239cH+*239dH, *246aH+*246bH+*246cH+*246dH, *250aH+*250bH+*250cH+*250dH, *254aH+*254bH+*254cH+*254dH, *255aH+*255bH+*255cH+*255dH, *269aH+*269bH+*269cH+*269dH

### Accessibility >130%, Min Distance to Active Site 6 Angstroms

*1aH+*1bH+*1cH+*1dH, *18aH+*18bH+*18cH+*18dH, *19aH+*19bH+*19cH+*19dH, *37aH+*37bH+*37cH+*37dH, *42aH+*42bH+*42cH+*42dH, *44aH+*44bH+*44cH+*44dH, *51aH+*51bH+*51cH+*51dH, *52aH+*52bH+*52cH+*52dH, *54aH+*54bH+*54cH+*54dH, *57aH+*57bH+*57cH+*57dH, *74aH+*74bH+*74cH+*74dH, *76aH+*76bH+*76cH+*76dH, *97aH+*97bH+*97cH+*97dH, *107aH+*107bH+*107cH+*107dH, *114aH+*114bH+*114cH+*114dH, *127aH+*127bH+*127cH+*127dH, *129aH+*129bH+*129cH+*129dH, *135aH+*135bH+*135cH+*135dH, *142aH+*142bH+*142cH+*142dH, *143aH+*143bH+*143cH+*143dH, *164aH+*164bH+*164cH+*164dH, *176aH+*176bH+*176cH+*176dH, *177aH+*177bH+*177cH+*177dH, *182aH+*182bH+*182cH+*182dH, *200aH+*200bH+*200cH+*200dH, *203aH+*203bH+*203cH+*203dH, *212aH+*212bH+*212cH+*212dH, *231aH+*231bH+*231cH+*231dH, *233aH+*233bH+*233cH+*233dH, *234aH+*234bH+*234cH+*234dH, *239aH+*239bH+*239cH+*239dH, *246aH+*246bH+*246cH+*246dH, *250aH+*250bH+*250cH+*250dH, *254aH+*254bH+*254cH+*254dH, *269aH+*269bH+*269cH+*269dH

### Accessibility >140%, Min Distance to Active Site 6 Angstroms

*1aH+*1bH+*1cH+*1dH, *18aH+*18bH+*18cH+*18dH, *19aH+*19bH+*19cH+*19dH, *37aH+*37bH+*37cH+*37dH, *42aH+*42bH+*42cH+*42dH, *44aH+*44bH+*44cH+*44dH, *51aH+*51bH+*51cH+*51dH, *54aH+*54bH+*54cH+*54dH, *57aH+*57bH+*57cH+*57dH, *74aH+*74bH+*74cH+*74dH, *76aH+*76bH+*76cH+*76dH, *97aH+*97bH+*97cH+*97dH, *107aH+*107bH+*107cH+*107dH, *114aH+*114bH+*114cH+*114dH, *127aH+*127bH+*127cH+*127dH, *129aH+*129bH+*129cH+*129dH, *135aH+*135bH+*135cH+*135dH, *142aH+*142bH+*142cH+*142dH, *164aH+*164bH+*164cH+*164dH, *177aH+*177bH+*177cH+*177dH, *182aH+*182bH+*182cH+*182dH, *212aH+*212bH+*212cH+*212dH, *231aH+*231bH+*231cH+*231dH, *233aH+*233bH+*233cH+*233dH, *234aH+*234bH+*234cH+*234dH, *239aH+*239bH+*239cH+*239dH, *246aH+*246bH+*246cH+*246dH, *250aH+*250bH+*250cH+*250dH, *254aH+*254bH+*254cH+*254dH, *269aH+*269bH+*269cH+*269dH

### Accessibility >40%, Min Distance to Active Site 6 Angstroms

*1aH+*1bH+*1cH+*1dH+*1eH, *2aH+*2bH+*2cH+*2dH+*2eH, *3aH+*3bH+*3cH+*3dH+*3eH, *4aH+*4bH+*4cH+*4dH+*4eH, *6aH+*6bH+*6cH+*6dH+*6eH, *9aH+*9bH+*9cH+*9dH+*9eH, *10aH+*10bH+*10cH+*10dH+*10eH, *12aH+*12bH+*12cH+*12dH+*12eH, *14aH+*14bH+*14cH+*14dH+*14eH, *15aH+*15bH+*15cH+*15dH+*15eH, *17aH+*17bH+*17cH+*17dH+*17eH, *18aH+*18bH+*18cH+*18dH+*18eH, *19aH+*19bH+*19cH+*19dH+*19eH, *20aH+*20bH+*20cH+*20dH+*20eH, *22aH+*22bH+*22cH+*22dH+*22eH, *24aH+*24bH+*24cH+*24dH+*24eH, *25aH+*25bH+*25cH+*25dH+*25eH, *27aH+*27bH+*27cH+*27dH+*27eH, *37aH+*37bH+*37cH+*37dH+*37eH, *39aH+*39bH+*39cH+*39dH+*39eH, *42aH+*42bH+*42cH+*42dH+*42eH, *43aH+*43bH+*43cH+*43dH+*43eH, *44aH+*44bH+*44cH+*44dH+*44eH, *45aH+*45bH+*45cH+*45dH+*45eH, *46aH+*46bH+*46cH+*46dH+*46eH, *48aH+*48bH+*48cH+*48dH+*48eH, *49aH+*49bH+*49cH+*49dH+*49eH, *51aH+*51bH+*51cH+*51dH+*51eH, *52aH+*52bH+*52cH+*52dH+*52eH, *53aH+*53bH+*53cH+*53dH+*53eH, *54aH+*54bH+*54cH+*54dH+*54eH, *55aH+*55bH+*55cH+*55dH+*55eH, *56aH+*56bH+*56cH+*56dH+*56eH, *57aH+*57bH+*57cH+*57dH+*57eH, *59aH+*59bH+*59cH+*59dH+*59eH, *73aH+*73bH+*73cH+*73dH+*73eH, *74aH+*74bH+*74cH+*74dH+*74eH, *75aH+*75bH+*75cH+*75dH+*75eH, *76aH+*76bH+*76cH+*76dH+*76eH, *77aH+*77bH+*77cH+*77dH+*77eH, *85aH+*85bH+*85cH+*85dH+*85eH, *87aH+*87bH+*87cH+*87dH+*87eH, *89aH+*89bH+*89cH+*89dH+*89eH, *96aH+*96bH+*96cH+*96dH+*96eH, *97aH+*97bH+*97cH+*97dH+*97eH, *98aH+*98bH+*98cH+*98dH+*98eH, *99aH+*99bH+*99cH+*99dH+*99eH, *100aH+*100bH+*100cH+*100dH+*100eH, *101aH+*101bH+*101cH+*101dH+*101eH, *102aH+*102bH+*102cH+*102dH+*102eH, *103aH+*103bH+*103cH+*103dH+*103eH, *107aH+*107bH+*107cH+*107dH+*107eH, *110aH+*110bH+*110cH+*110dH+*110eH, *111aH+*111bH+*111cH+*111dH+*111eH, *114aH+*114bH+*114cH+*114dH+*114eH, *115aH+*115bH+*115cH+*115dH+*115eH, *116aH+*116bH+*116cH+*116dH+*116eH, *118aH+*118bH+*118cH+*118dH+*118eH, *125aH+*125bH+*125cH+*125dH+*125eH, *126aH+*126bH+*126cH+*126dH+*126eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *138aH+*138bH+*138cH+*138dH+*138eH, *139aH+*139bH+*139cH+*139dH+*139eH, *142aH+*142bH+*142cH+*142dH+*142eH, *143aH+*143bH+*143cH+*143dH+*143eH, *144aH+*144bH+*144cH+*144dH+*144eH, *153aH+*153bH+*153cH+*153dH+*153eH, *154aH+*154bH+*154cH+*154dH+*154eH, *155aH+*155bH+*155cH+*155dH+*155eH, *156aH+*156bH+*156cH+*156dH+*156eH, *157aH+*157bH+*157cH+*157dH+*157eH, *158aH+*158bH+*158cH+*158dH+*158eH, *161aH+*161bH+*161cH+*161dH+*161eH, *164aH+*164bH+*164cH+*164dH+*164eH, *166aH+*166bH+*166cH+*166dH+*166eH, *167aH+*167bH+*167cH+*167dH+*167eH, *175aH+*175bH+*175cH+*175dH+*175eH, *176aH+*176bH+*176cH+*176dH+*176eH, *177aH+*177bH+*177cH+*177dH+*177eH, *178aH+*178bH+*178cH+*178dH+*178eH, *179aH+*179bH+*179cH+*179dH+*179eH, *180aH+*180bH+*180cH+*180dH+*180eH, *182aH+*182bH+*182cH+*182dH+*182eH, *183aH+*183bH+*183cH+*183dH+*183eH, *186aH+*186bH+*186cH+*186dH+*186eH, *188aH+*188bH+*188cH+*188dH+*188eH, *189aH+*189bH+*189cH+*189dH+*189eH, *198aH+*198bH+*198cH+*198dH+*198eH, *200aH+*200bH+*200cH+*200dH+*200eH, *203aH+*203bH+*203cH+*203dH+*203eH, *204aH+*204bH+*204cH+*204dH+*204eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *210aH+*210bH+*210cH+*210dH+*210eH, *211aH+*211bH+*211cH+*211dH+*211eH, *212aH+*212bH+*212cH+*212dH+*212eH, *229aH+*229bH+*229cH+*229dH+*229eH, *230aH+*230bH+*230cH+*230dH+*230eH, *231aH+*231bH+*231cH+*231dH+*231eH, *232aH+*232bH+*232cH+*232dH+*232eH, *233aH+*233bH+*233cH+*233dH+*233eH, *234aH+*234bH+*234cH+*234dH+*234eH, *235aH+*235bH+*235cH+*235dH+*235eH, *236aH+*236bH+*236cH+*236dH+*236eH, *238aH+*238bH+*238cH+*238dH+*238eH, *239aH+*239bH+*239cH+*239dH+*239eH, *241aH+*241bH+*241cH+*241dH+*241eH, *242aH+*242bH+*242cH+*242dH+*242eH, *243aH+*243bH+*243cH+*243dH+*243eH, *245aH+*245bH+*245cH+*245dH+*245eH, *246aH+*246bH+*246cH+*246dH+*246eH, *247aH+*247bH+*247cH+*247dH+*247eH, *249aH+*249bH+*249cH+*249dH+*249eH, *250aH+*250bH+*250cH+*250dH+*250eH, *251aH+*251bH+*251cH+*251dH+*251eH, *252aH+*252bH+*252cH+*252dH+*252eH, *253aH+*253bH+*253cH+*253dH+*253eH, *254aH+*254bH+*254cH+*254dH+*254eH, *255aH+*255bH+*255cH+*255dH+*255eH, *256aH+*256bH+*256cH+*256dH+*256eH, *257aH+*257bH+*257cH+*257dH+*257eH, *259aH+*259bH+*259cH+*259dH+*259eH, *265aH+*265bH+*265cH+*265dH+*265eH, *269aH+*269bH+*269cH+*269dH+*269eH

### Accessibility >45%, Min Distance to Active Site 6 Angstroms

*1aH+*1bH+*1cH+*1dH+*1eH, *2aH+*2bH+*2cH+*2dH+*2eH, *3aH+*3bH+*3cH+*3dH+*3eH, *4aH+*4bH+*4cH+*4dH+*4eH, *6aH+*6bH+*6cH+*6dH+*6eH, *9aH+*9bH+*9cH+*9dH+*9eH, *10aH+*10bH+*10cH+*10dH+*10eH, *12aH+*12bH+*12cH+*12dH+*12eH, *14aH+*14bH+*14cH+*14dH+*14eH, *15aH+*15bH+*15cH+*15dH+*15eH, *17aH+*17bH+*17cH+*17dH+*17eH, *18aH+*18bH+*18cH+*18dH+*18eH, *19aH+*19bH+*19cH+*19dH+*19eH, *20aH+*20bH+*20cH+*20dH+*20eH, *22aH+*22bH+*22cH+*22dH+*22eH, *24aH+*24bH+*24cH+*24dH+*24eH, *25aH+*25bH+*25cH+*25dH+*25eH, *27aH+*27bH+*27cH+*27dH+*27eH, *37aH+*37bH+*37cH+*37dH+*37eH, *39aH+*39bH+*39cH+*39dH+*39eH, *42aH+*42bH+*42cH+*42dH+*42eH, *43aH+*43bH+*43cH+*43dH+*43eH, *44aH+*44bH+*44cH+*44dH+*44eH, *45aH+*45bH+*45cH+*45dH+*45eH, *46aH+*46bH+*46cH+*46dH+*46eH, *48aH+*48bH+*48cH+*48dH+*48eH, *49aH+*49bH+*49cH+*49dH+*49eH, *51aH+*51bH+*51cH+*51dH+*51eH, *52aH+*52bH+*52cH+*52dH+*52eH, *53aH+*53bH+*53cH+*53dH+*53eH, *54aH+*54bH+*54cH+*54dH+*54eH, *55aH+*55bH+*55cH+*55dH+*55eH, *56aH+*56bH+*56cH+*56dH+*56eH, *57aH+*57bH+*57cH+*57dH+*57eH, *59aH+*59bH+*59cH+*59dH+*59eH, *73aH+*73bH+*73cH+*73dH+*73eH, *74aH+*74bH+*74cH+*74dH+*74eH, *75aH+*75bH+*75cH+*75dH+*75eH, *76aH+*76bH+*76cH+*76dH+*76eH, *77aH+*77bH+*77cH+*77dH+*77eH, *85aH+*85bH+*85cH+*85dH+*85eH, *87aH+*87bH+*87cH+*87dH+*87eH, *96aH+*96bH+*96cH+*96dH+*96eH, *97aH+*97bH+*97cH+*97dH+*97eH, *98aH+*98bH+*98cH+*98dH+*98eH, *99aH+*99bH+*99cH+*99dH+*99eH, *100aH+*100bH+*100cH+*100dH+*100eH, *101aH+*101bH+*101cH+*101dH+*101eH, *102aH+*102bH+*102cH+*102dH+*102eH, *103aH+*103bH+*103cH+*103dH+*103eH, *107aH+*107bH+*107cH+*107dH+*107eH, *110aH+*110bH+*110cH+*110dH+*110eH, *111aH+*111bH+*111cH+*111dH+*111eH, *114aH+*114bH+*114cH+*114dH+*114eH, *115aH+*115bH+*115cH+*115dH+*115eH, *116aH+*116bH+*116cH+*116dH+*116eH, *118aH+*118bH+*118cH+*118dH+*118eH, *125aH+*125bH+*125cH+*125dH+*125eH, *126aH+*126bH+*126cH+*126dH+*126eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *138aH+*138bH+*138cH+*138dH+*138eH, *139aH+*139bH+*139cH+*139dH+*139eH, *142aH+*142bH+*142cH+*142dH+*142eH, *143aH+*143bH+*143cH+*143dH+*143eH, *144aH+*144bH+*144cH+*144dH+*144eH, *153aH+*153bH+*153cH+*153dH+*153eH, *154aH+*154bH+*154cH+*154dH+*154eH, *156aH+*156bH+*156cH+*156dH+*156eH, *157aH+*157bH+*157cH+*157dH+*157eH, *158aH+*158bH+*158cH+*158dH+*158eH, *161aH+*161bH+*161cH+*161dH+*161eH, *164aH+*164bH+*164cH+*164dH+*164eH, *166aH+*166bH+*166cH+*166dH+*166eH, *167aH+*167bH+*167cH+*167dH+*167eH, *175aH+*175bH+*175cH+*175dH+*175eH, *176aH+*176bH+*176cH+*176dH+*176eH, *177aH+*177bH+*177cH+*177dH+*177eH, *178aH+*178bH+*178cH+*178dH+*178eH, *179aH+*179bH+*179cH+*179dH+*179eH, *180aH+*180bH+*180cH+*180dH+*180eH, *182aH+*182bH+*182cH+*182dH+*182eH, *183aH+*183bH+*183cH+*183dH+*183eH, *186aH+*186bH+*186cH+*186dH+*186eH, *188aH+*188bH+*188cH+*188dH+*188eH, *189aH+*189bH+*189cH+*189dH+*189eH, *198aH+*198bH+*198cH+*198dH+*198eH, *200aH+*200bH+*200cH+*200dH+*200eH, *203aH+*203bH+*203cH+*203dH+*203eH, *204aH+*204bH+*204cH+*204dH+*204eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *210aH+*210bH+*210cH+*210dH+*210eH, *211aH+*211bH+*211cH+*211dH+*211eH, *212aH+*212bH+*212cH+*212dH+*212eH, *229aH+*229bH+*229cH+*229dH+*229eH, *230aH+*230bH+*230cH+*230dH+*230eH, *231aH+*231bH+*231cH+*231dH+*231eH, *232aH+*232bH+*232cH+*232dH+*232eH, *233aH+*233bH+*233cH+*233dH+*233eH, *234aH+*234bH+*234cH+*234dH+*234eH, *235aH+*235bH+*235cH+*235dH+*235eH, *236aH+*236bH+*236cH+*236dH+*236eH, *238aH+*238bH+*238cH+*238dH+*238eH, *239aH+*239bH+*239cH+*239dH+*239eH, *241aH+*241bH+*241cH+*241dH+*241eH, *242aH+*242bH+*242cH+*242dH+*242eH, *243aH+*243bH+*243cH+*243dH+*243eH, *245aH+*245bH+*245cH+*245dH+*245eH, *246aH+*246bH+*246cH+*246dH+*246eH, *249aH+*249bH+*249cH+*249dH+*249eH, *250aH+*250bH+*250cH+*250dH+*250eH, *251aH+*251bH+*251cH+*251dH+*251eH, *252aH+*252bH+*252cH+*252dH+*252eH, *253aH+*253bH+*253cH+*253dH+*253eH, *254aH+*254bH+*254cH+*254dH+*254eH, *255aH+*255bH+*255cH+*255dH+*255eH, *256aH+*256bH+*256cH+*256dH+*256eH, *257aH+*257bH+*257cH+*257dH+*257eH, *259aH+*259bH+*259cH+*259dH+*259eH, *269aH+*269bH+*269cH+*269dH+*269eH

### Accessibility >50%, Min Distance to Active Site 6 Angstroms

*1aH+*1bH+*1cH+*1dH+*1eH, *2aH+*2bH+*2cH+*2dH+*2eH, *3aH+*3bH+*3cH+*3dH+*3eH, *4aH+*4bH+*4cH+*4dH+*4eH, *6aH+*6bH+*6cH+*6dH+*6eH, *9aH+*9bH+*9cH+*9dH+*9eH, *10aH+*10bH+*10cH+*10dH+*10eH, *12aH+*12bH+*12cH+*12dH+*12eH, *14aH+*14bH+*14cH+*14dH+*14eH, *15aH+*15bH+*15cH+*15dH+*15eH, *18aH+*18bH+*18cH+*18dH+*18eH, *19aH+*19bH+*19cH+*19dH+*19eH, *20aH+*20bH+*20cH+*20dH+*20eH, *24aH+*24bH+*24cH+*24dH+*24eH, *25aH+*25bH+*25cH+*25dH+*25eH, *27aH+*27bH+*27cH+*27dH+*27eH, *37aH+*37bH+*37cH+*37dH+*37eH, *39aH+*39bH+*39cH+*39dH+*39eH, *42aH+*42bH+*42cH+*42dH+*42eH, *43aH+*43bH+*43cH+*43dH+*43eH, *44aH+*44bH+*44cH+*44dH+*44eH, *49aH+*49bH+*49cH+*49dH+*49eH, *51aH+*51bH+*51cH+*51dH+*51eH, *52aH+*52bH+*52cH+*52dH+*52eH, *53aH+*53bH+*53cH+*53dH+*53eH, *54aH+*54bH+*54cH+*54dH+*54eH, *55aH+*55bH+*55cH+*55dH+*55eH, *56aH+*56bH+*56cH+*56dH+*56eH, *57aH+*57bH+*57cH+*57dH+*57eH, *59aH+*59bH+*59cH+*59dH+*59eH, *73aH+*73bH+*73cH+*73dH+*73eH, *74aH+*74bH+*74cH+*74dH+*74eH, *75aH+*75bH+*75cH+*75dH+*75eH, *76aH+*76bH+*76cH+*76dH+*76eH, *77aH+*77bH+*77cH+*77dH+*77eH, *85aH+*85bH+*85cH+*85dH+*85eH, *87aH+*87bH+*87cH+*87dH+*87eH, *96aH+*96bH+*96cH+*96dH+*96eH, *97aH+*97bH+*97cH+*97dH+*97eH, *98aH+*98bH+*98cH+*98dH+*98eH, *99aH+*99bH+*99cH+*99dH+*99eH, *100aH+*100bH+*100cH+*100dH+*100eH, *101aH+*101bH+*101cH+*101dH+*101eH, *102aH+*102bH+*102cH+*102dH+*102eH, *103aH+*103bH+*103cH+*103dH+*103eH, *107aH+*107bH+*107cH+*107dH+*107eH, *110aH+*110bH+*110cH+*110dH+*110eH, *111aH+*111bH+*111cH+*111dH+*111eH, *114aH+*114bH+*114cH+*114dH+*114eH, *115aH+*115bH+*115cH+*115dH+*115eH, *116aH+*116bH+*116cH+*116dH+*116eH, *118aH+*118bH+*118cH+*118dH+*118eH, *125aH+*125bH+*125cH+*125dH+*125eH, *126aH+*126bH+*126cH+*126dH+*126eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *138aH+*138bH+*138cH+*138dH+*138eH, *142aH+*142bH+*142cH+*142dH+*142eH, *143aH+*143bH+*143cH+*143dH+*143eH, *144aH+*144bH+*144cH+*144dH+*144eH, *153aH+*153bH+*153cH+*153dH+*153eH, *154aH+*154bH+*154cH+*154dH+*154eH, *156aH+*156bH+*156cH+*156dH+*156eH, *157aH+*157bH+*157cH+*157dH+*157eH, *158aH+*158bH+*158cH+*158dH+*158eH, *164aH+*164bH+*164cH+*164dH+*164eH, *166aH+*166bH+*166cH+*166dH+*166eH, *167aH+*167bH+*167cH+*167dH+*167eH, *175aH+*175bH+*175cH+*175dH+*175eH, *176aH+*176bH+*176cH+*176dH+*176eH, *177aH+*177bH+*177cH+*177dH+*177eH, *178aH+*178bH+*178cH+*178dH+*178eH, *179aH+*179bH+*179cH+*179dH+*179eH, *180aH+*180bH+*180cH+*180dH+*180eH, *182aH+*182bH+*182cH+*182dH+*182eH, *183aH+*183bH+*183cH+*183dH+*183eH, *186aH+*186bH+*186cH+*186dH+*186eH, *188aH+*188bH+*188cH+*188dH+*188eH, *189aH+*189bH+*189cH+*189dH+*189eH, *198aH+*198bH+*198cH+*198dH+*198eH, *200aH+*200bH+*200cH+*200dH+*200eH, *203aH+*203bH+*203cH+*203dH+*203eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *210aH+*210bH+*210cH+*210dH+*210eH, *211aH+*211bH+*211cH+*211dH+*211eH, *212aH+*212bH+*212cH+*212dH+*212eH, *229aH+*229bH+*229cH+*229dH+*229eH, *230aH+*230bH+*230cH+*230dH+*230eH, *231aH+*231bH+*231cH+*231dH+*231eH, *232aH+*232bH+*232cH+*232dH+*232eH, *233aH+*233bH+*233cH+*233dH+*233eH, *234aH+*234bH+*234cH+*234dH+*234eH, *235aH+*235bH+*235cH+*235dH+*235eH, *236aH+*236bH+*236cH+*236dH+*236eH, *238aH+*238bH+*238cH+*238dH+*238eH, *239aH+*239bH+*239cH+*239dH+*239eH, *241aH+*241bH+*241cH+*241dH+*241eH, *242aH+*242bH+*242cH+*242dH+*242eH, *243aH+*243bH+*243cH+*243dH+*243eH, *245aH+*245bH+*245cH+*245dH+*245eH, *246aH+*246bH+*246cH+*246dH+*246eH, *249aH+*249bH+*249cH+*249dH+*249eH, *250aH+*250bH+*250cH+*250dH+*250eH, *251aH+*251bH+*251cH+*251dH+*251eH, *252aH+*252bH+*252cH+*252dH+*252eH, *253aH+*253bH+*253cH+*253dH+*253eH, *254aH+*254bH+*254cH+*254dH+*254eH, *255aH+*255bH+*255cH+*255dH+*255eH, *256aH+*256bH+*256cH+*256dH+*256eH, *257aH+*257bH+*257cH+*257dH+*257eH, *259aH+*259bH+*259cH+*259dH+*259eH, *269aH+*269bH+*269cH+*269dH+*269eH

### Accessibility >60%, Min Distance to Active Site 6 Angstroms

*1aH+*1bH+*1cH+*1dH+*1eH, *2aH+*2bH+*2cH+*2dH+*2eH, *3aH+*3bH+*3cH+*3dH+*3eH, *4aH+*4bH+*4cH+*4dH+*4eH, *6aH+*6bH+*6cH+*6dH+*6eH, *9aH+*9bH+*9cH+*9dH+*9eH, *10aH+*10bH+*10cH+*10dH+*10eH, *12aH+*12bH+*12cH+*12dH+*12eH, *14aH+*14bH+*14cH+*14dH+*14eH, *18aH+*18bH+*18cH+*18dH+*18eH, *19aH+*19bH+*19cH+*19dH+*19eH, *20aH+*20bH+*20cH+*20dH+*20eH, *24aH+*24bH+*24cH+*24dH+*24eH, *25aH+*25bH+*25cH+*25dH+*25eH, *27aH+*27bH+*27cH+*27dH+*27eH, *37aH+*37bH+*37cH+*37dH+*37eH, *39aH+*39bH+*39cH+*39dH+*39eH, *42aH+*42bH+*42cH+*42dH+*42eH, *43aH+*43bH+*43cH+*43dH+*43eH, *44aH+*44bH+*44cH+*44dH+*44eH, *49aH+*49bH+*49cH+*49dH+*49eH, *51aH+*51bH+*51cH+*51dH+*51eH, *52aH+*52bH+*52cH+*52dH+*52eH, *53aH+*53bH+*53cH+*53dH+*53eH, *54aH+*54bH+*54cH+*54dH+*54eH, *55aH+*55bH+*55cH+*55dH+*55eH, *56aH+*56bH+*56cH+*56dH+*56eH, *57aH+*57bH+*57cH+*57dH+*57eH, *59aH+*59bH+*59cH+*59dH+*59eH,*73aH+*73bH+*73cH+*73dH+*73eH, *74aH+*74bH+*74cH+*74dH+*74eH, *75aH+*75bH+*75cH+*75dH+*75eH, *76aH+*76bH+*76cH+*76dH+*76eH, *77aH+*77bH+*77cH+*77dH+*77eH, *85aH+*85bH+*85cH+*85dH+*85eH, *97aH+*97bH+*97cH+*97dH+*97eH, *99aH+*99bH+*99cH+*99dH+*99eH, *101aH+*101bH+*101cH+*101dH+*101eH, *102aH+*102bH+*102cH+*102dH+*102eH, *103aH+*103bH+*103cH+*103dH+*103eH, *107aH+*107bH+*107cH+*107dH+*107eH, *110aH+*110bH+*110cH+*110dH+*110eH, *111aH+*111bH+*111cH+*111dH+*111eH, *114aH+*114bH+*114cH+*114dH+*114eH, *115aH+*115bH+*115cH+*115dH+*115eH, *116aH+*116bH+*116cH+*116dH+*116eH, *118aH+*118bH+*118cH+*118dH+*118eH, *125aH+*125bH+*125cH+*125dH+*125eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *138aH+*138bH+*138cH+*138dH+*138eH, *142aH+*142bH+*142cH+*142dH+*142eH, *143aH+*143bH+*143cH+*143dH+*143eH, *153aH+*153bH+*153cH+*153dH+*153eH, *154aH+*154bH+*154cH+*154dH+*154eH, *156aH+*156bH+*156cH+*156dH+*156eH, *157aH+*157bH+*157cH+*157dH+*157eH, *158aH+*158bH+*158cH+*158dH+*158eH, *164aH+*164bH+*164cH+*164dH+*164eH, *166aH+*166bH+*166cH+*166dH+*166eH, *167aH+*167bH+*167cH+*167dH+*167eH, *176aH+*176bH+*176cH+*176dH+*176eH, *177aH+*177bH+*177cH+*177dH+*177eH, *178aH+*178bH+*178cH+*178dH+*178eH, *179aH+*179bH+*179cH+*179dH+*179eH, *180aH+*180bH+*180cH+*180dH+*180eH, *182aH+*182bH+*182cH+*182dH+*182eH, *183aH+*183bH+*183cH+*183dH+*183eH, *186aH+*186bH+*186cH+*186dH+*186eH, *188aH+*188bH+*188cH+*188dH+*188eH, *189aH+*189bH+*189cH+*189dH+*189eH, *198aH+*198bH+*198cH+*198dH+*198eH, *200aH+*200bH+*200cH+*200dH+*200eH, *203aH+*203bH+*203cH+*203dH+*203eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *210aH+*210bH+*210cH+*210dH+*210eH, *211aH+*211bH+*211cH+*211dH+*211eH, *212aH+*212bH+*212cH+*212dH+*212eH, *229aH+*229bH+*229cH+*229dH+*229eH, *230aH+*230bH+*230cH+*230dH+*230eH, *231aH+*231bH+*231cH+*231dH+*231eH, *233aH+*233bH+*233cH+*233dH+*233eH, *234aH+*234bH+*234cH+*234dH+*234eH, *235aH+*235bH+*235cH+*235dH+*235eH, *236aH+*236bH+*236cH+*236dH+*236eH, *238aH+*238bH+*238cH+*238dH+*238eH, *239aH+*239bH+*239cH+*239dH+*239eH, *241aH+*241bH+*241cH+*241dH+*241eH, *242aH+*242bH+*242cH+*242dH+*242eH, *243aH+*243bH+*243cH+*243dH+*243eH, *245aH+*245bH+*245cH+*245dH+*245eH, *246aH+*246bH+*246cH+*246dH+*246eH, *249aH+*249bH+*249cH+*249dH+*249eH, *250aH+*250bH+*250cH+*250dH+*250eH, *251aH+*251bH+*251cH+*251dH+*251eH, *252aH+*252bH+*252cH+*252dH+*252eH, *253aH+*253bH+*253cH+*253dH+*253eH, *254aH+*254bH+*254cH+*254dH+*254eH, *255aH+*255bH+*255cH+*255dH+*255eH, *256aH+*256bH+*256cH+*256dH+*256eH, *269aH+*269bH+*269cH+*269dH+*269eH

### Accessibility >70%, Min Distance to Active Site 6 Angstroms

*1aH+*1bH+*1cH+*1dH+*1eH, *2aH+*2bH+*2cH+*2dH+*2eH, *3aH+*3bH+*3cH+*3dH+*3eH, *4aH+*4bH+*4cH+*4dH+*4eH, *6aH+*6bH+*6cH+*6dH+*6eH, *9aH+*9bH+*9cH+*9dH+*9eH, *10aH+*10bH+*10cH+*10dH+*10eH, *12aH+*12bH+*12cH+*12dH+*12eH, *14aH+*14bH+*14cH+*14dH+*14eH, *18aH+*18bH+*18cH+*18dH+*18eH, *19aH+*19bH+*19cH+*19dH+*19eH, *20aH+*20bH+*20cH+*20dH+*20eH, *24aH+*24bH+*24cH+*24dH+*24eH, *25aH+*25bH+*25cH+*25dH+*25eH, *27aH+*27bH+*27cH+*27dH+*27eH, *37aH+*37bH+*37cH+*37dH+*37eH, *39aH+*39bH+*39cH+*39dH+*39eH, *42aH+*42bH+*42cH+*42dH+*42eH, *44aH+*44bH+*44cH+*44dH+*44eH, *49aH+*49bH+*49cH+*49dH+*49eH, *51aH+*51bH+*51cH+*51dH+*51eH, *52aH+*52bH+*52cH+*52dH+*52eH, *53aH+*53bH+*53cH+*53dH+*53eH, *54aH+*54bH+*54cH+*54dH+*54eH, *55aH+*55bH+*55cH+*55dH+*55eH, *56aH+*56bH+*56cH+*56dH+*56eH, *57aH+*57bH+*57cH+*57dH+*57eH, *59aH+*59bH+*59cH+*59dH+*59eH, *73aH+*73bH+*73cH+*73dH+*73eH, *74aH+*74bH+*74cH+*74dH+*74eH, *75aH+*75bH+*75cH+*75dH+*75eH, *76aH+*76bH+*76cH+*76dH+*76eH, *77aH+*77bH+*77cH+*77dH+*77eH, *85aH+*85bH+*85cH+*85dH+*85eH, *97aH+*97bH+*97cH+*97dH+*97eH, *99aH+*99bH+*99cH+*99dH+*99eH, *101aH+*101bH+*101cH+*101dH+*101eH, *102aH+*102bH+*102cH+*102dH+*102eH, *103aH+*103bH+*103cH+*103dH+*103eH, *107aH+*107bH+*107cH+*107dH+*107eH, *110aH+*110bH+*110cH+*110dH+*110eH, *111aH+*111bH+*111cH+*111dH+*111eH, *114aH+*114bH+*114cH+*114dH+*114eH, *115aH+*115bH+*115cH+*115dH+*115eH, *116aH+*116bH+*116cH+*116dH+*116eH, *118aH+*118bH+*118cH+*118dH+*118eH, *125aH+*125bH+*125cH+*125dH+*125eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *138aH+*138bH+*138cH+*138dH+*138eH, *142aH+*142bH+*142cH+*142dH+*142eH, *143aH+*143bH+*143cH+*143dH+*143eH, *153aH+*153bH+*153cH+*153dH+*153eH, *154aH+*154bH+*154cH+*154dH+*154eH, *158aH+*158bH+*158cH+*158dH+*158eH, *164aH+*164bH+*164cH+*164dH+*164eH, *166aH+*166bH+*166cH+*166dH+*166eH, *167aH+*167bH+*167cH+*167dH+*167eH, *176aH+*176bH+*176cH+*176dH+*176eH, *177aH+*177bH+*177cH+*177dH+*177eH, *178aH+*178bH+*178cH+*178dH+*178eH, *179aH+*179bH+*179cH+*179dH+*179eH, *180aH+*180bH+*180cH+*180dH+*180eH, *182aH+*182bH+*182cH+*182dH+*182eH, *183aH+*183bH+*183cH+*183dH+*183eH, *186aH+*186bH+*186cH+*186dH+*186eH, *188aH+*188bH+*188cH+*188dH+*188eH, *189aH+*189bH+*189cH+*189dH+*189eH, *198aH+*198bH+*198cH+*198dH+*198eH, *200aH+*200bH+*200cH+*200dH+*200eH, *203aH+*203bH+*203cH+*203dH+*203eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *210aH+*210bH+*210cH+*210dH+*210eH, *211aH+*211bH+*211cH+*211dH+*211eH, *212aH+*212bH+*212cH+*212dH+*212eH, *230aH+*230bH+*230cH+*230dH+*230eH, *231aH+*231bH+*231cH+*231dH+*231eH, *233aH+*233bH+*233cH+*233dH+*233eH, *234aH+*234bH+*234cH+*234dH+*234eH, *235aH+*235bH+*235cH+*235dH+*235eH, *236aH+*236bH+*236cH+*236dH+*236eH, *238aH+*238bH+*238cH+*238dH+*238eH, *239aH+*239bH+*239cH+*239dH+*239eH, *242aH+*242bH+*242cH+*242dH+*242eH, *243aH+*243bH+*243cH+*243dH+*243eH, *245aH+*245bH+*245cH+*245dH+*245eH, *246aH+*246bH+*246cH+*246dH+*246eH, *249aH+*249bH+*249cH+*249dH+*249eH, *250aH+*250bH+*250cH+*250dH+*250eH, *251aH+*251bH+*251cH+*251dH+*251eH, *253aH+*253bH+*253cH+*253dH+*253eH, *254aH+*254bH+*254cH+*254dH+*254eH, *255aH+*255bH+*255cH+*255dH+*255eH, *256aH+*256bH+*256cH+*256dH+*256eH, *269aH+*269bH+*269cH+*269dH+*269eH

### Accessibility >80%, Min Distance to Active Site 6 Angstroms

*1aH+*1bH+*1cH+*1dH+*1eH, *3aH+*3bH+*3cH+*3dH+*3eH, *4aH+*4bH+*4cH+*4dH+*4eH, *9aH+*9bH+*9cH+*9dH+*9eH, *10aH+*10bH+*10cH+*10dH+*10eH, *12aH+*12bH+*12cH+*12dH+*12eH, *18aH+*18bH+*18cH+*18dH+*18eH, *19aH+*19bH+*19cH+*19dH+*19eH, *24aH+*24bH+*24cH+*24dH+*24eH, *27aH+*27bH+*27cH+*27dH+*27eH, *37aH+*37bH+*37cH+*37dH+*37eH, *39aH+*39bH+*39cH+*39dH+*39eH, *42aH+*42bH+*42cH+*42dH+*42eH, *44aH+*44bH+*44cH+*44dH+*44eH, *51aH+*51bH+*51cH+*51dH+*51eH, *52aH+*52bH+*52cH+*52dH+*52eH, *54aH+*54bH+*54cH+*54dH+*54eH, *55aH+*55bH+*55cH+*55dH+*55eH, *57aH+*57bH+*57cH+*57dH+*57eH, *59aH+*59bH+*59cH+*59dH+*59eH, *74aH+*74bH+*74cH+*74dH+*74eH, *75aH+*75bH+*75cH+*75dH+*75eH, *76aH+*76bH+*76cH+*76dH+*76eH, *77aH+*77bH+*77cH+*77dH+*77eH, *85aH+*85bH+*85cH+*85dH+*85eH, *97aH+*97bH+*97cH+*97dH+*97eH, *99aH+*99bH+*99cH+*99dH+*99eH, *101aH+*101bH+*101cH+*101dH+*101eH, *103aH+*103bH+*103cH+*103dH+*103eH, *107aH+*107bH+*107cH+*107dH+*107eH, *110aH+*110bH+*110cH+*110dH+*110eH, *111aH+*111bH+*111cH+*111dH+*111eH, *114aH+*114bH+*114cH+*114dH+*114eH, *115aH+*115bH+*115cH+*115dH+*115eH, *118aH+*118bH+*118cH+*118dH+*118eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *138aH+*138bH+*138cH+*138dH+*138eH, *142aH+*142bH+*142cH+*142dH+*142eH, *143aH+*143bH+*143cH+*143dH+*143eH, *153aH+*153bH+*153cH+*153dH+*153eH, *154aH+*154bH+*154cH+*154dH+*154eH, *158aH+*158bH+*158cH+*158dH+*158eH, *164aH+*164bH+*164cH+*164dH+*164eH, *166aH+*166bH+*166cH+*166dH+*166eH, *167aH+*167bH+*167cH+*167dH+*167eH, *176aH+*176bH+*176cH+*176dH+*176eH, *177aH+*177bH+*177cH+*177dH+*177eH, *179aH+*179bH+*179cH+*179dH+*179eH, *180aH+*180bH+*180cH+*180dH+*180eH, *182aH+*182bH+*182cH+*182dH+*182eH, *186aH+*186bH+*186cH+*186dH+*186eH, *188aH+*188bH+*188cH+*188dH+*188eH, *198aH+*198bH+*198cH+*198dH+*198eH, *200aH+*200bH+*200cH+*200dH+*200eH, *203aH+*203bH+*203cH+*203dH+*203eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *210aH+*210bH+*210cH+*210dH+*210eH, *211aH+*211bH+*211cH+*211dH+*211eH, *212aH+*212bH+*212cH+*212dH+*212eH, *230aH+*230bH+*230cH+*230dH+*230eH, *231aH+*231bH+*231cH+*231dH+*231eH, *233aH+*233bH+*233cH+*233dH+*233eH, *234aH+*234bH+*234cH+*234dH+*234eH, *235aH+*235bH+*235cH+*235dH+*235eH, *236aH+*236bH+*236cH+*236dH+*236eH, *238aH+*238bH+*238cH+*238dH+*238eH, *239aH+*239bH+*239cH+*239dH+*239eH, *242aH+*242bH+*242cH+*242dH+*242eH, *243aH+*243bH+*243cH+*243dH+*243eH, *246aH+*246bH+*246cH+*246dH+*246eH, *249aH+*249bH+*249cH+*249dH+*249eH, *250aH+*250bH+*250cH+*250dH+*250eH, *253aH+*253bH+*253cH+*253dH+*253eH, *254aH+*254bH+*254cH+*254dH+*254eH, *255aH+*255bH+*255cH+*255dH+*255eH, *256aH+*256bH+*256cH+*256dH+*256eH, *269aH+*269bH+*269cH+*269dH+*269eH

### Accessibility >90%, Min Distance to Active Site 6 Angstroms

*1aH+*1bH+*1cH+*1dH+*1eH, *3aH+*3bH+*3cH+*3dH+*3eH, *4aH+*4bH+*4cH+*4dH+*4eH, *9aH+*9bH+*9cH+*9dH+*9eH, *10aH+*10bH+*10cH+*10dH+*10eH, *12aH+*12bH+*12cH+*12dH+*12eH, *18aH+*18bH+*18cH+*18dH+*18eH, *19aH+*19bH+*19cH+*19dH+*19eH, *24aH+*24bH+*24cH+*24dH+*24eH, *37aH+*37bH+*37cH+*37dH+*37eH, *39aH+*39bH+*39cH+*39dH+*39eH, *42aH+*42bH+*42cH+*42dH+*42eH, *44aH+*44bH+*44cH+*44dH+*44eH, *51aH+*51bH+*51cH+*51dH+*51eH, *52aH+*52bH+*52cH+*52dH+*52eH, *54aH+*54bH+*54cH+*54dH+*54eH, *55aH+*55bH+*55cH+*55dH+*55eH, *57aH+*57bH+*57cH+*57dH+*57eH, *59aH+*59bH+*59cH+*59dH+*59eH, *74aH+*74bH+*74cH+*74dH+*74eH, *75aH+*75bH+*75cH+*75dH+*75eH, *76aH+*76bH+*76cH+*76dH+*76eH, *77aH+*77bH+*77cH+*77dH+*77eH, *97aH+*97bH+*97cH+*97dH+*97eH, *99aH+*99bH+*99cH+*99dH+*99eH, *101aH+*101bH+*101cH+*101dH+*101eH, *103aH+*103bH+*103cH+*103dH+*103eH, *107aH+*107bH+*107cH+*107dH+*107eH, *110aH+*110bH+*110cH+*110dH+*110eH, *114aH+*114bH+*114cH+*114dH+*114eH, *115aH+*115bH+*115cH+*115dH+*115eH, *118aH+*118bH+*118cH+*118dH+*118eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *142aH+*142bH+*142cH+*142dH+*142eH, *143aH+*143bH+*143cH+*143dH+*143eH, *153aH+*153bH+*153cH+*153dH+*153eH, *154aH+*154bH+*154cH+*154dH+*154eH, *158aH+*158bH+*158cH+*158dH+*158eH, *164aH+*164bH+*164cH+*164dH+*164eH, *166aH+*166bH+*166cH+*166dH+*166eH, *167aH+*167bH+*167cH+*167dH+*167eH, *176aH+*176bH+*176cH+*176dH+*176eH, *177aH+*177bH+*177cH+*177dH+*177eH, *179aH+*179bH+*179cH+*179dH+*179eH, *180aH+*180bH+*180cH+*180dH+*180eH, *182aH+*182bH+*182cH+*182dH+*182eH, *188aH+*188bH+*188cH+*188dH+*188eH, *198aH+*198bH+*198cH+*198dH+*198eH, *200aH+*200bH+*200cH+*200dH+*200eH, *203aH+*203bH+*203cH+*203dH+*203eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *210aH+*210bH+*210cH+*210dH+*210eH, *212aH+*212bH+*212cH+*212dH+*212eH, *231aH+*231bH+*231cH+*231dH+*231eH, *233aH+*233bH+*233cH+*233dH+*233eH, *234aH+*234bH+*234cH+*234dH+*234eH, *235aH+*235bH+*235cH+*235dH+*235eH, *238aH+*238bH+*238cH+*238dH+*238eH, *239aH+*239bH+*239cH+*239dH+*239eH, *242aH+*242bH+*242cH+*242dH+*242eH, *246aH+*246bH+*246cH+*246dH+*246eH, *249aH+*249bH+*249cH+*249dH+*249eH, *250aH+*250bH+*250cH+*250dH+*250eH, *253aH+*253bH+*253cH+*253dH+*253eH, *254aH+*254bH+*254cH+*254dH+*254eH, *255aH+*255bH+*255cH+*255dH+*255eH, *269aH+*269bH+*269cH+*269dH+*269eH

### Accessibility >100%, Min Distance to Active Site 6 Angstroms

*1aH+*1bH+*1cH+*1dH+*1eH, *3aH+*3bH+*3cH+*3dH+*3eH, *4aH+*4bH+*4cH+*4dH+*4eH, *9aH+*9bH+*9cH+*9dH+*9eH, *10aH+*10bH+*10cH+*10dH+*10eH, *12aH+*12bH+*12cH+*12dH+*12eH, *18aH+*18bH+*18cH+*18dH+*18eH, *19aH+*19bH+*19cH+*19dH+*19eH, *24aH+*24bH+*24cH+*24dH+*24eH, *37aH+*37bH+*37cH+*37dH+*37eH, *39aH+*39bH+*39cH+*39dH+*39eH, *42aH+*42bH+*42cH+*42dH+*42eH, *44aH+*44bH+*44cH+*44dH+*44eH, *51aH+*51bH+*51cH+*51dH+*51eH, *52aH+*52bH+*52cH+*52dH+*52eH, *54aH+*54bH+*54cH+*54dH+*54eH, *57aH+*57bH+*57cH+*57dH+*57eH, *74aH+*74bH+*74cH+*74dH+*74eH, *76aH+*76bH+*76cH+*76dH+*76eH, *97aH+*97bH+*97cH+*97dH+*97eH, *99aH+*99bH+*99cH+*99dH+*99eH, *103aH+*103bH+*103cH+*103dH+*103eH, *107aH+*107bH+*107cH+*107dH+*107eH, *110aH+*110bH+*110cH+*110dH+*110eH, *114aH+*114bH+*114cH+*114dH+*114eH, *115aH+*115bH+*115cH+*115dH+*115eH, *127aH+*127bH+*127cH+*127dH+*127eH, *128aH+*128bH+*128cH+*128dH+*128eH, *129aH+*129bH+*129cH+*129dH+*129eH, *131aH+*131bH+*131cH+*131dH+*131eH, *132aH+*132bH+*132cH+*132dH+*132eH, *134aH+*134bH+*134cH+*134dH+*134eH, *135aH+*135bH+*135cH+*135dH+*135eH, *142aH+*142bH+*142cH+*142dH+*142eH, *143aH+*143bH+*143cH+*143dH+*143eH, *153aH+*153bH+*153cH+*153dH+*153eH, *154aH+*154bH+*154cH+*154dH+*154eH, *164aH+*164bH+*164cH+*164dH+*164eH, *166aH+*166bH+*166cH+*166dH+*166eH, *176aH+*176bH+*176cH+*176dH+*176eH, *177aH+*177bH+*177cH+*177dH+*177eH, *179aH+*179bH+*179cH+*179dH+*179eH, *182aH+*182bH+*182cH+*182dH+*182eH, *188aH+*188bH+*188cH+*188dH+*188eH, *198aH+*198bH+*198cH+*198dH+*198eH, *200aH+*200bH+*200cH+*200dH+*200eH, *203aH+*203bH+*203cH+*203dH+*203eH, *205aH+*205bH+*205cH+*205dH+*205eH, *206aH+*206bH+*206cH+*206dH+*206eH, *207aH+*207bH+*207cH+*207dH+*207eH, *208aH+*208bH+*208cH+*208dH+*208eH, *212aH+*212bH+*212cH+*212dH+*212eH, *231aH+*231bH+*231cH+*231dH+*231eH, *233aH+*233bH+*233cH+*233dH+*233eH, *234aH+*234bH+*234cH+*234dH+*234eH, *238aH+*238bH+*238cH+*238dH+*238eH, *239aH+*239bH+*239cH+*239dH+*239eH, *242aH+*242bH+*242cH+*242dH+*242eH, *246aH+*246bH+*246cH+*246dH+*246eH, *250aH+*250bH+*250cH+*250dH+*250eH, *254aH+*254bH+*254cH+*254dH+*254eH, *255aH+*255bH+*255cH+*255dH+*255eH, *269aH+*269bH+*269cH+*269dH+*269eH

### Accessibility >110%, Min Distance to Active Site 6 Angstroms

*1aH+*1bH+*1cH+*1dH+*1eH, *10aH+*10bH+*10cH+*10dH+*10eH, *12aH+*12bH+*12cH+*12dH+*12eH, *18aH+*18bH+*18cH+*18dH+*18eH, *19aH+*19bH+*19cH+*19dH+*19eH, *37aH+*37bH+*37cH+*37dH+*37eH, *42aH+*42bH+*42cH+*42dH+*42eH, *44aH+*44bH+*44cH+*44dH+*44eH, *51aH+*51bH+*51cH+*51dH+*51eH, *52aH+*52bH+*52cH+*52dH+*52eH, *54aH+*54bH+*54cH+*54dH+*54eH, *57aH+*57bH+*57cH+*57dH+*57eH, *74aH+*74bH+*74cH+*74dH+*74eH, *76aH+*76bH+*76cH+*76dH+*76eH, *97aH+*97bH+*97cH+*97dH+*97eH, *99aH+*99bH+*99cH+*99dH+*99eH, *107aH+*107bH+*107cH+*107dH+*107eH, *114aH+*114bH+*114cH+*114dH+*114eH, *115aH+*115bH+*115cH+*115dH+*115eH, *127aH+*127bH+*127cH+*127dH+*127eH, *129aH+*129bH+*129cH+*129dH+*129eH, *132aH+*132bH+*132cH+*132dH+*132eH, *134aH+*134bH+*134cH+*134dH+*134eH, *135aH+*135bH+*135cH+*135dH+*135eH, *142aH+*142bH+*142cH+*142dH+*142eH, *143aH+*143bH+*143cH+*143dH+*143eH, *153aH+*153bH+*153cH+*153dH+*153eH, *154aH+*154bH+*154cH+*154dH+*154eH, *164aH+*164bH+*164cH+*164dH+*164eH, *166aH+*166bH+*166cH+*166dH+*166eH, *176aH+*176bH+*176cH+*176dH+*176eH, *177aH+*177bH+*177cH+*177dH+*177eH, *179aH+*179bH+*179cH+*179dH+*179eH, *182aH+*182bH+*182cH+*182dH+*182eH, *188aH+*188bH+*188cH+*188dH+*188eH, *198aH+*198bH+*198cH+*198dH+*198eH, *200aH+*200bH+*200cH+*200dH+*200eH, *203aH+*203bH+*203cH+*203dH+*203eH, *205aH+*205bH+*205cH+*205dH+*205eH, *206aH+*206bH+*206cH+*206dH+*206eH, *212aH+*212bH+*212cH+*212dH+*212eH, *231aH+*231bH+*231cH+*231dH+*231eH, *233aH+*233bH+*233cH+*233dH+*233eH, *234aH+*234bH+*234cH+*234dH+*234eH, *238aH+*238bH+*238cH+*238dH+*238eH, *239aH+*239bH+*239cH+*239dH+*239eH, *242aH+*242bH+*242cH+*242dH+*242eH, *246aH+*246bH+*246cH+*246dH+*246eH, *250aH+*250bH+*250cH+*250dH+*250eH, *254aH+*254bH+*254cH+*254dH+*254eH, *255aH+*255bH+*255cH+*255dH+*255eH, *269aH+*269bH+*269cH+*269dH+*269eH

### Accessibility >120%, Min Distance to Active Site 6 Angstroms

*1aH+*1bH+*1cH+*1dH+*1eH, *10aH+*10bH+*10cH+*10dH+*10eH, *18aH+*18bH+*18cH+*18dH+*18eH, *19aH+*19bH+*19cH+*19dH+*19eH, *37aH+*37bH+*37cH+*37dH+*37eH, *42aH+*42bH+*42cH+*42dH+*42eH, *44aH+*44bH+*44cH+*44dH+*44eH, *51aH+*51bH+*51cH+*51dH+*51eH, *52aH+*52bH+*52cH+*52dH+*52eH, *54aH+*54bH+*54cH+*54dH+*54eH, *57aH+*57bH+*57cH+*57dH+*57eH, *74aH+*74bH+*74cH+*74dH+*74eH, *76aH+*76bH+*76cH+*76dH+*76eH, *97aH+*97bH+*97cH+*97dH+*97eH, *99aH+*99bH+*99cH+*99dH+*99eH, *107aH+*107bH+*107cH+*107dH+*107eH, *114aH+*114bH+*114cH+*114dH+*114eH, *115aH+*115bH+*115cH+*115dH+*115eH, *127aH+*127bH+*127cH+*127dH+*127eH, *129aH+*129bH+*129cH+*129dH+*129eH, *134aH+*134bH+*134cH+*134dH+*134eH, *135aH+*135bH+*135cH+*135dH+*135eH, *142aH+*142bH+*142cH+*142dH+*142eH, *143aH+*143bH+*143cH+*143dH+*143eH, *153aH+*153bH+*153cH+*153dH+*153eH, *154aH+*154bH+*154cH+*154dH+*154eH, *164aH+*164bH+*164cH+*164dH+*164eH, *166aH+*166bH+*166cH+*166dH+*166eH, *176aH+*176bH+*176cH+*176dH+*176eH, *177aH+*177bH+*177cH+*177dH+*177eH, *179aH+*179bH+*179cH+*179dH+*179eH, *182aH+*182bH+*182cH+*182dH+*182eH, *188aH+*188bH+*188cH+*188dH+*188eH, *198aH+*198bH+*198cH+*198dH+*198eH, *200aH+*200bH+*200cH+*200dH+*200eH, *203aH+*203bH+*203cH+*203dH+*203eH, *212aH+*212bH+*212cH+*212dH+*212eH, *231aH+*231bH+*231cH+*231dH+*231eH, *233aH+*233bH+*233cH+*233dH+*233eH, *234aH+*234bH+*234cH+*234dH+*234eH, *239aH+*239bH+*239cH+*239dH+*239eH, *246aH+*246bH+*246cH+*246dH+*246eH, *250aH+*250bH+*250cH+*250dH+*250eH, *254aH+*254bH+*254cH+*254dH+*254eH, *255aH+*255bH+*255cH+*255dH+*255eH, *269aH+*269bH+*269cH+*269dH+*269eH

### Accessibility >130%, Min Distance to Active Site 6 Angstroms

*1aH+*1bH+*1cH+*1dH+*1eH, *18aH+*18bH+*18cH+*18dH+*18eH, *19aH+*19bH+*19cH+*19dH+*19eH, *37aH+*37bH+*37cH+*37dH+*37eH, *42aH+*42bH+*42cH+*42dH+*42eH, *44aH+*44bH+*44cH+*44dH+*44eH, *51aH+*51bH+*51cH+*51dH+*51eH, *52aH+*52bH+*52cH+*52dH+*52eH, *54aH+*54bH+*54cH+*54dH+*54eH, *57aH+*57bH+*57cH+*57dH+*57eH, *74aH+*74bH+*74cH+*74dH+*74eH, *76aH+*76bH+*76cH+*76dH+*76eH, *97aH+*97bH+*97cH+*97dH+*97eH, *107aH+*107bH+*107cH+*107dH+*107eH, *114aH+*114bH+*114cH+*114dH+*114eH, *127aH+*127bH+*127cH+*127dH+*127eH, *129aH+*129bH+*129cH+*129dH+*129eH, *135aH+*135bH+*135cH+*135dH+*135eH, *142aH+*142bH+*142cH+*142dH+*142eH, *143aH+*143bH+*143cH+*143dH+*143eH, *164aH+*164bH+*164cH+*164dH+*164eH, *176aH+*176bH+*176cH+*176dH+*176eH, *177aH+*177bH+*177cH+*177dH+*177eH, *182aH+*182bH+*182cH+*182dH+*182eH, *200aH+*200bH+*200cH+*200dH+*200eH, *203aH+*203bH+*203cH+*203dH+*203eH, *212aH+*212bH+*212cH+*212dH+*212eH, *231aH+*231bH+*231cH+*231dH+*231eH, *233aH+*233bH+*233cH+*233dH+*233eH, *234aH+*234bH+*234cH+*234dH+*234eH, *239aH+*239bH+*239cH+*239dH+*239eH, *246aH+*246bH+*246cH+*246dH+*246eH, *250aH+*250bH+*250cH+*250dH+*250eH, *254aH+*254bH+*254cH+*254dH+*254eH, *269aH+*269bH+*269cH+*269dH+*269eH

### Accessibility >140%, Min Distance to Active Site 6 Angstroms

*1aH+*1bH+*1cH+*1dH+*1eH, *18aH+*18bH+*18cH+*18dH+*18eH, *19aH+*19bH+*19cH+*19dH+*19eH, *37aH+*37bH+*37cH+*37dH+*37eH, *42aH+*42bH+*42cH+*42dH+*42eH, *44aH+*44bH+*44cH+*44dH+*44eH, *51aH+*51bH+*51cH+*51dH+*51eH, *54aH+*54bH+*54cH+*54dH+*54eH, *57aH+*57bH+*57cH+*57dH+*57eH, *74aH+*74bH+*74cH+*74dH+*74eH, *76aH+*76bH+*76cH+*76dH+*76eH, *97aH+*97bH+*97cH+*97dH+*97eH, *107aH+*107bH+*107cH+*107dH+*107eH, *114aH+*114bH+*114cH+*114dH+*114eH, *127aH+*127bH+*127cH+*127dH+*127eH, *129aH+*129bH+*129cH+*129dH+*129eH, *135aH+*135bH+*135cH+*135dH+*135eH, *142aH+*142bH+*142cH+*142dH+*142eH, *164aH+*164bH+*164cH+*164dH+*164eH, *177aH+*177bH+*177cH+*177dH+*177eH, *182aH+*182bH+*182cH+*182dH+*182eH, *212aH+*212bH+*212cH+*212dH+*212eH, *231aH+*231bH+*231cH+*231dH+*231eH, *233aH+*233bH+*233cH+*233dH+*233eH, *234aH+*234bH+*234cH+*234dH+*234eH, *239aH+*239bH+*239cH+*239dH+*239eH, *246aH+*246bH+*246cH+*246dH+*246eH, *250aH+*250bH+*250cH+*250dH+*250eH, *254aH+*254bH+*254cH+*254dH+*254eH, *269aH+*269bH+*269cH+*269dH+*269eH

### Accessibility >40%, Min Distance to Active Site 6 Angstroms

*1aH+*1bH+*1cH+*1dH+*1eH+*1fH, *2aH+*2bH+*2cH+*2dH+*2eH+*2fH, *3aH+*3bH+*3cH+*3dH+*3eH+*3fH, *4aH+*4bH+*4cH+*4dH+*4eH+*4fH, *6aH+*6bH+*6cH+*6dH+*6eH+*6fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *14aH+*14bH+*14cH+*14dH+*14eH+*14fH, *15aH+*15bH+*15cH+*15dH+*15eH+*15fH, *17aH+*17bH+*17cH+*17dH+*17eH+*17fH, *18aH+*18bH+*18cH+*18dH+*18eH+*18fH, *19aH+*19bH+*19cH+*19dH+*19eH+*19fH, *20aH+*20bH+*20cH+*20dH+*20eH+*20fH, *22aH+*22bH+*22cH+*22dH+*22eH+*22fH, *24aH+*24bH+*24cH+*24dH+*24eH+*24fH, *25aH+*25bH+*25cH+*25dH+*25eH+*25fH, *27aH+*27bH+*27cH+*27dH+*27eH+*27fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *43aH+*43bH+*43cH+*43dH+*43eH+*43fH, *44aH+*44bH+*44cH+*44dH+*44eH+*44fH, *45aH+*45bH+*45cH+*45dH+*45eH+*45fH, *46aH+*46bH+*46cH+*46dH+*46eH+*46fH, *48aH+*48bH+*48cH+*48dH+*48eH+*48fH, *49aH+*49bH+*49cH+*49dH+*49eH+*49fH, *51aH+*51bH+*51cH+*51dH+*51eH+*51fH, *52aH+*52bH+*52cH+*52dH+*52eH+*52fH, *53aH+*53bH+*53cH+*53dH+*53eH+*53fH, *54aH+*54bH+*54cH+*54dH+*54eH+*54fH, *55aH+*55bH+*55cH+*55dH+*55eH+*55fH, *56aH+*56bH+*56cH+*56dH+*56eH+*56fH, *57aH+*57bH+*57cH+*57dH+*57eH+*57fH, *59aH+*59bH+*59cH+*59dH+*59eH+*59fH, *73aH+*73bH+*73cH+*73dH+*73eH+*73fH, *74aH+*74bH+*74cH+*74dH+*74eH+*74fH, *75aH+*75bH+*75cH+*75dH+*75eH+*75fH, *76aH+*76bH+*76cH+*76dH+*76eH+*76fH, *77aH+*77bH+*77cH+*77dH+*77eH+*77fH, *85aH+*85bH+*85cH+*85dH+*85eH+*85fH, *87aH+*87bH+*87cH+*87dH+*87eH+*87fH, *89aH+*89bH+*89cH+*89dH+*89eH+*89fH, *96aH+*96bH+*96cH+*96dH+*96eH+*96fH, *97aH+*97bH+*97cH+*97dH+*97eH+*97fH, *98aH+*98bH+*98cH+*98dH+*98eH+*98fH, *99aH+*99bH+*99cH+*99dH+*99eH+*99fH, *100aH+*100bH+*100cH+*100dH+*100eH+*100fH, *101aH+*101bH+*101cH+*101dH+*101eH+*101fH, *102aH+*102bH+*102cH+*102dH+*102eH+*102fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *111aH+*111bH+*111cH+*111dH+*111eH+*111fH, *114aH+*114bH+*114cH+*114dH+*114eH+*114fH, *115aH+*115bH+*115cH+*115dH+*115eH+*115fH, *116aH+*116bH+*116cH+*116dH+*116eH+*116fH, *118aH+*118bH+*118cH+*118dH+*118eH+*118fH, *125aH+*125bH+*125cH+*125dH+*125eH+*125fH, *126aH+*126bH+*126cH+*126dH+*126eH+*126fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *138aH+*138bH+*138cH+*138dH+*138eH+*138fH, *139aH+*139bH+*139cH+*139dH+*139eH+*139fH, *142aH+*142bH+*142cH+*142dH+*142eH+*142fH, *143aH+*143bH+*143cH+*143dH+*143eH+*143fH, *144aH+*144bH+*144cH+*144dH+*144eH+*144fH, *153aH+*153bH+*153cH+*153dH+*153eH+*153fH, *154aH+*154bH+*154cH+*154dH+*154eH+*154fH, *155aH+*155bH+*155cH+*155dH+*155eH+*155fH, *156aH+*156bH+*156cH+*156dH+*156eH+*156fH, *157aH+*157bH+*157cH+*157dH+*157eH+*157fH, *158aH+*158bH+*158cH+*158dH+*158eH+*158fH, *161aH+*161bH+*161cH+*161dH+*161eH+*161fH, *164aH+*164bH+*164cH+*164dH+*164eH+*164fH, *166aH+*166bH+*166cH+*166dH+*166eH+*166fH, *167aH+*167bH+*167cH+*167dH+*167eH+*167fH, *175aH+*175bH+*175cH+*175dH+*175eH+*175fH, *176aH+*176bH+*176cH+*176dH+*176eH+*176fH, *177aH+*177bH+*177cH+*177dH+*177eH+*177fH, *178aH+*178bH+*178cH+*178dH+*178eH+*178fH, *179aH+*179bH+*179cH+*179dH+*179eH+*179fH, *180aH+*180bH+*180cH+*180dH+*180eH+*180fH, *182aH+*182bH+*182cH+*182dH+*182eH+*182fH, *183aH+*183bH+*183cH+*183dH+*183eH+*183fH, *186aH+*186bH+*186cH+*186dH+*186eH+*186fH, *188aH+*188bH+*188cH+*188dH+*188eH+*188fH, *189aH+*189bH+*189cH+*189dH+*189eH+*189fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *204aH+*204bH+*204cH+*204dH+*204eH+*204fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *210aH+*210bH+*210cH+*210dH+*210eH+*210fH, *211aH+*211bH+*211cH+*211dH+*211eH+*211fH, *212aH+*212bH+*212cH+*212dH+*212eH+*212fH, *229aH+*229bH+*229cH+*229dH+*229eH+*229fH, *230aH+*230bH+*230cH+*230dH+*230eH+*230fH, *231aH+*231bH+*231cH+*231dH+*231eH+*231fH, *232aH+*232bH+*232cH+*232dH+*232eH+*232fH, *233aH+*233bH+*233cH+*233dH+*233eH+*233fH, *234aH+*234bH+*234cH+*234dH+*234eH+*234fH, *235aH+*235bH+*235cH+*235dH+*235eH+*235fH, *236aH+*236bH+*236cH+*236dH+*236eH+*236fH, *238aH+*238bH+*238cH+*238dH+*238eH+*238fH, *239aH+*239bH+*239cH+*239dH+*239eH+*239fH, *241aH+*241bH+*241cH+*241dH+*241eH+*241fH, *242aH+*242bH+*242cH+*242dH+*242eH+*242fH, *243aH+*243bH+*243cH+*243dH+*243eH+*243fH, *245aH+*245bH+*245cH+*245dH+*245eH+*245fH, *246aH+*246bH+*246cH+*246dH+*246eH+*246fH, *247aH+*247bH+*247cH+*247dH+*247eH+*247fH, *249aH+*249bH+*249cH+*249dH+*249eH+*249fH, *250aH+*250bH+*250cH+*250dH+*250eH+*250fH, *251aH+*251bH+*251cH+*251dH+*251eH+*251fH, *252aH+*252bH+*252cH+*252dH+*252eH+*252fH, *253aH+*253bH+*253cH+*253dH+*253eH+*253fH, *254aH+*254bH+*254cH+*254dH+*254eH+*254fH, *255aH+*255bH+*255cH+*255dH+*255eH+*255fH, *256aH+*256bH+*256cH+*256dH+*256eH+*256fH, *257aH+*257bH+*257cH+*257dH+*257eH+*257fH, *259aH+*259bH+*259cH+*259dH+*259eH+*259fH, *265aH+*265bH+*265cH+*265dH+*265eH+*265fH, *269aH+*269bH+*269cH+*269dH+*269eH+*269fH

### Accessibility >45%, Min Distance to Active Site 6 Angstroms

*1aH+*1bH+*1cH+*1dH+*1eH+*1fH, *2aH+*2bH+*2cH+*2dH+*2eH+*2fH, *3aH+*3bH+*3cH+*3dH+*3eH+*3fH, *4aH+*4bH+*4cH+*4dH+*4eH+*4fH, *6aH+*6bH+*6cH+*6dH+*6eH+*6fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *14aH+*14bH+*14cH+*14dH+*14eH+*14fH, *15aH+*15bH+*15cH+*15dH+*15eH+*15fH, *17aH+*17bH+*17cH+*17dH+*17eH+*17fH, *18aH+*18bH+*18cH+*18dH+*18eH+*18fH, *19aH+*19bH+*19cH+*19dH+*19eH+*19fH, *20aH+*20bH+*20cH+*20dH+*20eH+*20fH, *22aH+*22bH+*22cH+*22dH+*22eH+*22fH, *24aH+*24bH+*24cH+*24dH+*24eH+*24fH, *25aH+*25bH+*25cH+*25dH+*25eH+*25fH, *27aH+*27bH+*27cH+*27dH+*27eH+*27fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *43aH+*43bH+*43cH+*43dH+*43eH+*43fH, *44aH+*44bH+*44cH+*44dH+*44eH+*44fH, *45aH+*45bH+*45cH+*45dH+*45eH+*45fH, *46aH+*46bH+*46cH+*46dH+*46eH+*46fH, *48aH+*48bH+*48cH+*48dH+*48eH+*48fH, *49aH+*49bH+*49cH+*49dH+*49eH+*49fH, *51aH+*51bH+*51cH+*51dH+*51eH+*51fH, *52aH+*52bH+*52cH+*52dH+*52eH+*52fH, *53aH+*53bH+*53cH+*53dH+*53eH+*53fH, *54aH+*54bH+*54cH+*54dH+*54eH+*54fH, *55aH+*55bH+*55cH+*55dH+*55eH+*55fH, *56aH+*56bH+*56cH+*56dH+*56eH+*56fH, *57aH+*57bH+*57cH+*57dH+*57eH+*57fH, *59aH+*59bH+*59cH+*59dH+*59eH+*59fH, *73aH+*73bH+*73cH+*73dH+*73eH+*73fH, *74aH+*74bH+*74cH+*74dH+*74eH+*74fH, *75aH+*75bH+*75cH+*75dH+*75eH+*75fH, *76aH+*76bH+*76cH+*76dH+*76eH+*76fH, *77aH+*77bH+*77cH+*77dH+*77eH+*77fH, *85aH+*85bH+*85cH+*85dH+*85eH+*85fH, *87aH+*87bH+*87cH+*87dH+*87eH+*87fH, *96aH+*96bH+*96cH+*96dH+*96eH+*96fH, *97aH+*97bH+*97cH+*97dH+*97eH+*97fH, *98aH+*98bH+*98cH+*98dH+*98eH+*98fH, *99aH+*99bH+*99cH+*99dH+*99eH+*99fH, *100aH+*100bH+*100cH+*100dH+*100eH+*100fH, *101aH+*101bH+*101cH+*101dH+*101eH+*101fH, *102aH+*102bH+*102cH+*102dH+*102eH+*102fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *111aH+*111bH+*111cH+*111dH+*111eH+*111fH, *114aH+*114bH+*114cH+*114dH+*114eH+*114fH, *115aH+*115bH+*115cH+*115dH+*115eH+*115fH, *116aH+*116bH+*116cH+*116dH+*116eH+*116fH, *118aH+*118bH+*118cH+*118dH+*118eH+*118fH, *125aH+*125bH+*125cH+*125dH+*125eH+*125fH, *126aH+*126bH+*126cH+*126dH+*126eH+*126fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *138aH+*138bH+*138cH+*138dH+*138eH+*138fH, *139aH+*139bH+*139cH+*139dH+*139eH+*139fH, *142aH+*142bH+*142cH+*142dH+*142eH+*142fH, *143aH+*143bH+*143cH+*143dH+*143eH+*143fH, *144aH+*144bH+*144cH+*144dH+*144eH+*144fH, *153aH+*153bH+*153cH+*153dH+*153eH+*153fH, *154aH+*154bH+*154cH+*154dH+*154eH+*154fH, *156aH+*156bH+*156cH+*156dH+*156eH+*156fH, *157aH+*157bH+*157cH+*157dH+*157eH+*157fH, *158aH+*158bH+*158cH+*158dH+*158eH+*158fH, *161aH+*161bH+*161cH+*161dH+*161eH+*161fH, *164aH+*164bH+*164cH+*164dH+*164eH+*164fH, *166aH+*166bH+*166cH+*166dH+*166eH+*166fH, *167aH+*167bH+*167cH+*167dH+*167eH+*167fH, *175aH+*175bH+*175cH+*175dH+*175eH+*175fH, *176aH+*176bH+*176cH+*176dH+*176eH+*176fH, *177aH+*177bH+*177cH+*177dH+*177eH+*177fH, *178aH+*178bH+*178cH+*178dH+*178eH+*178fH, *179aH+*179bH+*179cH+*179dH+*179eH+*179fH, *180aH+*180bH+*180cH+*180dH+*180eH+*180fH, *182aH+*182bH+*182cH+*182dH+*182eH+*182fH, *183aH+*183bH+*183cH+*183dH+*183eH+*183fH, *186aH+*186bH+*186cH+*186dH+*186eH+*186fH, *188aH+*188bH+*188cH+*188dH+*188eH+*188fH, *189aH+*189bH+*189cH+*189dH+*189eH+*189fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *204aH+*204bH+*204cH+*204dH+*204eH+*204fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *210aH+*210bH+*210cH+*210dH+*210eH+*210fH, *211aH+*211bH+*211cH+*211dH+*211eH+*211fH, *212aH+*212bH+*212cH+*212dH+*212eH+*212fH, *229aH+*229bH+*229cH+*229dH+*229eH+*229fH, *230aH+*230bH+*230cH+*230dH+*230eH+*230fH, *231aH+*231bH+*231cH+*231dH+*231eH+*231fH, *232aH+*232bH+*232cH+*232dH+*232eH+*232fH, *233aH+*233bH+*233cH+*233dH+*233eH+*233fH, *234aH+*234bH+*234cH+*234dH+*234eH+*234fH, *235aH+*235bH+*235cH+*235dH+*235eH+*235fH, *236aH+*236bH+*236cH+*236dH+*236eH+*236fH, *238aH+*238bH+*238cH+*238dH+*238eH+*238fH, *239aH+*239bH+*239cH+*239dH+*239eH+*239fH, *241aH+*241bH+*241cH+*241dH+*241eH+*241fH, *242aH+*242bH+*242cH+*242dH+*242eH+*242fH, *243aH+*243bH+*243cH+*243dH+*243eH+*243fH, *245aH+*245bH+*245cH+*245dH+*245eH+*245fH, *246aH+*246bH+*246cH+*246dH+*246eH+*246fH, *249aH+*249bH+*249cH+*249dH+*249eH+*249fH, *250aH+*250bH+*250cH+*250dH+*250eH+*250fH, *251aH+*251bH+*251cH+*251dH+*251eH+*251fH, *252aH+*252bH+*252cH+*252dH+*252eH+*252fH, *253aH+*253bH+*253cH+*253dH+*253eH+*253fH, *254aH+*254bH+*254cH+*254dH+*254eH+*254fH, *255aH+*255bH+*255cH+*255dH+*255eH+*255fH, *256aH+*256bH+*256cH+*256dH+*256eH+*256fH, *257aH+*257bH+*257cH+*257dH+*257eH+*257fH, *259aH+*259bH+*259cH+*259dH+*259eH+*259fH, *269aH+*269bH+*269cH+*269dH+*269eH+*269fH

### Accessibility >50%, Min Distance to Active Site 6 Angstroms

*1aH+*1bH+*1cH+*1dH+*1eH+*1fH, *2aH+*2bH+*2cH+*2dH+*2eH+*2fH, *3aH+*3bH+*3cH+*3dH+*3eH+*3fH, *4aH+*4bH+*4cH+*4dH+*4eH+*4fH, *6aH+*6bH+*6cH+*6dH+*6eH+*6fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *14aH+*14bH+*14cH+*14dH+*14eH+*14fH, *15aH+*15bH+*15cH+*15dH+*15eH+*15fH, *18aH+*18bH+*18cH+*18dH+*18eH+*18fH, *19aH+*19bH+*19cH+*19dH+*19eH+*19fH, *20aH+*20bH+*20cH+*20dH+*20eH+*20fH, *24aH+*24bH+*24cH+*24dH+*24eH+*24fH, *25aH+*25bH+*25cH+*25dH+*25eH+*25fH, *27aH+*27bH+*27cH+*27dH+*27eH+*27fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *43aH+*43bH+*43cH+*43dH+*43eH+*43fH, *44aH+*44bH+*44cH+*44dH+*44eH+*44fH, *49aH+*49bH+*49cH+*49dH+*49eH+*49fH, *51aH+*51bH+*51cH+*51dH+*51eH+*51fH, *52aH+*52bH+*52cH+*52dH+*52eH+*52fH, *53aH+*53bH+*53cH+*53dH+*53eH+*53fH, *54aH+*54bH+*54cH+*54dH+*54eH+*54fH, *55aH+*55bH+*55cH+*55dH+*55eH+*55fH, *56aH+*56bH+*56cH+*56dH+*56eH+*56fH, *57aH+*57bH+*57cH+*57dH+*57eH+*57fH, *59aH+*59bH+*59cH+*59dH+*59eH+*59fH, *73aH+*73bH+*73cH+*73dH+*73eH+*73fH, *74aH+*74bH+*74cH+*74dH+*74eH+*74fH, *75aH+*75bH+*75cH+*75dH+*75eH+*75fH, *76aH+*76bH+*76cH+*76dH+*76eH+*76fH, *77aH+*77bH+*77cH+*77dH+*77eH+*77fH, *85aH+*85bH+*85cH+*85dH+*85eH+*85fH, *87aH+*87bH+*87cH+*87dH+*87eH+*87fH, *96aH+*96bH+*96cH+*96dH+*96eH+*96fH, *97aH+*97bH+*97cH+*97dH+*97eH+*97fH, *98aH+*98bH+*98cH+*98dH+*98eH+*98fH, *99aH+*99bH+*99cH+*99dH+*99eH+*99fH, *100aH+*100bH+*100cH+*100dH+*100eH+*100fH, *101aH+*101bH+*101cH+*101dH+*101eH+*101fH, *102aH+*102bH+*102cH+*102dH+*102eH+*102fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *111aH+*111bH+*111cH+*111dH+*111eH+*111fH, *114aH+*114bH+*114cH+*114dH+*114eH+*114fH, *115aH+*115bH+*115cH+*115dH+*115eH+*115fH, *116aH+*116bH+*116cH+*116dH+*116eH+*116fH, *118aH+*118bH+*118cH+*118dH+*118eH+*118fH, *125aH+*125bH+*125cH+*125dH+*125eH+*125fH, *126aH+*126bH+*126cH+*126dH+*126eH+*126fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *138aH+*138bH+*138cH+*138dH+*138eH+*138fH, *142aH+*142bH+*142cH+*142dH+*142eH+*142fH, *143aH+*143bH+*143cH+*143dH+*143eH+*143fH, *144aH+*144bH+*144cH+*144dH+*144eH+*144fH, *153aH+*153bH+*153cH+*153dH+*153eH+*153fH, *154aH+*154bH+*154cH+*154dH+*154eH+*154fH, *156aH+*156bH+*156cH+*156dH+*156eH+*156fH, *157aH+*157bH+*157cH+*157dH+*157eH+*157fH, *158aH+*158bH+*158cH+*158dH+*158eH+*158fH, *164aH+*164bH+*164cH+*164dH+*164eH+*164fH, *166aH+*166bH+*166cH+*166dH+*166eH+*166fH, *167aH+*167bH+*167cH+*167dH+*167eH+*167fH, *175aH+*175bH+*175cH+*175dH+*175eH+*175fH, *176aH+*176bH+*176cH+*176dH+*176eH+*176fH, *177aH+*177bH+*177cH+*177dH+*177eH+*177fH, *178aH+*178bH+*178cH+*178dH+*178eH+*178fH, *179aH+*179bH+*179cH+*179dH+*179eH+*179fH, *180aH+*180bH+*180cH+*180dH+*180eH+*180fH, *182aH+*182bH+*182cH+*182dH+*182eH+*182fH, *183aH+*183bH+*183cH+*183dH+*183eH+*183fH, *186aH+*186bH+*186cH+*186dH+*186eH+*186fH, *188aH+*188bH+*188cH+*188dH+*188eH+*188fH, *189aH+*189bH+*189cH+*189dH+*189eH+*189fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *210aH+*210bH+*210cH+*210dH+*210eH+*210fH, *211aH+*211bH+*211cH+*211dH+*211eH+*211fH, *212aH+*212bH+*212cH+*212dH+*212eH+*212fH, *229aH+*229bH+*229cH+*229dH+*229eH+*229fH, *230aH+*230bH+*230cH+*230dH+*230eH+*230fH, *231aH+*231bH+*231cH+*231dH+*231eH+*231fH, *232aH+*232bH+*232cH+*232dH+*232eH+*232fH, *233aH+*233bH+*233cH+*233dH+*233eH+*233fH, *234aH+*234bH+*234cH+*234dH+*234eH+*234fH, *235aH+*235bH+*235cH+*235dH+*235eH+*235fH, *236aH+*236bH+*236cH+*236dH+*236eH+*236fH, *238aH+*238bH+*238cH+*238dH+*238eH+*238fH, *239aH+*239bH+*239cH+*239dH+*239eH+*239fH, *241aH+*241bH+*241cH+*241dH+*241eH+*241fH, *242aH+*242bH+*242cH+*242dH+*242eH+*242fH, *243aH+*243bH+*243cH+*243dH+*243eH+*243fH, *245aH+*245bH+*245cH+*245dH+*245eH+*245fH, *246aH+*246bH+*246cH+*246dH+*246eH+*246fH, *249aH+*249bH+*249cH+*249dH+*249eH+*249fH, *250aH+*250bH+*250cH+*250dH+*250eH+*250fH, *251aH+*251bH+*251cH+*251dH+*251eH+*251fH, *252aH+*252bH+*252cH+*252dH+*252eH+*252fH, *253aH+*253bH+*253cH+*253dH+*253eH+*253fH, *254aH+*254bH+*254cH+*254dH+*254eH+*254fH, *255aH+*255bH+*255cH+*255dH+*255eH+*255fH, *256aH+*256bH+*256cH+*256dH+*256eH+*256fH, *257aH+*257bH+*257cH+*257dH+*257eH+*257fH, *259aH+*259bH+*259cH+*259dH+*259eH+*259fH, *269aH+*269bH+*269cH+*269dH+*269eH+*269fH

### Accessibility >60%, Min Distance to Active Site 6 Angstroms

*1aH+*1bH+*1cH+*1dH+*1eH+*1fH, *2aH+*2bH+*2cH+*2dH+*2eH+*2fH, *3aH+*3bH+*3cH+*3dH+*3eH+*3fH, *4aH+*4bH+*4cH+*4dH+*4eH+*4fH, *6aH+*6bH+*6cH+*6dH+*6eH+*6fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *14aH+*14bH+*14cH+*14dH+*14eH+*14fH, *18aH+*18bH+*18cH+*18dH+*18eH+*18fH, *19aH+*19bH+*19cH+*19dH+*19eH+*19fH, *20aH+*20bH+*20cH+*20dH+*20eH+*20fH, *24aH+*24bH+*24cH+*24dH+*24eH+*24fH, *25aH+*25bH+*25cH+*25dH+*25eH+*25fH, *27aH+*27bH+*27cH+*27dH+*27eH+*27fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *43aH+*43bH+*43cH+*43dH+*43eH+*43fH, *44aH+*44bH+*44cH+*44dH+*44eH+*44fH, *49aH+*49bH+*49cH+*49dH+*49eH+*49fH, *51aH+*51bH+*51cH+*51dH+*51eH+*51fH, *52aH+*52bH+*52cH+*52dH+*52eH+*52fH, *53aH+*53bH+*53cH+*53dH+*53eH+*53fH, *54aH+*54bH+*54cH+*54dH+*54eH+*54fH, *55aH+*55bH+*55cH+*55dH+*55eH+*55fH, *56aH+*56bH+*56cH+*56dH+*56eH+*56fH, *57aH+*57bH+*57cH+*57dH+*57eH+*57fH, *59aH+*59bH+*59cH+*59dH+*59eH+*59fH, *73aH+*73bH+*73cH+*73dH+*73eH+*73fH, *74aH+*74bH+*74cH+*74dH+*74eH+*74fH, *75aH+*75bH+*75cH+*75dH+*75eH+*75fH, *76aH+*76bH+*76cH+*76dH+*76eH+*76fH, *77aH+*77bH+*77cH+*77dH+*77eH+*77fH, *85aH+*85bH+*85cH+*85dH+*85eH+*85fH, *97aH+*97bH+*97cH+*97dH+*97eH+*97fH, *99aH+*99bH+*99cH+*99dH+*99eH+*99fH, *101aH+*101bH+*101cH+*101dH+*101eH+*101fH, *102aH+*102bH+*102cH+*102dH+*102eH+*102fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *111aH+*111bH+*111cH+*111dH+*111eH+*111fH, *114aH+*114bH+*114cH+*114dH+*114eH+*114fH, *115aH+*115bH+*115cH+*115dH+*115eH+*115fH, *116aH+*116bH+*116cH+*116dH+*116eH+*116fH, *118aH+*118bH+*118cH+*118dH+*118eH+*118fH, *125aH+*125bH+*125cH+*125dH+*125eH+*125fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *138aH+*138bH+*138cH+*138dH+*138eH+*138fH, *142aH+*142bH+*142cH+*142dH+*142eH+*142fH, *143aH+*143bH+*143cH+*143dH+*143eH+*143fH, *153aH+*153bH+*153cH+*153dH+*153eH+*153fH, *154aH+*154bH+*154cH+*154dH+*154eH+*154fH, *156aH+*156bH+*156cH+*156dH+*156eH+*156fH, *157aH+*157bH+*157cH+*157dH+*157eH+*157fH, *158aH+*158bH+*158cH+*158dH+*158eH+*158fH, *164aH+*164bH+*164cH+*164dH+*164eH+*164fH, *166aH+*166bH+*166cH+*166dH+*166eH+*166fH, *167aH+*167bH+*167cH+*167dH+*167eH+*167fH, *176aH+*176bH+*176cH+*176dH+*176eH+*176fH, *177aH+*177bH+*177cH+*177dH+*177eH+*177fH, *178aH+*178bH+*178cH+*178dH+*178eH+*178fH, *179aH+*179bH+*179cH+*179dH+*179eH+*179fH, *180aH+*180bH+*180cH+*180dH+*180eH+*180fH, *182aH+*182bH+*182cH+*182dH+*182eH+*182fH, *183aH+*183bH+*183cH+*183dH+*183eH+*183fH, *186aH+*186bH+*186cH+*186dH+*186eH+*186fH, *188aH+*188bH+*188cH+*188dH+*188eH+*188fH, *189aH+*189bH+*189cH+*189dH+*189eH+*189fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *210aH+*210bH+*210cH+*210dH+*210eH+*210fH, *211aH+*211bH+*211cH+*211dH+*211eH+*211fH, *212aH+*212bH+*212cH+*212dH+*212eH+*212fH, *229aH+*229bH+*229cH+*229dH+*229eH+*229fH, *230aH+*230bH+*230cH+*230dH+*230eH+*230fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *235aH+*235bH+*235cH+*235dH+*235eH+*235fH, *236aH+*236bH+*236cH+*236dH+*236eH+*236fH, *238aH+*238bH+*238cH+*238dH+*238eH+*238fH, *239aH+*239bH+*239cH+*239dH+*239eH+*239fH, *241aH+*241bH+*241cH+*241dH+*241eH+*241fH, *242aH+*242bH+*242cH+*242dH+*242eH+*242fH, *243aH+*243bH+*243cH+*243dH+*243eH+*243fH, *245aH+*245bH+*245cH+*245dH+*245eH+*245fH, *246aH+*246bH+*246cH+*246dH+*246eH+*246fH, *249aH+*249bH+*249cH+*249dH+*249eH+*249fH, *250aH+*250bH+*250cH+*250dH+*250eH+*250fH, *251aH+*251bH+*251cH+*251dH+*251eH+*251fH, *252aH+*252bH+*252cH+*252dH+*252eH+*252fH, *253aH+*253bH+*253cH+*253dH+*253eH+*253fH, *254aH+*254bH+*254cH+*254dH+*254eH+*254fH, *255aH+*255bH+*255cH+*255dH+*255eH+*255fH, *256aH+*256bH+*256cH+*256dH+*256eH+*256fH, *269aH+*269bH+*269cH+*269dH+*269eH+*269fH

### Accessibility >70%, Min Distance to Active Site 6 Angstroms

*1aH+*1bH+*1cH+*1dH+*1eH+*1fH, *2aH+*2bH+*2cH+*2dH+*2eH+*2fH, *3aH+*3bH+*3cH+*3dH+*3eH+*3fH, *4aH+*4bH+*4cH+*4dH+*4eH+*4fH, *6aH+*6bH+*6cH+*6dH+*6eH+*6fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *14aH+*14bH+*14cH+*14dH+*14eH+*14fH, *18aH+*18bH+*18cH+*18dH+*18eH+*18fH, *19aH+*19bH+*19cH+*19dH+*19eH+*19fH, *20aH+*20bH+*20cH+*20dH+*20eH+*20fH, *24aH+*24bH+*24cH+*24dH+*24eH+*24fH, *25aH+*25bH+*25cH+*25dH+*25eH+*25fH, *27aH+*27bH+*27cH+*27dH+*27eH+*27fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *44aH+*44bH+*44cH+*44dH+*44eH+*44fH, *49aH+*49bH+*49cH+*49dH+*49eH+*49fH, *51aH+*51bH+*51cH+*51dH+*51eH+*51fH, *52aH+*52bH+*52cH+*52dH+*52eH+*52fH, *53aH+*53bH+*53cH+*53dH+*53eH+*53fH, *54aH+*54bH+*54cH+*54dH+*54eH+*54fH, *55aH+*55bH+*55cH+*55dH+*55eH+*55fH, *56aH+*56bH+*56cH+*56dH+*56eH+*56fH, *57aH+*57bH+*57cH+*57dH+*57eH+*57fH, *59aH+*59bH+*59cH+*59dH+*59eH+*59fH, *73aH+*73bH+*73cH+*73dH+*73eH+*73fH, *74aH+*74bH+*74cH+*74dH+*74eH+*74fH, *75aH+*75bH+*75cH+*75dH+*75eH+*75fH, *76aH+*76bH+*76cH+*76dH+*76eH+*76fH, *77aH+*77bH+*77cH+*77dH+*77eH+*77fH, *85aH+*85bH+*85cH+*85dH+*85eH+*85fH, *97aH+*97bH+*97cH+*97dH+*97eH+*97fH, *99aH+*99bH+*99cH+*99dH+*99eH+*99fH, *101aH+*101bH+*101cH+*101dH+*101eH+*101fH, *102aH+*102bH+*102cH+*102dH+*102eH+*102fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *111aH+*111bH+*111cH+*111dH+*111eH+*111fH, *114aH+*114bH+*114cH+*114dH+*114eH+*114fH, *115aH+*115bH+*115cH+*115dH+*115eH+*115fH, *116aH+*116bH+*116cH+*116dH+*116eH+*116fH, *118aH+*118bH+*118cH+*118dH+*118eH+*118fH, *125aH+*125bH+*125cH+*125dH+*125eH+*125fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *138aH+*138bH+*138cH+*138dH+*138eH+*138fH, *142aH+*142bH+*142cH+*142dH+*142eH+*142fH, *143aH+*143bH+*143cH+*143dH+*143eH+*143fH, *153aH+*153bH+*153cH+*153dH+*153eH+*153fH, *154aH+*154bH+*154cH+*154dH+*154eH+*154fH, *158aH+*158bH+*158cH+*158dH+*158eH+*158fH, *164aH+*164bH+*164cH+*164dH+*164eH+*164fH, *166aH+*166bH+*166cH+*166dH+*166eH+*166fH, *167aH+*167bH+*167cH+*167dH+*167eH+*167fH, *176aH+*176bH+*176cH+*176dH+*176eH+*176fH, *177aH+*177bH+*177cH+*177dH+*177eH+*177fH, *178aH+*178bH+*178cH+*178dH+*178eH+*178fH, *179aH+*179bH+*179cH+*179dH+*179eH+*179fH, *180aH+*180bH+*180cH+*180dH+*180eH+*180fH, *182aH+*182bH+*182cH+*182dH+*182eH+*182fH, *183aH+*183bH+*183cH+*183dH+*183eH+*183fH, *186aH+*186bH+*186cH+*186dH+*186eH+*186fH, *188aH+*188bH+*188cH+*188dH+*188eH+*188fH, *189aH+*189bH+*189cH+*189dH+*189eH+*189fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *210aH+*210bH+*210cH+*210dH+*210eH+*210fH, *211aH+*211bH+*211cH+*211dH+*211eH+*211fH, *212aH+*212bH+*212cH+*212dH+*212eH+*212fH, *230aH+*230bH+*230cH+*230dH+*230eH+*230fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *235aH+*235bH+*235cH+*235dH+*235eH+*235fH, *236aH+*236bH+*236cH+*236dH+*236eH+*236fH, *238aH+*238bH+*238cH+*238dH+*238eH+*238fH, *239aH+*239bH+*239cH+*239dH+*239eH+*239fH, *242aH+*242bH+*242cH+*242dH+*242eH+*242fH, *243aH+*243bH+*243cH+*243dH+*243eH+*243fH, *245aH+*245bH+*245cH+*245dH+*245eH+*245fH, *246aH+*246bH+*246cH+*246dH+*246eH+*246fH, *249aH+*249bH+*249cH+*249dH+*249eH+*249fH, *250aH+*250bH+*250cH+*250dH+*250eH+*250fH, *251aH+*251bH+*251cH+*251dH+*251eH+*251fH, *253aH+*253bH+*253cH+*253dH+*253eH+*253fH, *254aH+*254bH+*254cH+*254dH+*254eH+*254fH, *255aH+*255bH+*255cH+*255dH+*255eH+*255fH, *256aH+*256bH+*256cH+*256dH+*256eH+*256fH, *269aH+*269bH+*269cH+*269dH+*269eH+*269fH

### Accessibility >80%, Min Distance to Active Site 6 Angstroms

*1aH+*1bH+*1cH+*1dH+*1eH+*1fH, *3aH+*3bH+*3cH+*3dH+*3eH+*3fH, *4aH+*4bH+*4cH+*4dH+*4eH+*4fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *18aH+*18bH+*18cH+*18dH+*18eH+*18fH, *19aH+*19bH+*19cH+*19dH+*19eH+*19fH, *24aH+*24bH+*24cH+*24dH+*24eH+*24fH, *27aH+*27bH+*27cH+*27dH+*27eH+*27fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *44aH+*44bH+*44cH+*44dH+*44eH+*44fH, *51aH+*51bH+*51cH+*51dH+*51eH+*51fH, *52aH+*52bH+*52cH+*52dH+*52eH+*52fH, *54aH+*54bH+*54cH+*54dH+*54eH+*54fH, *55aH+*55bH+*55cH+*55dH+*55eH+*55fH, *57aH+*57bH+*57cH+*57dH+*57eH+*57fH, *59aH+*59bH+*59cH+*59dH+*59eH+*59fH, *74aH+*74bH+*74cH+*74dH+*74eH+*74fH, *75aH+*75bH+*75cH+*75dH+*75eH+*75fH, *76aH+*76bH+*76cH+*76dH+*76eH+*76fH, *77aH+*77bH+*77cH+*77dH+*77eH+*77fH, *85aH+*85bH+*85cH+*85dH+*85eH+*85fH, *97aH+*97bH+*97cH+*97dH+*97eH+*97fH, *99aH+*99bH+*99cH+*99dH+*99eH+*99fH, *101aH+*101bH+*101cH+*101dH+*101eH+*101fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *111aH+*111bH+*111cH+*111dH+*111eH+*111fH, *114aH+*114bH+*114cH+*114dH+*114eH+*114fH, *115aH+*115bH+*115cH+*115dH+*115eH+*115fH, *118aH+*118bH+*118cH+*118dH+*118eH+*118fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *138aH+*138bH+*138cH+*138dH+*138eH+*138fH, *142aH+*142bH+*142cH+*142dH+*142eH+*142fH, *143aH+*143bH+*143cH+*143dH+*143eH+*143fH, *153aH+*153bH+*153cH+*153dH+*153eH+*153fH, *154aH+*154bH+*154cH+*154dH+*154eH+*154fH, *158aH+*158bH+*158cH+*158dH+*158eH+*158fH, *164aH+*164bH+*164cH+*164dH+*164eH+*164fH, *166aH+*166bH+*166cH+*166dH+*166eH+*166fH, *167aH+*167bH+*167cH+*167dH+*167eH+*167fH, *176aH+*176bH+*176cH+*176dH+*176eH+*176fH, *177aH+*177bH+*177cH+*177dH+*177eH+*177fH, *179aH+*179bH+*179cH+*179dH+*179eH+*179fH, *180aH+*180bH+*180cH+*180dH+*180eH+*180fH, *182aH+*182bH+*182cH+*182dH+*182eH+*182fH, *186aH+*186bH+*186cH+*186dH+*186eH+*186fH, *188aH+*188bH+*188cH+*188dH+*188eH+*188fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *210aH+*210bH+*210cH+*210dH+*210eH+*210fH, *211aH+*211bH+*211cH+*211dH+*211eH+*211fH, *212aH+*212bH+*212cH+*212dH+*212eH+*212fH, *230aH+*230bH+*230cH+*230dH+*230eH+*230fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *235aH+*235bH+*235cH+*235dH+*235eH+*235fH, *236aH+*236bH+*236cH+*236dH+*236eH+*236fH, *238aH+*238bH+*238cH+*238dH+*238eH+*238fH, *239aH+*239bH+*239cH+*239dH+*239eH+*239fH, *242aH+*242bH+*242cH+*242dH+*242eH+*242fH, *243aH+*243bH+*243cH+*243dH+*243eH+*243fH, *246aH+*246bH+*246cH+*246dH+*246eH+*246fH, *249aH+*249bH+*249cH+*249dH+*249eH+*249fH, *250aH+*250bH+*250cH+*250dH+*250eH+*250fH, *253aH+*253bH+*253cH+*253dH+*253eH+*253fH, *254aH+*254bH+*254cH+*254dH+*254eH+*254fH, *255aH+*255bH+*255cH+*255dH+*255eH+*255fH, *256aH+*256bH+*256cH+*256dH+*256eH+*256fH, *269aH+*269bH+*269cH+*269dH+*269eH+*269fH

### Accessibility >90%, Min Distance to Active Site 6 Angstroms

*1aH+*1bH+*1cH+*1dH+*1eH+*1fH, *3aH+*3bH+*3cH+*3dH+*3eH+*3fH, *4aH+*4bH+*4cH+*4dH+*4eH+*4fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *18aH+*18bH+*18cH+*18dH+*18eH+*18fH, *19aH+*19bH+*19cH+*19dH+*19eH+*19fH, *24aH+*24bH+*24cH+*24dH+*24eH+*24fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *44aH+*44bH+*44cH+*44dH+*44eH+*44fH, *51aH+*51bH+*51cH+*51dH+*51eH+*51fH, *52aH+*52bH+*52cH+*52dH+*52eH+*52fH, *54aH+*54bH+*54cH+*54dH+*54eH+*54fH, *55aH+*55bH+*55cH+*55dH+*55eH+*55fH, *57aH+*57bH+*57cH+*57dH+*57eH+*57fH, *59aH+*59bH+*59cH+*59dH+*59eH+*59fH, *74aH+*74bH+*74cH+*74dH+*74eH+*74fH, *75aH+*75bH+*75cH+*75dH+*75eH+*75fH, *76aH+*76bH+*76cH+*76dH+*76eH+*76fH, *77aH+*77bH+*77cH+*77dH+*77eH+*77fH, *97aH+*97bH+*97cH+*97dH+*97eH+*97fH, *99aH+*99bH+*99cH+*99dH+*99eH+*99fH, *101aH+*101bH+*101cH+*101dH+*101eH+*101fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *114aH+*114bH+*114cH+*114dH+*114eH+*114fH, *115aH+*115bH+*115cH+*115dH+*115eH+*115fH, *118aH+*118bH+*118cH+*118dH+*118eH+*118fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *142aH+*142bH+*142cH+*142dH+*142eH+*142fH, *143aH+*143bH+*143cH+*143dH+*143eH+*143fH, *153aH+*153bH+*153cH+*153dH+*153eH+*153fH, *154aH+*154bH+*154cH+*154dH+*154eH+*154fH, *158aH+*158bH+*158cH+*158dH+*158eH+*158fH, *164aH+*164bH+*164cH+*164dH+*164eH+*164fH, *166aH+*166bH+*166cH+*166dH+*166eH+*166fH, *167aH+*167bH+*167cH+*167dH+*167eH+*167fH, *176aH+*176bH+*176cH+*176dH+*176eH+*176fH, *177aH+*177bH+*177cH+*177dH+*177eH+*177fH, *179aH+*179bH+*179cH+*179dH+*179eH+*179fH, *180aH+*180bH+*180cH+*180dH+*180eH+*180fH, *182aH+*182bH+*182cH+*182dH+*182eH+*182fH, *188aH+*188bH+*188cH+*188dH+*188eH+*188fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *210aH+*210bH+*210cH+*210dH+*210eH+*210fH, *212aH+*212bH+*212cH+*212dH+*212eH+*212fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *235aH+*235bH+*235cH+*235dH+*235eH+*235fH, *238aH+*238bH+*238cH+*238dH+*238eH+*238fH, *239aH+*239bH+*239cH+*239dH+*239eH+*239fH, *242aH+*242bH+*242cH+*242dH+*242eH+*242fH, *246aH+*246bH+*246cH+*246dH+*246eH+*246fH, *249aH+*249bH+*249cH+*249dH+*249eH+*249fH, *250aH+*250bH+*250cH+*250dH+*250eH+*250fH, *253aH+*253bH+*253cH+*253dH+*253eH+*253fH, *254aH+*254bH+*254cH+*254dH+*254eH+*254fH, *255aH+*255bH+*255cH+*255dH+*255eH+*255fH, *269aH+*269bH+*269cH+*269dH+*269eH+*269fH

### Accessibility >100%, Min Distance to Active Site 6 Angstroms

*1aH+*1bH+*1cH+*1dH+*1eH+*1fH, *3aH+*3bH+*3cH+*3dH+*3eH+*3fH, *4aH+*4bH+*4cH+*4dH+*4eH+*4fH, *9aH+*9bH+*9cH+*9dH+*9eH+*9fH, *10aH+*10bH+*10cH+*10dH+*10eH+*10fH, *12aH+*12bH+*12cH+*12dH+*12eH+*12fH, *18aH+*18bH+*18cH+*18dH+*18eH+*18fH, *19aH+*19bH+*19cH+*19dH+*19eH+*19fH, *24aH+*24bH+*24cH+*24dH+*24eH+*24fH, *37aH+*37bH+*37cH+*37dH+*37eH+*37fH, *39aH+*39bH+*39cH+*39dH+*39eH+*39fH, *42aH+*42bH+*42cH+*42dH+*42eH+*42fH, *44aH+*44bH+*44cH+*44dH+*44eH+*44fH, *51aH+*51bH+*51cH+*51dH+*51eH+*51fH, *52aH+*52bH+*52cH+*52dH+*52eH+*52fH, *54aH+*54bH+*54cH+*54dH+*54eH+*54fH, *57aH+*57bH+*57cH+*57dH+*57eH+*57fH, *74aH+*74bH+*74cH+*74dH+*74eH+*74fH, *76aH+*76bH+*76cH+*76dH+*76eH+*76fH, *97aH+*97bH+*97cH+*97dH+*97eH+*97fH, *99aH+*99bH+*99cH+*99dH+*99eH+*99fH, *103aH+*103bH+*103cH+*103dH+*103eH+*103fH, *107aH+*107bH+*107cH+*107dH+*107eH+*107fH, *110aH+*110bH+*110cH+*110dH+*110eH+*110fH, *114aH+*114bH+*114cH+*114dH+*114eH+*114fH, *115aH+*115bH+*115cH+*115dH+*115eH+*115fH, *127aH+*127bH+*127cH+*127dH+*127eH+*127fH, *128aH+*128bH+*128cH+*128dH+*128eH+*128fH, *129aH+*129bH+*129cH+*129dH+*129eH+*129fH, *131aH+*131bH+*131cH+*131dH+*131eH+*131fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *142aH+*142bH+*142cH+*142dH+*142eH+*142fH, *143aH+*143bH+*143cH+*143dH+*143eH+*143fH, *153aH+*153bH+*153cH+*153dH+*153eH+*153fH, *154aH+*154bH+*154cH+*154dH+*154eH+*154fH, *164aH+*164bH+*164cH+*164dH+*164eH+*164fH, *166aH+*166bH+*166cH+*166dH+*166eH+*166fH, *176aH+*176bH+*176cH+*176dH+*176eH+*176fH, *177aH+*177bH+*177cH+*177dH+*177eH+*177fH, *179aH+*179bH+*179cH+*179dH+*179eH+*179fH, *182aH+*182bH+*182cH+*182dH+*182eH+*182fH, *188aH+*188bH+*188cH+*188dH+*188eH+*188fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *205aH+*205bH+*205cH+*205dH+*205eH+*205fH, *206aH+*206bH+*206cH+*206dH+*206eH+*206fH, *207aH+*207bH+*207cH+*207dH+*207eH+*207fH, *208aH+*208bH+*208cH+*208dH+*208eH+*208fH, *212aH+*212bH+*212cH+*212dH+*212eH+*212fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *238aH+*238bH+*238cH+*238dH+*238eH+*238fH, *239aH+*239bH+*239cH+*239dH+*239eH+*239fH, *242aH+*242bH+*242cH+*242dH+*242eH+*242fH, *246aH+*246bH+*246cH+*246dH+*246eH+*246fH, *250aH+*250bH+*250cH+*250dH+*250eH+*250fH, *254aH+*254bH+*254cH+*254dH+*254eH+*254fH, *255aH+*255bH+*255cH+*255dH+*255eH+*255fH, *269aH+*269bH+*269cH+*269dH+*269eH+*269fH

### Accessibility >110%, Min Distance to Active Site 6 Angstroms

*1aH+*1bH+*1cH+*1dH+*1eH+*1fH, *10aH+*10bH+*10cH+*10dH+*10eH+*10fH, *12aH+*12bH+*12cH+*12dH+*12eH+*12fH, *18aH+*18bH+*18cH+*18dH+*18eH+*18fH, *19aH+*19bH+*19cH+*19dH+*19eH+*19fH, *37aH+*37bH+*37cH+*37dH+*37eH+*37fH, *42aH+*42bH+*42cH+*42dH+*42eH+*42fH, *44aH+*44bH+*44cH+*44dH+*44eH+*44fH, *51aH+*51bH+*51cH+*51dH+*51eH+*51fH, *52aH+*52bH+*52cH+*52dH+*52eH+*52fH, *54aH+*54bH+*54cH+*54dH+*54eH+*54fH, *57aH+*57bH+*57cH+*57dH+*57eH+*57fH, *74aH+*74bH+*74cH+*74dH+*74eH+*74fH, *76aH+*76bH+*76cH+*76dH+*76eH+*76fH, *97aH+*97bH+*97cH+*97dH+*97eH+*97fH, *99aH+*99bH+*99cH+*99dH+*99eH+*99fH, *107aH+*107bH+*107cH+*107dH+*107eH+*107fH, *114aH+*114bH+*114cH+*114dH+*114eH+*114fH, *115aH+*115bH+*115cH+*115dH+*115eH+*115fH, *127aH+*127bH+*127cH+*127dH+*127eH+*127fH, *129aH+*129bH+*129cH+*129dH+*129eH+*129fH, *132aH+*132bH+*132cH+*132dH+*132eH+*132fH, *134aH+*134bH+*134cH+*134dH+*134eH+*134fH, *135aH+*135bH+*135cH+*135dH+*135eH+*135fH, *142aH+*142bH+*142cH+*142dH+*142eH+*142fH, *143aH+*143bH+*143cH+*143dH+*143eH+*143fH, *153aH+*153bH+*153cH+*153dH+*153eH+*153fH, *154aH+*154bH+*154cH+*154dH+*154eH+*154fH, *164aH+*164bH+*164cH+*164dH+*164eH+*164fH, *166aH+*166bH+*166cH+*166dH+*166eH+*166fH, *176aH+*176bH+*176cH+*176dH+*176eH+*176fH, *177aH+*177bH+*177cH+*177dH+*177eH+*177fH, *179aH+*179bH+*179cH+*179dH+*179eH+*179fH, *182aH+*182bH+*182cH+*182dH+*182eH+*182fH, *188aH+*188bH+*188cH+*188dH+*188eH+*188fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *205aH+*205bH+*205cH+*205dH+*205eH+*205fH, *206aH+*206bH+*206cH+*206dH+*206eH+*206fH, *212aH+*212bH+*212cH+*212dH+*212eH+*212fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *238aH+*238bH+*238cH+*238dH+*238eH+*238fH, *239aH+*239bH+*239cH+*239dH+*239eH+*239fH, *242aH+*242bH+*242cH+*242dH+*242eH+*242fH, *246aH+*246bH+*246cH+*246dH+*246eH+*246fH, *250aH+*250bH+*250cH+*250dH+*250eH+*250fH, *254aH+*254bH+*254cH+*254dH+*254eH+*254fH, *255aH+*255bH+*255cH+*255dH+*255eH+*255fH, *269aH+*269bH+*269cH+*269dH+*269eH+*269fH

### Accessibility >120%, Min Distance to Active Site 6 Angstroms

*1aH+*1bH+*1cH+*1dH+*1eH+*1fH, *10aH+*10bH+*10cH+*10dH+*10eH+*10fH, *18aH+*18bH+*18cH+*18dH+*18eH+*18fH, *19aH+*19bH+*19cH+*19dH+*19eH+*19fH, *37aH+*37bH+*37cH+*37dH+*37eH+*37fH, *42aH+*42bH+*42cH+*42dH+*42eH+*42fH, *44aH+*44bH+*44cH+*44dH+*44eH+*44fH, *51aH+*51bH+*51cH+*51dH+*51eH+*51fH, *52aH+*52bH+*52cH+*52dH+*52eH+*52fH, *54aH+*54bH+*54cH+*54dH+*54eH+*54fH, *57aH+*57bH+*57cH+*57dH+*57eH+*57fH, *74aH+*74bH+*74cH+*74dH+*74eH+*74fH, *76aH+*76bH+*76cH+*76dH+*76eH+*76fH, *97aH+*97bH+*97cH+*97dH+*97eH+*97fH, *99aH+*99bH+*99cH+*99dH+*99eH+*99fH, *107aH+*107bH+*107cH+*107dH+*107eH+*107fH, *114aH+*114bH+*114cH+*114dH+*114eH+*114fH, *115aH+*115bH+*115cH+*115dH+*115eH+*115fH, *127aH+*127bH+*127cH+*127dH+*127eH+*127fH, *129aH+*129bH+*129cH+*129dH+*129eH+*129fH, *134aH+*134bH+*134cH+*134dH+*134eH+*134fH, *135aH+*135bH+*135cH+*135dH+*135eH+*135fH, *142aH+*142bH+*142cH+*142dH+*142eH+*142fH, *143aH+*143bH+*143cH+*143dH+*143eH+*143fH, *153aH+*153bH+*153cH+*153dH+*153eH+*153fH, *154aH+*154bH+*154cH+*154dH+*154eH+*154fH, *164aH+*164bH+*164cH+*164dH+*164eH+*164fH, *166aH+*166bH+*166cH+*166dH+*166eH+*166fH, *176aH+*176bH+*176cH+*176dH+*176eH+*176fH, *177aH+*177bH+*177cH+*177dH+*177eH+*177fH, *179aH+*179bH+*179cH+*179dH+*179eH+*179fH, *182aH+*182bH+*182cH+*182dH+*182eH+*182fH, *188aH+*188bH+*188cH+*188dH+*188eH+*188fH, *198aH+*198bH+*198cH+*198dH+*198eH+*198fH, *200aH+*200bH+*200cH+*200dH+*200eH+*200fH, *203aH+*203bH+*203cH+*203dH+*203eH+*203fH, *212aH+*212bH+*212cH+*212dH+*212eH+*212fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *239aH+*239bH+*239cH+*239dH+*239eH+*239fH, *246aH+*246bH+*246cH+*246dH+*246eH+*246fH, *250aH+*250bH+*250cH+*250dH+*250eH+*250fH, *254aH+*254bH+*254cH+*254dH+*254eH+*254fH, *255aH+*255bH+*255cH+*255dH+*255eH+*255fH, *269aH+*269bH+*269cH+*269dH+*269eH+*269fH

### Accessibility >130%, Min Distance to Active Site 6 Angstroms

*1aH+*1bH+*1cH+*1dH+*1eH+*1fH, *18aH+*18bH+*18cH+*18dH+*18eH+*18fH, *19aH+*19bH+*19cH+*19dH+*19eH+*19fH, *37aH+*37bH+*37cH+*37dH+*37eH+*37fH, *42aH+*42bH+*42cH+*42dH+*42eH+*42fH, *44aH+*44bH+*44cH+*44dH+*44eH+*44fH, *51aH+*51bH+*51cH+*51dH+*51eH+*51fH, *52aH+*52bH+*52cH+*52dH+*52eH+*52fH, *54aH+*54bH+*54cH+*54dH+*54eH+*54fH, *57aH+*57bH+*57cH+*57dH+*57eH+*57fH, *74aH+*74bH+*74cH+*74dH+*74eH+*74fH, *76aH+*76bH+*76cH+*76dH+*76eH+*76fH, *97aH+*97bH+*97cH+*97dH+*97eH+*97fH, *107aH+*107bH+*107cH+*107dH+*107eH+*107fH, *114aH+*114bH+*114cH+*114dH+*114eH+*114fH, *127aH+*127bH+*127cH+*127dH+*127eH+*127fH, *129aH+*129bH+*129cH+*129dH+*129eH+*129fH, *135aH+*135bH+*135cH+*135dH+*135eH+*135fH, *142aH+*142bH+*142cH+*142dH+*142eH+*142fH, *143aH+*143bH+*143cH+*143dH+*143eH+*143fH, *164aH+*164bH+*164cH+*164dH+*164eH+*164fH, *176aH+*176bH+*176cH+*176dH+*176eH+*176fH, *177aH+*177bH+*177cH+*177dH+*177eH+*177fH, *182aH+*182bH+*182cH+*182dH+*182eH+*182fH, *200aH+*200bH+*200cH+*200dH+*200eH+*200fH, *203aH+*203bH+*203cH+*203dH+*203eH+*203fH, *212aH+*212bH+*212cH+*212dH+*212eH+*212fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *239aH+*239bH+*239cH+*239dH+*239eH+*239fH, *246aH+*246bH+*246cH+*246dH+*246eH+*246fH, *250aH+*250bH+*250cH+*250dH+*250eH+*250fH, *254aH+*254bH+*254cH+*254dH+*254eH+*254fH, *269aH+*269bH+*269cH+*269dH+*269eH+*269fH

### Accessibility >140%, Min Distance to Active Site 6 Angstroms

*1aH+*1bH+*1cH+*1dH+*1eH+*1fH, *18aH+*18bH+*18cH+*18dH+*18eH+*18fH, *19aH+*19bH+*19cH+*19dH+*19eH+*19fH, *37aH+*37bH+*37cH+*37dH+*37eH+*37fH, *42aH+*42bH+*42cH+*42dH+*42eH+*42fH, *44aH+*44bH+*44cH+*44dH+*44eH+*44fH, *51aH+*51bH+*51cH+*51dH+*51eH+*51fH, *54aH+*54bH+*54cH+*54dH+*54eH+*54fH, *57aH+*57bH+*57cH+*57dH+*57eH+*57fH, *74aH+*74bH+*74cH+*74dH+*74eH+*74fH, *76aH+*76bH+*76cH+*76dH+*76eH+*76fH, *97aH+*97bH+*97cH+*97dH+*97eH+*97fH, *107aH+*107bH+*107cH+*107dH+*107eH+*107fH, *114aH+*114bH+*114cH+*114dH+*114eH+*114fH, *127aH+*127bH+*127cH+*127dH+*127eH+*127fH, *129aH+*129bH+*129cH+*129dH+*129eH+*129fH, *135aH+*135bH+*135cH+*135dH+*135eH+*135fH, *142aH+*142bH+*142cH+*142dH+*142eH+*142fH, *164aH+*164bH+*164cH+*164dH+*164eH+*164fH, *177aH+*177bH+*177cH+*177dH+*177eH+*177fH, *182aH+*182bH+*182cH+*182dH+*182eH+*182fH, *212aH+*212bH+*212cH+*212dH+*212eH+*212fH, *231aH+*231bH+*231cH+*231dH+*231eH+*231fH, *233aH+*233bH+*233cH+*233dH+*233eH+*233fH, *234aH+*234bH+*234cH+*234dH+*234eH+*234fH, *239aH+*239bH+*239cH+*239dH+*239eH+*239fH, *246aH+*246bH+*246cH+*246dH+*246eH+*246fH, *250aH+*250bH+*250cH+*250dH+*250eH+*250fH, *254aH+*254bH+*254cH+*254dH+*254eH+*254fH, *269aH+*269bH+*269cH+*269dH+*269eH+*269fH

One preferred embodiment relates a protease variant of a protease parent wherein the protease variant comprise an amino acid sequence which is at least 60% identical to the mature polypeptide with SEQ ID NO 2 and wherein the protease variant compared to the protease parent comprises 2 to 6 of the insertions selected from the group consisting of *20aH, *22aH, *89aH, *115aH, *135H, *139aH, *143H, *232aH, *238aH, *242aH and *246aH.

A particular embodiment relates to a protease variant having an amino acid sequence which is at least 60% identical to the mature polypeptide with SEQ ID NO 2 and wherein the protease variant compared to the protease parent comprises 2 to 6 of the insertions selected from the group consisting of: *1aH, *18aH, *19aH, *37aH, *42aH, *44aH, *51aH, *54aH, *57aH, *74aH, *76aH, *97aH, *107aH, *114aH, *127aH, *129aH, *135aH, *142aH, *164aH, *177aH, *182aH,*212aH,*231aH,*233aH,*234aH,*239aH,*246aH,*250aH and *254aH.

Another preferred embodiment relates to a method of producing a variant protease having at least 60% identity to SEQ ID NO 2 wherein the method comprises the insertion of 2 to 6 histidines at any one or more of the positions selected from the group consisting of: A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259 and R269.

One embodiment relates to a method of producing a variant protease having at least 60% identity to SEQ ID NO 2 wherein the method comprising insertion of histidine at any one or more of the positions selected from the group consisting of: A1, N18, R19, T37, N42, R44, P51, P54, Q57, N74, S76, S97, Q107, N114, P127, P129, Q135, S142, R164, N177, S182, N212, K231, P233, S234, Q239, N246, S250 and T254.

An even more preferred embodiment of the invention relates to a method of producing a variant protease having at least 60% identity to SEQ ID NO 2 wherein the method comprise insertion of histidine at any one or more of the positions selected from the group consisting of: *20aH, *22aH, *89aH, *115aH, *135H, *139aH, *143H, *232aH, *238aH, *242aH and *246aH.

The histidine insertion variants described above have increased solubility at pH 5 and preferably at least one additional improved property compared to the parent and/or compared to SEQ ID NO 3, such as improved properties are preferably improved wash performance, improved activity on specific substrates and/or improved stability such as improved storage stability. The histidine insertion variants may further comprise substitutions at one or more positions (e.g. several). One embodiment relates to a protease variant comprising an amino acid sequence which is at least 60% identical to SEQ ID NO 3 and which compared to SEQ ID NO 3 comprises 2 to 6 of the following insertions: *1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH, *110aH, *111aH, *114aH, *115aH,*116aH,*118aH,*125aH,*126aH,*127aH,*128aH,*129aH,*131aH,*132aH,*134aH, *135aH,*138aH,*139aH,*142aH,*143aH,*144aH,*153aH,*154aH,*156aH,*157aH,*158aH, *161aH,*164aH,*166aH,*167aH,*175aH,*176aH,*177aH,*178aH,*179aH,*180aH,*182aH, *183aH, *186aH, *188aH, *189aH, *198aH, *200aH, *203aH, *204aH, *205aH, *206aH, *207aH, *208aH,*210aH,*211aH,*212aH,*229aH,*230aH,*231aH,*232aH,*233aH,*234aH,*235aH, *236aH, *238aH, *239aH, *241aH, *242aH, *243aH, *245aH, *246aH, *249aH, *250aH, *251aH, *252aH, *253aH, *254aH, *255aH, *256aH, *257aH, *259aH and *269aH.

The present invention can also be described by the following particular embodiments.

One embodiment relates to a method of producing a variant of a parent protein wherein the variant has at least two additional histidines on the surface compared to the parent, the method comprising introducing into the parent protein alterations to obtain additional 2 to 6 histidines at the surface of the mature polypeptide of the parent protein, wherein each alteration is independently a substitution or insertion. One embodiment relates to the method of embodiment one wherein the histidines on the surface is situated internally in the protein i.e. not on the N- or C-terminal. One embodiment relates to embodiment one and/or two, wherein the variant has increased solubility below pH 5 compared to the parent protein and/or compared to SEQ ID NO 3. One embodiment relates to embodiment one, two and/or three, wherein the variant has increased performance such as in creased wash performance or increased performance one or more substrates selected from EnzChek Protease kit Red, DQ Red BSA, EMPA117EH, AZCL-Hemoglobin, PC-03 and PC-05 as shown in the examples below when compared to the parent protein, and/or when compared to SEQ ID NO 3. One embodiment according to any of the previous embodiments relates to a method, wherein the alteration is a substitution. One embodiment relates to a method according to any of the above embodiments wherein the alteration is an insertion. One embodiment relates to a method according to any of the previous embodiments, wherein two additional histidines are inserted or substituted at the surface of the mature polypeptide of the parent protein. One embodiment relates to a method according to any of the above embodiments wherein three additional histidines are inserted or substituted at the surface of the mature polypeptide of the parent protein. One embodiment relates to a method according to any of the above embodiments wherein four additional histidines are inserted or substituted at the surface of the mature polypeptide of the parent protein. One embodiment relates to a method according to any of the above embodiments wherein five additional histidines are inserted or substituted at the surface of the mature polypeptide of the parent protein. One embodiment relates to a method according to any of the above embodiments wherein six additional histidines are inserted or substituted at the surface of the mature polypeptide of the parent protein. One embodiment relates to a method according to any of the above embodiments wherein the parent protein is an enzyme. One embodiment relates to a method according to any of the above embodiments, wherein the parent protein is a protease. One embodiment relates to a method according to any of the above embodiments wherein the parent protein comprises at least 60% sequence identity to SEQ ID NO 3. One embodiment relates to a method according to any of the above embodiments wherein the protein is a protease with SEQ ID NO 3. One embodiment relates to a method according to any of the above embodiments wherein the surface correspond to positions at the surface of the mature polypeptide of SEQ ID NO 2 or to SEQ ID NO 3 selected from the group consisting of: A1, Q2, S3, V4, W6, S9, R10, Q12, P14, A15, H17, N18, R19, G20, T22, S24, G25, K27, T37, P39, N42, 143, R44, G45, G46, S48, F49, P51, G52, E53, P54, S55, T56, Q57, G59, L73, N74, N75, S76, 177, S85, E87, A96, S97, G98, S99, G100, S101, V102, S103, Q107, E110, W111, N114, N115, G116, H118, G125, S126, P127, S128, P129, A131, T132, E134, Q135, N138, S139, S142, R143, G144, N153, S154, A156, G157, S158, Y161, R164, A166, N167, D175, Q176, N177, N178, N179, R180, S182, F183, Y186, A188, G189, N198, Q200, Y203, P204, G205, S206, T207, Y208, S210, L211, N212, K229, Q230, K231, N232, P233, S234, W235, S236, V238, Q239, R241, N242, H243, K245, N246, T249, S250, L251, G252, S253, T254, N255, L256, Y257, S259, and R269.

One embodiment relates to a method according to any of the above embodiments, wherein the surface correspond to positions at the surface of the mature polypeptide of SEQ ID NO 2 or to SEQ ID NO 3 selected from the group consisting of: A1, N18, R19, T37, N42, R44, P51, P54, Q57, N74, S76, S97, Q107, N114, P127, P129, Q135, S142, R164, N177, S182, N212, K231, P233, S234, Q239, N246, S250, T254 and R269. One preferred embodiment relates to a method according to any of the proceeding embodiments wherein the surface correspond to positions at the surface of the mature polypeptide of SEQ ID NO 2 selected from the group consisting of: G20H, T22H, Y89H, N115H, Q135H, S139H, R143H, N232H, V238H, N242H or N246H. One embodiment relates to a method according to any of the above embodiments wherein the method comprise introducing 2 to 6 of the following insertions: *1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH,*110aH,*111aH,*114aH,*115aH,*116aH,*118aH,*125aH,*126aH,*127aH,*128aH, *129aH, *131aH, *132aH, *134aH, *135aH, *138aH, *139aH, *142aH, *143aH, *144aH, *153aH, *154aH, *156aH,*157aH, *158aH,*161aH, *164aH,*166aH, *167aH,*175aH, *176aH,*177aH, *178aH,*179aH, *180aH,*182aH,*183aH, *186aH, *188aH,*189aH, *198aH, *200aH,*203aH, *204aH, *205aH, *206aH, *207aH, *208aH, *210aH, *211aH, *212aH, *229aH, *230aH, *231aH, *232aH, *233aH, *234aH, *235aH, *236aH, *238aH, *239aH, *241aH, *242aH, *243aH, *245aH, *246aH, *249aH, *250aH, *251aH, *252aH, *253aH, *254aH, *255aH, *256aH, *257aH, *259aH, *269aH. One embodiment relates to a method according to any of the above embodiments, wherein the insertions are selected from the group consisting of *20aH, *22aH, *89aH, *115aH, *135H, *139aH, *143H, *232aH, *238aH, *242aH and *246aH. One embodiment relates to a method of producing a variant of a parent protease wherein the variant has at least two additional histidines on the surface compared to the parent protease, the method comprising introducing into the parent protease alterations to obtain additional 2 to 6 histidines at the surface of the mature polypeptide of the parent protease, wherein each the 2 to 6 histidines are introduced by the substitutions selected from the group consisting of: A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H, preferably the substitutions are selected form the group consisting of: G20H, T22H, Y89H, N115H, Q135H, S139H, R143H, N232H, V238H, N242H and N246H.

One embodiment relates to a method wherein a protein variant has at least two additional histidines on the surface compared to the parent protein comprising the steps of:

a) identifying amino acid positions at the surface of the mature polypeptide of the parent protein;

b) selecting at least one amino acid position among the positions identified in a) which is not occupied by a histidine in the parent protein; and

c) substituting amino acid selected in b) with histidine; and

d) expressing the modified nucleic acid sequence in a host cell to produce the variant.

One embodiment relates to a method wherein the protein variant has at least two additional histidines on the surface compared to the parent protein comprising the steps of:

a) identifying amino acid positions at the surface of the mature polypeptide of the parent enzyme;

b) selecting at least one amino acid position among the positions identified in a); and

c) inserting one or more histidine adjacent to the at least one amino acid position selected in b); and

d) expressing the modified nucleic acid sequence in a host cell to produce the variant,

Preferably step a) is performed using the method described in “solvent accessibility of residues in 3D model”. Preferably the protein is a protease and even more preferably a protease having at least 60%, such as at least 70%, such as at least 80%, such as at least 90% or such as at least 100% sequence identity to the mature polypeptide of SEQ ID NO 2 or SEQ ID NO 3.

One embodiment relates to a protein variant wherein, when compared to the parent protein the variant comprises 2 to 6 additional histidines at positions corresponding to positions at the surface of the mature polypeptide of the parent protein. The protein is preferably a protease and preferably, when compared to the parent protease the protease variant comprise 2 to 6 additional histidines at positions corresponding to positions at the surface of the mature polypeptide of SEQ ID NO: 2. Preferably the protease variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity to SEQ ID NO: 3 and preferably the protease variant comprises 2 to 6 of the following substitutions: A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, 5210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H. Even more preferably the protease variant comprises the substitutions selected from the group consisting of: G20H, T22H, Y89H, N115H, Q135H, S139H, R143H, N232H, V238H, N242H and N246H. One embodiment relates a protease variant, comprising 2 to 6 of the following insertions: *1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH, *110aH, *111aH, *114aH,*115aH,*116aH,*118aH,*125aH,*126aH,*127aH,*128aH,*129aH,*131aH,*132aH, *134aH,*135aH,*138aH,*139aH,*142aH,*143aH,*144aH,*153aH,*154aH,*156aH,*157aH, *158aH, *161aH, *164aH, *166aH, *167aH, *175aH, *176aH, *177aH, *178aH, *179aH, *180aH, *182aH,*183aH,*186aH,*188aH,*189aH,*198aH,*200aH,*203aH,*204aH,*205aH,*206aH, *207aH, *208aH, *210aH, *211aH, *212aH, *229aH, *230aH, *231aH, *232aH, *233aH, *234aH, *235aH, *236aH, *238aH, *239aH, *241aH, *242aH, *243aH, *245aH, *246aH, *249aH, *250aH, *251aH, *252aH, *253aH, *254aH, *255aH, *256aH, *257aH, *259aH or *269aH, wherein the protease variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity to SEQ ID NO: 3. Preferably the histidines are not at the C- or N-terminal, preferably the protease variant comprises the following insertions*2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH, *110aH,*111aH,*114aH,*115aH,*116aH,*118aH,*125aH,*126aH,*127aH,*128aH,*129aH, *131aH,*132aH,*134aH,*135aH,*138aH,*139aH,*142aH,*143aH,*144aH,*153aH,*154aH, *156aH,*157aH,*158aH,*161aH,*164aH,*166aH,*167aH,*175aH,*176aH,*177aH,*178aH, *179aH,*180aH,*182aH,*183aH,*186aH,*188aH,*189aH,*198aH,*200aH,*203aH,*204aH, *205aH, *206aH, *207aH, *208aH, *210aH, *211aH, *212aH, *229aH, *230aH, *231aH, *232aH, *233aH, *234aH, *235aH, *236aH, *238aH, *239aH, *241aH, *242aH, *243aH, *245aH, *246aH, *249aH, *250aH, *251aH, *252aH, *253aH, *254aH, *255aH, *256aH, *257aH or *259aH.

Preferably the protease variant comprises the insertions are selected from the group consisting of the following insertions: *20aH, *22aH, *89aH, *115aH, *135H, *139aH, *143H, *232aH, *238aH, *242aH and *246aH. One embodiment relates to protease variants further comprising one or more substitution at positions selected from the group consisting of positions: 3, 4, 9, 15, 24, 42, 59, 66, 74, 76, 97, 99, 101, 102, 118, 126, 127, 128, 154, 156, 157, 158, 161, 164, 176, 179, 182, 188, 198, 199, 200, 203, 210, 211, 212, 216, 239, 255 and 256, preferably positions 9, 15, 42, 66, 74, 97, 99, 154, 200, 203, 211, 212 and/or 256 (numbering according to SEQ ID NO: 3). Preferably the substitutions are selected from any of the following: S3T, V41, S9[E,D,K,R], A15T, S24G, N42[R,K], G59E, V66[G,A,S;T], N74[D,E], S76[N,Q], S97[D,E,A], S99[R,K,N,M,E,D,L,I], S101A, V1021, H118[D,E], 5126[L,I,V], P127[N,Q] S128A, S154[E,D], A156[D,E], G157P, S158[E,D], Y161A, R164S, Q176 [D,E], N179[D,E], S182[D,E], A188P, V199M, N198[D,E], V1991, Q200L, Y203W, S210V, L211[D,E], N212[D,E], M216S, Q239[K,R], N255[D,E] and L256[D,E] (numbering according to SEQ ID NO: 3). The histidine modified variants of the invention preferably has at least 60%, e.g., at least 65%, at least 70%, at least 75%, at least 80%, at least 85%, at least 90%, at least 95%, at least 96%, at least 97%, at least 98%, at least 99% sequence identity to SEQ ID NO 3. In one embodiment the protease parent comprises or consists of SEQ ID NO: 3. In one embodiment, the histidine modified protease variant comprises a total number of alterations of no more than 1-20, e.g., 1-10 and 1-5, such as 1, 2, 3, 4, 5, 6, 7, 8, 9 or 10 alterations. In one embodiment the protease variant has an increased solubility below pH 5 compared to the parent protease and/or compared to SEQ ID NO 3. In one embodiment, the protease variant has an increased solubility below pH 5 and/or improved wash performance compared to the parent protease and/or compared to SEQ ID NO 3. In one embodiment, the variant has increased solubility below pH 5 and/or increased performance such as increased wash performance or increased performance one or more substrates selected from EnzChek Protease kit Red, DQ Red BSA, EMPA117EH, AZCL-Hemoglobin, PC-03 and PC-05 as shown in the examples below when compared to the parent protein, and/or when compared to the protein comprising SEQ ID NO 3.

The histidine modified variants of the invention may further comprise additional alterations or amino acid changes, such amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. For Savinase (SEQ ID NO: 2) the catalytic triad comprising the amino acids S215, H62, and D32 is essential for protease activity of the enzyme.

The variants may consist of 50 to 100 amino acids, e.g., 100 to 150, 150 to 200, 200 to 250 and 250 to 269 amino acids.

In an embodiment, the variant has improved wash performance properties compared to the parent enzyme.

### Parent Enzymes

The parent enzyme may be any protein. In a preferred embodiment of the invention the parent enzyme is a protease and in an even more preferred embodiment the parent protease is a protease having an amino acid sequence which has at least 60% sequence identity to SEQ ID NO 2.

(a) a polypeptide having at least 60% sequence identity to the mature polypeptide of SEQ ID NO: 2; (b) a polypeptide encoded by a polynucleotide that hybridizes under low stringency conditions with (i) the mature polypeptide coding sequence of SEQ ID NO: 1 or (ii) the full-length complement of (i); or (c) a polypeptide encoded by a polynucleotide having at least 60% sequence identity to the mature polypeptide coding sequence of SEQ ID NO: 1.

In an aspect, the parent has a sequence identity to the mature polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have protease activity. In one aspect, the amino acid sequence of the parent differs by up to 10 amino acids, e.g., 1, 2, 3, 4, 5, 6, 7, 8, 9, or 10, from the mature polypeptide of SEQ ID NO: 2.

In another aspect, the parent comprises or consists of the amino acid sequence of SEQ ID NO: 3. In another aspect, the parent comprises or consists of the mature polypeptide of SEQ ID NO: 2. In another aspect, the parent comprises or consists of amino acids 1 to 269 of SEQ ID NO: 2.

In one embodiment, the parent is a fragment of the mature polypeptide of SEQ ID NO: 2 containing at least 150 amino acid residues, e.g., at least 200 and at least 250 amino acid residues.

In another embodiment, the parent is an allelic variant of the mature polypeptide of SEQ ID NO: 2.

In one embodiment, the parent is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1 or (ii) the full-length complement of (i) (Sambrook et al., 1989, Molecular Cloning, A Laboratory Manual, 2d edition, Cold Spring Harbor, New York).

The polynucleotide of SEQ ID NO: 1 or a subsequence thereof, as well as the polypeptide of SEQ ID NO: 2 or a fragment thereof may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

For purposes of the present invention, hybridization indicates that the polynucleotide hybridizes to a labeled nucleic acid probe corresponding to (i) SEQ ID NO: 1; (ii) the mature polypeptide coding sequence of SEQ ID NO: 1; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID NO: 1. In one embodiment, the nucleic acid probe is nucleotides 1 to 1140 of SEQ ID NO: 1. In one embodiment, the nucleic acid probe is a polynucleotide that encodes the polypeptide of SEQ ID NO: 2; the mature polypeptide thereof; or a fragment thereof. In one embodiment, the nucleic acid probe is SEQ ID NO: 1.

In another embodiment, the parent is encoded by a polynucleotide having a sequence identity to the mature polypeptide coding sequence of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

The polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent may be a bacterial enzyme. In a particular aspect the parent is a bacterial protease. For example, the parent protease may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma protease.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis protease.

In one embodiment, the parent is a Streptococcus equisimilis, Streptococcus pyogenes, Streptococcus uberis, or Streptococcus equi subsp. Zooepidemicus protease.

In one embodiment, the parent is a Streptomyces achromogenes, Streptomyces avermitilis, Streptomyces coelicolor, Streptomyces griseus, or Streptomyces lividans protease.

In a preferred aspect, the parent is a bacillus protease, e.g., the protease of SEQ ID NO: 2 or the mature polypeptide thereof.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to methods for obtaining a variant having enzyme activity, such as protease activity comprising: (a) introducing into a parent enzyme a substitution or insertion of histidine at 2 to 6 positions corresponding to positions at the surface of the mature polypeptide of SEQ ID NO: 2, wherein the variant has enzyme activity; and (b) recovering the variant. A preferred aspect of the invention a method for obtaining a variant having protease activity, comprising: (a) introducing into a parent protease e.g. a protease having at least 60% identity to the mature polypeptide of SEQ ID 2 a substitution or insertion at 2 to 6 positions corresponding to positions at the surface of the mature polypeptide of SEQ ID NO: 2, wherein the variant has enzyme activity; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a variant of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or 2 to 6 vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier.

The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK). The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocaffimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonaturn, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Compositions

The present invention also relates to compositions comprising the histidine modified variants of the present invention. Preferably, the compositions are enriched in such a variant. The term “enriched” indicates that the enzyme activity of the composition has been increased, e.g., with an enrichment factor of at least 1.1.

The compositions may comprise the histidine modified variants of the present invention as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase; more preferably the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

The compositions may be prepared in accordance with methods known in the art and may be in the form of a liquid or a dry composition. The compositions may be stabilized in accordance with methods known in the art.

Examples are given below of preferred compositions of the present invention and uses hereof. The dosage of the composition and other conditions under which the composition is used may be determined on the basis of methods known in the art.

One embodiment relates to a composition comprising at least one protein variant of a parent protein wherein when compared to the parent protein the protein variant comprises 2 to 6 additional histidines at positions corresponding to positions at the surface of the mature polypeptide of the parent protein. In one embodiment the composition comprises at least one of a first protein variant of a parent protein wherein, when compared to the parent protein the first protein variant comprises 2 to 6 additional histidines at positions corresponding to positions at the surface of the mature polypeptide of the parent protein and comprises at least one second protein variant wherein the protein variant is identical to the first protein variant except for the 2 to 6 additional histidines. The protein is preferably a protease and preferably, when compared to the parent protease the protease variant comprise 2 to 6 additional histidines at positions corresponding to positions at the surface of the mature polypeptide of SEQ ID NO: 2, wherein the protease variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity to SEQ ID NO: 3 and preferably the protease variant comprises 2 to 6 of the following substitutions: A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H. Even more preferably the protease variant comprises the substitutions selected from the group consisting of: G20H, T22H, Y89H, N115H, Q135H, S139H, R143H, N232H, V238H, N242H and N246H. According to one embodiment the composition comprises a protease variant, comprising 2 to 6 of the following insertions: *1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH, *110aH, *111aH, *114aH,*115aH,*116aH,*118aH,*125aH,*126aH,*127aH,*128aH,*129aH,*131aH,*132aH, *134aH,*135aH,*138aH,*139aH,*142aH,*143aH,*144aH,*153aH,*154aH,*156aH,*157aH, *158aH, *161aH, *164aH, *166aH, *167aH, *175aH, *176aH, *177aH, *178aH, *179aH, *180aH, *182aH,*183aH,*186aH,*188aH,*189aH,*198aH,*200aH,*203aH,*204aH,*205aH,*206aH, *207aH, *208aH, *210aH, *211aH, *212aH, *229aH, *230aH, *231aH, *232aH, *233aH, *234aH, *235aH, *236aH, *238aH, *239aH, *241aH, *242aH, *243aH, *245aH, *246aH, *249aH, *250aH, *251aH, *252aH, *253aH, *254aH, *255aH, *256aH, *257aH, *259aH, *269aH, wherein the protease variant has at least 60%, e.g., at least 65%, at least 70%, at least 75%, at least 80%, at least 85%, at least 90%, at least 95%, at least 96%, at least 97%, at least 98%, or at least 99%, but less than 100% sequence identity to SEQ ID NO: 3, preferably the variant comprises the insertions are selected from the group consisting of the following insertions: *20aH, *22aH, *89aH, *115aH, *135H, *139aH, *143H, *232aH, *238aH, *242aH and *246aH. The composition comprising at least one protein variant as described above preferably comprises at least one additional component such as a polyols, polymers and additional enzymes.

In one certain aspect, the histidine modified variants according to the invention have improved wash performance compared to the parent protein. In a preferred embodiment the protein is a protease and the histidine modified protease variants according to the invention have improved wash performance compared to SEQ ID NO: 3, wherein wash performance may be measured using the Automatic Mechanical Stress Assay (AMSA) for Automatic Dish Wash as described in the Materials and Methods section herein.

Thus, in a preferred embodiment the composition is a detergent composition, and one aspect of the invention relates to the use of a detergent composition comprising a histidine modified variant according to the invention in a cleaning process such as laundry or hard surface cleaning.

The choice of additional components is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment of the present invention, the a histidine modified variants of the present invention may be added to a detergent composition in an amount corresponding to 0.01-200 mg of enzyme protein per liter of wash liquor, preferably 0.05-50 mg of enzyme protein per liter of wash liquor, in particular 0.1-10 mg of enzyme protein per liter of wash liquor.

A composition for use in automatic dishwash (ADW), for example, may include 0.0001%-50%, such as 0.001%-30%, such as 0.01%-20%, such as 0.5-15% of enzyme protein by weight of the composition.

A composition for use in laundry granulation, for example, may include 0.0001%-50%, such as 0.001%-20%, such as 0.01%-10%, such as 0.05%-5% of enzyme protein by weight of the composition.

A composition for use in laundry liquid, for example, may include 0.0001%-10%, such as 0.001-7%, such as 0.1%-5% of enzyme protein by weight of the composition.

The proteins of the detergent composition of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO 92/19709 and WO 92/19708 or the histidine modified variants according to the invention may be stabilized using peptide aldehydes or ketones such as described in WO 2005/105826 and WO 2009/118375.

A variant of the present invention may also be incorporated in the detergent formulations disclosed in WO97/07202, which is hereby incorporated by reference.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein, the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein, the detergent will usually contain from about 0% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein, the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually contain from about 0% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein, the detergent will usually contain from about 0% to about 10% by weight of a zwitterionic surfactant. Non-limiting examples of zwitterionic surfactants include betaine, alkyldimethylbetaine, sulfobetaine, and combinations thereof.

### Hydrotropes

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 45% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-20% by weight, such as about 5% to about 10%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra-(methylenephosphonic acid) (EDTMPA), diethylenetriaminepentakis(methylenephosphonic acid) (DTPMPA or DTMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The detergent may contain 0-50% by weight, such as about 0.1% to about 25%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst, Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me₃-TACN) or 1,2,4,7-tetramethyl-1,4,7-triazacyclononane (Me₄-TACN), in particular Me₃-TACN, such as the dinuclear manganese complex [(Me₃-TACN)Mn(O)₃Mn(Me₃-TACN)](PF₆)₂, and [2,2′,2″-nitrilotris(ethane-1,2-diylazanylylidene-κN-methanylylidene)triphenolato-κ³O]manganese(III). The bleach catalysts may also be other metal compounds, such as iron or cobalt complexes.

In some embodiments, the bleach component may be an organic catalyst selected from the group consisting of organic catalysts having the following formulae:

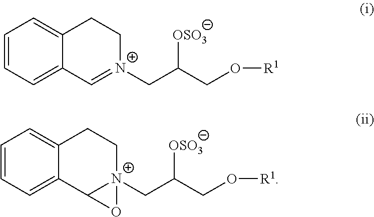

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g. in WO2007/087258, WO2007/087244, WO2007/087259 and WO2007/087242. Suitable photobleaches may for example be sulfonated zinc phthalocyanine

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when the fabric is contacted with a wash liquor comprising the detergent compositions and thus altering the tint of the fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Additional Enzymes

The detergent additive as well as the detergent composition may comprise one or more (additional) enzymes such as a protease, lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Examples of cellulases exhibiting endo-beta-1,4-glucanase activity (EC 3.2.1.4) are those having described in WO02/099091.

Other examples of cellulases include the family 45 cellulases described in WO96/29397, and especially variants thereof having substitution, insertion and/or deletion at one or more of the positions corresponding to the following positions in SEQ ID NO: 8 of WO 02/099091: 2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected among P19A, G20K, Q44K, N48E, Q119H or Q146 R. Commercially available cellulases include Celluzyme™, and Carezyme™ (Novozymes NS), Clazinase™, and Puradax HA™ (Genencor International Inc.), and KAC-500(B)™ (Kao Corporation).

### Proteases

Suitable additional proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the 51 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellulomonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens. Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263 and WO11/036264.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes NS), those sold under the tradename Maxatase®, Maxacal®, Maxapem®, Purafect®, Purafect Prime®, Purafect MA®, Purafect Ox®, Purafect Ox®, Puramax®, Properase®, FN2®, FN3®, FN4®, Excellase®, Ultimase®, Eraser®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes NS), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases which can be used together with the histidine modified variant of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

### M197T;

### H156Y+A181T+N190F+A209V+Q264S; or

### G48A+T491+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

### N128C+K178L+T182G+Y305R+G475K;

### N1280+K178L+T182G+F202Y+Y305R+D319T+G475K;

### S125A+N128C+K178L+T182G+Y305R+G475K; or

S125A+N128C+T131I+T1651+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes NS), and Rapidase™, Purastar™/Effectenz™, Powerase and Preferenz S100 (from Genencor International Inc./DuPont).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme® (Novozymes NS).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc.

Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants: The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents: The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent: The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

Soil release polymers: The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents: The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having 2 to 6 layers, a pouch having one or more compartments, a regular or compact powder, a granule, a paste, a gel, or a regular, compact or concentrated liquid. There are a number of detergent formulation forms such as layers (same or different phases), pouches, as well as forms for machine dosing unit.

Pouches can be configured as single or multicompartments. It can be of any form, shape and material which is suitable for hold the composition, e.g. without allowing the release of the composition from the pouch prior to water contact. The pouch is made from water soluble film which encloses an inner volume. The inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxyprpyl methyl cellulose (HPMC). Preferably the level of polymer in the film for example PVA is at least about 60%. Preferred average molecular weight will typically be about 20,000 to about 150,000. Films can also be of blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticisers like glycerol, ethylene glycerol, Propylene glycol, sorbitol and mixtures thereof. The pouches can comprise a solid laundry detergent composition or part components and/or a liquid cleaning composition or part components separated by the water soluble film. The compartment for liquid components can be different in composition than compartments containing solids. Ref: (US2009/0011970 A1). Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na+, K+ or NH4+ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g. a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, an enzyme, optionally one or more additional enzymes, a protease inhibitor, and a salt of a monovalent cation and an organic anion may be prepared and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

A granular detergent may be formulated as described in WO09/092699, EP1705241, EP1382668, WO07/001262, U.S. Pat. No. 6,472,364, WO04/074419 or WO09/102854. Other useful detergent formulations are described in WO09/124162, WO09/124163, WO09/117340, WO09/117341, WO09/117342, WO09/072069, WO09/063355, WO09/132870, WO09/121757, WO09/112296, WO09/112298, WO09/103822, WO09/087033, WO09/050026, WO09/047125, WO09/047126, WO09/047127, WO09/047128, WO09/021784, WO09/010375, WO09/000605, WO09/122125, WO09/095645, WO09/040544, WO09/040545, WO09/024780, WO09/004295, WO09/004294, WO09/121725, WO09/115391, WO09/115392, WO09/074398, WO09/074403, WO09/068501, WO09/065770, WO09/021813, WO09/030632, and WO09/015951. WO2011025615, WO2011016958, WO2011005803, WO2011005623, WO2011005730, WO2011005844, WO2011005904, WO2011005630, WO2011005830, WO2011005912, WO2011005905, WO2011005910, WO2011005813, WO2010135238, WO2010120863, WO2010108002, WO2010111365, WO2010108000, WO2010107635, WO2010090915, WO2010033976, WO2010033746, WO2010033747, WO2010033897, WO2010033979, WO2010030540, WO2010030541, WO2010030539, WO2010024467, WO2010024469, WO2010024470, WO2010025161, WO2010014395, WO2010044905, WO2010145887, WO2010142503, WO2010122051, WO2010102861, WO2010099997, WO2010084039, WO2010076292, WO2010069742, WO2010069718, WO2010069957, WO2010057784, WO2010054986, WO2010018043, WO2010003783, WO2010003792, WO2011023716, WO2010142539, WO2010118959, WO2010115813, WO2010105942, WO2010105961, WO2010105962, WO2010094356, WO2010084203, WO2010078979, WO2010072456, WO2010069905, WO2010076165, WO2010072603, WO2010066486, WO2010066631, WO2010066632, WO2010063689, WO2010060821, WO2010049187, WO2010031607, WO2010000636.

### Uses

The present invention is also directed to methods for using the histidine modified variants according to the invention or compositions thereof in laundering of textile and fabrics, such as house hold laundry washing and industrial laundry washing.

The invention is also directed to methods for using the histidine modified variants according to the invention or compositions thereof in cleaning hard surfaces such as floors, tables, walls, roofs etc. as well as surfaces of hard objects such as cars (car wash) and dishes (dish wash).

The variants of the present invention may be added to and thus become a component of a detergent composition.

A detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

The cleaning process or the textile care process may for example be a laundry process, a dishwashing process or cleaning of hard surfaces such as bathroom tiles, floors, table tops, drains, sinks and washbasins. Laundry processes can for example be household laundering, but it may also be industrial laundering. Furthermore, the invention relates to a process for laundering of fabrics and/or garments where the process comprises treating fabrics with a washing solution containing a detergent composition, and at least one histidine modified variant of the invention. The cleaning process or a textile care process can for example be carried out in a machine washing process or in a manual washing process. The washing solution can for example be an aqueous washing solution containing a detergent composition.

The last few years there has been an increasing interest in replacing components in detergents, which is derived from petrochemicals with renewable biological components such as enzymes and polypeptides without compromising the wash performance. When the components of detergent compositions change new enzyme activities or new enzymes having alternative and/or improved properties compared to the common used detergent enzymes such as proteases, lipases and amylases is needed to achieve a similar or improved wash performance when compared to the traditional detergent compositions.

The invention further concerns the use of histidine variants of the invention in a proteinaceous stain removing processes. The proteinaceous stains may be stains such as food stains, e.g., baby food, sebum, cocoa, egg, blood, milk, ink, grass, or a combination hereof.

Typical detergent compositions include various components in addition to the enzymes, these components have different effects, some components like the surfactants lower the surface tension in the detergent, which allows the stain being cleaned to be lifted and dispersed and then washed away, other components like bleach systems remove discolor often by oxidation and many bleaches also have strong bactericidal properties, and are used for disinfecting and sterilizing. Yet other components like builder and chelator softens, e.g., the wash water by removing the metal ions form the liquid.

In a particular embodiment, the invention concerns the use of a composition comprising a histidine modified variant of the invention and one or more detergent components, such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers.

In a particular embodiment, the invention concerns the use of a composition comprising a histidine modified variant of the invention and one or more additional enzymes selected from the group comprising of proteases, amylases, lipases, cutinases, cellulases, endoglucanases, xyloglucanases, pectinases, pectin lyases, xanthanases, peroxidaes, haloperoxygenases, catalases and mannanases, or any mixture thereof.

In a particular embodiment, the invention concerns the use of a composition comprising a histidine modified variant of the invention, one or more additional enzymes selected from the group comprising of proteases, amylases, lipases, cutinases, cellulases, endoglucanases, xyloglucanases, pectinases, pectin lyases, xanthanases, peroxidaes, haloperoxygenases, catalases and mannanases, or any mixture thereof and one or more detergent components, such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers.

The present invention is further described by the following examples that should not be construed as limiting the scope of the invention.

### EXAMPLES

### Materials and Methods

### Example 1: Preparation, Expression and Purification of Histidine Modified Variants

The following summarizes the mutation and introduction of an expression cassette into Bacillus subtilis. All DNA manipulations were done by PCR (e.g., Sambrook et al.; Molecular Cloning; Cold Spring Harbor Laboratory Press).

Recombinant B. subtilis constructs encoding histidine modified variants were used to inoculate shakeflasks containing a rich media (e.g., 100 g/L sucrose (Danisco cat. no. 109-0429), 40 g/L crust soy (soy bean flour), 10 g/L Na₂HPO₄.12H₂O (Merck cat. no. 6579), 0.1 ml/L replace-Dowfax63N10 (Dow). Cultivation typically takes 4 days at 30° C. shaking with 220 rpm.

### Fermentation of Variants

Fermentation may be performed by methods well known in the art or as follows. A B. subtilis strain harboring the relevant expression plasmid was streaked on a LB agar plate, and grown overnight at 37° C. The colonies were transferred to 100 ml PS-1 media in a 500 ml shaking flask. Cells and other undissolved material were removed from the fermentation broth by centrifugation at 4500 rpm for 20-25 minutes. Afterwards the supernatant was filtered to obtain a clear solution.

### Purification of Variants

Purification may be performed by methods well known in the art or as follows. The culture broth was centrifuged (26000×g, 20 min) and the supernatant was carefully decanted from the precipitate. The supernatant was filtered through a Nalgene 0.2 μm filtration unit in order to remove the rest of the Bacillus host cells. pH in the 0.2 μm filtrate was adjusted to pH 8 with 3M Tris base and the pH adjusted filtrate was applied to a MEP Hypercel column (from Pall corporation) equilibrated in 20 mM Tris/HCl, 1 mM CaCl2, pH 8.0. After washing the column with the equilibration buffer, the column was step-eluted with 20 mM CH3COOH/NaOH, 1 mM CaCl2, pH 4.5. Fractions from the column were analysed for protease activity (using the Suc-AAPF-pNA assay at pH 9) and peak-fractions were pooled. The pH of the pool from the MEP Hypercel column was adjusted to pH 6 with 20% (v/v) CH3COOH or 3M Tris base and the pH adjusted pool was diluted with deionized water to the same conductivity as 20 mM MES/NaOH, 2 mM CaCl2, pH 6.0. The diluted pool was applied to a SP-sepharose FF column (from GE Healthcare) equilibrated in 20 mM MES/NaOH, 2 mM CaCl2, pH 6.0. After washing the column with the equilibration buffer, the protease was eluted with a linear NaCl gradient (0-->0.5M) in the same buffer over five column volumes. Fractions from the column were analysed for activity (for protease using the Suc-AAPF-pNA assay at pH 9) and active fractions were analysed by SDS-PAGE. The fractions where only one band was seen on the coomassie stained SDS-PAGE gel, were pooled as the purified preparation and was used for further experiments.

### Example 2 Testing Solubility of Histidine Modified Variants

### Solubility Assay

In order to measure the solubility the fermentation broth is adjusted to pH 4.5 with 20% CH3COOH with good stirring, centrifuged at (20.000×g, 20 min) and the amount of protein activity in the supernatant is measured. The protein activity could be measured using a simple activity assay depending on the protein e.g. for proteases the protease activity assay can be used. When the solubility of two proteins are compared these are fermented under the same conditions. Thus the reference protein and variant protein is fermented under same conditions and their relative solubility is measured.

### Preparation and Expression of Variants

The following summarizes the mutation and introduction of an expression cassette into Bacillus subtilis. All DNA manipulations were done by PCR (e.g. Sambrook et al.; Molecular Cloning; Cold Spring Harbor Laboratory Press) and can be repeated by everybody skilled in the art.

Recombinant B. subtilis constructs encoding subtilase variants were used to inoculate shakeflasks containing a rich media (e.g. PS-1: 100 g/L Sucrose (Danisco cat. no. 109-0429), 40 g/L crust soy (soy bean flour), 10 g/L Na2HPO4.12H2O (Merck cat. no. 6579), 0.1 ml/L replace-Dowfax63N10 (Dow). Cultivation typically takes 4 days at 30° C. shaking with 220 rpm.

Following proteins were generated:

### Reference: SEQ ID NO: 4

1HIS: SEQ ID NO: 4 with 1 Histidine attached to N-terminus
2HIS: SEQ ID NO: 4 with 2 Histidine attached to N-terminus
3HIS: SEQ ID NO: 4 with 3 Histidine attached to N-terminus
4HIS: SEQ ID NO: 4 with 4 Histidine attached to N-terminus
3 intHIS: SEQ ID NO: 4+V238H+N242H+N246H

The reference and variants generated were fermented in standard lab scale fermentors using the method described in EP 1 520 012 B1, Example 2, without addition of MGP.

It was observed that the variants precipitated during the fermentation. The activities in the fermentations showed that the fermentations of Reference, 1HIS, 2HIS, 3HIS and 4HIS gave approximately same yield, whereas the 3intHIS variant fermentation gave a lower yield that the reference.

The fermentation broths were diluted three fold with water and pH were adjusted to pH 4.5 at 40° C. using HCl, and the fermentations broths were stirred for 60 minutes. Protease activities in the supernatant were determined immediately after pH adjustment and after 60 minutes using the Suc-AAPF-pNA assay as described below. The protease concentrations were determined relative to the concentration in the Reference immediately after pH adjustment was set to 1. Results are shown in table 1.

TABLE 1
Relative activities achieved:

| | T = 0 | T = 60 |
|---|---|---|
| Reference | 1 | 1.4 |
| SEQ ID NO 4 | | |
| 1HIS | 5.8 | 8.8 |
| 2HIS | 45 | 59 |
| 3HIS | 56 | 59 |
| 4HIS | 54 | 57 |
| 3IntHIS | 1.5 | 5 |

The results clearly show that all the histidine modified variants of the invention had higher solubility at low pH compared with the reference SEQ ID NO 4. The data also showed that even though the solubility for the histidine modified variants of the invention were high from the start even more enzyme come into solution during the holding period of 60 minutes.

### Example 3 Substrate Activity of Histidine Variants Measured in Various Protease Activity Assays

### Protease Assay (Suc-AAPF-pNA Assay)

pNA substrate: Suc-AAPF-pNA (Bachem L-1400).
Temperature: Room temperature (25° C.)
Assay buffer: 100 mM succinic acid, 100 mM HEPES, 100 mM CHES, 100 mM CABS, 1 mM CaCl2, 150 mM KCl, 0.01% Triton X-100, pH 9.0. 100 mM Tris/HCl, 0.01% Triton X-100, pH 9.0. 20 μl protease (diluted in 0.01% Triton X-100) was mixed with 100 μl assay buffer. The assay was started by adding 100 μl pNA substrate (50 mg dissolved in 1.0 ml DMSO and further diluted 45× with 0.01% Triton X-100). The increase in OD405 was monitored as a measure of the protease activity. The specific activity compared to the reference protease (SEQ ID NO 3) of histidine modified variants on the Suc-AAPF-pNA substrate is shown below in table 2.

TABLE 2

| | Protease variants with | |
|---|---|---|
| | histidines compared to | |
| SEQ ID NO | SEQ ID NO | pH 9 |
| SEQ ID NO 06 | Q135H, S139H, R143H | 1.87 |
| SEQ ID NO 07 | Q135H, S139H, R143H | 1.84 |
| SEQ ID NO 08 | Q135H, S139H, R143H | 1.57 |
| SEQ ID NO 09 | Q135H, S139H, R143H | 1.58 |
| SEQ ID NO 13 | A47H, N115H, N232H | 1.14 |
| SEQ ID NO 15 | A47H, N115H, N232H | 1.44 |
| SEQ ID NO 16 | A47H, N115H, N232H | 1.61 |
| SEQ ID NO 17 | N115H, N232H | 1.45 |
| SEQ ID NO 18 | A47H, N115H, N232H | 1.42 |
| SEQ ID NO 19 | A47H, N115H, N232H | 1.79 |
| SEQ ID NO 20 | Q135H, S139H, R143H | 1.06 |
| SEQ ID NO 21 | Q135H, S139H, R143H | 1.53 |
| SEQ ID NO 23 | A47H, N115H, N232H | 1.30 |
| SEQ ID NO 24 | G20H, T22H, S24H | 1.33 |
| SEQ ID NO 25 | A47H, N115H, N232H | 1.16 |
| SEQ ID NO 26 | Y89H, N115H, N232H | 1.29 |
| SEQ ID NO 27 | A47H, N115H, N232H | 1.21 |

The same assay was repeated at four different pH. For the pH assays a Multibuffer is used instead of Tris-buffer.

### Multibuffer:

| Reagent | MW/conc. | volumen | 50 mM |
|---|---|---|---|
| HEPES | 238.3 | 250 ml | 3.0 |
| CHES | 207.29 | 250 ml | 2.6 |
| CABS | 235.34 | 250 ml | 2.9 |
| 1 mM CaCl2 | 1M | 250 ml | 0.3 |
| 150 mM KCl | 74.55 | 250 ml | 0.9 |
| 0.01% Triton | 100% | 250 ml | 0.025 |

Table 3 shows the activity on the PNA substrate of histidine modified protease variants compared to reference protease with SEQ ID NO 3 (Savinase) at four different pH values.

TABLE 3

| protease | | pH | pH | pH | pH |
|---|---|---|---|---|---|
| SEQ ID NO | histidines | 8 | 9 | 10 | 11 |
| SEQ ID NO 15 | A47H, N115H, N232H | 1.48 | 1.25 | 1.34 | 1.31 |
| SEQ ID NO 17 | N115H, N232H | 1.15 | | | |
| SEQ ID NO 19 | A47H, N115H, N232H | 1.72 | 1.16 | 1.44 | 1.35 |
| SEQ ID NO 20 | Q135H, S139H, R143H | 1.42 | 1.22 | 1.33 | 1.28 |

### EnzChek Assay:

Residual activity of the proteases variants was determined using the EnzChek Protease Kit Red assay.

Substrate: EnzChek from Lifetechnologies (Cat. Nr.: E6639) a substrate based on casein with the fluorophor “Bodipy” attached.

EnzChek substrate is dissolved in 0.01% Triton x-100 to a stock concentration of 1 mg/ml. 10 μl of purified histidine modified protease variant normalized to a concentration of 10 μM (μmol/Liter) in Buffer (20 mM MES; 0.01% Triton 100-X; 2 mM CaCl₂ was mixed with 85 μl Multibuffer pH 8-11) and mixed on shaker 900 rpm for 30 seconds. 5 μl EnzChek red from the 1 mg/ml stock was added. For increasing the fluorescence signal white 96 well microtiter plate was used. Fluorescence was measured every 60 second for 20 minutes using Fluostar Optima reader with filter 584/620 Gain 1300. Table 4 shows the activity on EnzChek red of protease variants with histidines compared to reference protease with SEQ ID NO 3 at two or four different pH values.

TABLE 4

| protease | | pH | pH | pH | pH |
|---|---|---|---|---|---|
| SEQ ID NO | histidines | 8 | 9 | 10 | 11 |
| SEQ ID NO 15 | A47H, N115H, N232H | 1.13 | 1.05 | | |
| SEQ ID NO 19 | A47H, N115H, N232H | 1.34 | 1.37 | 1.09 | 1.27 |

### DQ Red BSA Assay:

Activity of histidine modified protease variants were determined using the DQ Red BSA assay Substrate “DQ Red BSA” from Lifetechnologies (Cat. Nr.: D12051) a substrate based on Bovine Serum Albumin conjugated the fluorophor “Bodipy”.

DQ Red BSA substrate was dissolved in 0.01% Triton x-100 to a stock concentration of 1 mg/ml. 2.5 μl DQ Red BSA (1 mg/ml) substrate was mixed with 85 μl Multibuffer pH 8-11 and shaken 900 rpm for 30 seconds, 10 μl of purified histidine modified protease variant normalized to a concentration of 10 μM (μmol/Liter) in Buffer: 20 mM MES; 0.01% Triton 100-X; 2 mM CaCl₂ was then added. For increasing the fluorescence signal white 96 well microtiter plate was used. Fluorescence was measured every 60 second for 20 minutes using Fluostar Optima reader with filter 584/620 Gain 1300. The histidine modified protease variants were tested in the DQ Red BSA assay as described above at pH 8 and/or pH 9, pH 10 and pH 11 the results are shown in table 5.

TABLE 5

| protease | | pH | pH | pH |
|---|---|---|---|---|
| SEQ ID NO | histidines | 9 | 10 | 11 |
| SEQ ID NO 19 | A47H, N115H, N232H | 1.05 | 1.19 | 1.16 |

### Example 4 Testing Performance of Histidine Modified Variants in Various Assays

### Detergent Model X pH Profile (on EMPA117EH)

The assay was performed in a 96 well microtiter plate 96 with two EMPA117 EH (blood/milk/ink extra heated) swatches and the measurement performed in a 384 well microtiter plate.

| | | | |
|---|---|---|---|
| Model detergent X | LAS | 17.6 | wt % |
| | AEO* | 2.0 | wt % |
| | Sodium carbonate | 20.1 | wt % |
| | Hydrous sodium silicate | 12.4 | wt % |
| | Zeolite A | 15.0 | wt % |
| | Sodium sulfate | 31.8 | wt % |
| | Polyacrylate | 1.1 | wt % |

*AEO is added separately

### 100 ml Detergent:

### 0.2 g Model X

### 233 μl 0.713M CaCl₂

### 233 μl 0.357M MgCl₂

### 700 μl 0.535M NaHCO₃

### 80 ml Multibuffer 50 mM pH 8-11

10 μl of purified histidine modified protease variant normalized to a concentration of 10 μM (μmol/Liter) in Buffer: 20 mM MES; 0.01% Triton 100-X; 2 mM CaCl₂ was diluted MilliQ/0.01% Triton 2000 times for a final concentration of 5 nM. The histidine modified protease variants were tested on the EMPA117 EH swatch. 96 well microtiter plates were shaken 1000 rpm for 30 min. Aspirate 50 μl from the 96 well microtiter plates to clear 384 well microtiter plates and measure OD590 endpoint. Improvement factor is the measured endpoint value for the variant divided by the measured endpoint value for the reference enzyme (SEQ ID NO 3).

Table 6 shows the performance of the protease variants in the assay described above at pH 8 and/or pH 9, pH 10 and pH 11 on the blood/ink stain EMPA117EH (Blood/milk/ink on cotton/polyester).

TABLE 6

| protease | | pH | pH | pH | pH |
|---|---|---|---|---|---|
| SEQ ID NO | histidines | 8 | 9 | 10 | 11 |
| SEQ ID NO 15 | A47H, N115H, N232H | | | | 1.09 |
| SEQ ID NO 17 | N115H, N232H | | | 1.15 | 1.07 |
| SEQ ID NO 19 | A47H, N115H, N232H | 1.08 | 1.14 | | 1.21 |

### AZCL-Hemoglobin Assay:

Substrate: “AZCL-Hemoglobin” (Megazyme) substrate based on a Azurine-crosslinking to the hemoglobin molecule.

The assay is performed in 96 well microtiter plates with AZCL-hemoglobin substrate and the measurement is performed in a 384 well microtiter plates.

10 μl of purified histidine modified protease variant normalized to a concentration of 10 μM (μmol/Liter) in Buffer: 20 mM MES; 0.01% Triton 100-X; 2 mM CaCl₂ was mixed with 90 μl 0.01% Triton 100X and 5 μl of this solution was mixed with 195 μl Substrate mix (AZCL-Hemoglobin 10 mg/ml in Multibuffer). The 96 well microtiter plates were shaken 1000 rpm for 30 min. Aspirate 50 μl from the 96 well microtiter plates to clear 384 well microtiter plates and measure OD590 endpoint. Improvement factor is the measured endpoint value for the variant divided by the measured endpoint value for the reference enzyme (SEQ ID NO 3). Table 6 show the performance of the protease variants in the assay described above at pH 8 and/or pH 9, pH 10 and pH 11 on EMPA117EH (Blood/milk/ink on cotton/polyester).

TABLE 6

| protease | | pH | pH | pH | pH |
|---|---|---|---|---|---|
| SEQ ID NO | histidines | 8 | 9 | 10 | 11 |
| SEQ ID NO 15 | A47H, N115H, N232H | 1.26 | 1.42 | 1.32 | 1.11 |
| SEQ ID NO 17 | N115H, N232H | 1.30 | 1.37 | 1.22 | 1.06 |
| SEQ ID NO 19 | A47H, N115H, N232H | 1.46 | 1.49 | 1.36 | 1.14 |

### Automatic Mechanical Stress Assay (AMSA) for Laundry

The wash performance of histidine modified protease variants in laundry was assessed using the Automatic Mechanical Stress Assay (AMSA), where the wash performance of many small volume enzyme-detergent solutions can be examined. The AMSA plate has a number of slots for test solutions and a lid that firmly squeezes the textile to be washed against the slot openings. During the wash, the plate, test solutions, textile and lid were vigorously shaken to bring the test solution in contact with the textile and apply mechanical stress in a regular, periodic, oscillating manner. For further description see WO 02/42740 especially the paragraph “Special method embodiments” at pages 23-24. The laundry experiments were conducted under the experimental conditions specified in Table 7.

TABLE 7

| | |
|---|---|
| Detergent dosage | 5 g/L (liquid detergent) |
| Test solution volume | 160 micro L |
| pH | Adjusted to pH 9 |
| Wash time | 20 minutes |
| Temperature | 20° C. |
| Water hardness | 15° dH |

Model detergents and test materials may be as follows:

| | | |
|---|---|---|
| CNS detergent | Alkylbenzenesulfonate (LAS) | 8.08 wt % |
| (Chinese national | Alcohol ethoxylate (AEO) | 4.01 wt % |
| standard) | Sodium lauryl ether sulfate (AES) | 3.86 wt % |
| | Triethanolamine; 2,2′,2″-nitrilotri(ethan- | 0.51 wt % |
| | 1-ol) (TEA) | |
| | Trisodium citrate dihydrate | 0.50 wt % |
| | Sodium hydroxide | 1.00 wt % |
| | Minor | 2.01 wt % |
| | Water | 80.03 wt % |
| | pH adjusted to 9 | |

| | |
|---|---|
| Test material | PC-05 (Blood/milk/ink on cotton/polyester) |
| | PC-03 (Chocolate-milk/soot on cotton/polyester) |

Test materials were obtained from Center For Testmaterials BV, P.O. Box 120, 3133 KT Vlaardingen, the Netherlands, SBL2004 and the other enzyme sensitive stains were obtained either from Center For Testmaterials BV, P.O. Box 120, 3133 KT Vlaardingen, the Netherlands, or EMPA Testmaterials AG, Mowenstrasse 12, CH-9015 St. Gallen, Switzerland or WFK Testgewebe GmbH, Christenfeld 10, D-41379 Brüggen, Germany. Water hardness was adjusted to 16° dH by addition of CaCl2, MgCl2, and NaHCO3 (Ca2+:Mg2+:CO32−)=5:1:6 for PC-03 and 5:1:11 for C-05 to the test system. After washing the textiles were flushed in tap water and dried. The performance of the histidine modified protease variants were measured as the brightness of the colour of the textile washed with that specific protease. Brightness can also be expressed as the intensity of the light reflected from the sample when illuminated with white light. When the sample is stained the intensity of the reflected light is lower, than that of a clean sample. Therefore the intensity of the reflected light can be used to measure wash performance of the histidine modified protease variants. Color measurements were made with a professional flatbed scanner (EPSON EXPRESSION 10000XL, Atea NS, Lautrupvang 6, 2750 Ballerup, Denmark), which is used to capture an image of the washed melamine tiles.

To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

The results are shown in Table 8-11 below. The results are given as relative performance compared to (SEQ ID NO: 3) and an average of two enzyme concentrations 30 and 60 nM of two different swatches PC-05 (Blood/milk/ink on cotton/polyester) and PC-03 (Chocolate-milk/soot on cotton/polyester).

Table 8 shows AMSA relative performance of 30 nM concentrations of protease variants on PC-05 compared to SEQ ID NO 3.

TABLE 8

| SEQ ID NO | histidines | pH 9 |
|---|---|---|
| SEQ ID NO 08 | Q135H, S139H, R143H | 1.06 |
| SEQ ID NO 15 | A47H, N115H, N232H | 1.08 |
| SEQ ID NO 19 | A47H, N115H, N232H | 1.15 |
| SEQ ID NO 24 | G20H, T22H, S24H | 1.05 |

Table 9 shows AMSA relative performance of 60 nM concentrations of protease variants on PC-05 compared to SEQ ID NO 3.

TABLE 9

| SEQ ID NO | histidines | pH 9 |
|---|---|---|
| SEQ ID NO 09 | Q135H, S139H, R143H | 1.06 |
| SEQ ID NO 22 | A47H, N115H, N232H | 1.09 |
| SEQ ID NO 23 | A47H, N115H, N232H | 1.08 |

Table 10 shows AMSA relative performance of 30 nM concentrations of protease variants on PC-03 compared to SEQ ID NO 3.

TABLE 10

| SEQ ID NO | histidines | pH 9 |
|---|---|---|
| SEQ ID NO 19 | A47H, N115H, N232H | 1.21 |

Table 11 shows AMSA relative performance of 60 nM concentrations of protease variants on PC-03 compared to SEQ ID NO 3.

TABLE 11

| SEQ ID NO | histidines | pH 9 |
|---|---|---|
| SEQ ID NO 09 | Q135H, S139H, R143H | 1.10 |
| SEQ ID NO 15 | A47H, N115H, N232H | 1.17 |
| SEQ ID NO 19 | A47H, N115H, N232H | 1.11 |

### Example 4: Accelerated Storage Stability Assay

Storage stability of histidine modified protease variants in liquid detergent was evaluated using an accelerated assay with incubation at 60° C. for up to 48 hours. Culture supernatants containing 25-75 μg/ml active protease were diluted 1, 2, 4 and 8 times using 0.01% Triton X-100. For each variant 2 wells with each dilution were included. 30 μl diluted protease sample was mixed with 270 μl CNS (Chinese national standard) detergent in the well of a microtiter plate (Nunc U96 PP 0.5 ml) using a magnetic bar (on Zephyr pipetting station (Caliper LifeSciences) for 30 min). 20 μl of this mixture was then transferred to another microtiter plate (Nunc U96 PP 0.5 ml with added magnetic bars) and mixed with 80 μl 100 mM Tris pH 8.6 (at least 5 min on Zephyr). 30 μl of this dilution was transferred to a Nunc F 96-MTP, and after addition of 70 μl substrate solution initial activity of unstressed sample was determined by measuring absorbance at 405 nm every 20 sec for 5 min (on a SpectraMax Plus). After sealing, the detergent plate was incubated at 60° C. in an Eppendorf Thermomixer (no shaking). After 1, 4, 23 and 47 hours incubation, 20 μl samples were withdrawn and residual activity of stressed sample was measured as with the initial, unstressed activity.

Decrease in activity during incubation with detergent was assumed to be exponential. Half-lifes (T½) were found from linear regression of Log(Activity) versus incubation time (0, 1, 4, 23 and 47 hours), and half-life improvement factors (T % IF) were calculated as half-life of protease variant relative to the half-life of SEQ ID NO: 3.

TABLE 12

| SEQ ID NO | SEQ ID NO + his mutations | 9 |
|---|---|---|
| SEQ ID NO 06 | Q135H, S139H, R143H | 75 |
| SEQ ID NO 07 | Q135H, S139H, R143H | 205 |
| SEQ ID NO 08 | Q135H, S139H, R143H | 58 |
| SEQ ID NO 09 | Q135H, S139H, R143H | 1513 |
| SEQ ID NO 10 | Q135H, S139H, R143H | 1157 |
| SEQ ID NO 11 | Q135H, S139H, R143H | 1161 |
| SEQ ID NO 12 | A47H, N115H, N232H | 1002 |
| SEQ ID NO 13 | A47H, N115H, N232H | 858 |
| SEQ ID NO 14 | A47H, N115H, N232H | 1147 |
| SEQ ID NO 15 | A47H, N115H, N232H | 423 |
| SEQ ID NO 16 | A47H, N115H, N232H | 429 |
| SEQ ID NO 17 | N115H, N232H | 519 |
| SEQ ID NO 18 | A47H, N115H, N232H | 281 |
| SEQ ID NO 19 | A47H, N115H, N232H | 609 |
| SEQ ID NO 20 | Q135H, S139H, R143H | 177 |
| SEQ ID NO 21 | Q135H, S139H, R143H | 200 |
| SEQ ID NO 22 | A47H, N115H, N232H | 965 |
| SEQ ID NO 23 | A47H, N115H, N232H | 803 |
| SEQ ID NO 24 | G20H, T22H, S24H | 241 |
| SEQ ID NO 25 | A47H, N115H, N232H | 674 |
| SEQ ID NO 26 | Y89H, N115H, N232H | 346 |
| SEQ ID NO 27 | A47H, N115H, N232H | 1054 |

1. A protease variant of a protease parent, which when compared to the parent protease comprises 2 to 6 additional histidines at positions corresponding to positions at the surface of the mature polypeptide of SEQ ID NO: 2, wherein said variant has at least 90%, but less than 100% sequence identity to SEQ ID NO: 3.

2. The protease variant according to claim 1, wherein said variant compared to SEQ ID NO: 3, comprises 2 to 6 of the following substitutions: A1H, Q2H, S3H, V4H, W6H, S9H, R10H, Q12H, P14H, A15H, N18H, R19H, G20H, T22H, S24H, G25H, K27H, T37H, P39H, N42H, 143H, R44H, G45H, G46H, S48H, F49H, P51H, G52H, E53H, P54H, S55H, T56H, Q57H, G59H, L73H, N74H, N75H, S76H, 177H, S85H, E87H, A96H, S97H, G98H, S99H, G100H, S101H, V102H, S103H, Q107H, E110H, W111H, N114H, N115H, G116H, G125H, S126H, P127H, S128H, P129H, A131H, T132H, E134H, Q135H, N138H, S139H, S142H, R143H, G144H, N153H, S154H, A156H, G157H, S158H, Y161H, R164H, A166H, N167H, D175H, Q176H, N177H, N178H, N179H, R180H, S182H, F183H, Y186H, A188H, G189H, N198H, Q200H, Y203H, P204H, G205H, S206H, T207H, Y208H, S210H, L211H, N212H, K229H, Q230H, K231H, N232H, P233H, S234H, W235H, S236H, V238H, Q239H, R241H, N242H, K245H, N246H, T249H, S250H, L251H, G252H, S253H, T254H, N255H, L256H, Y257H, S259H, R269H.

3. The protease variant according to claim 2, wherein the variant comprises one or more of the substitutions G20H, T22H, Y89H, N115H, Q135H, S139H, R143H, N232H, V238H, N242H or N246H.

4. The protease variant according to claim 1, wherein the variant when compared to SEQ ID NO: 3 comprises 2 to 6 of the following insertions: *1aH, *2aH, *3aH, *4aH, *6aH, *9aH, *10aH, *12aH, *14aH, *15aH, *17aH, *18aH, *19aH, *20aH, *22aH, *24aH, *25aH, *27aH, *37aH, *39aH, *42aH, *43aH, *44aH, *45aH, *46aH, *48aH, *49aH, *51aH, *52aH, *53aH, *54aH, *55aH, *56aH, *57aH, *59aH, *73aH, *74aH, *75aH, *76aH, *77aH, *85aH, *87aH, *96aH, *97aH, *98aH, *99aH, *100aH, *101aH, *102aH, *103aH, *107aH, *110aH,*111aH,*114aH,*115aH,*116aH,*118aH,*125aH,*126aH,*127aH,*128aH,*129aH, *131aH,*132aH,*134aH,*135aH,*138aH,*139aH,*142aH,*143aH,*144aH,*153aH,*154aH, *156aH,*157aH,*158aH,*161aH,*164aH,*166aH,*167aH,*175aH,*176aH,*177aH,*178aH, *179aH, *180aH, *182aH, *183aH, *186aH, *188aH, *189aH, *198aH, *200aH, *203aH, *204aH, *205aH, *206aH, *207aH, *208aH, *210aH, *211aH, *212aH, *229aH, *230aH, *231aH, *232aH, *233aH, *234aH, *235aH, *236aH, *238aH, *239aH, *241aH, *242aH, *243aH, *245aH, *246aH, *249aH, *250aH, *251aH, *252aH, *253aH, *254aH, *255aH, *256aH, *257aH, *259aH, *269aH.

5. The protease variant according to claim 4, wherein the variant comprises one or more of the insertions *20aH, *22aH, *89aH, *115aH, *135H, *139aH, *143H, *232aH, *238aH, *242aH or *246aH.

6. The protease variant according to claim 1, wherein the variant has increased solubility at pH 5 and/or improved wash performance compared to SEQ ID NO: 3.

7. The protease variant according to claim 1, wherein the protease parent is a protease comprising at least 90%, sequence identity to SEQ ID NO: 3.

8. The protease variant according to claim 7, wherein the protease parent comprises SEQ ID NO: 3.

9. The variant according to claim 1 further comprising one or more substitutions at positions selected from the group consisting of positions: 3, 4, 9, 15, 24, 42, 59, 66, 74, 76, 97, 99, 101, 102, 118, 126, 127, 128, 154, 156, 157, 158, 161, 164, 176, 179, 182, 188, 198, 199, 200, 203, 210, 211, 212, 216, 239, 255 and 256, preferably positions 9, 15, 42, 66, 74, 97, 99, 154, 200, 203, 211, 212 and/or 256 (numbering according to SEQ ID NO: 3).

10. A composition comprising at least one variant according to claim 1.

11. The composition according to claim 10, wherein the composition is a detergent composition.
